(19) United States
(12) Patent Application Publication
(10) Pub. No.: US 2017/0246481 A1
(43) Pub. Date: Aug. 31, 2017

(54) DEVICES AND METHODS FOR OPTIMIZED NEUROMODULATION AND THEIR APPLICATION

(71) Applicant: David J Mishelevich, Playa del Rey, CA (US)

(72) Inventor: David J Mishelevich, Playa del Rey, CA (US)

(21) Appl. No.: 15/444,268

(22) Filed: Feb. 27, 2017

Related U.S. Application Data
(63) Continuation-in-part of application No. 13/918,862, filed on Jun. 14, 2013, now abandoned.
Application No. 13/918,862 is a continuation-in-part of application No. 12/940,052, filed on Nov. 5, 2010, now abandoned.
Application No. 13/918,862 is a continuation-in-part of application No. 12/958,411, filed on Dec. 2, 2010, now abandoned.
Application No. 13/918,862 is a continuation-in-part of application No. 13/007,626, filed on Jan. 15, 2011, now abandoned.
Application No. 13/918,862 is a continuation-in-part of application No. 13/200,903, filed on Jan. 15, 2011, now abandoned.
Application No. 13/918,862 is a continuation-in-part of application No. 13/694,327, filed on Jan. 16, 2011, now abandoned.
Application No. 13/918,862 is a continuation-in-part of application No. 13/694,328, filed on Jan. 16, 2011, now abandoned.
Application No. 13/918,862 is a continuation-in-part of application No. 13/098,473, filed on May 1, 2011, now abandoned.
Application No. 13/918,862 is a continuation-in-part of application No. 13/360,600, filed on Jan. 27, 2012, now abandoned.
Application No. 13/918,862 is a continuation-in-part of application No. 13/252,054, filed on Oct. 3, 2011, now abandoned.
Application No. 13/918,862 is a continuation-in-part of application No. 13/625,677, filed on Sep. 24, 2012, now abandoned.
Application No. 13/918,862 is a continuation-in-part of application No. 13/689,178, filed on Nov. 29, 2012, now abandoned.
Application No. 13/918,862 is a continuation-in-part of application No. 13/718,245, filed on Dec. 18, 2012, now abandoned.
Application No. 13/718,245 is a continuation-in-part of application No. 13/898,401, filed on May 20, 2013, now abandoned.
Application No. 13/898,401 is a continuation-in-part of application No. 13/020,016, filed on Feb. 3, 2011, now abandoned.
Application No. 13/898,401 is a continuation-in-part of application No. 13/031,192, filed on Feb. 19, 2011, now abandoned.
Application No. 13/898,401 is a continuation-in-part of application No. 13/405,337, filed on Feb. 26, 2012, now abandoned.
Application No. 13/898,401 is a continuation-in-part of application No. 13/411,641, filed on Mar. 5, 2012, now abandoned.
Application No. 13/898,401 is a continuation-in-part of application No. 13/413,659, filed on Mar. 7, 2012, now abandoned.
Application No. 13/898,401 is a continuation-in-part of application No. 13/430,729, filed on Mar. 27, 2012, now abandoned.
Application No. 13/430,729 is a continuation-in-part of application No. 13/035,962, filed on Feb. 26, 2011, now Pat. No. 8,956,277.
Application No. 13/035,962 is a continuation-in-part of application No. 13/467,009, filed on May 8, 2012, now abandoned.
Application No. 13/467,009 is a continuation-in-part of application No. 13/475,985, filed on May 20, 2012, now abandoned.
Application No. 13/475,985 is a continuation-in-part of application No. 13/539,308, filed on Jun. 30, 2012, now abandoned.
Application No. 13/539,308 is a continuation-in-part of application No. 13/871,237, filed on Apr. 26, 2013.
(60) Provisional application No. 61/260,172, filed on Nov. 11, 2009.
(60) Provisional application No. 61/295,757, filed on Jan. 17, 2010.
(60) Provisional application No. 61/266,112, filed on Dec. 2, 2009.
(60) Provisional application No. 61/295,760, filed on Jan. 18, 2010.
(60) Provisional application No. 61/295,759, filed on Jan. 18, 2010.
(60) Provisional application No. 61/295,761, filed on Jan. 18, 2010.
(60) Provisional application No. 61/325,339, filed on Apr. 18, 2010.
(60) Provisional application No. 61/330,363, filed on May 2, 2010.
(60) Provisional application No. 61/436,607, filed on Jan. 27, 2011.
(60) Provisional application No. 61/389,280, filed on Oct. 4, 2010.
(60) Provisional application No. 61/538,934, filed on Sep. 25, 2011.
(60) Provisional application No. 61/564,856, filed on Nov. 29, 2011.
(60) Provisional application No. 61/577,095, filed on Dec. 19, 2011.
(60) Provisional application No. 61/666,825, filed on Jun. 30, 2012.
(60) Provisional application No. 61/300,828, filed on Feb. 3, 2010.
(60) Provisional application No. 61/306,531, filed on Feb. 21, 2010.
(60) Provisional application No. 61/447,081, filed on Feb. 27, 2011.
(60) Provisional application No. 61/449,714, filed on Mar. 6, 2011.
(60) Provisional application No. 61/450,627, filed on Mar. 9, 2011.
(60) Provisional application No. 61/666,825, filed on Jun. 30, 2012.
(60) Provisional application No. 61/308,987, filed on Feb. 28, 2010.
(60) Provisional application No. 61/483,734, filed on May 8, 2011.
(60) Provisional application No. 61/538,936, filed on Sep. 25, 2011.
(60) Provisional application No. 61/542,288, filed on Oct. 3, 2011.
(60) Provisional application No. 61/488,754, filed on May 22, 2011.
(60) Provisional application No. 61/547,679, filed on Oct. 15, 2011.
(60) Provisional application No. 61/638,497, filed on Apr. 26, 2012.

(51) Int. Cl. A61N 7/00

(52) U.S. Cl. CPC A61N 7/00; A61N 2007/0021

(57) ABSTRACT

Disclosed are methods and systems for optimized deep or superficial deep-brain stimulation using multiple therapeutic modalities impacting one or multiple points in a neural circuit to produce Long-Term Potentiation (LTP) or Long-Term Depression (LTD). Also disclosed are methods for treatment of clinical conditions and obtaining physiological impacts. Also disclosed are: methods and systems for Guided Feedback control of non-invasive deep brain or superficial neuromodulation; patterned neuromodulation, ancillary stimulation, treatment planning, focused shaped or steered ultrasound; methods and systems using intersecting ultrasound beams; non-invasive ultrasound-neuromodulation techniques to control the permeability of the blood-brain barrier; non-invasive neuromodulation of the spinal cord by ultrasound energy; methods and systems for non-invasive neuromodulation using ultrasound for evaluating the feasibility of neuromodulation treatment using non-ultrasound/ultrasound modalities; neuromodulation of the whole head, treatment of multiple conditions, and method and systems for neuromodulation using ultrasound delivered in sessions.

### CROSS REFERENCE TO RELATED APPLICATIONS

This application is a divisional U.S. patent application of U.S. patent application Ser. No. 14/324,208 filed Jul. 6, 2014 that is a continuation-in-part of U.S. patent application Ser. No. 13/918,862 filed Jun. 14, 2013, titled “NEUROMODULATION DEVICES AND METHODS,” that is a continuation-in-part of U.S. patent application Ser. No. 12/940,052 filed Nov. 5, 2010, titled “NEUROMODULATION OF DEEP-BRAIN TARGETS USING FOCUSED ULTRASOUND,” that claims priority to U.S. Provisional Patent Applications 61/260,172 filed Nov. 11, 2009 and 61/295,757 filed Jan. 17, 2010, and U.S. patent application Ser. No. 12/958,411 filed Dec. 2, 2010, titled “MULTI-MODALITY NEUROMODULATION OF BRAIN TARGETS,” that claims priority to U.S. Provisional Patent Application 61/266,112 filed Dec. 2, 2009, and U.S. patent application Ser. No. 13/007,626 filed Jan. 15, 2011, titled “PATIENT FEEDBACK FOR CONTROL OF ULTRASOUND DEEP-BRAIN NEUROMODULATION,” that claims priority to U.S. Provisional Patent Application 61/295,760 filed Jan. 18, 2010, and U.S. patent application Ser. No. 13/200,903 filed Jan. 15, 2011, titled “SHAPED AND STEERED ULTRASOUND FOR DEEP-BRAIN NEUROMODULATION,” that claims priority to U.S. Provisional Application 61/295,759 filed Jan. 18, 2010, and U.S. patent application Ser. No. 13/694,327 filed Jan. 16, 2011, titled “TREATMENT PLANNING FOR DEEP-BRAIN NEUROMODULATION,” that claims priority to U.S. Provisional Patent Application 61/295,761 filed Jan. 18, 2010, and U.S. patent application Ser. No. 13/694,328 filed Jan. 16, 2011, titled “ULTRASOUND NEUROMODULATION OF THE BRAIN, NERVE ROOTS, AND PERIPHERAL NERVES,” that claims priority to U.S. Provisional Patent Application 61/325,339 filed Apr. 18, 2010, and U.S. patent application Ser. No. 13/098,473 filed May 1, 2011, titled “ULTRASOUND MACRO-PULSE AND MICRO-PULSE SHAPES FOR NEUROMODULATION,” that claims priority to U.S. Provisional Patent Application 61/330,363 filed May 1, 2010, and U.S. patent application Ser. No. 13/360,600 filed Jan. 27, 2012, titled “PATTERNED CONTROL OF ULTRASOUND FOR NEUROMODULATION,” that claims priority to U.S. Provisional Patent Application 61/436,607 filed Jan. 27, 2011, and U.S. patent application Ser. No. 13/252,054 filed Oct. 3, 2011, titled “ULTRASOUND-INTERSECTING BEAMS FOR DEEP-BRAIN NEUROMODULATION,” that claims priority to U.S. Provisional Patent Application 61/389,280 filed Oct. 4, 2010, and U.S. patent application Ser. No. 13/625,677 filed Sep. 24, 2012, titled “ULTRASOUND-NEUROMODULATION TECHNIQUES FOR CONTROL OF PERMEABILITY OF THE BLOOD-BRAIN BARRIER,” that claims priority to U.S. Provisional Patent Application 61/538,934 filed Sep. 25, 2011, and U.S. patent application Ser. No. 13/689,178 filed Nov. 29, 2012, titled “ULTRASOUND NEUROMODULATION OF SPINAL CORD,” that claims priority to U.S. Provisional Patent Application 61/564,856 filed Nov. 29, 2011, and U.S. patent application Ser. No. 13/718,245 filed Dec. 18, 2012, titled “ULTRASOUND NEUROMODULATION FOR DIAGNOSIS AND OTHER-MODALITY PREPLANNING,” that claims priority to U.S. Provisional Patent Application 61/577,095 filed Dec. 19, 2011, and U.S. Provisional Patent Application 61/666,825 filed Jun. 30, 2012, titled “ULTRASOUND NEUROMODULATION DELIVERED IN SESSIONS,” each of which is herein incorporated by reference in its entirety.

This application is also a continuation-in-part of U.S. patent application Ser. No. 13/898,401 filed May 20, 2013, titled “ULTRASOUND NEUROMODULATION TREATMENT OF CLINICAL CONDITIONS,” that is a continuation-in-part of U.S. patent application Ser. No. 13/020,016 filed Feb. 3, 2011, titled “ULTRASOUND NEUROMODULATION OF THE SPHENOPALATINE GANGLION,” that claims priority to U.S. Provisional Patent Application No. 61/300,828 filed Feb. 3, 2010, and U.S. patent application Ser. No. 13/031,192 filed Mar. 19, 2011, titled “ULTRASOUND NEUROMODULATION OF THE RETICULAR ACTIVATING SYSTEM.” that claims priority to U.S. Provisional Patent Application No. 61/306,531 filed Feb. 21, 2010, and U.S. patent application Ser. No. 13/405,337 filed Feb. 26, 2012, titled “ULTRASOUND NEUROMODULATION FOR STROKE MITIGATION AND REHABILITATION.” that claims priority to U.S. Provisional Patent Application No. 61/447,081 filed Feb. 27, 2011, and U.S. patent application Ser. No. 13/411,641 filed Mar. 5, 2012, titled “ULTRASOUND NEUROMODULATION TREATMENT OF PAIN,” that claims priority to U.S. Provisional Patent Application No. 61/449,714 filed Mar. 6, 2011, and U.S. patent application Ser. No. 13/413,659 filed Mar. 7, 2012, titled “ULTRASOUND NEUROMODULATION TREATMENT OF TINNITUS,” that claims priority to U.S. Provisional Patent Application No. 61/450,627 filed Mar. 9, 2011, and U.S. patent application Ser. No. 13/430,729 filed Mar. 27, 2012, titled “ULTRASOUND NEUROMODULATION TREATMENT OF POST TRAUMATIC STRESS DISORDER,” each of which is herein incorporated by reference in its entirety.

This application is also a continuation-in-part of U.S. patent application Ser. No. 13/035,962 filed Feb. 26, 2011, titled “ORGASMATRON VIA DEEP-BRAIN MODULATION,” that claims priority to U.S. Provisional Patent Application No. 61/308,987 filed Feb. 28, 2010, and U.S. patent application Ser. No. 13/467,009 filed May 8, 2012, titled “ULTRASOUND NEUROMODULATION TREATMENT OF MOVEMENT DISORDERS, INCLUDING MOTOR TREMOR, TOURETTE'S SYNDROME, AND EPILEPSY,” that claims priority to U.S. Provisional Patent Applications 61/483,734 filed May 8, 2011, 61/538,936 filed Sep. 25, 2011, and 61/542,288 filed Oct. 3, 2011, and U.S. patent application Ser. No. 13/475,985 filed May 20, 2012, titled “ULTRASOUND NEUROMODULATION FOR TREATMENT OF AUTISM SPECTRUM DISORDER AND ALZHEIMER'S DISEASE AND OTHER DEMENTIAS,” that claims priority to U.S. Provisional Patent Applications 61/488,754 filed May 22, 2011, and U.S. patent application Ser. No. 13/539,308 filed Jun. 30, 2012, titled “ULTRASOUND NEUROMODULATION TREATMENT OF OBESITY AND EATING DISORDERS.” that claims priority to U.S. Provisional Patent Application 61/547,679 filed Oct. 15, 2011, and U.S. patent application Ser. No. 13/871,237 filed Apr. 26, 2013, titled “TARGETED OPTOGENETIC NEUROMODULATION FOR TREATMENT OF CLINICAL CONDITIONS,” that claims priority to U.S. Provisional Patent Application 61/638,497 filed Apr. 26, 2012, each of which is herein incorporated by reference in its entirety.

### INCORPORATION BY REFERENCE

All publications and patent applications mentioned in this specification are herein incorporated by reference to the same extent as if each individual publication or patent application had been specifically and individually designated to be incorporated by reference.

### FIELD

Described herein are systems and methods for one or more modalities of optimized neuromodulation of one or more superficial- or deep-brain targets to up-regulate and/or down-regulate neural activity and their application to the treatment of clinical conditions and providing physiological impacts.

### BACKGROUND

It has been demonstrated that a variety of methods can be employed to neuromodulate superficial or deep brain neural structures. Invasive examples are implanted deep-brain stimulators (DBS), Spinal Cord Stimulation (SCS) with implanted electrodes, optogenetics implanted optical stimulation, focused ultrasound, radiosurgery, Vagus Nerve Stimulation, Sphenopalatine Ganglion stimulation, occipital nerve stimulation, peripheral nerve stimulation, and stereotactic radio surgery. Non-invasive neuromodulation examples include Ultrasound Neuromodulation (US), Transcranial Magnetic Stimulation (TMS), transcranial Direct Current Stimulation (tDCS), Radio-Frequency (RF) stimulation, functional stimulation, or drugs. If neural activity is increased or excited, the neural structure is up regulated; if neural activated is decreased or inhibited, the neural structure is down regulated. Down regulation means that the firing rate of the neural target has its firing rate decreased and thus is inhibited and up regulation means that the firing rate of the neural target has its firing rate increased and thus is excited. Neural structures are usually assembled in circuits. For example, nuclei and tracts connecting them make up a neural circuit.

Transcranial Magnetic Stimulation (TMS) involves electromagnet coils that are powered by brief stimulator pulses (e.g., Mishelevich and Schneider, “Trajectory-Based Deep-Brain Stereotactic Transcranial Magnetic Stimulation,” International Application Number PCT/US2007/010262, International Publication Number WO 2007/130308, Nov. 15, 2007).

Radiosurgery involves permanent change to neural structures by applying focused ionizing radiation in such a way that tissue and thus function are modified but without destroying tissue. A quantity of 60 to 80 grey is typically applied at rates on the order of 5 Gy per minute (e.g., Schneider, Adler, Borchers, “Radiosurgical Neuromodulation Devices, Systems, and Methods for Treatment of Behavioral Disorders by External Application of Ionizing Radiation,” U.S. patent application Ser. No. 12/261,347, Publication No.” US2009/0114849, May 7, 2009).

Radio-Frequency (RF) stimulation utilizes RF energy as opposed to ultrasound (e.g., Deisseroth & Schneider, “Device and Method for Non-Invasive Neuromodulation,” U.S. patent application Ser. No. 12/263,026, Pub. No.: US2009/0112133. Apr. 30, 2009).

Pilla (U.S.2012/0116149) teaches treatment of a neurological injury via applying a pulsed electromagnetic field to the region of the injury to reduce a physiological response such as inflammation or intracranial pressure. This type of treatment is not neuromodulation. Pilla also describes the use of radiofrequency signal to modulate systems (e.g., physiological, central nervous system, cardiac system, pulmonary system, brain, circadian rhythm, biological system) generated by a coil apparatus that encircles the region to be treated; the embodiment does not appear to support targeted neuromodulation as covered by the current inventions. Ragauskas et. al (U.S. Pat. No. 5,388,583) teaches a noninvasive ultrasonic technique to measure pulsatility of intracranial arteries or variation in the pressure of brain tissue. Neuromodulation is not covered. Jarvik et. al (U.S. 2012/0108918) teaches acoustic palpation using a focused ultrasound probe to elicit a response from targets below the skin in patients with chronic pain disorders. In addition to assessing scope and severity of the pain disorders, the acoustic probe is employed to localize nerves or other sensitized tissues for needle or other delivery-device guidance. Jarvik et. al does not teach neuromodulation.

Vagus nerve stimulation involves a programmer in the upper left chest, under the clavicle, with leads wrapped around the Vagus nerve with brain stimulation occurring by the vagus connections to brain structures.

Optical stimulation involves methods for stimulating target cells using a photosensitive protein that allows the target cells to be stimulated in response to light (e.g., Zhang, Deisseroth, Mishelevich, and Schneider, “System for Optical Stimulation of Target Cells,” PCT/US2008/050627, International Publication Number WO 2008/089003, Jul. 24, 2008).

Ultrasound stimulation is accomplished with focused transducers (e.g., Bystritsky, “Methods for Modifying Electrical Currents in Neuronal Circuits,” U.S. Pat. No. 7,283,861, Oct. 16, 2007 and others including previous filings of the inventor noted above). The effect of ultrasound is at least two fold. First, increasing temperature will increase neural activity. An increase up to 42° C. (say in the range of 39 to 42° C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation as explained in Tyler (Tyler, William, James P., PCT/US2009/050560, WO 2010/009141, published Jan. 21, 2011) in which voltage gating of sodium channels in neural membranes is described. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound mediated opening of calcium channels was also observed by Tyler and colleagues. The above approach is incorporated in a patent application submitted by Tyler (PCT/US2009/050560, WO 2010/009141). Alternative mechanisms for the effects of ultrasound may be discovered as well. In fact, multiple mechanisms may come into play, but, in any case, this would not effect this invention. Finsterwald (U.S. 2008/0045882) discloses a neuromodulation method for therapeutically treating cells, wherein an acoustic frequency of the ultrasound is greater than about 100 kHz and less than about 10 MHz.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap placed over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med, Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as typically below 400 Hz) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using sound (including use of a curved transducer and a lens) or RF. The impact of Long-Term Potentiation (LTP) and Long-Term Depression (LTD) for durable effects is emphasized. It is noted that sound produces stimulation by both thermal and mechanical impacts. The use of ionizing radiation also appears in the claims.

Adequate penetration of ultrasound through the skull has also been demonstrated (Hynynen, K. and F A Jolesz, “Demonstration of potential noninvasive ultrasound brain therapy through an intact skull,” Ultrasound Med Biol, 1998 February; 24(2):275-283). Ultrasound can be focused to 0.5 to 2 mm as compared to TMS that can be focused to 1 cm at best.

Drugs can be used for central nervous system effects as well.

One or a plurality of neural elements can be neuromodulated.

While motor-system functions performed using TMS are valuable, they use expensive units, typically costing on the order of $50,000 in 2014 that are large, take a relatively high power, require cooling of the electromagnet stimulation coils, and may be noisy. It would be highly beneficial to be able to perform the same functions using lower-cost stimulation mechanism.

Targeting can be done with one or more of known external landmarks, an atlas-based approach (e.g., Tailarach or other atlas used in neurosurgery) or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Cingulate Gyrus, are elongated and would be more effectively served with an elongated ultrasound field at the target.

It would be preferable to not only stimulate single or multiple targets synchronously, but to have patterns applied both to a single ultrasound transducer and to the stimulation relationships among multiple such transducers.

As mentioned, it has been demonstrated that focused ultrasound directed at neural structures can stimulate those structures. If neural activity is increased or excited, the neural structure is up regulated; if neural activated is decreased or inhibited, the neural structure is down regulated. Preliminary clinical work by universities (Ben-Gurion University and the University of Rome) using Brainsway Transcranial Magnetic Stimulation (TMS) systems has shown that deep-brain neuromodulation can open up the blood-brain barrier to allow more effective penetration of drugs (e.g., for the treatment of malignant tumors).

While the ultrasonic frequencies for neural stimulation are known, it would be preferable to use macro- and micro-pulse shapes optimized for neuromodulation.

Because of the utility of ultrasound in the neuromodulation of deep-brain structures, application of those techniques to alteration of the permeability of the blood-brain barrier is both logical and desirable even though the target is the blood-brain barrier and not necessarily involving the neuromodulation of the neural target itself.

The power needed for stimulation of the spinal cord is significantly less than needed for deep-brain neuromodulation. Alternative mechanisms for the effects of ultrasound may be discovered as well. In fact, multiple mechanisms may come into play, but, in any case, this would not effect this invention.

Methods and systems for delivering ultrasound energy to neural targets with mechanical perturbations are described in applicant's earlier patent publications including US2011/0208094; US2011/0190668; and US2011/0270138.

The treatment of neuropathic pain has been demonstrated using electrical spinal cord stimulation (SCS) using electrodes to suppress hyperexcitability of the neurons via alteration of dorsal horn neurochemistry including the release of serotonin, Substance P, and GABA. For treatment of ischemic pain, it has been suggested that the oxygen supply may be restored via sympathetic stimulation and/or vasodilation.

Although it has been demonstrated that focused ultrasound directed at neural structures can stimulate those structures, the prior methods and apparatus have lead to less than ideal results in at least some instances.

Many patients suffer from diseases and conditions that may be less than ideally treated. For example, patient conditions having similar symptoms can make it difficult to determine the underlying cause of the patient's symptoms. Also, at least some therapies may provide less than ideal results in at least some instances, and it would be helpful to use presently available therapies more effectively, irrespective of neuromodulation modality.

On a practical basis, non-invasive neuromodulation via ultrasound, Transcranial Magnetic Stimulation, or transcranial Direct Current Stimulation is more is more applicable for patient care than invasive mechanisms such electrical Deep Brain Stimulation (DBS) or optogenetics since the process is much less risky and much less expensive. Given the huge number of patients with neurological conditions treatable by neuromodulation, concentrating on non-invasive means is paramount. Regardless of whether neuromodulation is invasive or non-invasive or of what modality, optimization of techniques is key for improved outcomes. Getting neuromodulation parameters right early is critical. Thus being able to benefit from feedback in real time from patients, where possible, including the capability of providing parameter guidance, during the application of neuromodulation is desirable, including, where applicable, use of one modality of neuromodulation, say ultrasound, for preplanning of another modality of neuromodulation, say, Deep Brain Stimulation is highly desirable. In like manner, success in acute non-invasive neuromodulation of one type, say ultrasound neuromodulation, can used to preplan the application of another form of non-invasive neuromodulation, say Transcranial Magnetic stimulation.

### SUMMARY OF THE INVENTION

The purpose of the inventions disclosed herein are to apply ultrasound neuromodulation for the treatment of neurological condition or to impact normal neurological function to optimize the applicable of any form of neuromodulation, whether that form is applied singly or to the application of multiple forms of neuromodulation either simultaneously or serially.

In general, described herein are systems, devices and methods for neuromodulation, including software, hardware, firmware, and the like. This disclosure is broken up into two sections, the first with 16 parts and the second with 27 parts, summarized below, which may be understood individually, and also in context with one or more other parts. Thus, although this disclosure is divided into two sections, each with multiple parts, illustrating a variety of different devices, systems and methods, any of the information contained in one or more of the other sections may be applied to any of the other sections, individually or collectively. Alternatively, each section and parts may be considered independent of the other sections.

While many of the inventions herein described are related to ultrasound neuromodulation, either alone, or combined with other forms of neuromodulation, many of the inventions are applicable to other forms of neuromodulation. The choice or which modality or modalities of neuromodulation to be applied is influenced by such factors as the achievable functional, patient choice (e.g., between non-invasive versus invasive neuromodulation), availability in a given local (either geographic in general and/or clinical facility versus home), and cost. For example, with respect to non-invasive neuromodulation, ultrasound neuromodulation has the benefit over Transcranial Magnetic Stimulation in that the equipment for performing the neuromodulation costs less and is smaller, could be used at home, work, school, and could be shared over time for tune-ups. Because it is noninvasive and relatively low cost, ultrasound neuromodulation appears to be the modality capable of reaching the deep brain that could potentially be cleared by the FDA or other organizations for over-the-counter purchase.

With increasing knowledge and techniques for neuromodulation, there is increased likelihood of successful application of multi-modality neuromodulation.

For any of the parts, disorders may be treated by neuromodulation, the method comprising modulating the activity of one target brain region or simultaneously modulating the activity of two or more target brain regions, wherein the target brain regions are selected from the group consisting of NeoCortex, any of the subregions of the Pre-Frontal Cortex, Orbito-Frontal Cortex (OFC), Cingulate Genu, subregions of the Cingulate Gyrus, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets. Targets may also be selected from the Sphenopalatine Ganglion, Occipital Nerves, peripheral nerves, Spinal Cord, and the Reticular Activating System.

For any of the parts, in some variations, the disorder treated is selected from the group consisting of: addiction (including treatment for smoking cessation), Alzheimer's Disease, Anorgasmia, Attention Deficit Hyperactivity Disorder, autism, Huntington's Chorea, Obsessive Compulsive Disorder, Impulse Control Disorder, autism, anxiety Disorder, Social Anxiety Disorder, Parkinson's Disease and other motor disorders, Post-Traumatic Stress Disorder, depression, bipolar disorder, pain, insomnia, spinal cord injuries, neuromuscular disorders, tinnitus, panic disorder, Tourette's Syndrome, schizophrenia, GI Motility disorders, Compulsive Sexual Behavior, amelioration of brain cancers, dystonia, obesity, eating disorders, stuttering, ticks, head trauma, stroke, Traumatic Brain Injury & Concussion, and epilepsy. The neuromodulation may also be applied to elicit an orgasm or applied for cognitive enhancement, emotional catharsis, Autonomous Sensory Meridian Response, hedonic stimulation, enhancement of neural plasticity, improvement in wakefulness, brain mapping, diagnostic applications, and research functions. In addition to stimulation or depression of individual targets, the invention can be used to globally depress neural activity, which can have benefits, for example, in the early treatment of head trauma or other insults to the brain.

In some variations, a feedback mechanism is applied, wherein the feedback mechanism is selected from the group consisting of patient, functional Magnetic Resonance Imaging (fMRI), Positive Emission Tomography (PET) imaging, electroencephalogram (EEG), video-electroencephalogram (V-EEG), acoustic monitoring, measurement of tremor or other physiological measurements, and thermal monitoring.

In some variations, a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, Sphenopalatine Ganglion stimulation, occipital nerve stimulation, peripheral nerve stimulation, radiosurgery, Radio-Frequency (RF) stimulation, Vagus Nerve Stimulation (VNS), other-implant stimulation, functional stimulation, and/or drugs is replaced by or combined with one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, Sphenopalatine Ganglion stimulation, occipital nerve stimulation, peripheral nerve stimulation, radiosurgery, Radio-Frequency (RF) stimulation, Vagus nerve stimulation, other-implant stimulation, functional stimulation, and/or drugs. The optimization methods and devices described here in are applicable to multiple modalities of neuromodulation.

In some variations, the output is on-line, real time where neuromodulation parameters are changed immediately under direct control of the Feedback Control System or through the use of Guided Feedback.

Also described herein are systems and methods for Ultrasound Stimulation including one or a plurality of ultrasound sources for stimulation of target deep brain regions to up-regulate or down-regulated neural activity.

Also described herein are systems and methods for treatment planning for ultrasound neuromodulation and other treatment modalities for up-regulation or down-regulation of neural activity.

Also described herein are systems and methods for using ultrasound-neuromodulation techniques for the treatment of medical conditions or impacting normal physiological function.

Also described herein are systems and methods for neuromodulation and more particularly to systems and methods for diagnosis and treatment with ultrasound.

Also described herein are systems and methods for neuromodulation delivering optimized deep-brain or superficial deep-brain neuromodulation impacting one or a plurality of points in a neural circuit to produce acute effects or Long-Term Potentiation (LTP) or Long-Term Depression (LTD) using up-regulation or down-regulation.

One of the mechanisms of neuromodulation is the retraining of neural pathways, positively impacting some functionality and negatively impacting other functionality to foster a given clinical or physiological result. The use of one or multiple modalities or neuromodulation can, in some, cases allow for the treatment of two or more conditions.

### Organization

The specification is divided into two sections, Section I related to optimized neuromodulation and Section II related to the application of those techniques to specific clinical applications or to obtain physiologic effects.

### Section I: Optimized Neuromodulation

The methods and systems included in this section are applicable to multiple modalities of neuromodulation. TABLE 1 serves as a table of contents as to what inventions are applicable to which neuromodulation modalities. For superficial nerves like the Occipital Nerve or peripheral nerves, local electrical stimulation with the neuromodulation characteristics like DBS or SCS is applicable. Some of the inventions are specifically directed in whole or in part to ultrasound neuromodulation.

TABLE 1

| | | | | | | | | | | Stereotactic | |
|---|---|---|---|---|---|---|---|---|---|---|---|
| PART | TITLE | DBS | SCS | VNS | TMS | USnd | RF | tDCS | Optogenetics | Radiosurgery | Ancillary |
| I | MULTIMODALITY | X | X | X | X | X | X | X | X | X | X |
| II | FOCUSED ULTRASOUND | | | | | X | | | | | |
| III | SHAPED AND STEERED | | | | | X | | | | | |
| | ULTRASOUND | | | | | | | | | | |
| IV | MECHANICAL PERTURBATIONS | | | | X | X | X | | | | |
| V | INTERSECTING BEAMS | | | | | X | | | | | |
| VI | MACRO- AND MICRO-PULSE | X | X | X | X | X | X | | X | | X |
| | SHAPING | | | | | | | | | | |
| VII | PATTERNED CONTROL | X | X | X | X | X | X | | X | | X |
| VIII | ANCILLARY STIMULATIONS | X | X | X | X | X | X | X | X | | |
| IX | PLANNING AND USING | | | | X | X | X | X | | | X |
| | SESSIONS | | | | | | | | | | |
| X | PATIENT FEEDBACK | X | X | X | X | X | X | X | X | | X |
| XI | DIAGNOSIS/OTHER-MODALITY | X | X | X | X | X | X | | X | | X |
| | PREPLANNING | | | | | | | | | | |
| XII | TREATMENT PLANNING | X | X | X | X | X | X | | X | X | X |
| XIII | SPINAL CORD | | X | | X | X | X | | | | |
| XIV | BRAIN, NERVE ROOTS, | | X | | X | X | X | | | | |
| | PERIPHERAL NERVES | | | | | | | | | | |
| XV | BLOOD BRAIN BARRIER | | | | X | X | X | | | | |
| XVI | WHOLE HEAD | X | | X | X | X | X | | | | X |
| | NEUROMODULATION | | | | | | | | | | |

### Part I: Multi-Modality Neuromodulation of Brain Targets

In some variations, is the purpose of this invention to provide methods and systems for non-invasive deep brain or superficial stimulation using multiple modalities simultaneously or on an interleaved/sequential basis. This approach is particularly of benefit because impacting multiple points in a neural circuit or multiple points in multiple neural circuits to produce Long-Term Potentiation (LTP) or Long-Term Depression (LTD) to treat indications such as neurologic and psychiatric conditions. In some variations, alternative targets in an applicable neural circuit are substituted.

Multiple modalities considered are deep-brain stimulators (DBS) with implanted electrodes, Spinal Cord Stimulation (SCS) with implanted electrodes, Sphenopalatine Ganglion stimulation, occipital nerve stimulation, peripheral nerve stimulation, Transcranial Magnetic Stimulation (TMS), transcranial Direct Current Stimulation (tDCS), implanted optical stimulation (including optogenetics), focused ultrasound, radiosurgery, Radio-Frequency (RF) stimulation, Vagus Nerve stimulation (VNS), other-implant stimulation, ancillary (functional stimulation), and drugs. Note that VNS is representative of other implanted modalities where nerves located outside the cranium are stimulated and these other implanted modalities are covered by this invention. An example is stimulation of the Sphenopalatine Ganglion to abort a migraine headache. Wagner et al. (U.S. 2012/0109020) addresses applying two forms of noninvasive energy to a region of tissue whereby the combined effect modifies a pattern or neural transmission between cells of the neural tissue in that region. Either energy could be selected from thermal, optical, mechanical, electromagnetic, and electrical. Combination of noninvasive neuromodulation (TMS, tDCS, ultrasound, RF, ancillary (functional) stimulation, drugs) with invasive forms of neuromodulation (DBS, SCS, implanted optical stimulation (including optogenetics), VNS) or combination of two invasive modalities of neuromodulation is not covered.

For example, described herein are methods of modulating deep-brain targets using multiple therapeutic modalities, the method comprising: applying a plurality of therapeutic modalities to a deep-brain target, applying power to each of the on-line therapeutic modalities via a control circuit thereby neuromodulating the activity of the deep brain target regions, and working in coordination with the off-line therapeutic modalities.

Some targets may be up regulated and others down regulated. Coordinated control is provided, as applicable, for control of the direction of the energy emission, intensity, session duration, frequency, pulse-train duration, phase, and numbers of sessions, if and as applicable, for neuromodulation of neural targets. Use of ancillary monitoring or imaging to provide feedback may be applied as well as or instead of patient feedback, either direct or through Guided-Feedback Neuromodulation.

In some variations, the one or a plurality of targets are hit by a plurality of therapeutic modalities.

In some variations, the on-line, real-time neuromodulators are selected from the group consisting of ultrasound transducers, TMS stimulators.

In some variations, the output is on-line prescriptive where neuromodulation parameters are directly set in programmers and the effect is both reversible and seen immediately.

In some variations, the on-line, prescriptive neuromodulators are selected from the group consisting of on-line, real-time programmable DBS programmers, Vagus Nerve Stimulation programmers, and neuromodulators with similar characteristics to existing DBS programmers, Vagus Nerve Stimulation programmers, and other-implant programmers.

In some variations, the output is off-line prescriptive adjustable where instructions are generated for users to adjust programmers and the effect is reversible but the effect is seen at a later time after the programmers have been so adjusted.

In some variations, the off-line, prescriptive adjustable neuromodulators are selected from the group consisting of off-line prescriptive adjustable DBS programmers, Vagus Nerve Stimulation programmers, other-implant programmers, and neuromodulators with similar characteristics to existing DBS programmers, Vagus Nerve Stimulation programmers, and other-implant programmers.

In some variations, the output is off-line prescriptive permanent where neuromodulation parameters are instructions are generated for users to adjust parameters and the effect is not reversible and the effect is seen at a later time after the change has been made.

In some variations, the off-line, prescriptive permanent neuromodulators are selected from the group consisting of radiosurgery, neuromodulators with characteristics similar to radiosurgery.

In some variations, the treatment planning and control system varies, as applicable, a plurality of elements selected from the group consisting of direction of energy emission, intensity, pulse-train duration, mechanical perturbations, session durations, numbers of sessions, frequency, phase, firing patterns, number of sessions, relationship to other controlled modalities.

In some variations, real-time modalities are applied simultaneously.

In some variations, real-time modalities are applied sequentially.

In some variations, multiple indications are treated simultaneously or sequentially.

In some variations, the multiple conditions have one or more common targets.

In some variations, the multiple conditions have no common targets.

Also described herein are methods of modulating deep-brain targets using multiple therapeutic modalities for the treatment of pain, the method comprising: applying down-regulation via ultrasound to the Dorsal Anterior Cingulate Gyrus, applying down-regulation via ultrasound to the Cingulate Genu, applying down-regulation via Transcranial Magnetic Stimulation to the Insula, applying down-regulation via ultrasound to the Caudate Nucleus, and applying down-regulation via Deep Brain Stimulation of the Thalamus.

### Part II: Neuromodulation of Deep-Brain Targets Using Focused Ultrasound

It is the purpose of this invention to provide methods and systems for non-invasive deep brain or superficial neuromodulation using ultrasound impacting one or multiple points in a neural circuit to produce acute effects or Long-Term Potentiation (LTP) or Long-Term Depression (LTD). Ultrasound transducers are positioned by spinning them around the head on a track with under control of direction of the energy emission, control of intensity for up-regulation or down-regulation, and control of frequency and phase for focusing on neural targets. The transducer may also rotate while it is moving around the track to enhance ultrasound targeting and delivery. Note that this invention includes circulating ultrasound transducers around a track that has not been previously described, including turning the one or more ultrasound transducers so they face their designated targets, including at all times. Vitek (2006/0058678 A1) describes a set of ultrasound transducers arranged around a circular track, but those transducers are simply adjusted back and forth, as needed, to adjust their position, they are not constantly moved around the track nor do the held transducers turn in such a manner to face their designated targets. Alternatively the ultrasound transducers may be fixed to the track. Use of ancillary monitoring or imaging to provide feedback is optional. In embodiments employing concurrent imaging, the device of the invention is to be constructed of non-ferrous material. A shell can also optionally cover the apparatus.

For example, described herein are methods of neuromodulating one or a plurality of deep-brain targets using ultrasound stimulation, the method comprising: aiming one or a plurality of ultrasound transducers at one or a plurality of deep-brain targets, applying power to each of the ultrasound transducers via a control circuit thereby neuromodulating the activity of the deep brain target region, moving one or a plurality of transducers around a track surrounding the mammal's head.

In some variations, the method further comprises identifying a deep-brain target.

In some variations, the method further comprises where neuromodulation of a plurality of targets is selected from the group consisting of up-regulating all neuronal targets, down-regulating all neuronal targets, up-regulating one or a plurality of neuronal targets and down-regulating the other targets.

In some variations, the step of aiming comprising orienting the ultrasound transducer and focusing the ultrasound so that it hits the target.

In some variations, the acoustic ultrasound frequency is in the range of 0.3 MHz to 0.8 MHz.

In some variations, the power applied is selected from group consisting of less than 180 mW/cm·sup·2 and greater than 180 mW/cm·sup·2 but less than that causing tissue damage.

In some variations, a stimulation frequency of 400 Hz or lower is applied for inhibition of neural activity.

In some variations, the stimulation frequency is in the range of 500 Hz to 5 MHz for excitation.

In some variations, the focus area of the pulsed ultrasound is selected from the group consisting of 0.5 to 500 mm in diameter and 500 to 1500 mm in diameter.

In some variations, the number of ultrasound transducers is between 1 and 25.

In some variations, mechanical perturbations are applied radially or axially to move the ultrasound transducers.

In some variations, one or a plurality of ultrasound transducers moving around a track surrounding the mammal's had are rotated as they go around the track to maintain focus for a longer period of time.

In some variations, the position of one or a plurality of ultrasound transducers are mounted on the track surrounding the mammal's head in a fixed position.

In some variations, there are contradictory effects relative to clinical indications, the method comprising: (a) identifying other targets in the neural circuits that impact those clinical indications that are not in common, and (b) up-regulating or down-regulating one or a plurality of those targets, whereby the contradictory effects are minimized.

Thus, disclosed are methods and systems for non-invasive deep brain or superficial neuromodulation for up-regulation or down-regulation using ultrasound impacting one or multiple points in a neural circuit to produce Long-Term Potentiation (LTP) or Long-Term Depression (LTD) to treat indications such as neurologic and psychiatric conditions. Ultrasound transducers are positioned by spinning them around the head on a track, as well as individually rotated or not, with control of direction of the energy emission, intensity, frequency, mechanical perturbations, and phase/intensity relationships to targeting and accomplishing up-regulation and/or down-regulation. Alternatively the ultrasound transducers may be at fixed locations on the track. Use of ancillary monitoring or imaging to provide is optional.

### Part III: Shaped and Steered Ultrasound for Deep-Brain Neuromodulation

It is the purpose of this invention to provide a device for producing shaped or steered ultrasound for non-invasive deep brain or superficial stimulation impacting one or a plurality of points in a neural circuit to produce acute effects or Long-Term Potentiation (LTP) or Long-Term Depression (LTD) using up-regulation or down-regulation.

For example, described herein are ultrasound transducers for neuromodulation of a deep-brain target comprising: (a) an ultrasound-generation array with a curvature matched to the depth of the target, and (b) a shape matched to the shape of the target, whereby said ultrasound transducer neuromodulates the targeted neural structures producing regulation selected from the group consisting of up-regulation and down-regulation. King et al. (U.S. Pat. No. 5,127,410) address ultrasound transducer probes including lens assemblies for medical scanning, but does not address therapeutic neuromodulation.

In some variations, the ultrasound transducer is elongated to match an elongated target.

In some variations, the ultrasound transducer is a hemispheric cup shaped to match a point target.

In some variations, a plurality of ultrasound transducers is employed to neuromodulate targets selected from the group consisting of multiple targets in a single neural circuit and multiple targets in multiple neural circuits.

In some variations, one or plurality of ultrasound transducers are used with one or a plurality of controlled elements selected from the group consisting of direction of the energy emission, intensity, frequency, firing patterns, mechanical perturbations, and phase/intensity relationships for beam steering and focusing on neural targets.

In some variations, a separate lens used in conjunction with an ultrasound-generating transducer array used in conjunction with the Transcranial Magnetic Stimulation electromagnet has an attachment selected from the group consisting of the bonded to the ultrasound-generating transducer array and not bonded to the ultrasound-generating transducer array.

In some variations, the separate lens used in conjunction with the ultrasound generator is interchangeable.

In some variations, the separate lens is elongated to match an elongated target.

In some variations, the separate ultrasound lens is a hemispheric cup shaped to match a point target.

Also described herein are ultrasound transducers for neuromodulation of a deep-brain target comprising: (a) a flat ultrasound-generation array, (b) an ultrasound controller generating varying the phase/intensity relationships to steer and shape the ultrasound beam, whereby said ultrasound transducer neuromodulates the targeted neural structures producing regulation selected from the group consisting of up-regulation and down-regulation.

In some variations, the ultrasound transducer has a curved ultrasound-generation array instead of a flat ultrasound-generation array.

In some variations, the separate lens used in conjunction with the ultrasound-generating array that is used in conjunction with the Transcranial Magnetic Stimulation electromagnet is interchangeable.

In some variations, a plurality of combination ultrasound-generating transducer arrays and Transcranial Magnetic Stimulation electromagnets are employed to neuromodulate targets selected from the group consisting of multiple targets in a neural circuit and multiple targets in multiple neural circuits.

In some variations, the combination ultrasound-generating transducer arrays and Transcranial Magnetic Stimulation electromagnets are used with control for the ultrasound-generating transducer arrays of one or a plurality of control elements selected from the group consisting of direction of the energy emission, control of intensity, control of frequency for regulation selected from the group consisting of up-regulation and down-regulation, mechanical perturbations, and control of phase/intensity relationships for beam steering and focusing on neural targets

In some variations, the control for the Transcranial Magnetic Stimulation are one or a plurality of control elements selected from the group consisting of intensity, frequency, pulse shape, and timing patterns of the stimulation of the Transcranial Magnetic Stimulation electromagnets.

In some variations, the combination of a Transcranial Magnetic Stimulation means of stimulation and a coaxial ultrasound transducer array aimed at a neural target increases the neuromodulation of the target to a greater degree than obtainable by either means used alone.

Thus, disclosed are devices for producing shaped or steered ultrasound for non-invasive deep brain or superficial neuromodulation impacting one or a plurality of points in a neural circuit. Depending on the application this can produce short-term effects (as in the treatment of post-surgical pain) or long-term effects in terms of Long-Term Potentiation (LTP) or Long-Term Depression (LTD) to treat indications such as neurologic and psychiatric conditions. The ultrasound transducers are used with control of direction of the energy emission, control of intensity, control of frequency for up-regulation or down-regulation, mechanical perturbations, and control of phase/intensity relationships for focusing on neural targets.

### Part IV: Mechanical Perturbations

Mechanical perturbations are a novel mechanism for shaping non-invasive neuromodulation. The impact of mechanical perturbations is to broaden the footprint of the neuromodulation in three dimensions. While mechanical perturbations have been described before in connection with Transcranial Magnetic Stimulation, it has not been previously described in ultrasound and not combined with patterned stimulation in either case.

Mechanical Perturbations were introduced in U.S. patent application Ser. No. 12/940,052 filed Nov. 5, 2010, titled “NEUROMODULATION OF DEEP-BRAIN TARGETS USING FOCUSED ULTRASOUND,” that claims priority to U.S. Provisional Patent Applications 61/260,172 filed Nov. 11, 2009 and 61/295,757 filed Jan. 17, 2010 and further developed in U.S. patent application Ser. No. 13/689,178 filed Nov. 29, 2012, titled “ULTRASOUND NEUROMODULATION OF SPINAL CORD,” that claims priority to U.S. Provisional Patent Application 61/564,856 filed Nov. 29, 2011 AND U.S. patent application Ser. No. 13/734,216 filed Jan. 4, 2013, titled “ULTRASOUND NEUROMODULATION FOR COGNITIVE ENHANCEMENT.”

It is important to note that these are not the mechanical perturbations of the underlying neural membranes that are considered one of the potential mechanisms at the membrane level by which ultrasound neuromodulation works. The invention is also not movement of ultrasound transducers to position them as taught by Vitek (U.S. PC Pub. No. 2006/0058678 A1). Vitek discloses an ultrasound method, wherein transducers can move circumferentially around the subject to allow transducers to be manually adjusted and better positioned to provide energy to a target. The purpose of mechanical perturbations in the invention covered by the current patent application is not to position the transducers but to move them radially or axially to broaden the focal point of the ultrasound field and apply the ultrasound neuromodulation to a larger region with increased action on the neural membranes.

The concept of using mechanical perturbations or oscillations of TMS electromagnets appears in U.S. patent application Ser. No. 12/990,235 (PCT/US2009/045109), Mishelevich and Schneider, “Transcranial Magnetic Stimulation by Enhanced Magnetic Field Perturbations,” published May 5, 2011. The mechanical motions were applied at greater than 1 kHz. The oscillatory perturbing motion was selected to be at a frequency within the range of the pulsing frequency of a typical static TMS electromagnet within 0.1 to 9 mm in movement to provide a change, dB/dt to the magnetic field at the target tissue and at a slightly larger region.

Given that mechanical perturbations had been described previously there are three elements that make the invention in the current context novel. These are application to ultrasound, mechanical-perturbation frequency less than 1 kHz, and the combination of mechanical perturbations with pulse patterns, an important novel element. Patterned neuromodulation is described in Section I Part VII below.

A benefit of mechanical perturbations is that their implementation is likely to be less expensive than implementing shaped phasing transducers that would be an advantage in the marketplace, although the techniques can be used in conjunction with each other.

The range of mechanical-perturbation motion in each of the x, y, and z directions is approximately 0 to approximately 25.4 mm and the frequency from approximately 0.1 Hz to approximately 999 Hz. The use of these mechanical perturbations can be used to match that shape of the neuromodulation to the shape of the target.

A distinct advantage of the mechanical perturbations here is that the cost of the associated ultrasound transducer is significantly less. This is of particular benefit because ultrasound neuromodulation can be applied in the home, work, school, and non-specialist clinical locations where cost concerns will be greater.

### Part V: Ultrasound-Intersecting Beams for Deep-Brain Neuromodulation

Described herein are methods for ultrasound neuromodulation of one or a plurality of deep-brain targets comprising: (a) attaching a plurality of ultrasound transducers to a positioning frame, and (b) aiming the beams from the ultrasound transducers so said beams intersect at the one or plurality of targets, whereby the combination of said ultrasound beams neuromodulates the targeted neural structures producing one or a plurality of regulations selected from the group consisting of up-regulation and down-regulation. A novel element is to have one ultrasound beam emanating from an ultrasound transducer hit more than one target and have that ultrasound beam being intersected with by individual ultrasound beams emanating from two other ultrasound transducers. In one embodiment, the target is neuromodulated by intersecting beams, each with a different neuromodulation characteristic such as a different pattern, see Section I, Part VII. Donald Cohen (U.S. 2009/0149782) teaches intersecting beams, but focused on a single target.

In some variations, the widths of the ultrasound transducer and resultant beam are matched to the size of the target.

In some variations, a plurality of ultrasound transducers is employed to neuromodulate multiple targets in multiple neural circuits.

In some variations, one or a plurality of ultrasound transducers is used with control of selected from the group consisting of direction of the energy emission, intensity, frequency (carrier frequency and/or neuromodulation frequency), pulse duration, pulse pattern, mechanical perturbations, and phase/intensity relationships to targeting.

In some variations, one or plurality of targets is up regulated and one or a plurality of targets is down regulated.

In some variations, one or a plurality of targets is hit with a single ultrasound beam.

In some variations, a combination of a plurality of ultrasound transducers and Transcranial Magnetic Stimulation electromagnets is employed to neuromodulate one or a plurality of targets in one or a plurality of neural circuits.

Also described herein are devices for ultrasound neuromodulation of one or a plurality of deep-brain targets comprising: (a) attaching a plurality of ultrasound transducers to a positioning frame, and (b) aiming the beams from the ultrasound transducers so said beams intersect at the one or plurality of targets, whereby the combination of said ultrasound beams neuromodulates the targeted neural structures producing one or a plurality of regulations selected from the group consisting of up-regulation and down-regulation.

Thus, disclosed are methods and devices for ultrasound-mediated non-invasive deep brain neuromodulation impacting one or a plurality of points in a neural circuit using intersecting ultrasound beams. Depending on the application, this can produce short-term effects (as in the treatment of post-surgical pain) or long-term effects in terms of Long-Term Potentiation (LTP) or Long-Term Depression (LTD) to treat indications such as neurologic and psychiatric conditions. Multiple beams intersect and summate at one or a plurality of targets. The ultrasound transducers are used with control of direction of the energy emission, intensity, frequency (carrier frequency and/or neuromodulation frequency), pulse duration, pulse pattern, mechanical perturbations, and phase/intensity relationships to targeting and accomplishing up-regulation and/or down-regulation.

### Part VI: Ultrasound Macro-Pulse and Micro-Pulse Shapes for Neuromodulation

It is one purpose of this invention to provide methods and systems and methods for optimizing the macro- and micro-pulse shapes used for ultrasound neuromodulation of the brain and other neural structures. Ultrasound neuromodulation is accomplished superimposing pulse trains on the base ultrasound carrier. For example, pulses spaced at approximately 1 Hz of approximately 250 μsec in duration may be superimposed on an ultrasound carrier of approximately 0.65 MHz. Macro-pulse shaping refers to the overall shaping of the individual pulses delivered at so many Hz (e.g., the pulses appearing at approximately 1 Hz). Micro-pulse shaping refers to the shaping of the individual constituent waveforms in the (e.g., approximately 0.65 MHz). Either the macro-pulse shapes or the micro-pulse shapes can be sine waves, square waves, triangular waves, or arbitrarily shaped waves. Neither needs to consistent, that is all being the same shape (e.g., all sine waves); heterogeneous mixtures are permitted (e.g., sine waves mixed with square waves) either within the macro or micro or between the macro and micro. Functional output and/or Positron Emission Tomography (PET) or fMRI imaging can judge the results. In addition, the effect on a readily observable function such as stimulation of the palm and assessing the impact on finger movements can be done and the effect of changing of the macro-pulse and/or micro-pulse characteristics observed. Kenyon et al. (U.S. Pat. No. 4,723,552) deals with Transcutaneous Electrical Stimulation using triangular pulses and Lee et al. (U.S. 2009/0024189) describe various pulse shapes used in electrical Spinal Cord Stimulation, but neither address ultrasound neuromodulation. Hoffman (U.S. 2010/0087698) addresses repetitive Transcranial Magnetic Stimulation for movement disorders with pulsing (e.g., 300 microseconds in length at 0.2 to 0.5 Hz), but does not describe pulse shapes.

For example, described herein are systems of non-invasively stimulating neural structures such as the brain using ultrasound stimulation, the system comprising: aiming an ultrasound transducer at the selected neural target, macro-shaping the pulse outline of the tone burst, applying pulsed power to said ultrasound transducer via a control circuit thereby whereby the neural structure is neuromodulated.

In some variations, the macro-pulse is intensity modulated.

In some variations, the macro-pulse shape is selected from the group consisting of sine wave, square wave, triangular wave, and arbitrary wave.

In some variations, the macro pulses are selected from the group consisting of homogeneous and heterogeneous.

In some variations, the macro-pulse shape is made up of micro-pulse shapes selected from the group consisting of sine wave, square wave, triangular wave, and arbitrary wave.

In some variations, the micro pulses are selected from the group consisting of homogeneous and heterogeneous.

In some variations, the mechanism for focus of the ultrasound is selected from the group of fixed ultrasound array, flat ultrasound array with lens, non-flat ultrasound array with lens, flat ultrasound

In some variations, the efficacy of the macro-pulse neuromodulation is judged via an imaging mechanism selected from the group consisting of fMRI, Positron Emission Tomography, and other.

In some variations, the effectiveness of macro-pulse neuromodulation is judged via stimulating motor cortex and assessing the magnitude of motor evoked potentials.

In some variations, the effectiveness of macro-pulse neuromodulation is judged by stimulation the palm and assessing the impact of finger movements.

In some variations, the Transcranial Magnetic Stimulation pulses rather than ultrasound pulses are shaped.

Thus, disclosed are methods and systems for non-invasive ultrasound stimulation of neural structures, whether the central nervous systems (such as the brain), nerve roots, or peripheral nerves using macro- and micro-pulse shaping. Which macro-pulse and micro-pulse shapes are most effect depends on the target. This can be assessed either by functional results (e.g., doing motor cortex stimulation and seeing which macro- and micro-pulse shape combination causes the greatest motor response) or by imaging (e.g., PET of fMRI) results. The methods and systems described here for macro- and micro-pulse shaping are applicable to all forms of neuromodulation, whether non-invasive or invasive.

### Intensity-Modulated Pulsing

While basic pulsing is well known in the art, intensity modulating the pulse such that the macro-pulse amplitudes vary is novel. Such amplitudes may vary in saw tooth, sinusoidal, triangular, or arbitrary fashion. Repeated groups of the same profile may also vary in the same way. This invention is applicable to all modalities of neuromodulation except stereotactic radiosurgery that causes a permanent structural change and tDCS that is non-pulsed. For multiple targets, can have the various targets have the same or different Intensity-Modulated Pulsing Profiles.

### Part VII: Patterned Control of Ultrasound for Neuromodulation

It is one purpose of this invention to provide an ultrasound device delivering enhanced non-invasive superficial or deep-brain neuromodulation using pulse patterns impacting one or a plurality of points in a neural circuit to produce acute effects or Long-Term Potentiation (LTP) or Long-Term Depression (LTD) using up-regulation or down-regulation. Multiple points in a neural circuit can all up regulated, all down regulated or there can be a mixture. Typically LTP is obtained by up-regulation obtained through neuromodulation and LTD obtained by down-regulation obtained through neuromodulation. Two different targets may have different optimal frequency stimulations (even if both up-regulated and down-regulated). John (U.S. 2007/0043401) notes patterns used in electrical stimulation in brain networks but does not describe the patterns.

In this invention, this is achieved by individually controlling the pulse pattern applied to each of the ultrasound transducers generating ultrasound beams impacting individual targets. The pulse patterns can be applied to individual ultrasound transducers hitting individual targets or sets of transducers applying ultrasound neuromodulation on a given target using non-intersecting or intersecting ultrasound beams. Pulse patterns can vary in one or both of timing or intensity. Timing patterns may vary either in frequency or inter-pulse or inter-train intervals (e.g., one pulse followed by two pulses with a shorter inter-pulse interval and repeat) for each individual ultrasound transducer.

To assess the efficacy of the patterned neuromodulation, ancillary monitoring or imaging may be employed.

For example, described herein are methods for ultrasound neuromodulation of one or a plurality of deep-brain targets comprising: (a) providing one or a plurality of ultrasound transducers; (b) aiming the beams of said ultrasound transducers at one or a plurality of applicable neural targets; (c) modulating the ultrasound transducers with patterned stimulation, whereby the one or a plurality of neural targets are each neuromodulated producing regulation selected from the group consisting of up-regulation and down-regulation.

In some variations, the variation is of one or a plurality selected from the group consisting of inter-pulse intervals and inter-train intervals.

In some variations, the pulse-burst trains are selected from the group consisting of fixed and varied.

In some variations, the inter-pulse-train intervals are selected from the group consisting of fixed and varied.

In some variations, the applied intensity pattern is selected from the group consisting of fixed and varied.

In some variations, the pattern applied is selected from the group consisting of random, theta-burst stimulation.

In some variations, the control system used for control of the patterns is selected from one or a plurality of inputs selected from the group consisting of user input, feedback from imaging system, feedback from functional monitor, and patient input.

In some variations, the relationship among applied frequency pattern, applied timing pattern, and applied intensity pattern is selected from the group consisting of independently varied, dependently varied, independently fixed, and dependently fixed.

In some variations, the pattern is varied during the course of neuromodulation.

In some variations, the effect of patterned ultrasonic neuromodulation is selected from one or more of the group consisting of acute effect, Long-Term Potentiation and Long-Term Depression.

In some variations, the applied pattern is selected from the group of synchronous with all ultrasound transducers using the same pattern and asynchronous with not all ultrasound transducers using the same pattern.

In some variations, the locations of the targets are selected from the group consisting of in the same neural circuit and in different neural circuits.

In some variations, the use of multiple ultrasound transducers is selected from one or a plurality of the group consisting of neuromodulation of the same target and neuromodulation of different targets.

In some variations, the pattern applied in used to avoid side effects elicited by neuromodulation of one or a plurality of structures selected from the group consisting of unintended structures and structures that need to be protected from neuromodulation.

In some variations, the applied pattern is selected from the group of where all targets receive the same pattern and all targets do not receive the same pattern.

In some variations, one set of applied patterns applied to a given neural circuit to provide treatment for one condition and an alternative set of applied patterns is applied to that neural circuit to provide treatment for another condition.

In some variations, any of the patterns described may be varied during the course of neuromodulation.

The methods and systems described here for pulse-patterned neuromodulation are applicable to all forms of neuromodulation, whether non-invasive or invasive.

Patterned Transcranial Magnetic Stimulation has been described previously in Mishelevich and Schneider (Mishelevich, David J. and M. Bret Schneider, “Firing Patterns for Deep Brain Transcranial Magnetic Stimulation,” PCT/US2008/073751 filed 20 Aug. 2008), but does not include the novel elements of patterned neuromodulation described below.

### Fixed Pulse Pattern

In this pulse pattern, both the pulse width and inter-pulse intervals are fixed.

### Random Pulse Patterns

While traditional pulse trains used in neuromodulation occur at fixed intervals, this invention includes a random pattern as an alternative. In selected situations, use of random pulsing can eliminate potential problems with habituation.

### Fibonacci Sequence Pulsing

In this type of patterned neuromodulation, the novel pattern is determined by a Fibonacci sequence applied to the number of space elements between pulse elements. The duration of each space element can vary between approximately 0.1 ms and approximately 5 sec, but not limited to this range. The duration of each pulse element can vary between approximately 0.01 ms and approximately 1 sec, but not limited to this range. In a given pattern, the duration of each space element need not be the same as the duration of each pulse element and the durations of each pulse element need not be equal. In generating the Fibonacci sequence, the beginning numbers can be 0, 1 or 1, 1. For the Fibonacci sequence, the number of space elements between pulse elements is selected in order or randomly from the first k terms of Fibonacci sequence. Examples are first 12 terms with first two numbers 0 and 1 or first six terms with the first two numbers 1 and 1. The number of terms, k, used can vary between 1 and 25. In the Fibonacci sequence, the value of element n is calculated by adding the values of elements (n−1) and (n−2). If the initial numbers are 0 and 1, the sequence runs 0, 1, 1, 2, 3, 5, 8, 13, 21, 34, 55, etc. If the initial numbers are 1 and 1, the sequence runs 1, 1, 2, 3, 5, 8, 13, 21, 34, 55, etc. For example, if the initial numbers in the Fibonacci sequence are 1, 1, and the number of terms k to be used is 7, then the number of space elements to be applied is 1, 1, 2, 3, 5, 8, and 13. If the resultant numbers of space elements (1, 1, 2, 3, 5, 8, 13) are to be applied in order then the generated pulses, starting at time 0 will occur at positions 2, 4, 7, 11, 17, 26, and 40. This is because there will be one space between positions 0 and 2, one space between positions 2 and 4, 2 spaces between positions 4 and 7, 3 spaces between positions 7 and 11, 5 spaces between positions 11 and 17, 8 spaces between positions 17 and 26 and 13 spaces between positions 26 and 40. The actual length of time for those 40 spaces is essentially 33 spaces because the 7 pulses are likely to be so short (e.g., approximately 0.2 ms) so if the duration of a space is 20 ms each then the length of time for the 7 pulses is 33 times 20 ms equals 660 ms. To get the average frequency in Hz, the number of pulses in one second is ((1,000 ms/sec)/660 ms) times 7=10.6 pulses/sec=10.6 Hz. This 10.6 Hz rate is in the range of up regulation. If the order to be applied is random, one would use a pseudo random number generator to randomly generate the order that the space elements from the Fibonacci sequence will be applied by picking the nth element as per the pseudo random number generator. The average frequency will be the same. Again, as for the other patterns, this pattern can be applied to and improve the performance of all modalities of neuromodulation except transcranial Direct Current Stimulation that is not pulsed.

### Continuous, Non-Pulsed

In this pattern, the neuromodulation is not pulsed but continuous. A modality such as optogenetics can be used in a continuous mode while one like Transcranial Magnetic Stimulation (TMS) cannot. Optogenetics depends on the wavelength of light to neuromodulate the neural membrane, not necessarily pulsating that light. If not pulsed, the Transcranial Magnetic Stimulation magnets would cause a static magnetic field at the neural target and cause no net change in the target neural membrane. The intensity of the neuromodulation, however, can vary.

### Burst-Mode Pattern

In another embodiment, instead of having a constant stream of pulses, the ultrasound neuromodulation is turned on for a period of time and then turned off and repeated after a period of time. For example, the burst pattern may be turned on and off every three seconds. The duration of the on time of the burst and duration of the off time of the burst may not be symmetric. Thus each of the on and off interval times may be selected from 1 (or a fraction thereof), 2, 3, 4, 5 seconds ranging up to 500 seconds and not necessarily integer time so 0.06, 1.05, 2.3, 5.0002 sec, etc. are permissible. Theta-burst neuromodulation is one form of applicable burst-mode pattern. The pulses contained in the burst can be any of the neuromodulation pulse patterns. In one embodiment, a bang-bang mode is used in which a set of bursts (say four seconds in duration) is directed towards one target and then that neuromodulation halted and a set of bursts (say again four seconds) directed at another target.

### Multiple-Frequency Amplitude Modulation

Neuromodulation systems to date deliver pulses of a single frequency (say 900 Hz) and pulse interval (say every 0.2 ms) superimposed on a carrier frequency (say 0.65 MHz) to the target. In the current invention, pulses of two or more frequencies (e.g., for two frequencies, 1000 Hz every 0.2 ms and 1500 Hz every 0.2 ms, but offset by 0.1 ms so they do not overlap) are delivered simultaneously on a single carrier. In some embodiments, there can be a mixture of frequencies and inter-pulse intervals whether directed to single or different targets of any number with recognition that with varying pulse intervals that some pulses may overlap. The range of either of the two frequencies will be between approximately 10 Hz to 400 Hz for down regulation and approximately 500 Hz to 5 MHz for up regulation with any set of endpoints within those approximate ranges or outside them. The adjective approximately is used because depending on the patient the frequency break between up regulation and down regulation (for example, in some cases the frequency for down regulation might go up to 600 Hz and the neuromodulation frequency for up regulation begin at 900 Hz, but in any case wherever the break would be determined through neuromodulation of the specific patient without reservation).

### Sweep Amplitude-Modulation Frequency

In this embodiment the neuromodulation frequency (as contained within the envelope of the pulses) is varied or swept through a range. For example, the frequency for down regulation may be sinusoidal (or other fashion) varied periodically from 200 Hz to 400 Hz. Embodiments are not limited to this range. Such variation in time can repeatedly occur over any time period from approximately zero seconds to approximately 60 seconds or higher, without reservation. The profile can be of any shape (e.g., sinusoidal or triangular).

The initial state (say 100 Hz if the frequency is being swept from 100 Hz to 300 Hz) can be synchronized with the beginning of each of the square (or other wave) (i.e., started from 100 Hz at the initiation of each square-wave pulse) or left at whatever the frequency that would normally occur at that time if the wave were continuous. In some embodiments the range of the swept frequency would be adjusted such that it would reach the maximum of the range (e.g., 300 Hz in the center of the square-wave pulse and return to the initial value (e.g., 100 Hz) at the end of the square-wave pulse.

### Sweep Pulse Frequency

In this embodiment, the inter-pulse interval varies. For example, the inter-pulse interval may vary between approximately 0.1 ms or less and approximately 1 second or more. The change can follow a variety of profiles, for example, sine wave, triangle eave, saw-tooth, or other, including arbitrary. The variation will occur over a length of time, say between, but not limited to, 1 sec to 10 sec.

### Sweep Duty Cycle

In this embodiment, the pulse duty cycle (the proportion of the inter-pulse interval that is with filled neuromodulation pulse) may be either fixed at different values or swept through a set of values over a period of time. For example, in the former case, the pulses can be generated with an inter-pulse interval of 10 per second (one every 100 ms) but if the duty cycle were 50% the duration of the pulses would be 50 ms or if the duty cycle were 10% the duration of the pulses would be 10 ms. If the duty cycle were swept, the percentage of on time could vary according to a profile (e.g., sine wave, saw tooth wave, etc. including arbitrary) over a duration of time, say between, but not limited to, 1% to 100% of the inter-pulse interval with the sweeping occurring over 1 Hz to 20 kHz.

One consideration is that by varying the duty cycle one can increase the level energy delivered without having to increase the neuromodulation amplitude. This can have safety benefits.

### Multiple-Target Patterns

In one embodiment of neuromodulation of multiple targets, the neuromodulation of each of the multiple targets has the same pattern. In an alternative embodiment, the neuromodulation of at least one of the multiple targets has a different pattern.

### Cumulative Energy Delivered

Impact of neuromodulation using any modality can be quantitated in terms of the number of pulses delivered or, considering the duty cycle, the number of pulses times the duty cycle. For example if pulses are delivered at 2 Hz (one pulse every 500 ms), there will be 120 pulses per minute and therefore 6000 pulses in a 50-minute session. This is one metric. If the length of the delivered pulses were 0.1 ms, the duty cycle would 0.1 ms divided by 500 ms or 0.02%, and active time over the 50-minute session would be 0.02% times 50 minutes=0.01 minutes. This is another metric. The length of a session can vary. In the case of Transcranial Magnetic Stimulation (TMS), a typical session may be 50 minutes in length; in the case of Deep Brain Stimulation (DBS) or other implanted electrical stimulation neuromodulation, sessions may be infinite in length or a number of hours per day. In one embodiment of this invention neuromodulation is delivered in the range of, but not limited to, approximately 1,000 to approximately 100,000 per session and active time from 0.001 to 10 minutes.

In other embodiments of the above, the amplitudes of the neuromodulation pulses are varied or swept through a range per the pattern profile. For example, the amplitude may vary in the range of approximately 10% of full-scale power of the generator to 100% of full-scale power or varied from 1 percent to 500 percent of the nominal pulse amplitude in a sinusoidal fashion at 50 Hz.

A common element to the application of pulse patterns in neuromodulation is that they can be applied to single or multiple targets. In the case of multiple targets, the same or different patterns can be applied to each of the individual targets. An aspect of this is that targets can be neuromodulated simultaneously or interleaved. For example pulses, irrespective of neuromodulation modality, can be delivered first to one target and then another, in a bang-bang fashion with rotation among multiple targets and including the case where one or more targets are hit simultaneously and one or more other targets are hit at unique times.

### Part VIII: Ancillary Stimulation

In this embodiment, ultrasound neuromodulation is augmented with one or more additional stimulations such as visual, auditory, tactile, vibration, pain, proprioceptive stimulations or any other form of energy input. Such ancillary stimulations (ancillary to neuromodulation) were introduced in U.S. patent application Ser. No. 13/035,962 filed Feb. 26, 2011, titled “ORGASMATRON VIA DEEP-BRAIN MODULATION,” that claims priority to U.S. Provisional Patent Application No. 61/308,987 filed Feb. 28, 2010.

In one embodiment, the ancillary stimulation is directed to one or more specific targets related to the physiological result to be achieved (e.g., clinical condition). In another embodiment, the ancillary stimulation will increase the background (see Mishelevich, U.S. patent application Ser. No. 13/031,192 filed Mar. 19, 2011, titled “ULTRASOUND NEUROMODULATION OF THE RETICULAR ACTIVATING SYSTEM.” that claims priority to U.S. Provisional Patent Application No. 61/306,531 filed Feb. 21, 2010) so a lower energy level of ultrasound neuromodulation will work effectively. Such ancillary stimulation is that it can allow simultaneous neuromodulation to be delivered at a lower level even if the background level of neural activity is not increased or even if the neuromodulation were delivered at even the maximum safe level allow impart of the neuromodulation to work at increased depth than would otherwise be possible.

In one embodiment, the ancillary audio stimulation is not restricted to a single tone or combination of tones. Music or other sounds (e.g., voices, waves, animal sounds) can be effective for up-regulation or down-regulation. For example use of Tchaikovsky's 1812 overture, rapid-tempo march, or other upbeat music can aid in the treatment of depression. Soothing or downbeat music can aid in the treatment of anxiety. In some cases, the presence of the ancillary stimulation can serve, even if not overtly tied to the condition being treated.

In like manner, visual stimulation can be tied to up-regulation or down-regulation. In the case of depression, for example a funny cartoon could be used while a video of a calm brook could aid in the treatment of anxiety. Other stimuli such as the application (dry or wet) of cold or warm temperatures, or vibration can serve. The part of the body may influence the effect like the affected limb in the rehabilitation of stroke. Application of such stimuli is not limited to skin, the tongue can be targeted as well. In yet another embodiment, the ancillary stimulation will be directed at one or more targets in the relevant neural circuit that are not targeted by the incident ultrasound neuromodulation. In still another embodiment, the (acute) clinical response to the ancillary stimulus is used to indicate which targets for the specific patient would likely respond to ultrasound neuromodulation (see also Part XI).

Ancillary stimulation has at least two additional functions, one is to be part of feedback assessment (see Part X) and the other is to augment neuromodulation in the “focusing” mode of whole-head neuromodulation (see Part XVI).

### Part IX: Planning and Using Sessions of Ultrasound for Neuromodulation

Also disclosed are systems and methods for non-invasive neuromodulation using ultrasound delivered in sessions. Examples of session types include periodic over extended time, periodic over compressed time, and continuous. Maintenance sessions are either periodic maintenance sessions or as-needed maintenance tune-up sessions. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, and phase/intensity relationships to targeting and accomplishing up regulation and/or down regulation.

It is the purpose of some variations of the inventions described herein to provide methods and systems for non-invasive neuromodulation using ultrasound delivered in sessions. This is important because different conditions and patients need different treatment regimens. Examples of session types include periodic over extended time, periodic over compressed time, and continuous. Periodic sessions over extended time typically means a single session of length on the order of 30 to 60 minutes repeated daily or five days per week over a four to six weeks. Other lengths of session or number of weeks of neuromodulation are applicable, such as session lengths up to 2.5 hours and number of weeks ranging from one to eight. Periodic sessions over compressed time typically means a single session of length on the order of 30 to 60 minutes repeated during awake hours with inter-session times of 30 minutes to 60 minutes over one to two days. Other inter-session times such as 15 minutes to three hours and days of compressed therapy such as one to five days are applicable.

In addition, considerations include both periodic maintenance sessions and/or as-needed maintenance tune-up sessions. Maintenance categories are Maintenance Post Completion of Original Treatment at Fixed Intervals and Maintenance Post Completion of Original Treatment with As-Needed Maintenance Tune-Ups. An example of the former are with one or more 50-minutes sessions during week 2 of months four and eight, and of the latter is one or more 50-minute sessions during week 7 because a tune up is needed at that time as indicated by return of symptoms. Sessions using ultrasound neuromodulation are not just applicable to deep-brain neuromodulation. Size and cost of the ultrasound neuromodulation equipment in many circumstances may make it impractical to deliver the energy continuously. An example of an exception is the case where patient being treated is comatose and the energy can be delivered continuously. Another example is the control of hypertension during a hypertensive crisis and the patient cooperates by remaining relative stationary. Of course, for configurations (e.g., superficial targets) requiring less power and fewer ultrasound transducers, ambulatory use is practical (continuous neuromodulation or otherwise). Ultrasound neuromodulation can produce acute effects or Long-Term Potentiation (LTP) or Long-Term Depression (LTD). Included is control of direction of the energy emission, intensity, frequency (carrier frequency and/or neuromodulation frequency), pulse duration, pulse pattern, mechanical perturbations, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

Any target is applicable. Multiple targets can be neuromodulated singly or in groups. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS). Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets. The effectiveness of the neuromodulation will depend on session characteristics in terms of how frequently and how long the neuromodulation is applied.

Transcranial Magnetic Stimulation is typically delivered in the periodic over extended time mode (e.g., the Neuronetics recommended protocol is 5 days per week, 40 to 50 minutes per day, for six weeks). There are studies underway for accelerated treatment (periodic over compressed time). An example is the Veteran's Administration Trial (clinicaltrials.gov ID NCT00248768) whose purpose is to determinate if accelerated rTMS (repetitive Transcranial Magnetic Stimulation) treatment over 1.5 days is effective for ameliorating depression in Parkinson's disease. The rTMS Treatments consist of 1000 total pulses at 10 Hz and 100% motor threshold administered hourly for 1.5 days, totaling 15 sessions. Of course, 1.5 days is significantly shorter than four to six weeks. Positive results for the trial were reported (Holtzheimer PE 3rd, McDonald W M, Mufti M, Kelley M E, Quinn S, Corso G, and C M Epstein, “Accelerated repetitive transcranial magnetic stimulation for treatment-resistant depression,” Depress Anxiety. 2010 October; 27(10):960-3).

Continuous pulsed stimulation as opposed to breaking into sessions is not practical with TMS because of the large cost and large size of the equipment required. As to maintenance therapy, approaches vary, but post-maintenance can range from periodic (even beginning short term like once per week beginning just after the end of the initial treatment) to on an as-needed basis (e.g., can involve two to 10 treatments delivered when symptoms return (e.g., 6 months to two years after initial treatment)).

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target.

In some variations, the length of session is between 15 minutes and two and a half hours.

In some variations, the type of session is selected from the group consisting of periodic over extended time, periodic over compressed time, and continuous.

In some variations, the extended time involves daily sessions daily or five days per week over a period of one to six weeks.

In some variations, the compressed time is one to five days.

In some variations, the compressed time included inter-session time between 15 minutes to three hours.

In some variations, the maintenance mode is selected from the group consisting of maintenance post-completion of original treatment at fixed intervals and maintenance post-completion of original treatment with as-needed maintenance tune-ups.

In some variations the maintenance or tune-up is triggered when the patient's symptoms deteriorate in the range of 5% to 1000% or more.

In some variations days in either base or tune-up sessions are skipped based on the first few elements of a Fibonacci Sequence beginning with (0, 1) or on a number selected by the operator.

The methods and systems described here for use of sessions are applicable to all forms of neuromodulation, whether non-invasive or invasive, although more likely to be applied to non-invasive neuromodulation.

While sessions have been known in non-invasive neuromodulation, instead of doing it daily on weekends, the invention here is novel in that days are selected on the a Fibonacci Sequence (or other mathematical sequences) applied days on which neuromodulation is applied. In generating the Fibonacci sequence, the beginning can be the numbers 0.1 or 1. For the Fibonacci sequence, the number of days between days in a session is selected in order or randomly from the first k terms of Fibonacci sequence. Examples are first three terms with first two numbers either 0 and 1 or 1 and 1. The number of terms, k, used can vary between 1 and 5. In the Fibonacci sequence, the value of element n is calculated by adding the values of elements (n−1) and (n−2). If the initial numbers are 0 and 1, the sequence runs 0, 1, 1, 2, 3, 5, 8, 13, etc. If the initial numbers are 1 and 1, the sequence runs 1, 1, 2, 3, 5, 8, 13, etc.

In another embodiment, sessions are constructed in such a way that a variable number of the five days per week is selected from pre-specified numbers such as 3, 4, and 5, or selected in order or randomly five days minus a number selected from the first k terms of Fibonacci sequence, where k equals four and the first two numbers are either 0 and 1 or 1 and 1. In still another embodiment the operator specifies a single specific number or sequence of numbers.

Another aspect of the invention is the scheduling of the tune-up session for neuromodulation if the tune-up session has not already been triggered by return of patient symptoms. In one embodiment, the number of weeks that the tune-up session occurs after the last session of the initial series is selected in order or randomly from the first k terms of Fibonacci sequence. Examples are first three terms with first two numbers either 0 and 1 or 1 and 1. The number of terms, k, used can vary between approximately 6 and 10. In the Fibonacci sequence, the value of element n is calculated by adding the values of elements (n−1) and (n−2). If the initial numbers are 0 and 1, the sequence runs 0, 1, 1, 2, 3, 5, 8, 13, 21, 34, 55, etc. If the initial numbers are 1 and 1, the sequence runs 1, 1, 2, 3, 5, 8, 13, 21, 34, 55, etc. Skipping of days within a session by an operator-selected number or by application of a Fibonacci Sequence is also applicable to maintenance/tune-up sessions.

### Part X: Patient Feedback for Control of Ultrasound Deep-Brain Neuromodulation

For example, described herein are methods of modulating a deep-brain targets using ultrasound neuromodulation, the method comprising: a mechanism for aiming one or a plurality of ultrasound transducers at one or more a deep-brain targets; applying power to each of the ultrasound transducers via a control circuit thereby modulating the activity of the deep brain target region; providing a mechanism for feedback from the patient based on the acute sensory or motor conditions of the patient; and using that feedback to control one or more parameters to maximize the desired effect. In another embodiment Guided Feedback is employed. The methods and systems described for feedback are applicable to all forms of neuromodulation, whether non-invasive or invasive. The objective is to the “right” neuromodulation, regardless of modality, to improve patient outcomes and increase the return on investment of performing the treatment. Using the feedback of this invention with its immediacy is key so as to not spend weeks with sub-optimal neuromodulation.

Immediate feedback by the patient and/or the healthcare provider can guide the process in real time. This is particularly important in consideration of non-invasive neuromodulation where it can take multiple sessions for positive effects to be realized. Direct, immediate feedback where one does not have to wait and see what works is of significant benefit. While feedback appears in the prior art (e.g., Mishelevich, David J. and M. Bret Schneider, “Intra-Session Control of Transcranial Magnet Stimulation,” PCT/US2008/081048, filing date 2008-10-24), it only covers direct patient feedback, not Guided Feedback. A variety of input tools can be used such as mouse, joystick, bars or spinners, voice-command input, and, on touchscreens, the ability to move directionally. Patient Feedback can be augmented with automatic or semi-automatic algorithmic Support as described in the following embodiment.

Parameters to be changed are selected from any of the parameters covered in this invention, including being applied to see whether up regulation or down regulation would suit the particular application better. As to order in which changes are to be applied by one with ordinary skill in the art, higher priority change to be made is the repetition rate of the pulsing, pulse duty cycle, and neuromodulation frequency, and, if applicable to the given modality, changing the shape of the neuromodulation by using mechanical perturbations or changing the aiming of the energy transducers. The order or the parameters to be adjusted are not limited to these, however.

With direct patient feedback, the patient or operator can adjust different parameters, pulse parameters, frequency, and/or other parameters.

TABLE 2 lists assessment mechanisms for evaluating feedback for a variety of conditions to be treated or physiological impacts.

TABLE 2

| | CONDITION OR PHYSIOLOGICAL | |
|---|---|---|
| PART* | IMPACT | ASSESSMENT |
| I | Orgasm Elicitation | Arousal |
| II | Stroke and Stroke Rehabilitation | Movement of affected limb |
| III | Pain | Pain characterization (e.g., |
| | | Visual Analog Scale) |
| IV | Tinnitus | Tinnitus level |
| V | Depression and Bipolar Disorder | Depression scale |
| VI | Addiction | Craving for applicable substance |
| | | in light of image or odor of |
| | | addictive substance |
| VII | PTSD | Response to viewing applicable |
| | | inciting image |
| VIII | Motor (Tremor) Disorders | Tremor |
| IX | Autism Spectrum Disorders | Test response to situation with |
| | | spontaneous situation |
| X | Obesity | Craving for applicable food in |
| | | light of image or odor of that |
| | | food |
| XI | Alzheimer's Disease | Performance on memory test |
| XII | Anxiety including Panic Disorder | Response to frenetic images |
| | | and/or audio |
| XIII | OCD | Response to video or obsessive |
| | | behavior |
| XIV | GI Motility | Response to inciting food for |
| | | diarrhea or intestinal feeling for |
| | | constipation/Satiety |
| XV | Tourette's Syndrome | Verbal outburst to inciting |
| | | situation |
| XVI | Schizophrenia | Paranoia response to inciting |
| | | visual and/or audio |
| XVII | Epilepsy | Reaction to eliciting image |
| XVIII | Attention Deficit Hyperactivity Disorder | Hyperactivity response to |
| | (ADHD) | inciting visual and/or audio |
| XIX | Eating Disorders | Reaction to food/Satiety |
| XX | Cognitive Enhancement | Performance on problem-solving |
| | | test or video gaming |
| XXI | Traumatic Brain Injury (TBI) including | Ability to perform repetitive |
| | Concussion | physical activity |
| XXII | Compulsive Sexual Disorders | Reaction to explicit visual |
| | | and/or audio sexual material |
| XXIII | Emotional Catharsis | Reaction to release trigger |
| XXIV | Autonomous Sensory Meridian Response | Reaction to ASMR-eliciting |
| | (ASMR) | known phenomenon for the |
| | | given individual |
| XXV | Occipital Nerve | Pain-level measurement (e.g., |
| | | Visual Analog Scale) |
| XXVI | Sphenopalatine Ganglion (SPG) | Pain-level measurement (e.g., |
| | | Visual Analog Scale) and/or |
| | | measurement of aura |
| XXVII | Reticular Activating System (RAS) | Physiological reaction to pain |
| | | stimulation or measurement of |
| | | ocular microtremor (OMT) |

*Numbers reflect those in Section II below

Feedback cannot only modify the change in neuromodulation of a given single target, it can drive change balance among different targets and/or different modalities. The operator can use Diagnosis and Other Preplanning (Section I Part XI) and/or Treatment Planning (Section I Part XII) to change patterns and intensity, balancing among targets and/or modalities.

### Guided Feedback

As a key element, the current invention includes a novel feature that has not been previously described: Guided Feedback. In this case, the patient or other feedback (e.g., operator or physiological measurement such as EEG or EMG) is not employed to change the neuromodulation parameters (e.g., neuromodulation frequency or pulse interval) directly, but an optimization algorithm is applied (e.g., hill climbing) and the patient or operator provides feedback to the system about whether the change dictated by the was either better, worse, or unchanged than the last neuromodulation and/or what relative level on say a numerical scale. The choice of parameters for the next segment of neuromodulation is guided or selected by an optimization algorithm such as the hill climbing algorithm, the greedy algorithm, simulated annealing, or other such algorithm. The use of reports of relative level is particularly useful when the system jumps from the exploration of the search space for one minima or maxima region for neuromodulation parameters to another. A critical consideration is that while the Guided Feedback System can and will jump to another region of the search space rather than continuing to explore a local region, a patient, operator, or agent will not at all or, if attempted, will not know what would be a reasonable other region to explore. When manual feedback is used, the one with ordinary skill in the art providing input to the system does not know how to change the variables. Guided-Feedback Neuromodulation optimization is key.

Whether one uses a minimum or maximum depends on the framing of the results for the patient, operator, or agent providing the feedback. In one view, the minimum is applicable because the patient, operator, or agent is judging decrease in symptoms. In the alternative view, the maximum is applicable because the patient, operator, or agent is judging increase in symptom relief. The judgments can be made either on a continuous basis or a periodic basis such as after each minute or two of neuromodulation. Examples of the judgments are level of depression, level of craving, level of anxiety, and magnitude of tremor.

Another important element is an embodiment in which a by-product of the optimization process, a derived signal is generated representing the change in neuromodulation parameters, and/or the relative change in symptoms. That signal can be used to control via the mind of imagery on a computer screen related to the symptomatology or to control an actuator such as one operating a bionic limb or other actuator, or even play a computer game. Another is a patient with a transected spinal cord directly turning on the neuromodulation to empty a neurogenic bladder.

This novel approach of Guided-Feedback Neuromodulation is applicable to the optimization of any modality of neuromodulation and application to other modalities besides ultrasound neuromodulation is a component of the invention. It is true that ultrasound neuromodulation uses more parameters that can be practically adjusted. Even invasive neuromodulation forms with fixed-location energy emitters like Deep-Brain Modulation, Vagal Nerve Stimulation, Spinal Cord Stimulation, or optogenetics still can be made more effective by optimizing the pulse rates and patterns. In those cases with fixed emitters, one can apply the adjustments of Guided-Feedback Neuromodulation over a longer period of time because one is not limited in duration to sessions (e.g., 50 minutes) that occur in most applications of non-invasive neuromodulation such as ultrasound neuromodulation or Transcranial Magnetic Stimulation. The use of feedback as described is novel in part because other inventors previously have been focusing on implementing basic mechanisms to accomplish neuromodulation rather than making the given neuromodulation more effective.

In Guided-Feedback Neuromodulation, a set of neuromodulation parameters/variables is applied in a given segment, the patient, operator, or agent (intelligent judge of input from physiological sensors) judges the result, and based on that input an algorithm is applied to determine the neuromodulation parameters/variables to be applied in the next segment. Parameters to be changed are selected from any of the parameters covered in this invention, including being applied to see whether up regulation or down regulation would suit the particular application better. As to order in which changes are to be applied in the Guided Feedback by one with ordinary skill in the art, higher priority change to be made is the repetition rate of the pulsing, pulse duty cycle, and neuromodulation frequency, and, if applicable to the given modality, changing the shape of the neuromodulation by using mechanical perturbations or changing the aiming of the energy transducers. The order or the parameters to be adjusted are not limited to these, however.

TABLE 8 lists the variable parameters for neuromodulation that can be used individually or make up sets that can be change on the basis of Guided-Feedback Neuromodulation and the neuromodulation modalities to which they would apply. The applicable neuromodulation modalities are both non-invasive and invasive.

An example of a parameter set that would be varied during Guided Feedback processing is a combination pulse duration (varying in the range between 0.10 ms to 0.25 ms in increments of 0.05 ms), pulse frequency with choices of 15, 30, and 45 Hz and pulse pattern using a the first 3 or 5 elements in Fibonacci sequence with initial elements of 0 and 1. This sample set is applicable to multiple modalities. The Hill Climbing Algorithm is one example of guidance algorithms to be applied; the Greedy Algorithm and Simulated Annealing are others. The object is to find a global minimum for the symptoms rather than a local minimum. After the currently optimal parameter set is identified it can not only be applied for the rest of the session but saved to be used to at least start the subsequent session.

The initial set of parameters will start with a standard seed template as determined by the operator or saved from the previous session. From that point, the application of the guidance algorithm will manage the process regardless of the type of neuromodulation.

Patient symptoms judged may be either the symptoms of the disease being treated or the effect being sought (such as cognitive enhancement) or surrogates for the symptom or effect (such as itching for pain). In some cases, there may be a visible change such as magnitude of tremor that can be judged, and feedback input, by another person such as a healthcare professional or a measuring device. Feedback information may come not from a physiological response as judged by a person but by a sensor or set of sensors (e.g., for measurement of EEG or heart rate). The patient or other person or measurement indicates after a given segment whether the result of the neuromodulation was better or worse than the just previous segment and a numerical estimate, say on a scale of, but not limited to, one to 10. Based on that judgment, the algorithm adjusts the parameters for the following segment of neuromodulation. This approach may only locate the local minimum (or maximum). The algorithm will therefore shift based on a random or planned basis to try another set of parameters. The results will be judged for that given location and if after the applied period of time the results were better at a previous region of the parameter search space the system returns to best parameters from that previous region. The reason that a numerical estimate is given in addition to whether the last neuromodulation condition was better or worse is to cover the case where more than one extrema is being explored. The strategy is changed after m minutes (e.g., every 2 minutes) where m will typically be in the range, but not limited to, approximately 0.5 to approximately 6 minutes.

Examples of symptoms to be judged are shown in TABLE 2. An important consideration is that a strategy to use is to have the patient visualize symptom or effect in question (e.g., pain, anxiety, depression, paranoia, drug craving, food craving, obsessive thinking or memory (e.g., for Alzheimer's disease or cognitive enhancement) and judge whether the visualized. This is true whether Guided-Feedback Neuromodulation or direct feedback is used. In the case of some symptoms, the patient can be prepared to judge by training in the Visual Analog Scale (VAS) to allow them to calibrate their level of pain. Ancillary stimulation as covered elsewhere can be used to contribute to the feedback assessment in addition to the ancillary stimulation to augment the base neuromodulation itself. One application of ancillary stimulation is to apply pain, say with capsaicin, to inject a level of pain whose level is to be judged. Other examples are to excite the level of depression (say a photo of a loved one who was a recent loss), anxiety (photo promoting anxiousness such as a phobia or a person that the patient fears, and Obsessive Compulsive Disorder by displaying a photograph of a door knob or other object that promotes the patient's obsessive-compulsive behavior. One measure of cognitive performance is capabilities in playing video games and another is memory performance.

After n trials in that session, the parameters are maintained for the rest of the session. The successful parameters and strategy used in one session are saved and used at the start of the next neuromodulation session. At a later session (say the third session after the initial session) the search is tried again to see if even a better parameter set can be identified. The session numbers can he selected in the range of approximately 2 to approximately 30, but not limited to that range, with the option to have assistance of a Fibonacci sequence as covered elsewhere. Libbus (U.S. 2008/0051839) uses sensors to detect whether there is a side effect such as cough and then adjust electrical stimulation parameters to minimize the side effect. There is neither direct patient feedback nor Guided Feedback and ultrasound neuromodulation is not included. Foley (U.S. 2005/0240126)) describes the operator monitoring patient condition (e.g., spasticity) or asking the patient (e.g., whether the patient has less spasticity or pain), but Guided Feedback is not included.

In some variations, one effect is used as a surrogate for another effect.

In some variations, the first effect is acute pain and the second effect is chronic pain.

In some variations, Transcranial Magnetic Stimulation coils are used in place of ultrasound transducers.

### Part XI: Ultrasound Neuromodulation for Diagnosis and Other-Modality Preplanning

The embodiments described herein provide improved methods and systems for patient diagnosis or patient treatment planning. The systems and methods may provide non-invasive neuromodulation using ultrasound for diagnosis or treatment of the patient. The systems and methods can be well suited for diagnosing one or more conditions of the patient from among a plurality of possible conditions having one or more similar symptoms. The treatment planning may comprise pre-treatment planning based on ultrasonic assessment with focused ultrasonic pulses directed to one or more target locations of the patient. Based on the evaluation of symptoms or other outcomes in response to targeting a location with ultrasound, the patient treatment at the target location can be confirmed before the patient is treated.

In a first aspect, embodiments provide a method of neuromodulation of a patient. A pulsed ultrasound is provided to one or more neural targets. A neural disorder is identified or treatment is planned for the neural disorder based on a response of the one or more neural targets to the pulsed ultrasound.

In another aspect, embodiments provide a system for neuromodulation. The system comprises circuitry coupled to one or more ultrasound transducers to provide pulsed ultrasound to one or more neural targets. A processor is coupled to the circuitry. The processor is configured to identify a neural disorder or plan for treatment of the neural disorder based on a response of the one or more neural targets to the pulsed ultrasound.

The ultrasound pulses as described herein can be used in many ways. The pulses can be used at one or more sessions to diagnose the patient, confirm subsequent treatment, or treat the patient, and combinations thereof. The pulses can be shaped in one or more ways, and can be shaped with macro pulse shaping, amplitude modulation of the pulses, and combinations thereof, for example.

In same embodiments, the spinal cord can be treated. Target regions in the spinal cord which can be treated using the ultrasound neuromodulation protocols of the present invention comprise the same locations targeted by electrical SCS electrodes for the same conditions being treated, e.g., a lower cervical-upper thoracic target region for angina, a T5-7 target region for abdominal/visceral pain, and a T10 target region for sciatic pain. Ultrasound neuromodulation in accordance with the present invention can stimulate pain inhibition pathways that in turn can produce acute and/or long-term effects. Other clinical applications of ultrasound neuromodulation of the spinal cord include non-invasive assessment of neuromodulation at a particular target region in a patient's spinal cord prior to implanting an electrode for electrical spinal cord stimulation for pain or other conditions.

In many embodiments the ultrasound neuromodulation of the target may include non-invasive assessment of neuromodulation at a particular target neural region in a patient prior to implanting an electrode for electrical stimulation for pain or other conditions as described herein.

In many embodiments, the feasibility of using Deep Brain Stimulation (DBS) is determined for treatment of depression and to test whether depression symptoms can be mitigated with stimulation of the Cingulate Genu. Dramatic results may occur in some patients (e.g., description as having “lifted the void”). Such results, however, may not occur, so neuromodulation of the Cingulate Genu with ultrasound and determining the patient's response can identify those who would benefit from DBS of that target so as to confirm treatment of the Cingulate Genu target.

In many embodiments, the target site for DBS for the treatment of motor symptoms (e.g., bradykinesia, stiffness, tremor) of Parkinson's Disease (PD) comprises the Subthalamic Nucleus (STN). Stimulation of the STN may well have side effects (e.g., problems with speech, swallowing, weakness, cramping, double vision) because sensitive structures are close to it. An alternative target for the treatment of Parkinson's Disease is the Globus Pallidus interna (GPi) which can be effective in motor symptoms as well as dystonia (e.g., posturing and painful cramping). Which of these two targets will overall be best for a given patient depends on that patient and can be determined based on the patient response to DBS. Stimulation of either the GPi or STN improves many features of advanced PD, and even though STN stimulation can be effective, stimulation of the GPi can be an appropriate DBS target to determine whether the STN or GPi should be treated.

In many embodiments, the target comprises the Ventral Intermediate Nucleus of the Thalamus (Vim), which is related to motor symptoms such as essential tremor. In some embodiments, patients with tremor as their dominant symptom benefit from Vim stimulation even though other symptoms are not ameliorated, since such stimulation can deliver the best “motor result.”

In many embodiments, DBS is used on both the STN and the Vim on the same side, such that a plurality of target sites is confirmed and treated.

In many embodiments, ultrasound neuromodulation is used to select the best target for the given patient with the given condition based on testing the results of stimulating different targets. DBS stimulation of each of the potential Parkinson's Disease targets may elicit side effects that are patient specific, for example targets comprising one or more of STN, GPi, or Vim. Alternatively or in combination, ultrasound neuromodulation of the spinal cord can be used to assess whether pain has been relieved and to evaluate the potential effectiveness of or parameters for Spinal Cord Stimulation (SCS) using invasive electrode stimulation.

In many embodiments related to diagnosis and preplanning, patient feedback can be used to adjust ultrasound neuromodulation parameters for at least some conditions as described herein. In some embodiments, ultrasound neuromodulation can be used to retrain neural pathways over time, such that the patient can be treated without constant stimulation of DBS.

Alternatively or in combination with preplanning, ultrasound neuromodulation can be used to diagnosis the patient. In many embodiments, an accurate diagnosis may be difficult with prior methods and apparatus because of the way the disorder manifests itself. In many embodiments, diagnostic the methods and apparatus as described herein provide differentiation between the tremor of Parkinson's Disease and essential tremor. In many embodiments, the tremor of Parkinson's Disease typically occurs at rest and essential tremor does not or is accentuated by movement. An area of confusion is that some patients with Parkinson's Disease have tremor at rest as well.

The methods and apparatus as described herein provide a higher probability of getting the correct diagnosis and can differentiate between essential tremor and the tremor of Parkinson's Disease, such that the patient can be provided with proper treatment. The drug treatments are different for Parkinson's disease and essential tremor. The treatment of Parkinson's Disease in accordance with embodiments comprises treatment with one or more of levodopa, dopamine agonists, MAO-B inhibitors, and other drugs such as amantadine and anticholinergics. The treatment of essential tremor comprises one or more of beta blockers, propranolol, antiepileptic agents, primidone, or gabapentin. The higher probability of getting the right diagnosis can be beneficial with respect to drug treatment in a number of people with essential tremor who may also suffer fear of public situations. In at least some embodiments, medicines used to treat essential tremor may also increase a person's risk of becoming depressed. Embodiments as described herein can improve surgical treatments, as pallidotomy or thalamotomy can be used for either Parkinson's Disease or essential tremor but pallidotomy is generally not effective for essential tremor. The diagnostic methods and apparatus can differentiate between Parkinson's disease and essential tremor, for example when imaging by one or more of CT or MRI scans is insufficient to make a diagnosis. Many embodiments provide the ability to allow the correct selection of therapies selected from among one or more of surgical, neuromodulation, or drug therapies.

While ultrasound neuromodulation can produce acute effects or Long-Term Potentiation (LTP) or Long-Term Depression (LTD), the acute effects are used in many embodiments as described herein. The embodiments as described herein provide control of direction of the energy emission, intensity, frequency (carrier frequency and/or neuromodulation frequency), pulse duration, pulse pattern, mechanical perturbations, and phase/intensity relationships to targeting and accomplishing up-regulation and/or down-regulation. Ancillary monitoring or imaging to provide feedback can be optionally and beneficially combined with the ultrasonic systems and methods as described herein. In many embodiments where concurrent imaging is performed, such as MRI imaging, the systems and methods may comprise non-ferrous material.

In many embodiments, single or multiple targets in groups can be neuromodulated to evaluate the feasibility of treatment and to preplan treatment using neuromodulation modalities, which may comprise non-ultrasonic or ultrasonic modalities, for example. To accomplish this evaluation, in some embodiments the neural targets will be up regulated and in some embodiments down regulated, and combinations thereof, depending on the identified neural target under evaluation. In many embodiments, one or more of PET imaging, fMRI imaging, clinical response to Deep-Brain Stimulation (DBS), or Transcranial Magnetic Stimulation (TMS) can identify the targets.

In many embodiments, the identified targets depend on the patient and the relationships among the targets of the patient. In some embodiments, multiple neuromodulation targets will be bilateral and in other embodiments ipsilateral or contralateral. The specific targets identified and/or whether the given target is up regulated or down regulated, can depend upon the individual patient and the relationships of up regulation and down regulation among targets, and the patterns of stimulation applied to the targets identified for the patient.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and in terms of the cost of administering the therapy.

While ultrasound can be focused down to a diameter on the order of one to a few millimeters (depending on the frequency), whether such a tight focus is required depends on the configuration of the neural target. In order to determine feasibility or preplan treatment by an invasive neuromodulation modality a non-invasive mechanism must be used. Among non-invasive methods, ultrasound neuromodulation is more focused than Transcranial Magnetic Stimulation so it inherently offers more capability to demonstrate the feasibility of and preplan treatment planning for invasive and in many cases highly focused neuromodulation modalities such as Deep-Brain Stimulation (DBS).

For example, described herein are methods of neuromodulation of a patient, the method comprising: providing pulsed ultrasound to one or more neural targets of a neural disorder; and identifying the neural disorder or planning for treatment of the neural disorder based on a response of the one or more neural targets to the pulsed ultrasound.

In some variations, planning for treatment of the neural disorder comprises determining parameters of the pulsed ultrasound in order to confirm a neuromodulation therapy in order to treat the neural disorder based on a response of the one or more neural targets to the parameters.

In some variations, planning for treatment comprises preplanning for a neuromodulation therapy comprising one or more of surgical, invasive neuromodulation, non-invasive neuromodulation, behavioral therapy, or drugs.

In some variations, patient feedback is used to adjust symptoms selected from the group of pain, depression, tremor, voiding from neurogenic bladder; and wherein the symptoms are adjusted based on the one or more neural targets and parameters of the pulsed ultrasound.

In some variations, the identifying the neural disorder comprising differentiating between the tremor of Parkinson's Disease and essential tremor.

In some variations, the planning for treatment comprises identifying a response to neuromodulation of the Cingulate Genu for the purpose of treating depression.

In some variations, planning for treatment comprises identifying a response to neuromodulation of the spinal cord for the purpose of reducing pain.

In some variations, the one or more targets are neuromodulated in a manner selected from the group consisting of ipsilateral neuromodulation, contralateral neuromodulation, and bilateral neuromodulation.

In some variations, the processor comprises instructions to plan for treatment of the neural disorder, including determining parameters of the pulsed ultrasound in order to confirm a neuromodulation therapy in order to treat the neural disorder based on a response of the one or more neural targets to the parameters.

In some variations, the processor comprises instructions to plan for treatment, including preplanning for a neuromodulation therapy comprising one or more of surgical, invasive neuromodulation, non-invasive neuromodulation, behavioral therapy, or drugs.

In some variations, the processor comprises instructions to receive patient feedback in order to adjust symptoms selected from the group of pain, depression, tremor, voiding from neurogenic bladder; and wherein the symptoms are adjusted based on the one or more neural targets and parameters of the pulsed ultrasound.

In some variations, the processor comprises instructions to identify the neural disorder comprising differentiating between the tremor of Parkinson's Disease and essential tremor.

In some variations, the processor comprises instructions to plan for treatment, including identifying a response to neuromodulation of the Cingulate Genu for the purpose of treating depression.

In some variations, the processor comprises instructions to plan for treatment, including identifying a response to neuromodulation of the spinal cord for the purpose of reducing pain.

In some variations, the processor comprises instructions to neuromodulate the one or more targets in a manner selected from the group consisting of ipsilateral neuromodulation, contralateral neuromodulation, and bilateral neuromodulation.

In some variations, the processor comprises instruction to preplan for treatment based on one or more energy sources which is used to treat the neural disorder, the one or more energy sources selected from the group consisting of Transcranial Magnetic Stimulation (TMS) and transcranial Direct Current Stimulation (tDCS).

In some variations, the processor system comprises instructions of an applied feedback mechanism, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, and a subjective patient response.

In some variations, the processor system comprises instructions to preplan for treatment of the neural disorder and wherein the neural disorder comprises one or more of depression, Parkinson's disease, essential tremor, bipolar disorder or spinal cord pain and wherein the target site evaluated prior to treatment comprises one or more of a Cingulate—Genu, DBS, STN, GPi, Vim, Nucleus Accumbens, Area 25 of subcallosal cingulate, one or more levels of a spinal column, white matter or ganglia.

In some variations, the processor system comprises instructions to diagnose the neural disorder and wherein a symptom of the neural disorder comprises one or more of depression, tremor, bipolar behavior or pain and wherein the target site evaluated comprises one or more of Cingulate Genu, DBS, STN, GPi, Vim, Nucleus accumbens, area of 25 of subcallosal cingulate, one or more levels of the spinal column, whiter matter or ganglia.

Work in relation to embodiments as described herein suggests that differences in FUP phase, frequency, and amplitude produce different neural effects. Low frequencies (defined as below approximately 400 Hz but not limited thereto) can be inhibitory in at least some embodiments. High frequencies (defined as being approximately in the range of 500 Hz to 5 MHz but not limited thereto) can be excitatory and activate neural circuits in at least some embodiments. In many embodiments, this targeted inhibition or excitation based on frequency works for the targeted region comprising one or more of gray or white matter. Repeated sessions may result in long-term effects. The cap and transducers to be employed can be preferably made of non-ferrous material to reduce image distortion in fMRI imaging, for example. In many embodiments, if after treatment the reactivity as judged with fMRI of the patient with a given condition becomes more like that of a normal patient, this clinical assessment may be indicative of treatment effectiveness. In many embodiments, the FUP is to be applied 1 ms to 1 s before or after the imaging. Alternatively or in combination, a CT (Computed Tomography) scan can be run to gauge the bone density and structure of the skull, which can be used to determine one or more of the carrier wave frequency, the pulse intensity, the pulse energy, the pulse duration, the pulse repetition rate, or the pulse phase, for a series of pulses as described herein, for example.

Thus, disclosed are methods and systems for non-invasive neuromodulation using ultrasound for diagnosis to evaluate the feasibility of and preplan neuromodulation treatment using other modalities. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, mechanical perturbations, and phase/intensity relationships to targeting and accomplishing up regulation and/or down regulation.

The methods and systems described here for diagnosis and preplanning are applicable to multiple forms neuromodulation.

### Part XII: Treatment Planning for Deep-Brain Neuromodulation

The invention provides methods and systems for treatment planning for non-invasive deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Effectiveness of the application of ultrasound and other non-invasive, non-reversible modalities producing deep-brain neuromodulation such as Transcranial Magnetic Stimulation (TMS), Sphenopalatine Ganglion stimulation, occipital nerve stimulation, peripheral nerve stimulation, transcranial Direct Current Stimulation (tDCS), Radio-Frequency (RF), or functional stimulation can be improved with treatment planning Treatment-plan recommendations for the application of non-reversible and/or invasive modalities such as Deep Brain Stimulation (DBS), stereotactic radiosurgery, optical stimulation, Sphenopalatine Ganglion or other localized stimulation, Vagus Nerve Stimulation (VNS), or future means of neuromodulation can be included.

Ultrasound transducers or other energy sources are positioned and the anticipated effects on up-regulation and/or down-regulation of their direction of energy emission, intensity, frequency, mechanical perturbations, phase/intensity relationships, dynamic-sweep configuration, and timing patterns mapped onto treatment-planning targets. The maps of treatment-planning targets onto which the mapping occurs can be atlas (e.g., Tailarach Atlas) based or image (e.g., fMRI or PET) based. Maps may be representative and applied directly or scaled for the patient or may be specific to the patient.

While rough targeting can be done with one or more of known external landmarks, or the landmarks combined with an atlas-based approach (e.g., Tailarach or other atlas used in neurosurgery) or imaging (e.g., fMRI or Positron Emission Tomography), explicit treatment planning adds benefit.

For example, described herein are methods for treatment planning for neuromodulation of deep-brain targets using ultrasound neuromodulation, the method comprising: setting up sets of applications and supported transducer configurations with associated capabilities, executing treatment-planning sessions including setting parameters for the session, system recommendations and user acceptance of changes to applications, targets, up- or down-regulation, stimulation frequencies, iterating through set of applications; iterating through set of targets; iterating through and applying in designated order one or more variables selected from the group consisting of position, intensity, firing-timing pattern, mechanical perturbations, phase/intensity relationships, dynamic sweeps; presenting treatment plan to user who accepts or changes; whereby the treatment to be delivered is tailored to the patient.

In some variations, the one or plurality of treatment modalities are selected from the group consisting of ultrasound, Deep Brain Stimulation, stereotactic radiosurgery, optical stimulation, Sphenopalatine Ganglion stimulation, other localized stimulation including occipital nerve, Vagus Nerve Stimulation, and future means of neuromodulation.

In some variations, the maps of treatment-planning targets onto which the mapping are selected from the group consisting of atlas based or image based.

In some variations, the maps are selected from the group consisting of specific to the patient, representative and applied directly, and representative where scaled for the patient.

Also described herein are systems for treatment planning for neuromodulation of deep-brain targets using ultrasound or other forms of neuromodulation, the method comprising: setting up sets of applications and supported transducer configurations with associated capabilities, executing treatment-planning sessions including setting parameters for the session, system recommendations and user acceptance of changes to applications, targets, up- or down-regulation, stimulation frequencies, iterating through set of applications; iterating through set of targets; iterating through and applying in designated order one or more variables selected from the group consisting of position, intensity, firing-timing pattern, mechanical perturbations, phase/intensity relationships, dynamic sweeps; presenting treatment plan to user who accepts or changes; whereby the treatment to be delivered is tailored to the patient.

In some variations, the one or plurality of treatment modalities are selected from the group consisting of ultrasound, Deep Brain Stimulation, stereotactic radiosurgery, optical stimulation (including optogenetics), Sphenopalatine Ganglion stimulation, occipital nerve or other localized stimulation, Vagus Nerve Stimulation, and future means of neuromodulation.

In some variations, the maps of treatment-planning targets onto which the mapping are selected from the group consisting of atlas based or image based.

In some variations, the maps are selected from the group consisting of specific to the patient, representative and applied directly, and representative where scaled for the patient.

Thus, disclosed are methods and systems for treatment planning for deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Ultrasound transducers or other energy sources are positioned and the anticipated effects on up-regulation and/or down-regulation of their direction of energy emission, intensity, frequency, firing/timing pattern, mechanical perturbations, and phase/intensity relationships mapped onto the recommended treatment-planning targets. The maps of treatment-planning targets onto which the mapping occurs can be atlas (e.g., Tailarach Atlas) based or image (e.g., fMRI or PET) based. Atlas and imaged-based maps may be representative and applied directly or scaled for the patient or may be specific to the patient.

### Part XIII: Ultrasound Neuromodulation of Spinal Cord

One purpose of this invention to provide methods and systems for neuromodulation of the spinal cord to treat certain types of pain. Such applicable conditions are non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), and refractory ischemic pain (e.g., angina). For pain treatment, the ultrasound energy is targeted to the dorsal column of the spinal cord. In certain embodiments that employ ultrasound neuromodulation, pain is replaced by tingling parathesias. In certain embodiments ultrasound neuromodulation stimulates pain inhibition pathways and can produce acute or long-term effects. The latter can be achieved through long-term potentiation (LTP) or long-term depression (LTD) via training. The other parts of Section I apply to neuromodulation of the spinal cord.

The ultrasound energy may be directed at the same target regions in the spinal cord that have been targeted by electrical spinal cord stimulation. For example, for sciatic pain (typically dermatome level L5-S1), ultrasound stimulation can be directed at T10. For angina, the ultrasound energy can be directed at the lower cervical and upper thoracic region. For the abdominal/visceral pain, the ultrasound can be directed at T5-7. Acute and chronic vasculitis can be treated and associated pain by stimulation of regions of the spinal cord as taught in the literature with regard to SCS (Raso, R. and T. Deer, “Spinal Cord Stimulation in the Treatment of Acute and Chronic Vasculitis: Clinical Discussion and Synopsis of the Literature,” Neuromodulation 14:225-228, 2011).

In addition to pain treatment, ultrasound treatment of the spinal cord according to the present invention can treat other conditions such as refractory overactive bladder (e.g., urgency/frequency and urge incontinence) via sacral neuromodulation or stimulation of a neurogenic bladder to cause emptying.

Another clinical application of the ultrasound treatments of the present invention comprises the reduction of pain caused by functional bowel disorders such as GI visceral pain and irritable bowel syndrome where myeloperoxidase activity is decreased, inflammation is suppressed, and abdominal relax contractions are inhibited. Suitable target regions in the spinal cord are taught in Greenwood Van Meerveld (U.S. Pat. No. 7,251,529) using Spinal Cord Stimulation.

The present invention further includes control of focus, direction, intensity, frequency (carrier frequency and/or amplitude modulation frequency), pulse duration, pulse pattern, mechanical perturbations, and phase/intensity relationships of the ultrasound energy as well as accomplishing up-regulation and/or down-regulation of the target region of the spinal cord. Use of ancillary monitoring or imaging to provide feedback is optional. In embodiments where concurrent imaging is performed, the device of the invention may be constructed of non-ferrous material. Ronald R. Manna (U.S. 2006/0184072) provides for an elongated ultrasound field provided by an elongated transducer that can include an epoxy lens. Manna teaches a High Intensity Focused Ultrasound (HIFU) for tissue ablation and not ultrasound neuromodulation. Klopotek (U.S. Pat. No. 6,113,559) teaches the use of an elongated transducer generating Low Intensity Focused Ultrasound (LIFU), not at depth, for the treatment of skin but does not address neuromodulation. Michael Gertner (U.S. 2011/0092781) uses Low Intensity Focused Ultrasound (LIFU) for imaging to determine treatment location for High Intensity Focused Ultrasound (HIFU) for renal nerve ablation and does not cover elongated transducers not an application to the spinal cord. In like manner, Foley et al. (U.S. 2005/0240126) uses ultrasound imaging to guide nerve ablation using High Intensity Focused Ultrasound rather than neuromodulation and does not teach elongated transducers. Further, Sharkey et al. (U.S. Pat. No. 6,436,129) uses ultrasound stimulation to generate a thermal effect for neural regeneration (e.g., sciatic nerve) and includes elongated stimulation but does not address the spinal cord except that it has nerve cells. Donald Cohen (U.S. 2009/0149782) teaches intersecting beams, but focused on a single target rather than an elongated shape.

The specific targets and/or whether the given target is up regulated or down regulated, can depend on the individual patient and relationships of up regulation and down regulation among targets, and the patterns of stimulation applied to the targets. While ultrasound can be focused down to a diameter on the order of one to a few millimeters (depending on the frequency), whether such a tight focus is required depends on the conformation of the neural target.

In a first aspect of the present invention, a method to alleviate a disease condition comprises aiming at least one ultrasound transducer at a target region of a patient's spinal cord. Pulsed power is applied to the transducer to deliver pulsed ultrasound energy to the target region. The disease condition is usually pain where the target region in the spinal cord is typically within the dorsal column. In specific embodiments, the ultrasound transducer is configured to deliver ultrasound energy having an elongated tubular focus aligned with an axis of the spinal cord. Optionally, the ultrasound will be focused where the focus may optionally be mechanically perturbed to enhance the stimulatory effect of the energy.

In other specific aspects of the methods of the present invention, aiming may comprise aiming a plurality of ultrasonic transducers whose beams intersect at or over the target region. The aiming may alternatively comprise steering a phased array to scan a beam along a segment of the spinal cord. The pulsed ultrasound may provide up-regulation of the target region, e.g. where the ultrasound energy has a modulation frequency of approximately 500 Hz or higher, a pulse duration from approximately 0.1 ms to approximately 20 ms and a repetition frequency of approximately 2 Hz or higher where none of the ranges are limited thereto. Alternatively, the pulsed ultrasound may provide down-regulation of the target region, e.g. where the ultrasound energy has a modulation frequency of approximately 400 Hz or less, a pulse duration from approximately 0.1 ms to approximately 20 ms, and a repetition frequency of approximately 2 Hz or less where none of the ranges are limited thereto. In still other specific aspects of the methods of the present invention, the ultrasound energy provides acute, long-term potentiation of the target region. Alternatively, the ultrasound energy may provide acute, long-term depression of the target region. The methods may further comprise the patient providing feedback as well providing a concurrent therapy selected from the group consisting of transcranial magnetic stimulation (TMS), electrical spinal cord stimulation (SCS), and medication.

The pain disease condition being treated may be selected from the group consisting of non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), refractory pain due to ischemia (e.g. angina), acute vasculitis, chronic vasculitis, hyperactive bladder, and neurogenic bladder.

Dorsal lateral lower motor neurons are associated with the lateral corticospinal tract. Ventromedial lower motor neurons are associated with the anterior corticospinal tract. In an embodiment of the current invention, ultrasound neuromodulation exciting of those motor neurons or their associated tracts results in contractions of the connected muscles. Thus in some embodiments, the ultrasound energy can be employed to restore motor neuron function.

In a second aspect of the present invention, apparatus for delivering ultrasound energy to a target region of a patient's spinal cord comprises an ultrasound transducer assembly and control circuitry and/or supporting structure for delivering ultrasound energy from the transducer assembly to the target region of the spinal cord. The ultrasound energy delivery control circuitry and/or supporting structure preferably focus the ultrasound along a tubular target region aligned with an axis of the spinal cord. The transducer may comprise an elongated transducer having an active surface formed over a partial tubular groove for focusing the ultrasound energy along the tubular target region. The transducer body may consist of a single piezoelectric element or alternatively may include an array of individual transducer elements, e.g. arranged as a phased array for focusing the energy in the tubular focus or other desired focus geometry. The ultrasound transducer may be supported or controlled to mechanically perturb the ultrasound energy, e.g. the ultrasound transducers may be moved to apply mechanical perturbations radially and/or axially. In specifically preferred aspects, the ultrasound transducer and the energy delivery means may be configured to deliver ultrasound energy to the patient's dorsal column for the treatment of pain.

In still other aspects of the present invention, the ultrasound transducer and the energy delivery structure may be configured to deliver ultrasound energy to up-regulate or down-regulate the target region. The ultrasound transducer and the energy delivery control and support structure may be configured to deliver ultrasound energy with a modulation frequency of approximately 400 Hz or less, a pulse duration from approximately 0.1 ms to 20 ms, and a repetition frequency of approximately 2 Hz or less to down regulate the target region where none of the ranges are limited thereto. Alternatively the ultrasound transducer and the energy delivery control and support structure may be configured to deliver ultrasound energy with a modulation frequency of approximately 500 Hz or higher, a pulse duration from approximately 0.1 ms to 20 ms and a repetition frequency of approximately 2 Hz or higher to up regulate the target region where none of the ranges are limited thereto.

The spinal cord can be configurable targeted with ultrasound neuromodulation by shaping the ultrasound field or steering the ultrasound beam (both covered under in Part III above and below) or by mechanical perturbations of the ultrasound transducer as covered in Part IV above and below).

Apparatus of the present invention may be further configured to deliver ultrasound energy that provides long-term potentiation of the target region long-term depression of the target region. Apparatus may further comprise a patient feedback mechanism and may further be combined with system elements for delivering transcranial magnetic stimulation (TMS), electrical spinal cord stimulation (SCS).

In some variations, the disease condition is pain and the target region comprises the dorsal column.

In some variations, the ultrasound transducer is configured to deliver ultrasound energy having an elongated tubular focus aligned with an axis of the spinal cord.

In some variations, the method further comprises mechanically perturbing the ultrasound energy.

In some variations, aiming comprises aiming a plurality of ultrasonic transducers whose beams intersect at or over the target region.

In some variations, aiming comprises steering an ultrasound beam from a phased ultrasound array.

In some variations, the disease treated is selected from the group consisting of non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), refractory pain due to ischemia (e.g. angina), acute vasculitis, chronic vasculitis, hyperactive bladder, and neurogenic bladder.

In some variations, the pulsed ultrasound energy impacts motor neurons.

In some variations, the method further comprises the patient providing feedback.

In some variations, the method further comprises providing a concurrent therapy selected from the group consisting of transcranial magnetic stimulation (TMS), electrical spinal cord stimulation (SCS), and medication.

Also described herein are Apparatuses for delivering ultrasound energy to a target region of a patient's spinal cord, said apparatus comprising: an ultrasound transducer assembly, and means for delivering ultrasound energy from the transducer assembly to the target region of the spinal cord.

In some variations, the ultrasound energy deliver means focuses the ultrasound along a tubular target region aligned with an axis of the spinal cord.

In some variations, the transducer comprises an elongated transducer having an active surface formed over a partial tubular groove for focusing the ultrasound energy along the tubular target region.

In some variations, the transducer body consists of a single piezoelectric element.

In some variations, the transducer comprises a phased array having a length and width that impacts a segment of a spinal cord.

In some variations, the means for delivering ultrasound energy from the transducer assembly to the target region of the spinal cord is configured to mechanically perturb the ultrasound energy.

In some variations, the ultrasound transducers are moved to apply mechanical perturbations radially and/or axially.

In some variations, the ultrasound transducer and the energy delivery means are configured to deliver ultrasound energy to the patient's dorsal column for the treatment of pain.

In some variations, the apparatus further comprises a patient feedback mechanism.

In some variations, the apparatus further comprises a means for delivering transcranial magnetic stimulation (TMS) or electrical spinal cord stimulation (SCS).

One embodiment focuses an elongate tubular ultrasound beam that can be aligned with a target region of the spinal cord.

The methods and systems described here spinal cord stimulation are applicable to all non-invasive forms of neuromodulation.

### Part XIV: Ultrasound Neuromodulation of the Brain, Nerve Roots, and Peripheral Nerves

It is the purpose of this section to provide methods and systems and methods for ultrasound stimulation of the cortex, nerve roots, and peripheral nerves, and noting or recording muscle responses to clinically assess motor function. In addition, just like Transcranial Magnetic Stimulation, ultrasound neuromodulation can be used to treat depression by stimulating cortex and indirectly impacting deeper centers such as the cingulate gyrus through the connections from the superficial cortex to the appropriate deeper centers. Ultrasound can also be used to hit those deeper targets directly. Positron Emission Tomography (PET) or fMRI imaging can be used to detect which areas of the brain are impacted. Compared to Transcranial Magnetic Stimulation, Ultrasound Stimulation systems cost significantly less and do not require significant cooling.

For example, described herein are systems of non-invasively neuromodulating the brain using ultrasound stimulation, the system comprising: aiming an ultrasound transducer at superficial cortex, applying pulsed power to said ultrasound transducer via a control circuit thereby neuromodulating the target, whereby results are selected from the group consisting of functional and diagnostic.

In some variations, the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

In some variations, the level ultrasound stimulation is used to assess the excitability of the cortex. Dawson (U.S. Pat. No. 7,350,522) teaches the use of acoustic signals aimed at cortex to create sensory experiences and mentions pulse shaping, but does not list sound parameters. Use of single-pulse signals to modify nerve excitability is referenced in the patent but determination of cortical excitability is not addressed; the focus is on sensory experience. Johnson (U.S. 2006/0184022) teaches ultrasound imaging of a nerve like the median nerve followed by treatment through heating using ultrasound up to 1.0 w/cm². As to conduction velocity, Johnson does not measure the value directly but estimates through estimating via cross-sectional area. With respect to anesthesia, Johnson teaches about neural function related to carpal tunnel syndrome, but not anesthesia level.

Also described herein are system for non-invasively neuromodulating the brain using ultrasound stimulation, the system comprising: aiming an ultrasound transducer at a neural target, applying pulsed power to said ultrasound transducer via a control circuit thereby stimulating the target, placement of one or a plurality of sensors at a distance from the target, whereby results are selected from the group consisting of diagnostic and monitoring.

In some variations, the plurality of control elements is selected from the group consisting of intensity, frequency, pulse duration, mechanical perturbations, phase/intensity relationships, and firing pattern.

In some variations, the time from stimulation to the time of detection is measured at a sensor where the sensor is placed a location selected from the group consisting of spinal-cord nerve root, peripheral nerve and muscle.

In some variations, the system is used for determination of conduction velocity.

In some variations, the system is used for monitoring of the level of anesthesia.

In some variations, the system is used for monitoring of neural function related to spinal cord surgery.

Also described herein are methods of non-invasively neuromodulating the brain using ultrasound stimulation, the method comprising: aiming an ultrasound transducer at superficial cortex, applying pulsed power to said ultrasound transducer via a control circuit thereby neuromodulating the target, whereby results are selected from the group consisting of functional and diagnostic.

In some variations, the plurality of control elements is selected from the group consisting of intensity, frequency, pulse duration, mechanical perturbations, phase/intensity relationships, and firing pattern.

In some variations, the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

In some variations, the level ultrasound stimulation is used to assess the excitability of the cortex.

Also described herein are methods of non-invasively neuromodulating the brain using ultrasound stimulation, the system comprising: aiming an ultrasound transducer at a neural target, applying pulsed power to said ultrasound transducer via a control circuit thereby stimulating the target, placement of one or a plurality of sensors at a distance from the target, whereby results are selected from the group consisting of diagnostic and monitoring.

In some variations, the time from stimulation to the time of detection is measured at a sensor where the sensor is placed a location selected from the group consisting of spinal-cord nerve root, peripheral nerve and muscle.

Thus, disclosed are methods and systems for non-invasive ultrasound neuromodulation of superficial cortex of the brain or stimulation of nerve roots or peripheral nerves. Such stimulation is used for such purposes as determination of motor threshold, demonstrating whether connectivity to peripheral nerves or motor neurons exists and performing nerve conduction-speed studies. Neuromodulation of the brain allows treatment of conditions such as depression via stimulating superficial neural structures that have connections to deeper involved centers. Imaging is optional.

### Part XV: Ultrasound-Neuromodulation Techniques for Control of Permeability of the Blood-Brain Barrier

It is the purpose of this part to provide methods and systems using non-invasive ultrasound-neuromodulation techniques to selectively alter the permeability of the blood-brain barrier (either brain or spinal cord). Early work at Ben-Gurion University and the University of Rome using Brainsway in Transcranial Magnetic Stimulation (TMS) systems has shown that deep-brain neuromodulation techniques can alter the permeability of the blood-brain barrier to allow more effective penetration of drugs (e.g., for the treatment of malignant tumors). Tumors to which opening of the blood-brain barrier using other techniques has been applied are gliomas, CNS lymphoma and metastatic cancer to the brain. The equipment employed in the current invention also costs less and can be portable for use in a variety of settings, including within the home, work, or school of the patient. Rise (U.S. Pat. No. 6,176,242) combines infusion of a drug with infusion of a drug with Deep Brain Stimulation (DBS). Jolesz et al. (U.S. Pat. No. 5,752,515) teaches modification of the blood-brain barrier using imaging to verify the location of the point where the blood-brain barrier is to be penetrated, but does not elucidate specific targets.

Such neuromodulation can produce acute effects or Long-Term Potentiation (LTP) or Long-Term Depression (LTD). Included is control of direction of the energy emission, intensity, frequency (carrier and/or neuromodulation frequency), pulse duration, firing pattern, mechanical perturbations, and phase/intensity relationships for beam steering and focusing on targets and accomplishing up-regulation and/or down-regulation. Use of ancillary monitoring or imaging to provide feedback is optional. In embodiments where concurrent imaging is performed, the device of the invention is constructed of non-ferrous material.

Multiple targets can be neuromodulated singly or in groups to control the permeability of the blood-brain barrier. To accomplish the treatment, in some cases the neural targets will be up regulated and in some cases down regulated, depending on the given target. The targeting can be done with one or more of known external landmarks, an atlas-based approach or imaging (e.g., fMRI or Positron Emission Tomography).

While ultrasound can be focused down to a diameter on the order of one to a few millimeters (depending on the frequency), whether such a tight focus is required depends on the conformation of the target.

For example, described herein are methods for altering a permeability of a blood-brain barrier in a patient, the method comprising: aiming at least one ultrasound transducer at least one target in a brain or a spinal cord of a human or animal, and energizing at least one transducer to deliver pulsed ultrasound energy to the at least one target, wherein permeability of the blood-brain barrier in the vicinity of the target is altered.

In some variations, the transducer is controlled to deliver ultrasound pulsed power that increases the permeability of the blood-brain barrier.

In some variations, the method further comprises administering a drug to the patient wherein the effectiveness of the drug is enhanced by increased penetration of that drug into the target because of the increase in permeability of the blood-brain barrier.

In some variations, the transducer is controlled to deliver ultrasound pulsed power which decreases the permeability of the blood-brain barrier.

In some variations, the method further comprises administering a drug to the patient wherein the side effects of the drug are reduced due to decreased penetration of the drug into the target because of the decrease in permeability of the blood-brain barrier.

In some variations, a target is selected to have permeability to a drug increased to improve the effectiveness of the drug.

In some variations, a target is selected to have permeability to a drug decreased to protect the target and decrease the side effects of the drug.

In some variations, the ultrasound further provides coincident neuromodulation of a neural target.

In some variations, said at least one of ultrasound transducers delivers a defocused beam to alter the permeability of large volumes of a target in a brain.

Thus, disclosed are methods and systems and methods employing non-invasive ultrasound-neuromodulation techniques to control the permeability of the blood-brain barrier. For example, such an alteration can permit increased penetration of a medication to increase its therapeutic effect. The neuromodulation can produce acute or long-term effects. The latter occur through Long-Term Depression (LTD) and Long-Term Potentiation (LTP) via training. Included is control of direction of the energy emission, intensity, frequency (carrier and/or neuromodulation frequency), pulse duration, firing pattern, mechanical perturbations, and phase/intensity relationships for beam steering and focusing on targets and accomplishing up-regulation and/or down-regulation.

The methods and systems described here for impacting the blood-brain barrier are applicable to all forms of non-invasive neuromodulation.

### Part XVI: Whole Head

This part is directed to non-invasive neuromodulation of the whole head in combination with another neuromodulation means and/or ancillary stimulation to treat conditions were a target or a set of targets is actually involved. The whole-head neuromodulation sets the stage and the additional means of neuromodulation and/or ancillary stimulation makes selections on that stage. Thus the mechanism for treating specific conditions is to provide stimulation of the relevant target or targets though some other means that molds the whole-head neuromodulation of essentially all targets and transforms it into treatment of a specific clinical effect. The same approach can be used with stimulation of the spinal cord.

This is “focusing” the whole-head neuromodulation for a specific purpose. Stimulation of the relevant target or targets can be accomplished by application of ultrasound via one or more ultrasound transducers and/or application of another form of neuromodulation (e.g., adding Transcranial Magnetic Stimulation) and/or ancillary stimulation. This can extend to the simultaneous application of invasive forms of neuromodulation such as Deep Brain Stimulation or optogenetics. In the case of invasive forms, they typically will be neuromodulation a single target due to the cost and risk, but the whole-head approach can positively impact other targets in the relevant neural circuit.

An example of ancillary stimulation is to move the affected limb of a stroke victim when treating stroke with neuromodulation. Another example is the use of ideations like a depressed patient imagining something that lifts their spirit or an anxious patient imagining something that calms them. Other examples of visualize are described above in the section on feedback. Surrogates that impact the relevant target(s) can be used as well such as using pain instead of proprioceptive stimulation for the ancillary stimulation. Ancillary stimulation can be imaging by the patient, say moving a limb or being calm instead of anxious and thus need not be an external ancillary stimulus such as moving a limb.

While ultrasound as a non-invasive neuromodulation modality has the benefit of being more focused than Transcranial Magnetic Stimulation or transcranial Direct Current Stimulation, neuromodulation, in a defocused mode it allows powerful whole-head neuromodulation at a lower cost than say Transcranial Magnetic Stimulation. Whole-head neuromodulation can be accomplished with Transcranial Magnetic Stimulation, Ultrasound Neuromodulation, or Radio-Frequency (RF) neuromodulation. Transcranial Direct Current Stimulation can contribute to whole-head neuromodulation but does not penetrate deeply enough to provide this function alone.

Section I covered optimized neuromodulation, many of the parts of which are applicable to multiple modalities of neuromodulation.

### Section II: Clinical and Physiogical-Impact Applications of Neuromodulation

The following sections describe specific clinical applications of neuromodulation provided by the novel optimized neuromodulation described above including neuromodulation as cover in previous ultrasound and optogenetic patent applications included in this continuation-in-part as individual neuromodulation modalities alone or in combination with other neuromodulation modalities and ancillary stimulation. Multiple targets can be neuromodulated singly or in groups to treat each condition.

TABLE 3

| PART | CONDITION |
|---|---|
| I | Orgasm Elicitation |
| II | Stroke and Stroke Rehabilitation |
| III | Pain |
| IV | Tinnitus |
| V | Depression and Bipolar Disorder |
| VI | Addiction |
| VII | PTSD |
| VIII | Motor (Tremor) Disorders |
| IX | Autism Spectrum Disorders |
| X | Obesity |
| XI | Alzheimer's Disease |
| XII | Anxiety including Panic Disorder |
| XIII | OCD |
| XIV | GI Motility |
| XV | Tourette's Syndrome |
| XVI | Schizophrenia |
| XVII | Epilepsy |
| XVIII | ADHD |
| XIX | Eating Disorders |
| XX | Cognitive Enhancement |
| XXI | Traumatic Brain Injury (TBI) including |
| | Concussion |
| XXII | Compulsive Sexual Disorders |
| XXIII | Emotional Catharsis |
| XXIV | Autonomous Sensory Meridian Response |
| | (ASMR) |
| XXV | Occipital Nerve |
| XXVI | Sphenopalatine Ganglion (SPG) |
| XXVII | Reticular Activating System (RAS) |

### Part I: Orgasm Elicitation

Komisaruk, Whipple, and their colleagues have provided significant information about the correlation between orgasms and imaging for both women and men using vaginal-cervical mechanical self-stimulation (CSS) or imagining in intact women and in other areas where there has been spinal cord injury (Komisaruk B. R. and B. Whipple, “Functional MRI of the brain during orgasm in women,” Annu Rev Sex Res., 16:62-86, 2005 and Komisaruk, B. R., Whipple, B., Crawford, A., Grimes, S., Liu, W.-C., Kalnin, A, and K. Mosier, “Brain activation during vaginocervical self-stimulation and orgasm in women with complete spinal cord injury: fMRI evidence of mediation by the Vagus nerves,” Brain Research 1024 (2004) 77-88, 2004). There is not much difference between the sexual responses of men's and women's brains.

In both women and men, the brain regions that activated (as judged by PET or fMRI scanning) are:
1. Cingulate Gyrus (pain circuit)
2. Insula (pain circuit)
3. Amygdala (regulates emotions)
4. Nucleus Accumbens (controls dopamine release)
5. Ventral Tegmental Area (VTA) (actually releases the dopamine)
6. Hippocampus (memory)
7. Cerebellum (controls muscle function)
8. Paraventricular Nucleus of the Hypothalamus and Pituitary Gland (beta-endorphin release (decreases pain), oxytocin release (increases feelings of trust), and vasopressin (increases bonding)

In women there is activation of the Periaqueductal Gray (PAG) (controlling the “flight or fight” response). The Amygdala and Hippocampus (which deal with fear and anxiety) show decreased activity—perhaps because women have more of a need to feel safe and relaxed in order to enjoy sex. In both women and men, the Left Lateral Orbitofrontal Cortex and the Temporal Lobes shut down during orgasm.

Sexually related sensory signals come from the vagina, cervix, clitoris, and uterus in women. In terms of transmission through nerve distribution:
1. Hypogastric Nerve (uterus and the cervix in women; prostate in men)
2. Pelvic Nerve (vagina and cervix in women; rectum in both sexes)
3. Pudendal Nerve (clitoris in women; scrotum and penis in men)
4. Vagus Nerve (cervix, uterus and vagina (true whether or not the spinal cord is intact)

Women can also have orgasms from stimulation of many parts of their bodies are stimulated (e.g., mouth, the nipples, the anus, hand). In women and men with spinal cord injuries, orgasms have been described when skin is stimulated around the level of the injury because of the heightened sensitivity there. Women can have orgasms without touching their body through imagery alone.

A peripheral orgasm elicitation is known in that in 2004 Dr. Stuart Meloy, an anesthesiologist and pain expert in Winston-Salem, N.C., reported that sacral nerve stimulation with an implanted electrode resulted in an orgasm in ten of eleven women being treated for other conditions (Meloy, T. S. & Southern, J. P. “Neurally Augmented Sexual Function in Human Females: A Preliminary Investigation,” Neuromodulation Volume 9, No. 1 (2006): 34-40), Depression, 1077-1085. Jolesz et al. (U.S. Pat. No. 5,752,515) mentions sexual activity (not mentioning orgasms) as one of the possible indication that a compound has been transferred across the blood-brain barrier, but does not teach elicitation of an orgasm.

It would be desirable to apply ultrasound neuromodulation to the treatment of anorgasmia, hypo-orgasmia, and for the production of orgasms.

It is the purpose of this invention to provide methods and systems for non-invasive deep brain neuromodulation using ultrasound for the treatment of anorgasmia, hypo-orgasmia, and for the production of orgasms. One source of anorgasmia or hypo-orgasmia in men is the impact of treatment for prostate cancer.

### Part II: Stroke

It is the purpose of this invention to provide methods and systems neuromodulation of selected portions of the brain to mitigate the impacts of stroke and foster stroke rehabilitation.

### Part III: Pain

It is the purpose of this invention to provide methods and systems for neuromodulation using ultrasound to treat acute or chronic pain.

### Part IV: Tinnitus

It is the purpose of this invention to provide methods and systems for non-invasive neuromodulation using ultrasound to treat tinnitus. Lenhardt (U.S. 2002/0173697) employs ultrasound for masking tinnitus that is an entirely different than the Applicant's invention using ultrasound to train the neural target, in this case, the Primary Auditory Cortex. Lenhardt teaches embodiments in which the ultrasound is converted to vibration applied just behind the ear that then impacts the cochlea of inner ear or applied to the occipital skull to impact the auditory cortex. This is different than the Applicant's invention that directly impacts the Primary Auditory Cortex rather than doing so via an ultrasound-to-vibration conversion. In either Lenhardt case, masking of the tinnitus is the objective. In his first two embodiments, the amplitude-modulated carrier is set in or swept through the range of approximately 20 kHz to approximately 200 kHz and in the second embodiment from approximately 10 kHz through approximately 200 kHz. In the third embodiment, the frequency range is approximately 200 kHz to approximately 5 MHz with none of the ranges limited thereto. In each case, the amplitude-modulated carrier is multiplied by an audio tone in the range of approximately 1 kHz to approximately 20 kHz (the audible range). Thus it is clear than an audio tone is inherent in Lenhardt's approach, but is not included in the current invention. De Ridder (U.S. 2006/0095090) teaches treating of auditory dysfunction such as tinnitus, hyperacousis, phonophobia, auditory agnosia, auditory hallucinations, and other auditory conditions by electrically stimulating peripheral nerves such as the C2 and C3 dermatome areas comprising cranial nerves, and more specifically the occipital nerve.

### Part V: Depression and Bipolar Disorder

It is the purpose of this invention to provide methods and systems using neuromodulation to treat depression (including Major Depressive Disorder (MDD)) and bipolar disorder. As to treatment, the manic phase is treated with neuromodulation causing down-regulation and the depressive phase is treated with neuromodulation causing up-regulation.

### Part VI: Addiction

It is the purpose of this invention to provide methods and systems using neuromodulation to treat addiction. This includes smoking cessation.

### Part VII: Post Traumatic Stress Disorder (PTSD)

It is the purpose of this invention to provide methods and systems using neuromodulation to treat Post Traumatic Stress Disorder (PTSD).

### Part VIII: Motor Disorders

It is the purpose of this invention to provide methods and systems using neuromodulation to treat motor disorders (e.g., tremor disorders such as Parkinson's Disease and essential tremor).

### Part IX: Autism Spectrum Disorders

It is the purpose of this invention to provide methods and systems using neuromodulation to treat Autism Spectrum Disorders. Such disorders include Autism, Asperger's Syndrome, and Atypical Autism. Sometimes Rett Syndrome and Childhood Disintegrative Disorder are included.

### Part X: Obesity

It is the purpose of this invention to provide methods and systems using neuromodulation to treat obesity.

### Part XI: Alzheimer's Disease

It is the purpose of this invention to provide methods and systems using neuromodulation to treat obesity Alzheimer's Disease and other dementias.

### Part XII: Anxiety Including Panic Disorder

It is the purpose of this invention to provide methods and systems using neuromodulation to treat anxiety including panic disorder.

### Part XIV: GI Motility

It is the purpose of this invention to provide methods and systems using neuromodulation of abdominal and/or pelvic targets to treat gastrointestinal motility disorders, including constipation and diarrhea. It can also be used to treat gastrointestinal-system cramping, including reducing the constriction of GI ducts such as the bile duct and the duct to the gall bladder. Application of the ultrasound neuromodulation can be on the external surface of the body and/or within the GI tract.

Gastrointestinal activity can be assessed objectively by myoelectric activity, measurement of pressure changes, and detection of motion, say by movement of accelerometers. Such sensors can be built in to a neuromodulation device passing through the GI tract, can be placed in a separate sensing device passing through or inserted into the GI tract, or for myoelectric signals can be detected by sensors external to the body such as myoelectric signals captured by electrodes placed on the skin.

### Part XV: Tourette's Syndrome

It is the purpose of this invention to provide methods and systems using neuromodulation to treat Tourette's Syndrome. Also included are the Tourette's vocalizations.

### Part XVI: Schizophrenia

It is the purpose of this invention to provide methods and systems using neuromodulation to treat schizophrenia.

### Part XVII: Epilepsy

It is the purpose of this invention to provide methods and systems using neuromodulation to treat epilepsy.

### Part XVIII: Attention Deficit Hyperactivity Disorder (ADHD)

It is the purpose of this invention to provide methods and systems using neuromodulation to treat Attention Deficit Hyperactivity Disorder (ADHD).

### Part XIX: Eating Disorders

It is the purpose of this invention to provide methods and systems using neuromodulation to treat eating disorders. Such disorders include, but are not limited to, Anorexia Nervosa and Bulimia Nervosa.

### Part XX: Cognitive Enhancement

It is the purpose of this invention to provide methods and systems using neuromodulation to provide cognitive enhancement. Cognitive Enhancement includes such elements as sharpening thinking and memory, facilitating ability to learn, facilitating solving of problems, improved performance in video games, and increased ability to be a warfighter. Cognitive enhancement can be used for mitigation of abnormal conditions such as stroke or for such enhancement in a normal individual. Bystritsky (U.S. Pub. No. 2003/0204135) addresses ultrasound neuromodulation to treat depression, but depression is not a cognitive disease as used in the current invention nor did Bystritsky deal with enhancement of normal cognitive function. Rezai et al. (U.S. PG Pub. No. 2005/0283200) discloses a neurostimulation method in which cognitive capacities including learning and memory are enhanced but uses an electrical stimulation approach without the patterned neuromodulation, Guided Feedback, and other elements of the included invention, or the additional benefits of ultrasound noninvasive neuromodulation.

Multiple targets can be neuromodulated singly or in groups for cognitive enhancement. Cognitive enhancement can be applied for two broad purposes, first that involving cognitive enhancement where cognitive faculties have been diminished (e.g., Alzheimer's Disease, Alzheimer's Disease, Parkinson's disease, Creutzfeld-Jacob disease, Attention Deficit Hyperactivity Disorder, dementia and stroke) and second that involving enhancement of cognitive function in a normal individual. Thus the type of application of cognitive enhancement can be to abnormal function or normal function.

Rezai and Machado (U.S. 2005/0283200) teach the use of electrical stimulation for enhancing memory and learning but do not address ultrasound neuromodulation.

One application of the invention is to provide a tune up to concretize learning for a student studying for a test. This is an example of a tune up for a specific event.

### Part XXI: Traumatic Brain Injury Including Concussion

It is the purpose of this invention to provide methods and systems using neuromodulation to treat Traumatic Brain Injury (TBI), including concussion.

### Part XXII: Compulsive Sexual Disorders

It is the purpose of this invention to provide methods and systems using neuromodulation to treat compulsive disorders.

### Part XXIII: Emotional Catharsis

It is the purpose of this invention to provide methods and systems using neuromodulation to elicit emotional catharsis. Such elicitation depends on triggering of emotion that is most effectively accomplished by neuromodulating the limbic system.

### Part XXIV: Autonomous Sensory Meridian Response (ASMR)

It is the purpose of this invention to provide methods and systems using neuromodulation to elicit Autonomous Sensory Meridian Response (ASMR).

### Part XXV: Occipital Nerve

It is the purpose of this invention to provide methods and systems using neuromodulation of the occipital nerve to treat pain and other disorders. Transcranial Magnetic Stimulation (TMS) has been successfully used in occipital nerve stimulation for migraine headache and other headaches. Implanted electrical stimulation such as Jaax, Whitehurst, Carbunaru, and Makous (U.S. Pat. No. 6,735,475), but this uses an invasive technique

Electrical stimulation, including autonomic nervous system stimulation, has been associated with treatment of headaches and associated symptoms such as nausea and vomiting. A variety of non-invasive treatments have been used for headache treatment such as medication, diet, trigger avoidance, acupuncture, anesthetic agents, biofeedback, and physical therapy. Invasive treatments have been used as well such as ganglion resection, ganglion block, radiosurgery, and cryotherapy. Electrical stimulation has been applied by implanted electrodes or implanted stimulator. A stimulator can be set to deliver a predetermined pattern of stimulation, or the patient may control the amplitude, pulse width, and frequency using a remote-control device.

Such stimulation has also been associated with the treatment of a number of other conditions including neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

Many of the sensory and motor nerves of the neck are contained in C2 and C3, including the Greater Occipital Nerve (GON) and these have been stimulated for treatment of headaches such as migraine, cluster, and hemicrania continua. Blocks of the occipital nerve have had success in treatment of headache in its various forms. An important aspect is that positive effect of the treatment outlasts the impact of the neural block. This indicates that there is some longer-term neuromodulation. Such blocks, while effective in a majority of cases, are not always predictive of whether longer-term occipital nerve electrical stimulation will be successful. In some cases, there is a delayed effect (which may be two to six months and may involve the patient's symptoms getting worse before they get better) so a short-term trial stimulation does not mean longer-term stimulation will not be successful. The length of time to achieve therapeutic effect means that the mechanism of impact involves neural plasticity. Also that anterior-pain symptoms decrease as well as posterior-pain symptoms indicates that a central mechanism is involved. In addition, for hemicrania continua, pain remediation may be separate from autonomic symptoms such as rhinorrhea and tearing excess that can remain after headache symptoms decrease. Meningeal and Greater Occipital Nerve inputs come together, not peripherally but centrally at the second-order neuron in the spinal cord indicating involvement of the caudal trigeminal nucleus and the upper cervical segments and suggesting a mechanism for referred pain.

A suggested mechanism for the etiology of headache is sensitization of the brainstem because of the sensory input from the occipital nerve causing altered neural processing.

For the treatment of migraine and cluster headaches and other conditions, it would be of benefit to apply a non-invasive treatment modality. As indicated by previous work noted above for electrical stimulation, the positive effect of treatment, so that in addition to any acute positive effect, there will be a long-term “training effect” with Long-Term Depression (LTP) and Long-Term Potentiation (LTD) depending on the central intracranial targets to which the occipital nerve is connected.

The invention can be applied to a number of conditions including headaches in various forms, migraine headaches in various forms, cluster headaches in various forms, neuralgias, facial, and other pain or tension syndromes.

Kovacs et al. (Kovacs, S. Peeters, R., De Ridder, D., Plazier, M., Menovsky, T. and S. Sunaert, “Central Effects of Occipital Nerve Electrical Stimulation Studied by Functional Resonance Imaging,” Neuromodulation: Technology at the Neural Interface, Vol 14, Issue 1, pages 46-57, January/February 2011, Article first published online: 7 Dec. 2010 DOI: 10.1111/j.1525-1403.2010.00312.x) applied electrical stimulation of the occipital nerve and looked at the impact on neural structures as determined through fMRI. As shown in the fMRI, major areas of activation were the hypothalami, the thalami, the orbito-frontal cortex, the prefrontal cortex, periaqueductal gray, the inferior parietal lobe, and the cerebellum. As to deactivation, the major areas were in the primary motor area (M1) the primary visual area (V1), the primary auditory area (A1), and the somatosensory (S1), the amygdala, the paracentral lobule, the hippocampus, the secondary somatosensory area (S2), and the supplementary motor area (SMA). Ultrasound neuromodulation provided by the current invention would have activate and deactivate the same structures and thus can provide therapeutic effects related to the neuromodulation of those structures.

### Part XXVI: Sphenopalatine Ganglion

It is the purpose of this invention to provide methods and systems using neuromodulating the Sphenopalatine Ganglion.

While Transcranial Magnetic Stimulation (TMS) is an effective means of non-invasive neuromodulation when used intracranially, current systems have delivered footprints that are too large for neural structures like the Sphenopalatine Ganglion. Ultrasound can be focused to approximately 0.5 to 2 mm while TMS can be focused to 1 cm at best. Also, if TMS were used to stimulate the Sphenopalatine Ganglion there would be intolerable side effects such local muscle stimulation, and, in some cases stimulation of other nerves.

Sphenopalatine Ganglion and other autonomic nervous system stimulation has been associated with treatment of headaches and associated symptoms such as nausea and vomiting. A variety of non-invasive treatments have been used for headache treatment such as medication, diet, avoidance of triggers, acupuncture, anesthetic agents, biofeedback, and physical therapy. Invasive treatments have been used as well such as ganglion resection, ganglion block, radiosurgery, and cryotherapy. In addition, electrical stimulation has been applied by implanted electrodes or implanted stimulator.

Such stimulation has also been associated with the treatment of a number of other conditions including neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

In addition, stimulation of the Sphenopalatine Ganglion has been described for modification the properties of the Blood-Brain Barrier (BBB) and cerebral blood flow.

The Sphenopalatine Ganglion is a parasympathetic ganglion the largest of the parasympathetic ganglia associated with the branches of the trigeminal nerve. Stimulation of the Sphenopalatine Ganglion (SPG) for a number of maladies has been addressed previously. Examples are Pless (B.D. Pless, “Method and Device for the Treatment of Headache,” U.S. Patent Application Pub. No. 2009/0276005), Yun and Lee (Yun, A. J., and P. Y-b Lee, “Treatment of conditions through modulation of the autonomic nervous system,” U.S. Pat. No. 7,363,076), and Ansarinia (Ansarinia, M. M., “Stimulation Method for the Sphenopalatine Ganglia, Sphenopalatine Nerve, or Vidian Nerve for Treatment of Medical Conditions,” U.S. Pat. No. 6,526,318, Feb. 25, 2003). It would desirable to have neuromodulation of the SPG and related structures without using invasive means such as implanted electrodes.

### Part XXVII: Reticular Activating System

It is the purpose of this invention to provide methods and systems for neuromodulating the Reticular Activating System (RAS).

The above material summarizes the inventions for both optimized neuromodulation for the various neuromodulation modalities and the application of those optimized methods and devices to the treatment of specific clinical conditions or achievement of physiological effects.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the characteristics of the various neuromodulation modalities.

FIG. 2 is a table of Indications versus Targets.

FIG. 3 shows a table for Therapeutic-Modality Combinations for Selected Indications.

FIG. 4 shows the physical layout of the combination of therapeutic modalities for the treatment of pain.

FIG. 5 shows a block diagram of the treatment planning and control system.

FIG. 6 illustrates the flow of the treatment planning and control system.

FIGS. 7A-7C show top and frontal views of the track around the head on which transducers run.

FIGS. 8A-8D illustrate the frontal and side views of an example of the transducer with its hemispheric ultrasound array.

FIG. 9 shows an alternative embodiment in which the transducer is rotated while it is going around the track.

FIG. 10 illustrates an embodiment in which the apparatus is enclosed within a shell.

FIG. 11 shows a block diagram of the control circuit.

FIG. 12 shows an alternative block diagram of a control circuit that incorporates feedback.

FIG. 13 illustrates targeting multiple targets in a neural circuit for addiction.

FIG. 14 demonstrates using a patient-specific holder to fix the transducers relative to the targets.

FIG. 15 shows an embodiment where the transducers can be moved in and out for refined patient-specific targeting.

FIG. 16 shows an embodiment where the transducers can be moved in and out for automatically adjusting refined patient-specific targeting.

FIGS. 17A-17C show an ultrasound transducer configured to produce an elongated pencil-shaped focused field.

FIG. 18 illustrates the elongated ultrasound transducer array with sound conduction medium.

FIG. 19 shows physical target layout for addiction.

FIGS. 20A-20C demonstrate two ultrasound transducer arrays with different radii.

FIGS. 21A-21C demonstrate flat transducer array with interchangeable lenses.

FIGS. 22A-22B show a linear ultrasound phased array with a steered-beam linearly moving field.

FIGS. 23A-23B demonstrates the combination of ultrasound transducer with TMS Coil.

FIGS. 24A and 24B show the mechanism for mechanical perturbations and examples the resultant ultrasound field shapes.

FIG. 25 shows a flat ultrasound transducer producing a parallel beam.

FIG. 26 shows three flat ultrasound transducers using global ultrasound conduction medium with beams intersecting on a Dorsal Anterior Cingulate Gyrus (DACG) target.

FIG. 27 shows three flat ultrasound transducers using individual ultrasound conduction media with beams intersecting on a Dorsal Anterior Cingulate Gyrus (DACG) target.

FIG. 28 shows two sets of flat ultrasound transducers using global ultrasound conduction medium with beams intersecting on Dorsal Anterior Cingulate Gyrus (DACG) and Insula targets.

FIG. 29 shows a block diagram of the mechanism for controlling the multiple ultrasound beams.

FIGS. 30A-30D show diagrams of macro-pulse shaping.

FIGS. 31A-31C show diagrams of micro-pulse shaping.

FIG. 32 shows a block diagram of the system for generating the output incorporating macro- and micro-pulse shaping.

FIG. 33A-33B illustrate sine-shaped Intensity-Modulated Pulsing.

FIG. 34A-34B illustrate ramp-shaped Intensity-Modulated Pulsing.

FIGS. 35A-35F illustrate a table of neuromodulation patterns.

FIG. 36 illustrates the neural circuit allowing alternative effects depending on whether the circuit is up regulated or down regulated.

FIG. 37 shows application of a Fibonacci Sequence pulse pattern.

FIG. 38 illustrates a Burst-Mode pulse pattern.

FIG. 39 illustrates simultaneous delivery of two neuromodulation frequencies.

FIG. 40 shows swept neuromodulation frequency.

FIG. 41 shows swept pulse frequency.

FIGS. 42A-C illustrate the swept pulse duty cycle.

FIG. 43 demonstrates Cumulative Energy Delivery.

FIG. 44 illustrates a circuit for Ancillary Stimulation.

FIGS. 45A-45E show a diagram of exemplar session types for both initial treatment and maintenance sessions.

FIG. 46 shows a block diagram of a Feedback Control Circuit, in accordance with embodiments.

FIG. 47 shows a multi-target configuration for treatment of pain using feedback.

FIG. 48 shows a diagram of an algorithm for processing patient feedback to control neuromodulation.

FIG. 49 illustrates application of the Hill Climbing Algorithm for Guided Feedback Neuromodulation.

FIG. 50 shows a flow chart for the application of Guided Feedback Neuromodulation.

FIG. 51 illustrates an overall block diagram for Guided Feedback Neuromodulation.

FIG. 52 shows ultrasound-transducer targeting of the STN and the GPi to test the feasibility of using DBS for treatment of Parkinson's Disease, in accordance with embodiments.

FIG. 53 shows targeting of the Cingulate Genu to test the feasibility of using DBS for the treatment of Depression, in accordance with embodiments.

FIG. 54 demonstrates ultrasound neuromodulation of the spinal cord to test the feasibility of using Spinal-Cord Stimulation (SCS) for the treatment of neuropathic or ischemic pain, in accordance with embodiments.

FIG. 55 illustrates a method and steps for preplanning, in accordance with embodiments.

FIG. 56 illustrates a method and steps for diagnosis, in accordance with embodiments.

FIG. 57 shows a block diagram of an apparatus to one or more of diagnose or treat the patient, in accordance with embodiments.

FIG. 58 shows a block diagram of treatment planning.

FIG. 59 illustrates an exemplary pain-target configuration to which treatment planning is applied.

FIG. 60 shows a graphic displayed to healthcare professional after treatment planning to guide treatment.

FIG. 61 illustrates the treatment-planning algorithm.

FIG. 62 shows ultrasound-transducer targeting of the spinal cord from the perspective view of the spinal column.

FIG. 63 shows ultrasound-transducer targeting of the spinal cord from the cross-section view of the spinal column.

FIG. 64 shows ultrasound transducers and EMG sensors at various portions of the nervous system.

FIG. 65 shows exemplar blood-brain barrier targets on which ultrasound is focused.

FIG. 66 illustrates an embodiment for whole-head neuromodulation.

FIG. 67 shows set-up in the non-imaging phase for Orgasm Elicitation.

FIG. 68 shows set-up imaging without targeting phase for Orgasm Elicitation

FIG. 69 shows set-up imaging with targeting phase for Orgasm Elicitation.

FIG. 70 illustrates Orgasm Elicitation utilization.

FIG. 71 illustrates the Primary Motor Cortex related to stroke.

FIG. 72 shows an ultrasound transducer array over the Primary Motor Cortex.

FIG. 73 illustrates gastrointestinal lumen.

FIG. 74 shows set of gastrointestinal organs that can be neuromodulated.

FIG. 75 shows a feedback control diagram for neuromodulation of the GI tract.

FIG. 76 illustrates a diagram of elements to abort epileptic seizures.

FIGS. 77A-77F show configurations for neuromodulation of the occipital nerve.

FIG. 78 illustrates the anatomy of the location of the occipital nerves.

FIGS. 79A-79E show a frontal view of a configuration for neuromodulation of the Sphenopalatine Ganglion.

FIG. 80 illustrates the configuration of nerves surrounding the Sphenopalatine Ganglion.

FIG. 81 shows anatomical relationships related to the Sphenopalatine Ganglion.

FIGS. 82A-B illustrate the configuration for the neuromodulation of the Reticular Activating System.

FIG. 83 shows the configuration of FIG. 82 viewed from the top of the patient head.

### DETAILED DESCRIPTION OF THE INVENTION

Described herein are methods, systems, and devices of neuromodulation including optimization thereof. Each of the sections below describes different aspects, devices, methods, and systems directed to neuromodulation and associated techniques. References to “the invention” may refer to one of the various inventions described herein; elements of one of the inventions need not be incorporated or necessary for other inventions but may be included, as applicable.

Certain elements are common to all the ultrasound elements of inventions and will not be repeated in all the sections. The common material includes the following. Ultrasound is acoustic energy with a frequency above the normal range of human hearing (typically greater than 20 kHz). The stimulation of deep-brain structures with ultrasound has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton describes a strong DC magnetic field as well and describes the mechanism as that given that the tissue to be stimulated is conductive that particle motion induced by an ultrasonic wave will induce an electric current density generated by Lorentz forces, such that ultrasound is suitable for combination with TMS in accordance with embodiments as described herein.

Different elements are combined in ultrasound neuromodulation as shown in TABLE 4.

TABLE 4

| | | | Typical |
|---|---|---|---|
| | | | (approximate, |
| | | Range (approximate, | but not limited |
| Element | Definition | but not limited to) | to) |
| (Acoustic) Carrier | Base Frequency | .3 MHz to .85 MHz | .65 MHz |
| Frequency | allowing penetration | | |
| | through skull, spinal | | |
| | cord; can also work | | |
| | in soft tissue | | |
| Neuromodulation | Amplitude or | 300 Hz to 5 MHz | 400 Hz or less |
| Frequency | Frequency | | for |
| | Modulation | | inhibition/down |
| | impacting neural | | regulation; 500 Hz |
| | structures | | or greater for |
| | | | excitation/up |
| | | | regulation |
| Pulse Frequency | Monopolar or | .1 msec to 2 sec in | .2 ms pulses at 2 Hz |
| | bipolar gating of | length at .5 Hz to 50 Hz | or less for |
| | Neuromodulation | repetition | inhibition/down |
| | Frequency | | regulation; 5 Hz |
| | | | or greater for |
| | | | excitation/up |
| | | | regulation |

For all inventions covered herein sets of endpoints within the approximate ranges listed in TABLE 4 or otherwise covered in this application or outside those approximate ranges are covered. In these inventions, the ultrasound acoustic carrier frequency is in range of approximately but not limited to 0.3 MHz to 0.8 MHz to permit effective transmission through the skull with power generally applied less than 180 mW/cm² but also at higher target- or patient-specific levels at which no tissue damage is caused. The acoustic carrier frequency (e.g., 0.44 MHz) is amplitude modulated by a lower frequency called here the neuromodulation frequency to impact the neuronal structures as desired (typically 400 Hz for inhibition (down-regulation) or 500 Hz and up for excitation (up-regulation) depending on the target, condition, and patient. The stimulation frequency for excitation is in the range of approximately but not limited to 500 Hz to 5 MHz. There are not sharp borders at 400 and 500 Hz, however. The neuromodulation frequency (superimposed on the carrier frequency of say 0.5 MHz or similar) may be divided into pulses approximately but not limited to 0.1 to 20 ms. repeated at frequencies of approximately but not limited to 2 Hz or lower for down regulation and higher than approximately but not limited to 2 Hz for up regulation) although again this will be target, condition, and patient specific. Either monopolar or bipolar pulses may be used and continuous neuromodulation can be used as well. In one embodiment, frequency modulation is used for neuromodulation instead of amplitude modulation.

If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency. For larger targets, larger spot sizes will be used and, depending on the shape of the targeted area, different shapes of ultrasound fields will be used.

In an embodiment of the invention, the acoustic carrier frequency is modulated (neuromodulated) so as to impact the neuronal structures as desired (e.g., say typically 400 Hz or lower for inhibition (down-regulation) or 500 Hz or higher, up to 5 MHz for excitation (up-regulation), for example). In many embodiments, the neuromodulation frequency may be divided into pulses 0.1 to 20 ms, and the modulation frequency may be superimposed on the ultrasound carrier frequency, which can be about 0.5 MHz, for example. In an embodiment, the pulses are repeated at frequencies of 2 Hz or lower for down regulation and higher than 2 Hz for up regulation although this will be both patient and condition specific.

The number of ultrasound transducers can vary between one and five hundred. Keramos-Etalon can supply a known commercially available 1-inch diameter ultrasound transducer and a focal length of 2 inches that will deliver a focused spot with a diameter (6 dB) of 0.29 inches with 0.4 MHz excitation. In many embodiments, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer) (Fleury G., Bernet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of sound transducers of 300 or more. Keramos-Etalon and Blatek in the U.S. are other custom-transducer suppliers. The power applied will determine whether the ultrasound is high intensity or low intensity (or medium intensity) and because the sound transducers are custom, any mechanical or electrical changes can be made, if and as required.

Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well. Ultrasound conduction medium will be required to fill the space where the transducer is not directly in contact with the skin.

The locations and orientations of the transducers in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. The ultrasonic firing patterns can be tailored to the response type of a target or the various targets hit within a given neural circuit.

Ultrasound therapy can be combined with therapy using other devices (e.g., Transcranial Magnetic Stimulation (TMS), Sphenopalatine Ganglion stimulation, occipital nerve stimulation, peripheral nerve stimulation, transcranial Direct Current Stimulation (tDCS), and/or Deep Brain Stimulation (DBS) using implanted electrodes, Spinal Cord Stimulation using implanted electrodes, Vagus Stimulation, implanted optical stimulation (optogenetics), stereotactic radiosurgery, Radio-Frequency (RF)), other local stimulation, or functional stimulation, behavioral therapy, or medications.

### Section I: Optimized Neuromodulation

### Part I: Multi-Modality Neuromodulation of Brain Targets

It is the purpose of some of the inventions described to provide methods and systems and methods for deep brain or superficial stimulation using multiple therapeutic modalities to impact one or multiple points in a neural circuit to produce Long-Term Potentiation (LTP) or Long-Term Depression (LTD). Some of the modalities (e.g., TMS) will cause training or retraining to bring about long-term change. Radiosurgery (or a surgical ablation) on the other hand will cause a permanent effect and DBS must remain applied or the effect will terminate. Such permanent changes usually will result in down-regulation. Another consideration is that in some cases one does not need a terribly long-term effect such as the application of one or more reversible non-invasive modalities for treatment of an acute condition such as acute pain related to a dental procedure or outpatient surgery.

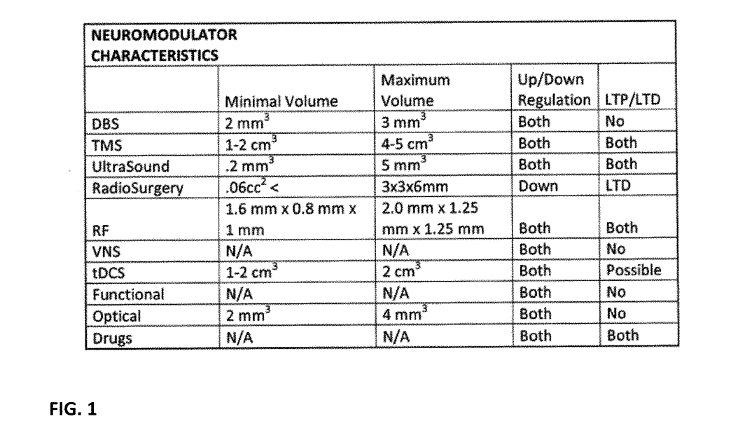

FIG. 1 shows the characteristics of the various neuromodulation modalities. The values for the parameters are approximate and not meant to be absolute. Which treatment modality is to be used in what position for what target depends on such factors as the size of the target (e.g., ultrasound can be focused to 0.5 to 2 mm³ while TMS can be limited to 1-2 cm³ at best), target accessibility, the presence of critical neural structures for which stimulation is to be avoided in proximity to the target, whether side effects will be elicited, local characteristics of the neural tissue (e.g., tDCS can only be used on superficial targets, DBS is not applicable to structures like the Insula that have a high degree of vascularity), whether up or up regulation is to be performed, whether Long-Term Potentiation (LTP) or Long-Term Depression (LTD) is desired, and whether there is physically enough room for the physical combination of neuromodulation elements. Another critical element is whether an invasive modality (e.g., DBS, VNS, optical) is acceptable or not. It is to be noted that radiosurgery can only down-regulate. A fundamental consideration of this invention that a given target may best targeted by one or a set of modalities. For example, a long structure like the DACG may be amenable to deep-brain TMS stimulation while a relatively small target such as the Nucleus Accumbens may be best targeted by DBS. Another consideration is that as the overall clinical therapeutic approach develops, one or more additional modalities may be considered at the point where one or more modalities are already in place. The principles of this invention are important and the invention is not limited to the currently available modalities, because existing techniques will be improved, new techniques will be discovered, and additional targets for given indications will be identified.

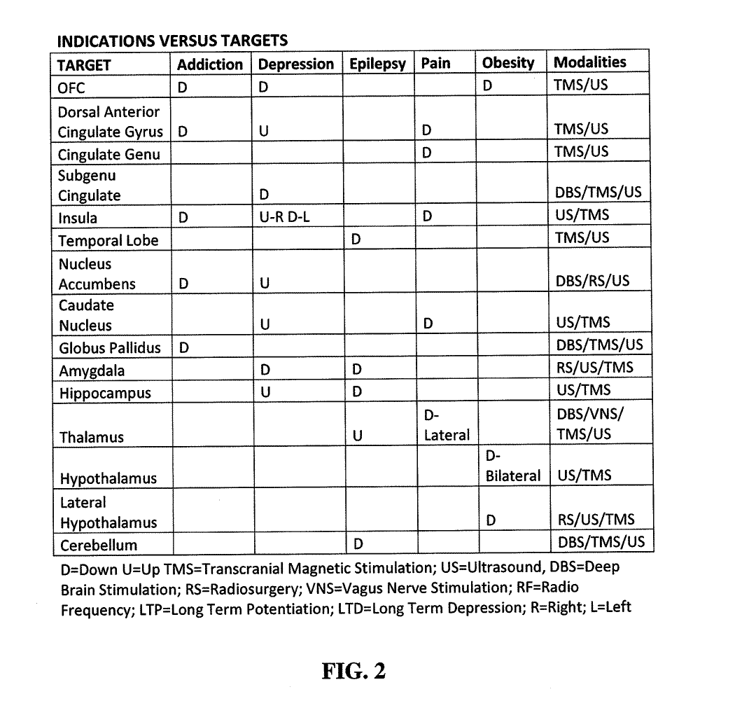

FIG. 2 is a table of Indications versus Targets. Many of these are shown on brainmaps.com. Not all targets for each indication is listed, only the main ones according to current understanding. As additional knowledge is discovered targets or which modality is or modalities are preferable may change. Not all the targets listed need to be hit for treatment to be effective. The entries in each of the indication columns represent either down-regulation (D) or up-regulation (U) for that given target for that indication. Not all targets will be regulated one way or the other for all indications. For example, the Dorsal Anterior Cingulate Gyms (DACG) is up regulated for depression and down regulated for addiction and pain. Likely modalities are listed in the last column of the table. While there may be some preference for the order listed for a given modality according to one judgment the order is by no means mandatory. In some cases, the most effective combination may even be patient specific. In addition, it is possible that other modalities could be used effectively either instead of, or perhaps in addition to a listed modality. Depending on the target set, it may be that using a single modality may also work. An important consideration is that even though many targets are available, in practice one would not necessarily choose to hit all the targets but might well choose a subset. In some cases, there may be too many targets to permit all too be targeted so choices will need to be made. In other cases, it might be possible to set up a combined mechanism to hit all the targets, but it may be too expensive to do so relative to additional benefit to be obtained. In any case, new targets may be discovered as more knowledge is developed.

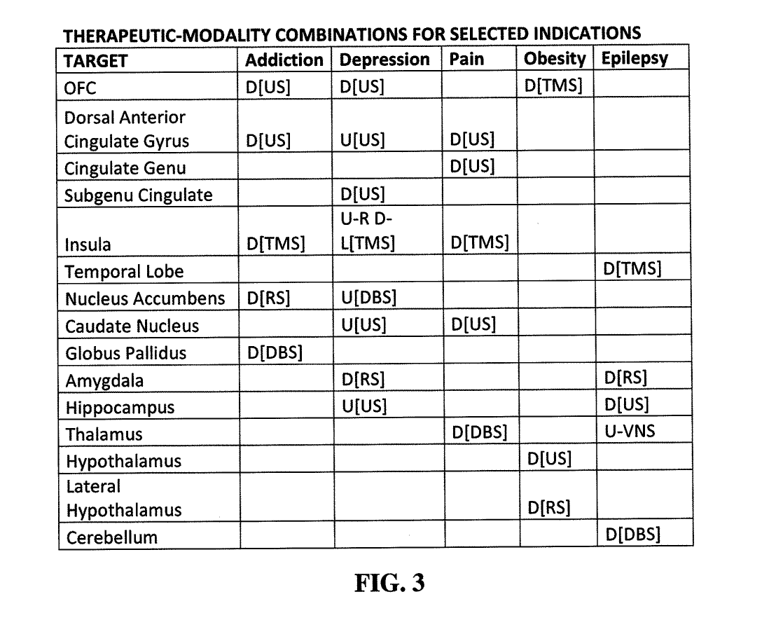

FIG. 3 shows a table for Therapeutic-Modality Combinations for Selected Indications. These represent one combination for each of the five covered indications, pain, depression, addiction, obesity, and epilepsy. The entries in each of the indication columns represent either down-regulation (D) or up-regulation (U) for that given target for that indication plus the particular therapeutic modality to be used. An important consideration is the physical space required for each of the energy sources. In some cases moving them off to a different plane and/or orientation may allow tighter packing.

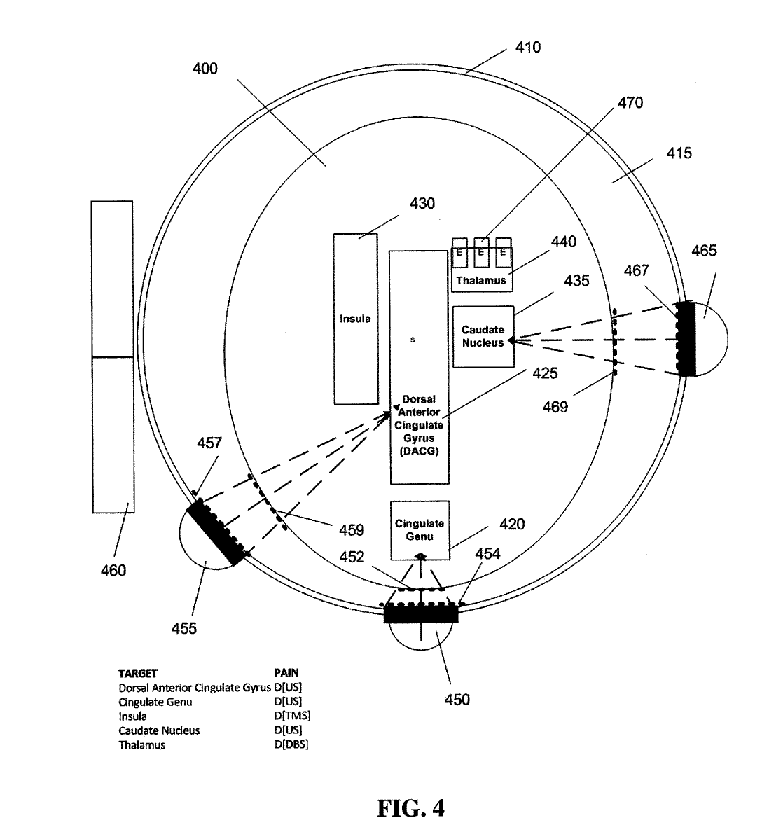

FIG. 4 shows the physical layout of the combination of therapeutic modalities as listed in the table of FIG. 3 for the treatment of pain. The entries from that table just for pain are shown in the lower left-hand corner of the figure for reference. A frame 410 for holding energy sources surrounds head 400. The targets Cingulate Genu 420 neuromodulated by ultrasound transducer 450, Dorsal Anterior Cingulate Gyms (DACG) 425 neuromodulated by ultrasound transducer 455, Insula 430 neuromodulated by TMS coil 460, Caudate Nucleus 435 neuromodulated by ultrasound source 465, and Thalamus 440 neuromodulated by DBS stimulating electrodes 470 are illustrated. In the case of ultrasonic transducers, the space between frame 410 and head 400 is filled with an ultrasonic conduction medium 415 such as Dermasol from California Medical Innovations with the interfaces between the head and the ultrasonic conduction medium and the ultrasonic medium and the ultrasound transducer are provided by layers of ultrasonic conduction gel, 452 and 454 for ultrasound transducer 450, 457 and 459 for ultrasound transducer 455, and 467 and 469 for ultrasound transducer 465. Note that while specific modalities for the targets are given, appropriate substitutions (i.e., target appropriate to modality, modality physically will fit with the mechanism for the other targets, etc.) can be made. Also, alternative targets to treat a given indication may be appropriate. The preceding points, while included on this section of pain, apply to the indications covered in the following paragraphs and other indications as well. For any of the indications the positions and orientations of the energy sources are set according to the particular needs of the targets and physical configuration. In another embodiment, more than one modality can be used to hit a single target to increase the effect. For example, both ultrasound and TMS could be used to simultaneously or sequentially hit the Dorsal Anterior Cingulate Gyrus.

Note that where bilateral targets for any indication exist, both sides could be stimulated in other embodiments if the neuromodulation elements can be physically accommodated. Some embodiments may incorporate sequential rather than simultaneous application of on-line, real-time modalities such as ultrasound and TMS. In still other embodiments, multiple indications can be treated simultaneously or sequentially.

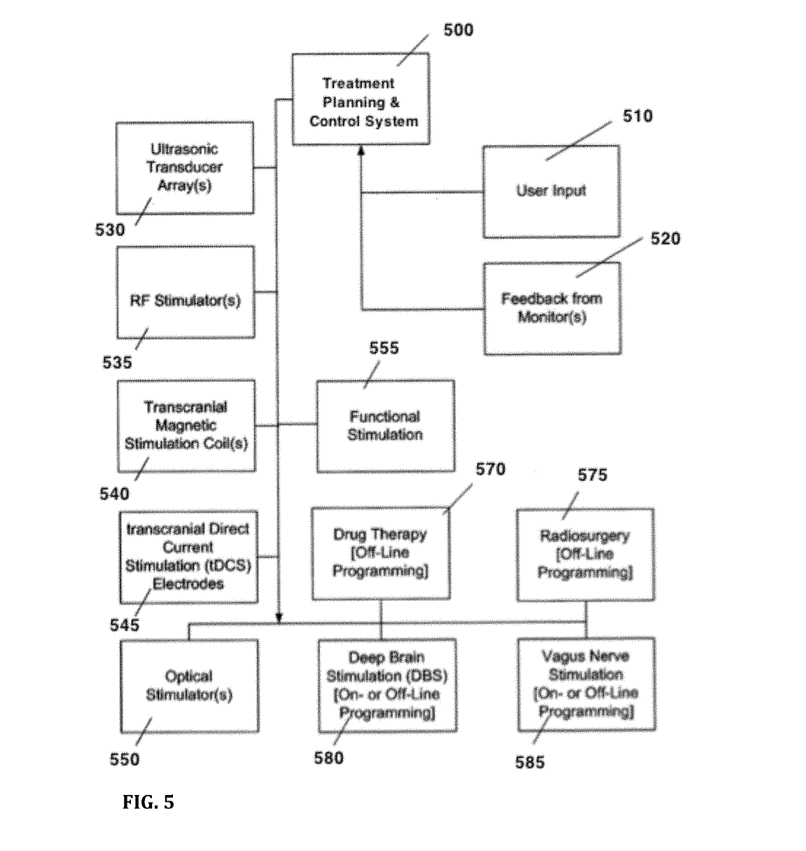

The imaging can be done as a one-time set-up or at each session although not using imaging or using it sparingly is a benefit, both functionally and the costs of administering the therapy, over approaches like Bystritsky (U.S. Pat. No. 7,283,861) which teaches consistent concurrent imaging. A block diagram is shown in FIG. 5 that depicts the Treatment Planning and Control System that has inputs from the user and monitoring systems (e.g., energy levels for one or more therapeutic modalities and imaging) and outputs to the various modalities. The treatment planning and control system varies, as applicable, the direction of energy emission, intensity, session duration, frequency, pulse-train duration, mechanical perturbations, phase/intensity, firing patterns, numbers of sessions, and relationship to other controlled modalities. Use of ancillary monitoring or imaging to provide feedback is optional. Treatment Planning and Control System 500 receives input from User Input 510 and Feedback from Monitor(s) 520 and provides control output (either real-time or instructions for programming) to Transducer Array(s) 530, RF Stimulator(s) 535, Transcranial Magnetic Stimulation Coil(s) 540, transcranial Direct Current Stimulation (tDCS) Electrodes 545, Optical Simulator(s) 550, Functional Stimulation 555, Drug Therapy 570 [Off-Line Programming], Radiosurgery 575 [Off-Line Programming], Deep Brain Stimulation (DBS) 580 [On- or Off-Line Programming], and Vagus Nerve Stimulation (VNS) 585 [On- or Off-Line Programming] There are four categories of output modalities:

a) on-line-real-time where neuromodulation parameters are changed immediately under direct control of the Treatment Planning and Control System (e.g., ultrasound transducers or TMS stimulators),

b) on-line-prescriptive where neuromodulation parameters are directly set in programmers (e.g., DBS or Vagus Nerve Stimulation programmers) and the effect is both reversible and seen immediately,

c) off-line-prescriptive-adjustable where instructions are generated for users to adjust drug dosages or adjust programmers and the effect is reversible but the effect is seen at a later time after the programmers (e.g., DBS or Vagus Nerve Stimulation programmers) have been so adjusted, and

d) off-line-prescriptive-permanent where neuromodulation parameters are instructions are generated for users to adjust parameters and the effect is not reversible (e.g., radiosurgery) and the effect is seen at a later time after the change has been made. Examples of types of control exercised are positioning transducers, controlling pulse frequencies, durations and numbers of sessions, pulse-train duration, mechanical perturbations, firing patterns, and coordinating firing so that hitting of multiple targets in the neural circuit using firing patterns is done with optimal effects. In addition, in some cases, firing patterns (Mishelevich, D. J. and M. B. Schneider, “Firing Patterns for Deep Brain Transcranial Magnetic Stimulation,” PCT Patent Application PCT/US2008/073751, published as WIPO Patent Application WO/2009/026386) can be used where multiple energy sources of the same or different types are impacting a single target. This strategy can be used to avoid over-stimulating neural tissues between an energy source and the target to avoid undesirable side effects such as seizures. Positioning of neuromodulators and their settings may be patient specific in terms of (a) the actual position(s) of the target(s), (b) the neuromodulation parameters for the targets, and (c) the functional interactions among the targets. In some case performing imaging or other monitoring, may help in determining adjustments to be made, whether those adjustments are made manually or automatically.

In some cases, an off-line procedure will have already been permanently done (e.g., radiosurgery) and for that modality what occurred would only appear as an input. Control will involve such aspects such as the firing patterns that are employed in each of the applicable modalities, the pattern of stimulation among the employed modalities, and whether simultaneous or sequential neuromodulation is employed (including off-line modalities which will automatically mean sequential neuromodulation is done, if any of the therapeutic modalities in the combination are applied in real-time).

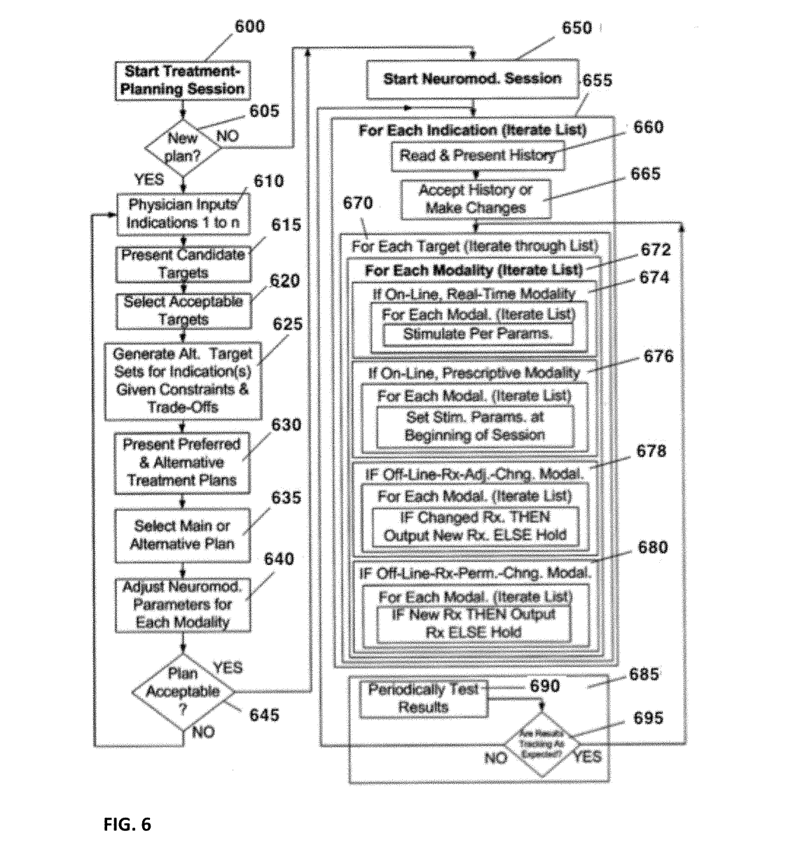

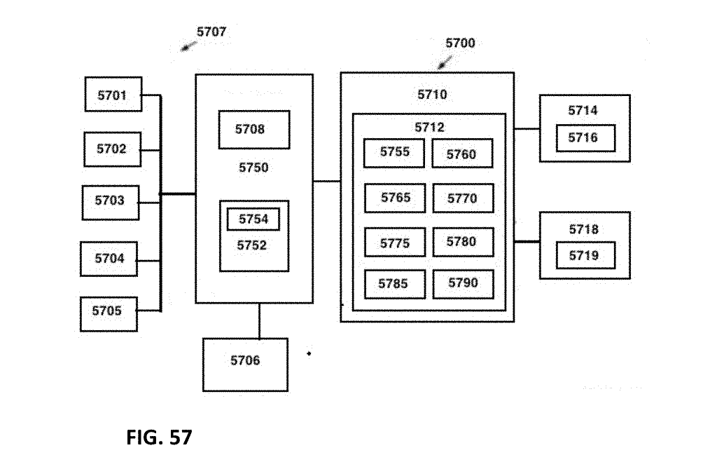

FIG. 6 illustrates the flow for the Multi-Modality Treatment Planning and Control System. Just after the start of the Treatment-Planning Session 600, a branch 605 occurs which depending on whether this is a new plan (for a new patient) proceeds (if the result is yes) to the physician putting in the indications to be treated 610 or proceeds (if the result is no) to the start of the Neuromodulation Session 650. The execution of the flow in FIG. 6 is covered in FIG. 57 with it accompanying description.

The flow for the development of the new plan is for in 610 the physician to input the desired indications followed by the presentation of candidate targets to the physician in 615. There may be only a single indication. The physician selects the acceptable targets in 620 and then the system generated alternative target sets associated with the selected indication(s) in 625 given that physical constraints are satisfied. Trade-offs are given in terms of risk, anticipated relative benefits, possible side effects, and other factors. The resultant preferred treatment plan plus alternative plans are presented to the physician in 630 and the physician makes the selection of what is to be done in 635 and adjusts the neuromodulation parameters for each of the modalities in 640. A branch 645 follows related to whether the resultant plan is acceptable to the physician. If the answer is no, then the process is repeated with the physician again inputting the desired indications in 610. If the answer is yes and the results plan is acceptable, then the Neuromodulation Session is started in 650.

The Neuromodulation Session consists of iterating through each of the designated indications in 655. For each indication, the system reads and presents the history in 660 and the physician in 665 accepts the historical values or makes changes. Then in 670 the system iterates through each of the designated targets and, then within target, in 672, the system iterates through each of the appropriate modalities. The actions depend on the category of the modality. If the case involves an On-Line, Real-Time Modality in 674, the modalities are iterated through, and the given modality is stimulated according to the parameter set. If the case involves an On-Line Prescriptive Modality 676, then for each of the modalities, the stimulation parameters are set in the given programmer at the beginning of the session. Not all programmers can be automatically set by another system such as the Multi-Modality Treatment-Planning and Control system of the invention, so this mechanism may not be available. In any case if such a modality (e.g., DBS or VNS) can be controlled in this way, the set stimulation will usually continue after the On-Line, Real-Time Modalities such as TMS or Ultrasound session is complete. If the case involves an Off-Line-Prescriptive-Adjustable-Change Modality 678, then for each of the modalities the stimulation parameters for the programmer are changed if there is new prescription or held if there is not. Finally, if the case involves an Off-Line-Prescriptive-Change Modality, then for each of the modalities if there now is a prescription, the prescription is output; otherwise the prescription is held. There may be more than one such a modality of that type (e.g., two or more radiosurgery modalities), each related to a different target.

An evaluation of the results occurs in 685. Periodically (either within a neuromodulation session or days, weeks, months, or perhaps even years apart) the functional results are tested in 690. A branch 695 is executed related to whether the results are tracking as expected. If the answer is no, then the flow returns to 655 and each of the indications is iterated through including reading and presenting the history 660 with physician accepting the historical parameter sets or altering them in 665 prior to executing the overall program in 670. If the answer is yes, then no parameter-set changes are required and the flow returns directly to executing the overall program in 670.

A key aspect of the invention described above is that multiple conditions may be treated at the same time. This can be because the indications to be treated share a single target (e.g., the Dorsal Anterior Cingulate Gyrus (DACG) is down regulated in the treatment of both addiction and pain), or multiple targets in multiple circuits are neuromodulated. The treatment of multiple conditions is likely to become increasingly important as the average age of a given population increases. For example when stroke is being treated, in some cases, it will be practical to treat another condition as well. In treating indications with a common target, one most consider whether that target is neuromodulated in the same direction for both conditions. Otherwise, if for one condition the target is to be up regulated and for the other condition the target is to be down regulated, there is a conflict.

All of the embodiments above are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD). In addition, this approach can decrease the number of treatment sessions required for a demonstrated effect and to sustain a long-term effect. Follow-up tune-up sessions at one or more later times may be required.

### Part II: Neuromodulation of Deep-Brain Targets Using Focused Ultrasound

It is the purpose of some of the inventions described herein to provide methods and systems and methods for deep brain or superficial neuromodulation using ultrasound impacting one or multiple points in a neural circuit to produce acute effects or Long-Term Potentiation (LTP) or Long-Term Depression (LTD).

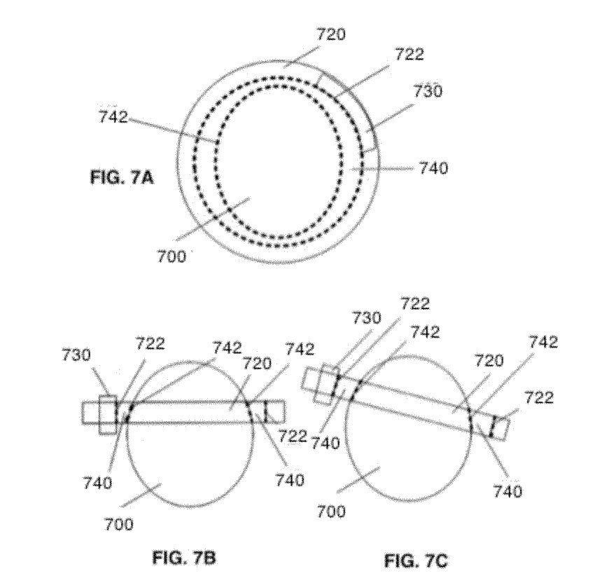

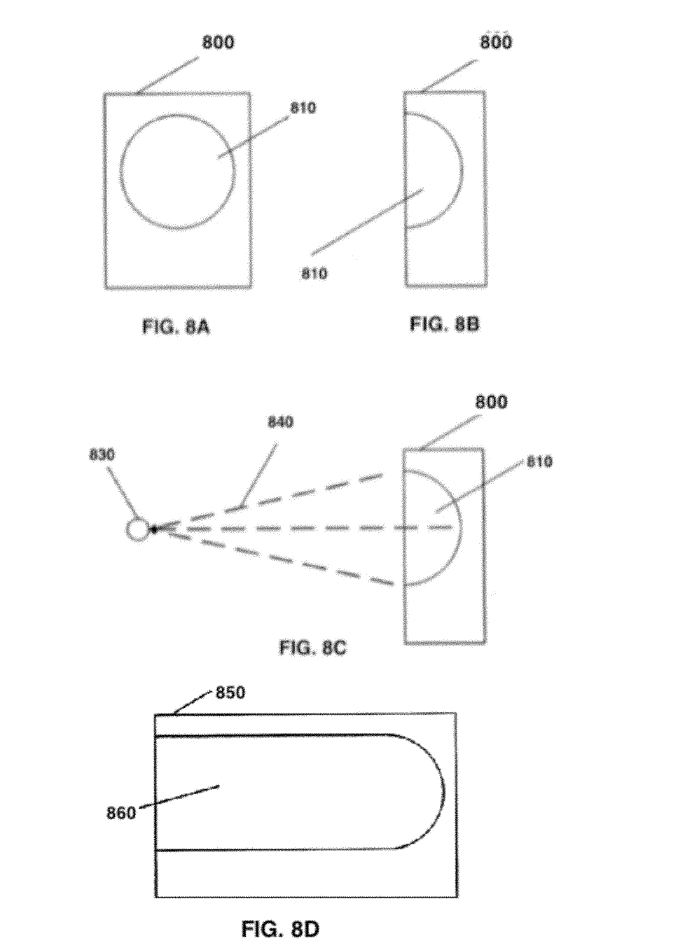

FIG. 7A shows the top view of one embodiment in which a track 720 surrounding human or animal head 700. Riding around track 720 is ultrasound transducer 330. This is a unique and novel feature of this invention. In this embodiment, the face of transducer 730 always faces head 700. Track 720 includes rails for electrical connections to the ultrasound transducers 730. Transducer 730 can ride above the track 720, on the inside of the track 720, or below the track 720. In the latter case, the patient would have less of the apparatus covering their face. In some embodiments, more than one transducer 730 can ride on track 720. For the ultrasound to be effectively transmitted to and through the skull and to brain targets, coupling must be put into place. Ultrasound transmission medium (e.g., silicone oil in a containment pouch) 740 is interposed with one mechanical interface to the ultrasound transducer 730 (completed by a layer of ultrasound transmission gel 722) and the other mechanical interface to the head 700 (completed by a layer of ultrasound transmission gel 742). FIG. 7B shows the frontal view of FIG. 7A for the case where transducer 730 is riding on the inside of track 720. The sound-conduction path between ultrasound transducer 730 and head 700 by conductive-gel layer 722, sound-conduction medium 740 and conductive-gel layer 742. FIG. 7C illustrates the situation where track 7120 is tilted to allow better positioning for some targets or sets of targets if more than one neural structure is targeted in a given configuration. Again, ultrasound transmission medium 740 is interposed with one mechanical interface to the ultrasound transducer 730 (completed by a layer of ultrasound transmission gel 722) and the other mechanical interface to the head 700 (completed by a layer of ultrasound transmission gel 742). The depth of the point where the ultrasound is focused depends on the shape of the transducer and setting of the phase and amplitude relationships of the elements of the ultrasound transducer array discussed in relation to FIGS. 8A-8C. In another embodiment, a non-beam-steered-array ultrasound transducer can be used with the transducer only activated when it is correctly positioned to effectively aim at the target. As noted previously, in any case, the ultrasound transducer must be coupled to the head by an ultrasound transmission medium, including gel, if appropriate for effective ultrasound transmission can occur.

In another embodiment of the configuration shown in FIGS. 7A-C, instead of the transducer or transducers 730 riding around on the track 720, they may fixed in place at a given location or locations on the track suitable to hit the desired target(s). In this case, in an alternative embodiment, a non-beam-steered-array ultrasound transducer can be used. Again, ultrasound transmission medium must be used for energy coupling.

FIGS. 8A-8C show the face of transducer 800 with an array of ultrasound transducers distributed over the face of transducer array assembly 810. FIG. 8A shows the front of the transducer as would face the target and FIG. 8B shows a side view. FIG. 8C illustrates the ultrasound field represented by dashed lines 840 striking target neural structure 800 with the control of phase and amplitude producing the focus. Depending on the focal length of the ultrasound field, the length of the ultrasound transducer assembly can be increased with a corresponding increase in the length of ultrasound-conduction-medium insert. For example, FIG. 8D shows a longer ultrasound transducer body 850 and a longer ultrasound-conduction-medium insert 860.

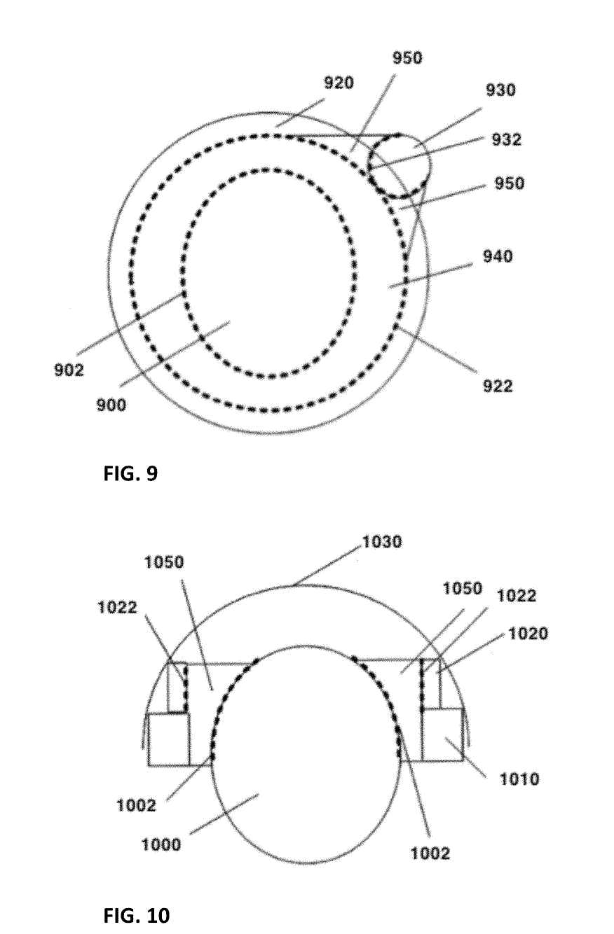

FIG. 9 illustrates an alternative embodiment where track 920 surrounds head 900 now has a transducer 930 whose face can be rotated so it can be aimed towards the intended target(s) rather than always facing perpendicularly to the head. Track 920 includes rails for electrical connections to the sound transducers 930. As transducer 930 reaches a given point on track 900, transducer 930 can be rotated toward the target(s). Again, in some embodiments, more than one transducer 930 can ride on track 920. For the ultrasound to be effectively transmitted to and through the skull and to brain targets, coupling must be put into place. Ultrasound transmission medium 940 is interposed with one mechanical interface to the ultrasound transducer 932 (completed by a layer of ultrasound transmission gel 922) and the other mechanical interface to the head 900 (completed by a layer of ultrasound transmission gel 902). For the rotating element 930, completion of the coupling is achieved with transmission coupling medium 950 is in place (completed by a layer of ultrasound transmission gel 922). In another embodiment, one or more transducers 930 can be fixed in position on track 920, but one or more of transducers 930 can still be rotated to it can be aimed towards the target. Such rotation can either allow sweeping over an elongated target or can periodically alternatively aimed toward each of more than one target. In some embodiments, one or more transducers fixed in position on the track are not rotated. The transducer arrays incorporated in transducer 730 in FIGS. 7A-7C and 930 in FIG. 9 can both of the form of FIGS. 8A-8C or other suitable configuration. In addition the tracks in the configurations shown in FIGS. 7A-7C, FIG. 9 and their alternative embodiments can be raised and lowered vertically as required for optimal targeting. The track can be tilted side-to-side, front to back, diagonal, or in any direction according to the targeting need. The tracks can be tilted back and forth according to the targeting need. Also there may be transducer carriers containing a plurality of transducers so the combination can target more than one target simultaneously. Other embodiments may be smaller versions covering only a portion of the skull with the ability to target fewer (simultaneously) or perhaps only one target that can be used both in an increased number of clinical settings or at home, school, or work. Another embodiment incorporates a transducer-holding device, which is not a track, which holds the ultrasound transducers in fixed positions relative to the target or targets. The locations and orientations of the holders can be calculated by locating the applicable targets relative to atlases of brain structure such as the Tailarach atlas. As noted above, in each case, transmission-coupling medium must be in place.

In another embodiment, either of the implementations in FIGS. 7A-7C or FIG. 9 can be enclosed in a shell as shown in FIG. 10 where head 1000 is shown in a frontal view with transducer 1020 riding on track 1010 all enclosed in shell 430. In this embodiment, there are two transducers 1020, placed 180 degrees apart. In this case, as for the other configurations, for the effective ultrasound transmission to and through the skull and to brain targets, coupling must be put into place. Ultrasound transmission medium 1050 is interposed with one mechanical interface to the ultrasound transducer 1020 (completed by a layer of ultrasound transmission gel 1022) and the other mechanical interface to the head 1000 (completed by a layer of ultrasound transmission gel 1002). In another embodiment, mechanical perturbations are applied radially or axially to move the ultrasound transducers. This is applicable to a variety of transducer configurations.

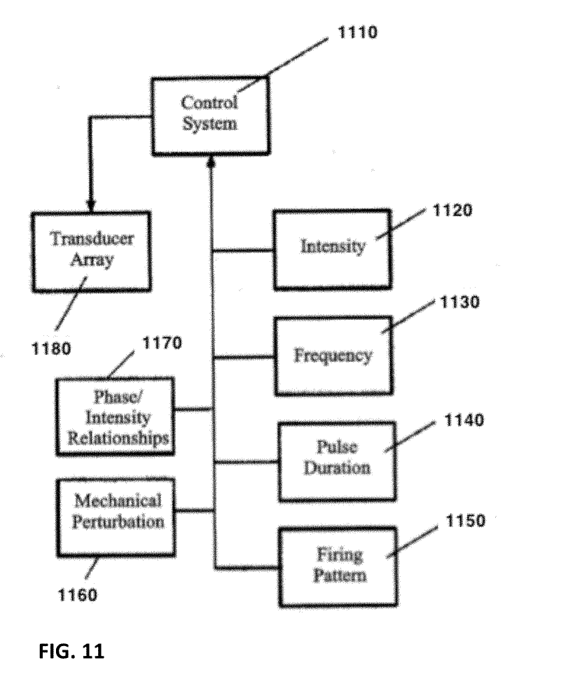

FIG. 11 shows an embodiment of a control circuit. The positioning and emission characteristics of transducer array 1180 are controlled by control system 1110 with control input with neuromodulation characteristics determined by settings of intensity 1120, frequency (including carrier frequency) 1130, pulse duration 1140, firing pattern 1150, mechanical perturbations 560, and phase/intensity relationships 1170 for beam steering and focusing on neural targets. Control of the flow in FIG. 11 can occur as in FIG. 57 with it accompanying description.

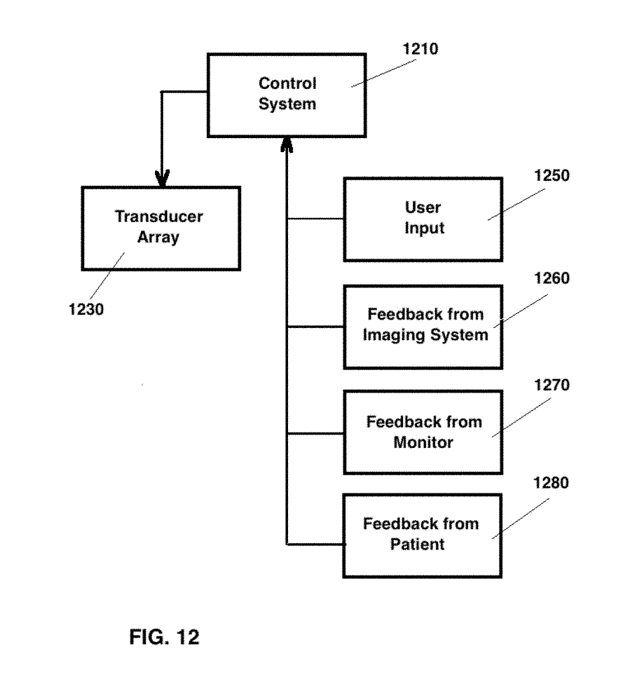

FIG. 12 shows another embodiment of a control circuit. The positioning and emission characteristics of transducer array 1230 are controlled by control system 1210 with control input from either user by user input 1250 and/or from feedback from imaging system 1260 (either automatically or display to the user with actual control through user input 1250) and/or feedback from a monitor (sound and/or thermal) 1270, and/or the patient 1280. Control can be provided, as applicable, for direction of the energy emission, intensity, frequency for up-regulation or down-regulation, firing patterns, mechanical perturbations, and phase/intensity relationships for beam steering and focusing on neural targets.

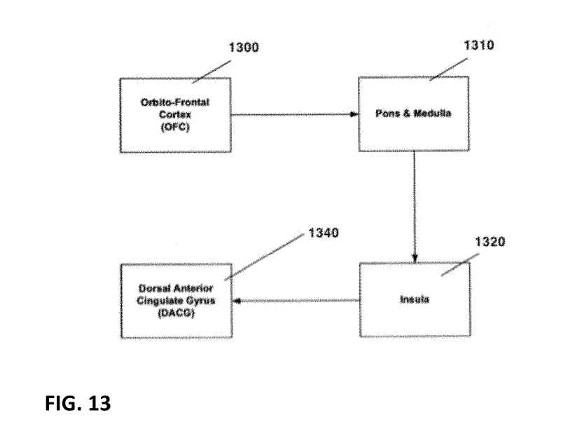

An example of a neural circuit for a condition, in this case addiction is shown in FIG. 13. In this circuit, the elements are Orbito-Frontal Cortex (OFC) 1300, Pons & Medulla 1310, Insula 1320, and Dorsal Anterior Cingulate Gyrus (DACG) 1340. One or more targets can be targeted simultaneously or sequentially. Down regulation means that the firing rate of the neural target has its firing rate decreased and thus is inhibited and up regulation means that the firing rate of the neural target has its firing rate increased and thus is excited. For the treatment of addiction, the OFC 1300, Insula 1320, and DACG 1340 would all be down regulated. The ultrasonic firing/timing patterns can be tailored to the response type of a target or the various targets hit within a given neural circuit.

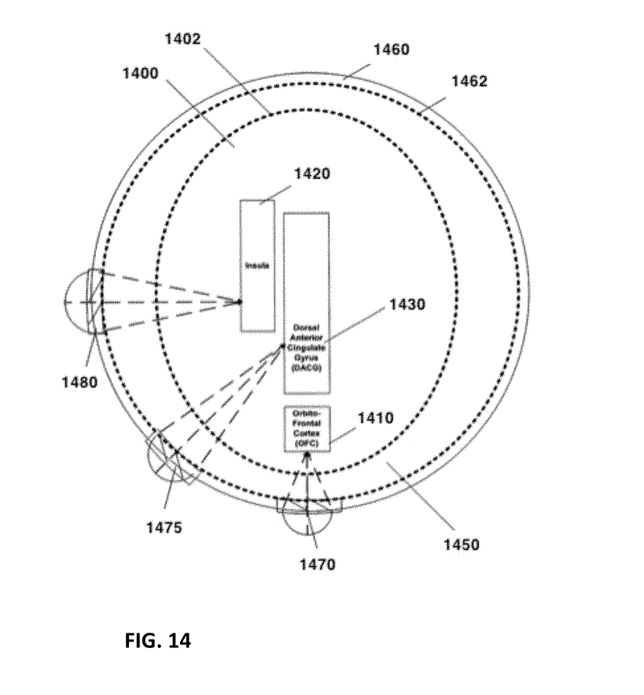

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) and enhances acute effects as well. In addition, this approach can decrease the number of treatment sessions required for a demonstrated effect and to sustain a long-term effect. Follow-up tune-up sessions at one or more later times may be required. FIG. 14 shows a multi-target configuration. The head 1400 contains the three targets, Orbito-Frontal Cortex (OFC) 1410, Insula 1420, and Dorsal Anterior Cingulate Gyrus (DACG) 1430, also shown in FIG. 13 (the Pons and Medulla set shown in FIG. 13 is not shown in FIG. 14 because that set is not targeted). Ultrasound transducers 1470, 1475, and 1480, fixed to track 1460 (or running around track 1460) hit these targets. Ultrasound transducer 1470 is shown targeting the OFC 1410, transducer 1475 is shown targeting the DACG, 1430 and transducer 1480 is shown targeting the Insula 1420. For the ultrasound to be effectively transmitted to and through the skull and to brain targets, coupling must be put into place. Ultrasound transmission medium 1450 is interposed with one mechanical interface to the ultrasound transducers 1470, 1475, 1480 (completed by a layer of ultrasound transmission gel 1462) and the other mechanical interface to the head 1400 (completed by a layer of ultrasound transmission gel 1402). In some cases, the neural structures will be targeted bilaterally (e.g., both the right and the left Insula) and other cases, only one will targeted (e.g., the right Insula in the case of addiction).

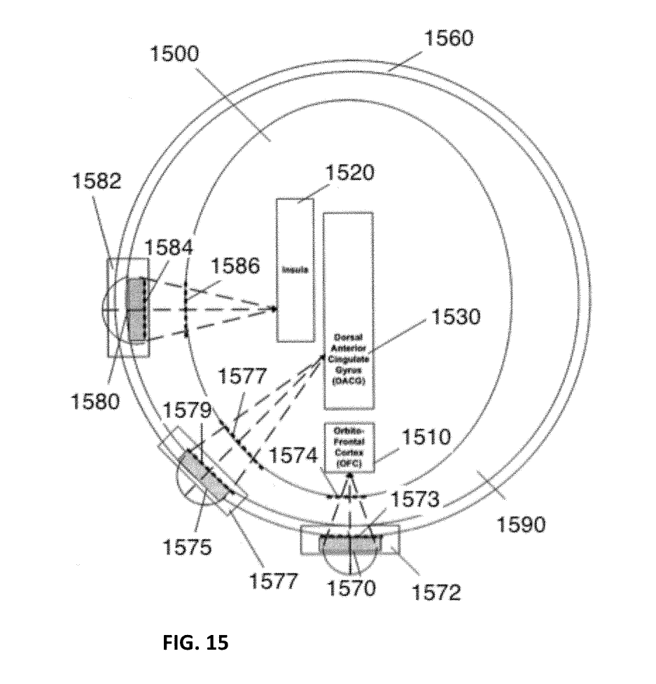

FIG. 15 shows a fixed configuration where the appropriate radial (in-out) positions have determined through patient-specific imaging (e.g., PET or fMRI) and the holders positioning the ultrasound transducers are fixed in the determined positions. The head 1500 contains the three targets, Orbito-Frontal Cortex (OFC) 1510, Insula 1520, and Dorsal Anterior Cingulate Gyrus (DACG) 1530. Ultrasound transducers 1570, 1575, and 1580, fixed to track 1560, hit these targets. Ultrasound transducer 1570 is shown targeting the OFC 1510, transducer 1575 is shown targeting the DACG 1530, and transducer 1580 is shown targeting the Insula 1520. Transducer 1570 is moved radially in or out of holder 1572 and fixed into position. In like manner, transducer 1575 is moved radially in or out of holder 1577 and fixed into position and transducer 1580 is moved radially in or out of holder 1582 and fixed into position. For ultrasound to be effectively transmitted to and through the skull and to brain targets, coupling must be put into place. Ultrasound transmission medium 1590 is interposed with one mechanical interface to the ultrasound transducers 1570, 1575, 1580 (completed by a layers of ultrasound transmission gel 1573, 1579, 1584) and the other mechanical interface to the head 1500 (completed by a layers of ultrasound transmission gel 1574, 1577, 1586). To support this embodiment, treatment-planning software is used taking the image-determined target positions and output instructions for manual or computer-aided manufacture of the holders. Alternatively positioning instructions can be output for the operator to position the blocks holding the transducers to be correctly placed relative to the support track. In one embodiment, the transducers positioned using this methodology can be aimed up or down and/or left or right for correct flexible targeting.

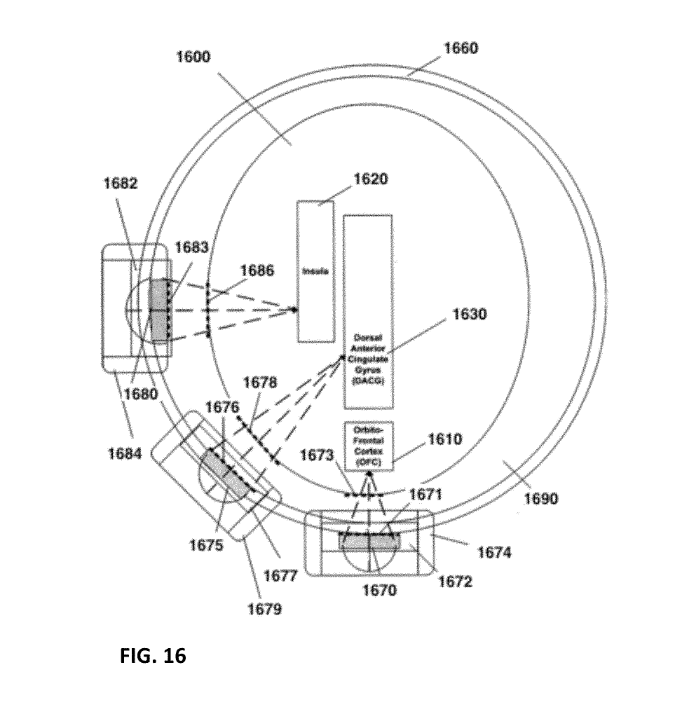

FIG. 16 illustrates an automatically adjustable configuration where based on the image-determined target positions discussed relative to FIG. 15, the transducer holders are moved in or out to the correct positions for the given target without a fixed patient-specific holder having been fabricated or manually adjusted relative to the track or other frame. The head 1600 contains the three targets, Orbito-Frontal Cortex (OFC) 1610, Insula 1620, and Dorsal Anterior Cingulate Gyrus (DACG) 1630, also shown in FIG. 13. Ultrasound transducers 1670, 1675, and 1680, fixed to track 1660, hit these targets. Transducer 1670 mounted on support 1672 is moved radially in or out of holder 1674 by a motor (not shown) to the correct position under control of treatment planning software or manual control. In like manner, transducer 1675 mounted on support 1677 is moved radially in or out of holder 1679 by a motor (not shown) to the correct position under control of treatment planning software or manual control. In like manner, transducer 1680 mounted on support 1682 is moved radially in or out of holder 1684 by a motor (not shown) to the correct position under control of the treatment planning software or manual control. Ultrasound transducer 1670 is shown targeting the OFC 1610, transducer 1675 is shown targeting the DACG 1630, and transducer 1680 is shown targeting the Insula 1620. For the ultrasound to be effectively transmitted to and through the skull and to brain targets, coupling must be put into place. Ultrasound transmission medium 1690 is interposed with one mechanical interface to the ultrasound transducers 1670, 1675, 1680 (completed by a layers of ultrasound transmission gels 1671, 1676, 1683) and the other mechanical interface to the head 1600 (completed by a layers of ultrasound transmission gel 1673, 1678, and 1686). An embodiment involving the latter would use a single or fewer-than-the-number-of-targets transducers to hit multiple targets since the or fewer-than-the-number-of-targets transducers can be moved in and out or rotated left and right and/or up and down to hit the multiple targets.

The invention allows stimulation adjustments in variables such as, but not limited to, intensity, firing pattern, frequency, mechanical perturbations, phase/intensity relationships, mechanical perturbations, dynamic sweeps, and position to be adjusted so that if a target is in two neuronal circuits the transducer or transducers can be adjusted to get the desired effect and avoid side effects. The side effects could occur because for one indication the given target should be up regulated and for the other down regulated. An example is where a target or a nearby target would be down regulated for one indication such as pain, but up-regulated for another indication such as depression. This scenario applies to either the Dorsal Anterior Cingulate Gyrus (DACG) or Caudate Nucleus. Even when a common target is neuromodulated, adjustment of stimulation parameters may moderate or eliminate a problem because of differential effects on the target relative to the involved clinical indications.

The invention also contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyrus (DACG). To counterbalance the down-regulation of the DACG during treatment for pain that negatively impacts the treatment for depression, one would up-regulate the Nucleus Accumbens or Hippocampus that are other targets in the depression neural circuit. A plurality of such applicable targets could be stimulated as well.

Another applicable scenario is the Nucleus Accumbens that is down regulated to treat addiction, but up regulated to treat depression. To counteract the down-regulation of the Nucleus Accumbens to treat depression but will negatively impact the treatment of depression that would like the Nucleus Accumbens to be up regulated, one would up-regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

### Part III: Shaped and Steered Ultrasound for Deep-Brain Neuromodulation

It is the purpose of some of the inventions described herein to provide a device for producing shaped or steered ultrasound for non-invasive deep brain or superficial stimulation impacting one or multiple points in a neural circuit to produce acute effects or Long-Term Potentiation (LTP) or Long-Term Depression (LTD) using up-regulation or down-regulation.

If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption.

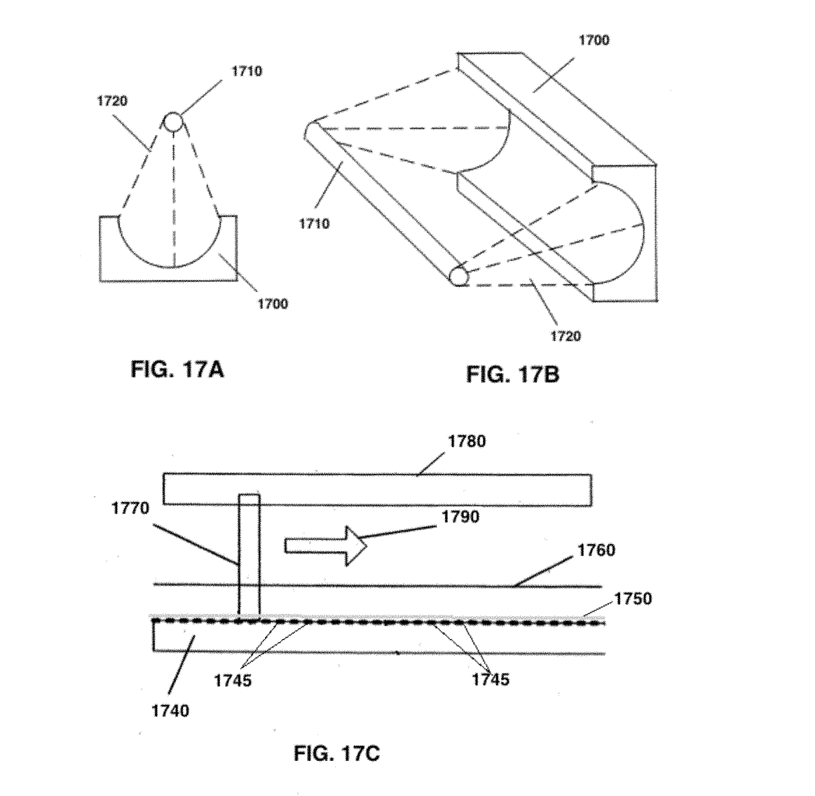

FIGS. 17A and 17B show an ultrasound transducer array configured to produce an elongated pencil-shaped focused field. Such an array would he applied to stimulate an elongated target such as the Dorsal Anterior Cingulate Gyms (DACG) or the Insula. Note that one embodiment is a swept-beam transducer with the capability of sweeping the sound field over any portion of the length of the ultrasound transducer. Thus it is possible to determine over what length of a target that the ultrasound is applied. For example, one could apply ultrasound to only the anterior portion of the target. Also, by rotating or tilting a transducer in a holder, one can vertically target such as aiming the sound field at the superior portion of a target. In FIG. 17A, an end view of the array is shown with curved-cross section ultrasonic array 1700 forming a sound field 1720 focused on target 1710. FIG. 17B shows the same array in a side view showing an end view with its curved cross section of the ultrasound array, again with ultrasound array 1700, target 1710, and focused field 1720. The exemplary ultrasound transducer assembly 1700 may be a shaped piezoelectric transducer body or may comprise an array of individual transducer elements configured to produce an elongated tubular (e.g. pencil-shaped) focused field.

FIG. 17C shows a linear ultrasound phased array 1740 which can “steer” an ultrasound beam 1770 by changing the phase/intensity relationships of a plurality of individual transducer elements 1745. In this way, ultrasound beams can be moved (steered) and focused without physically displacing the array 1740 of transducers 1745. The beam direction can be directed at angles that are perpendicular or non-perpendicular to the surface of the transducer array, and beam direction is thus not restricted to being aimed perpendicularly from the face of the transducer or array. In FIG. 17C, the transducer array 1740 is flat and emits ultrasound conducted by a conducting gel layer 1750 providing the physical interface to skin. The beam 1770 of ultrasound energy moves linearly from left to right as shown by arrow 1790 so it moves its focus along intended target (e.g., spinal cord) 1780. In another embodiment, the surface of the transducer array is not flat but curved.

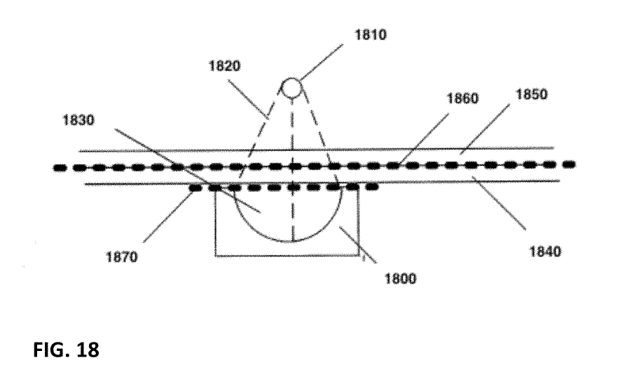

FIG. 18 illustrates the elongated ultrasound transducer array shown in FIGS. 17A-17B (now with ultrasound-transducer array 1800, target 1810, and focused ultrasound field 1820), but in this case showing head layer 1850 and sound-conduction medium 1830 in place. Ultrasound is transmitted through fitted sound-conduction medium 1830, a layer of conduction gel 1870 providing the interface to solid sound-conduction medium 1840, and a layer of conduction gel 1860 providing interface to the head layer. Examples of sound-conduction media are Dermasol from California Medical Innovations or silicone oil in a containment pouch.

In FIG. 19, the physical target layout for addiction for the targets shown in FIG. 13 has within head 1900 targets Orbito-Frontal Cortex (OFC) 1910, Dorsal Anterior Cingulate Gyms (DACG) 1930, and Insula 1920. Sound field 1911 emanating from ultrasound transducer 1970 is focused on Orbito-Frontal Cortex (OFC) 1910. Sound field 1976 emanating from ultrasound transducer 1975 is focused on Dorsal Anterior Cingulate Gyms (DACG) 1930. Sound field 1981 emanating from ultrasound transducer 1980 is focused on Insula 1920. All of the ultrasound transducers are mounted on frame 1960 with the ultrasound conducted through conductive gel layer 1962, conductive medium 1950, and conductive gel layer 1902 that provides the interface to head 1900.

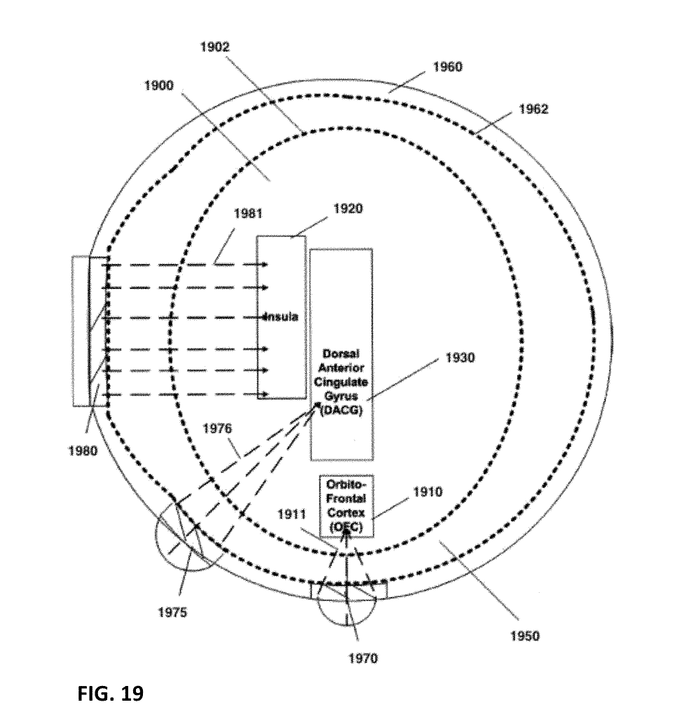

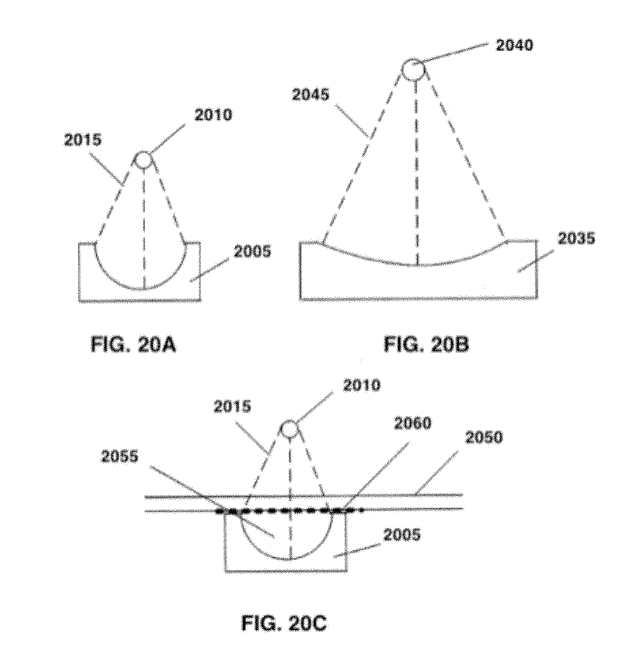

FIGS. 20A-20C demonstrates two ultrasound transducer arrays with different radii. The array with the shorter focal length in FIG. 20A has transducer array 2005 focusing sound field 2015 at target 2010. In FIG. 20B, the array with the longer focal length because of the larger radius has transducer array 2035 focusing sound field 2045 at target 2040. In order to work, there must be a medium between the transducer array and the head to conduct the sound. In FIG. 20C shows the transducer array 2005 of FIG. 20A with sound field 2015 focused on target 2010 with sound conduction media in place between array 2005 and head 2050. The conduction mechanism consists of hemispheric conduction medium 2055 and conducting-gel layer 2060 providing the physical interface to head 2050.

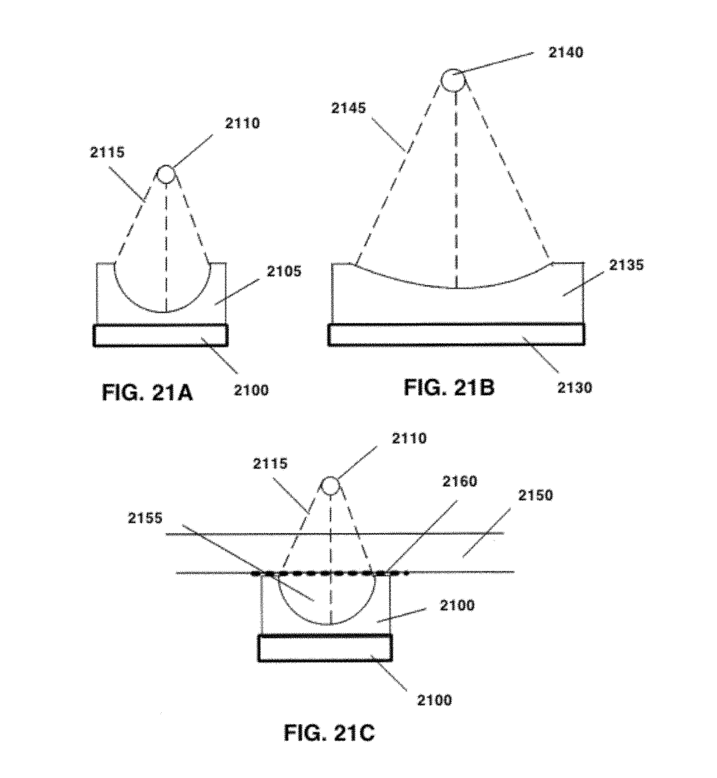

FIGS. 21A-21C demonstrate an embodiment where a flat transducer array is used in conjunction with interchangeable lenses. The configurations are the same as those in FIGS. 20A-20C with the curved transducer array replaced by a combination of a flat transducer array and a curved lens. In FIG. 21A, flat transducer array 2100 has its sound field focused by curved lens 2105 with sound field 2115 focused on target 2110. In FIG. 21B, flat transducer array 2130 has its sound field focused by curved lens 2135 with sound field 2145 focused on target 2140. FIG. 21C shows the transducer array 2100 with lens 2105 of FIG. 21A with sound field 2115 focused on target 2110 with sound conduction media in place between lens 2105 and head 2150. The conduction mechanism consists of hemispheric conduction medium 2155 and conducting-gel layer 2160 providing the physical interface to head 2150. These lenses can be bonded to flat transducers or non-permanently affixed. With fixed transducer radii configured to not require beam steering, simpler driving electronics can be used. In some embodiments, a portion of a hemisphere can be used as opposed to a full hemisphere, but in these cases, the power required to achieve a given depth will typically be larger. Different focal depths can be achieved by alterations in transducer configuration and different field shapes can be achieved by different array-transducer shapes (e.g., curved elongated as opposed to flat linear, square, or hemispheric).

An important reason to use the flat transducer with either a fixed or interchangeable lens is that a simple fixed or variable function generator or equivalent can be used (cost in hundreds to low thousands of dollars) as opposed a beam-steering variable amplitude and phase generator (costs in the tens of thousands of dollars). Representative materials for lens construction are metal or epoxy. In an alternative embodiment, a focusable ultrasound lens can be used (G. A. Brock-Fisher and G. G. Vogel, “Multi-Focus Ultrasound Lens”, U.S. Pat. No. 5,738,098).

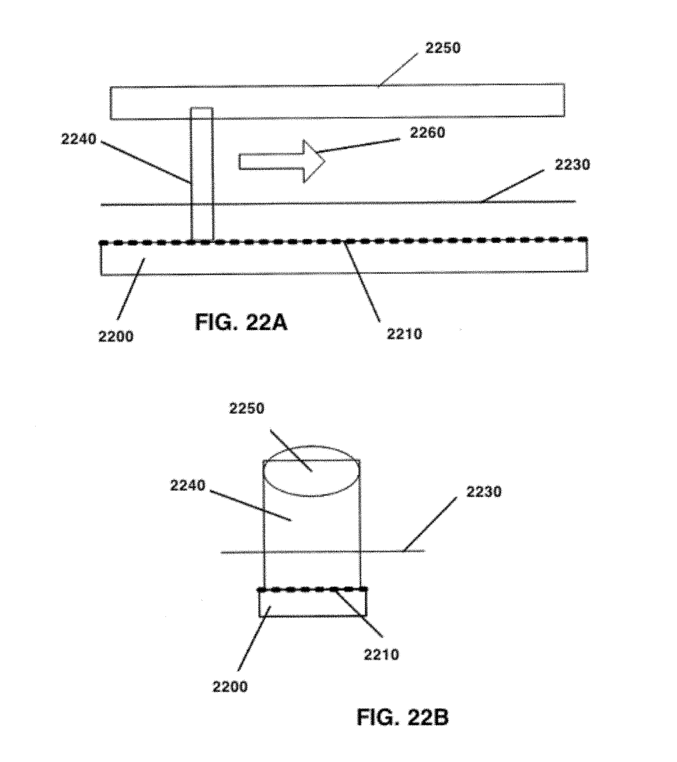

FIGS. 22A and 22B show a linear ultrasound phased array with a steered-beam linearly moving field generated by changing the phase/intensity relationships. Beams can also be focused or steered without motion or with non-linear motion. They also can be directed at an angle and not restricted to being aimed perpendicular to the face of the array. FIG. 22A shows a side view and FIG. 22B shows an end view. In FIG. 22A, flat transducer array 2200 has its ultrasound conducted by conducting gel layer 2210 providing the physical interface to head 2230. Sound field 2240 moves linearly from left to right as shown by arrow 2260 so it moves its focus along target 2250. FIG. 22B shows the end view of the configuration looking at the end of flat transducer 2200 with conduction of ultrasound to the head 2230 provided by conduction layer 2210 and sound field 2240 focused on target 2250. In comparison to FIG. 22A, the sound field 2240, which moves, left to right in FIG. 22A moves back into the page in FIG. 22B. In another embodiment, the transducer array is not flat but curved.

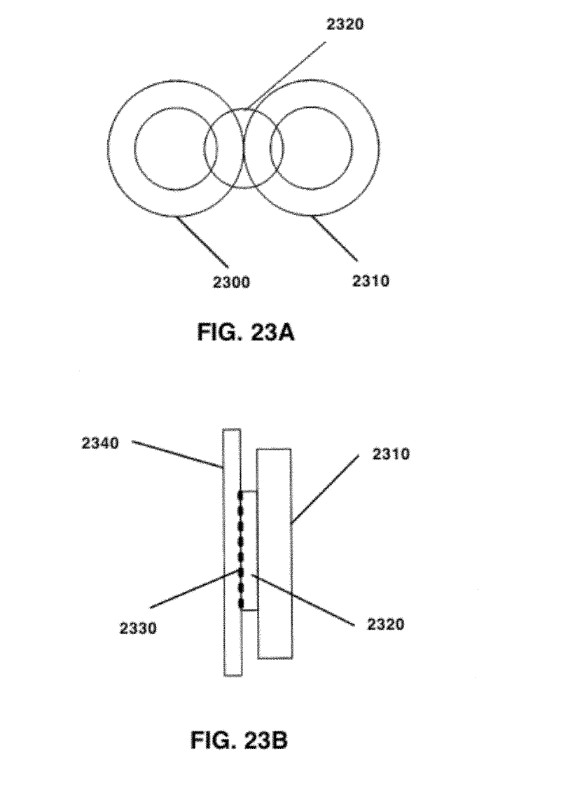

FIGS. 23A and 23B demonstrates the combination of an ultrasound transducer with a figure-8 Transcranial Magnetic Stimulation (TMS) Coil in both front and side views. FIG. 23A shows the front view of the TMS electromagnet with its component coils 2300 and 2310 and the face of ultrasonic transducer. The side view of the configuration with the head 2340 included is shown in FIG. 23B with the end view of the TMS electromagnet as to side of coil 2310, the side of the ultrasound transducer 2320. Conductive-gel layer 2330 providing the physical interface between ultrasound transducer array 2320, and head 2340 provides the ultrasound conduction. MRI-compatible ultrasound generators are available (e.g., from Imasonic) so that the presence of the ultrasound transducer will have minimal impact on the magnetic field generated by the TMS electromagnet.

Any shape of array such as those described above may have its sound field steered or focused. The depth of the point where the ultrasound is focused depends on the setting of the phase and amplitude relationships of the elements of the ultrasound transducer array. The same is true for the lateral position of the focus relative to the central axis of the ultrasound transducer array. An example of directing ultrasound is found in Cain and Frizzell (C. A. Cain and L. A. Frizzell, “Apparatus for Generation and Directing Ultrasound,” U.S. Pat. No. 4,549,533). In another embodiment a viewing hole can be placed in an ultrasound transduction to provide an imaging port. Both Imasonic and Keramos-Etalon supply such configurations.

In other embodiments the transducer can be moved back and forth to cover a long target or vibrate in-and-out or in any direction off the central axis to increase the local effects on neural-structure membranes.

FIG. 11 shows a control block diagram. In one embodiment control is also provided for a Transcranial Magnetic Stimulation (TMS) coil as integrated with an ultrasound transducer as shown in FIGS. 23A-23B.

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) or enhances acute effects. In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in other cases only one will targeted (e.g., the right Insula in the case of addiction).

### Part IV: Mechanical Perturbations

It is the purpose of some of the inventions described herein to provide a device and method for producing shaped ultrasound sound fields by applying mechanical perturbations to move ultrasound transducers for non-invasive deep brain or superficial stimulation impacting one or multiple points in a neural circuit to produce acute effects or Long-Term Potentiation (LTP) or Long-Term Depression (LTD) using up-regulation or down-regulation.

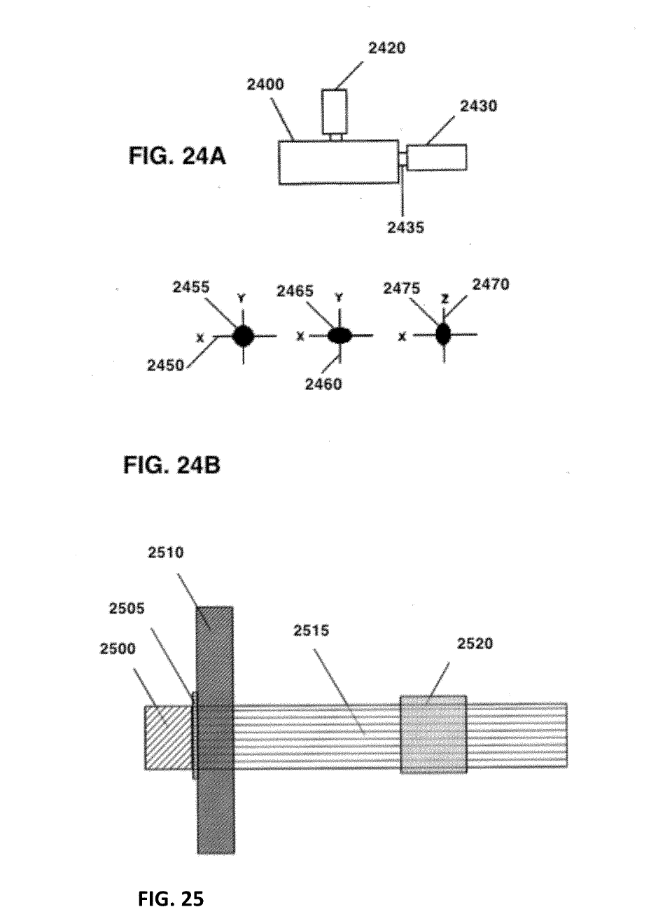

FIGS. 24A and 24B show the mechanism for mechanical perturbations of the ultrasound transducer. In FIG. 24A illustrating a plan view with mechanical actuators 2420 and 2430 moving ultrasound transducer 2400 in and out and left respectively. Actuator rod 2435 provides the mechanical interface between mechanical actuator 2430 and ultrasound transducer 2400 as an example. Not shown is an equivalent mechanical actuator moving ultrasound transducer 2400 along an axis perpendicular to the page. Such mechanical actuators can have alternative configurations such as motors, vibrators, solenoids, magnetostrictive, electrorestrictive ceramic and shape memory alloys. Piezo-actuators such as those provided by DSM can have very fine motions of 0.1% length change. FIG. 24B shows effects on the focused ultrasound modulation focused at the target. The axes are 2450 (x,y), 2460 (x,y,) and 2470 (x,z). As demonstrated on 450 the excursions along x and y from 2430 and 2420 are equal so the resultant pattern is a circle. As demonstrated on 2460 the excursion due to 2430 is greater than that if 2420 so the resultant pattern is longer along the x axis than the y axis. As demonstrated on 2470, the excursion is longer along the z axis than the x axis to the resultant pattern is long along the z axis than the x axis. Not shown is the inclusion of the impacts of actuation along the axis perpendicular to the page. In each case, the pattern would be matched to the shape of the target of the modulation.

### Part V: Ultrasound-Intersecting Beams for Deep-Brain Neuromodulation

One invention described herein is an ultrasound device using intersecting beams delivering enhanced non-invasive deep brain or superficial deep-brain neuromodulation impacting one or a plurality of points in a neural circuit to produce acute effects (as in the treatment of post-surgical pain) or Long-Term Potentiation (LTP) or Long-Term Depression (LTD) using up-regulation or down-regulation.

FIG. 25 shows a flat ultrasound transducer producing a parallel beam intersecting a single target. Flat ultrasound transducer 2500 produces ultrasound beam 2515. To be practical, ultrasound beam 2515 passes through skull section 2510 with coupling medium 2505 interposed between transducer 2500 and skull section 2510 to support effective transmission. Ultrasound beam 2515 hits target 2520.

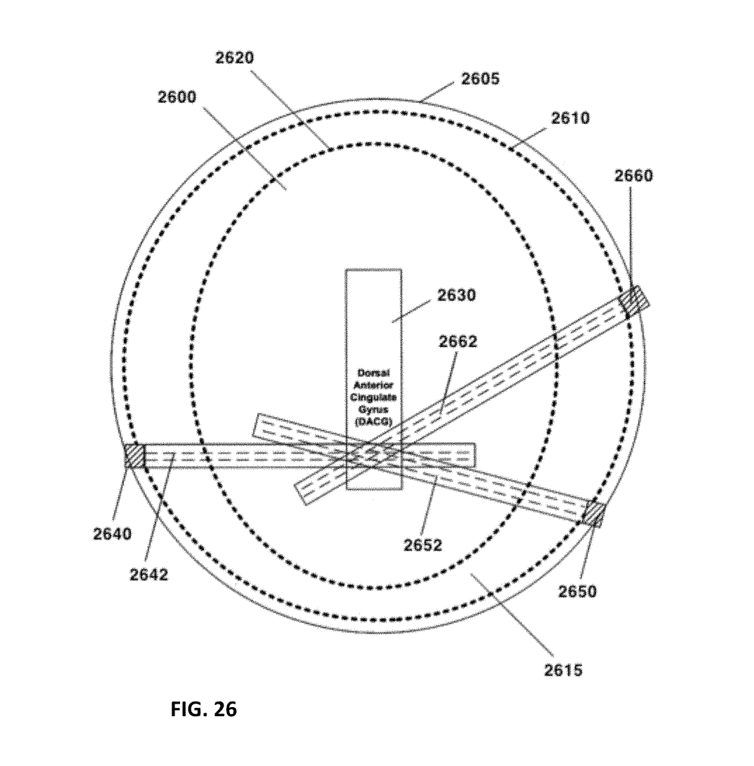

FIG. 26 illustrates head 2600 containing target Dorsal Anterior Cingulate Gyrus (DACG) 2630. Frame 2605 holds three ultrasound transducers 2640, 2650, and 2660. The beam from each ultrasound transducer passes though an ultrasound-conduction medium 2615 with ultrasound-conduction gel interfaces 2610 at the transducer face and 2620 at the head. Ultrasound transducer 2640 generates ultrasound beam 2642, ultrasound transducer 2650 generates ultrasound beam 2652, and ultrasound transducer 2660 generates ultrasound beam 2662. Ultrasound beams 2642, 2652, and 2662 intersect at Dorsal Anterior Cingulate Gyrus target 2630 and neuromodulate the DACG. The effects of beams 2642, 2652, and 2662 are additive. Examples of ultrasound conduction media include Dermasol from California Medical Innovations and silicone oil in a containment pouch. Ultrasound-conjunction gel (not shown) can be placed just at the interfaces between any of the ultrasound transducers and the band of ultrasonic-conduction medium 2615 and that band and head 2600 as long as the beam regions are covered. One or more of the plurality of the ultrasound transducers can also be used with an acoustic lens (not shown). For elongated targets such as the DACG, the intersecting beams can be spread to cover a broader neural region. In addition the width of the ultrasound transducer and thus the width of the beam can be varied.

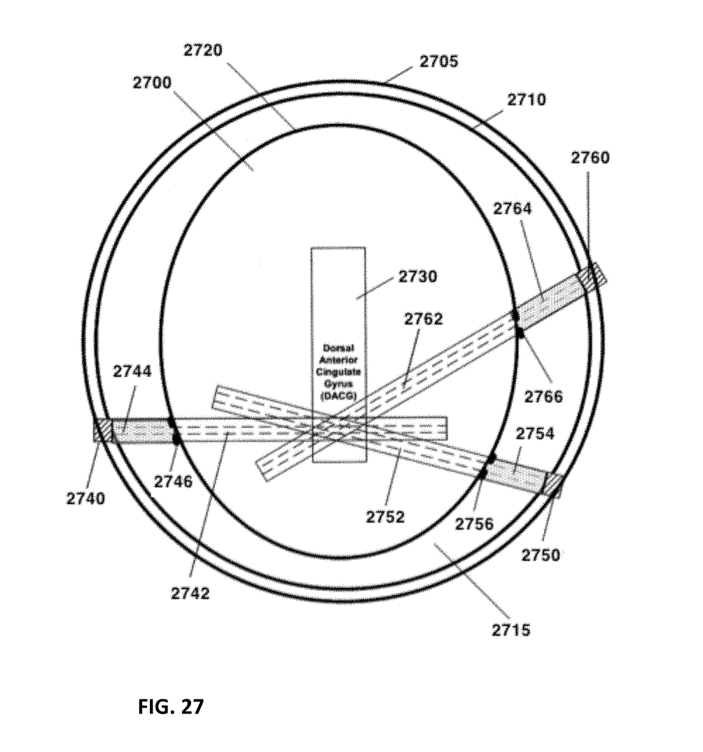

In another embodiment, the ultrasound-conduction medium is not incorporated in a continuous band around the head (2615 in FIG. 26), but instead is configured as a single ultrasound conduction medium for each ultrasound transducer. FIG. 27 illustrates head 2700 containing target Dorsal Anterior Cingulate Gyrus (DACG) 2730. Frame 2705 holds three ultrasound transducers 2740, 2750, and 2760. The beam from each ultrasound transducer passes though individual ultrasound-conduction media. For ultrasound transducer 2740, beam 2742 passes through ultrasound-conduction medium 2744 and then through ultrasound-conduction gel 2746 at the interface with head 2700. There also can be a layer ultrasound-conduction gel (not shown) at the interface between ultrasound transducer 2740 and ultrasound-conduction medium 2744. For ultrasound transducer 2750, beam 2752 passes through ultrasound-conduction medium 2754 and then through ultrasound-conduction gel 2756 at the interface with head 2700. There also can be a layer of ultrasound-conduction gel (not shown) at the interface between ultrasound transducer 2750 and ultrasound-conduction medium 2754. In like manner, for ultrasound transducer 2760, beam 2762 passes through ultrasound-conduction medium 2764 and then through ultrasound-conduction gel 2766 at the interface with head 2700. There also can be a layer of ultrasound-conduction gel (not shown) at the interface between ultrasound transducer 2760 and ultrasound-conduction medium 2764. Ultrasound beams 2742, 2752, and 2762 intersect at Dorsal Anterior Cingulate Gyrus target 2730 and neuromodulate the DACG. The effects of beams 2742, 2742, and 2762 are additive. Each ultrasound transducer can also be used with an acoustic lens (not shown). For elongated targets such as the DACG, the intersecting beams can be spread to cover a broader neural region. In addition the width of the ultrasound transducer and thus the width of the beam can be varied.

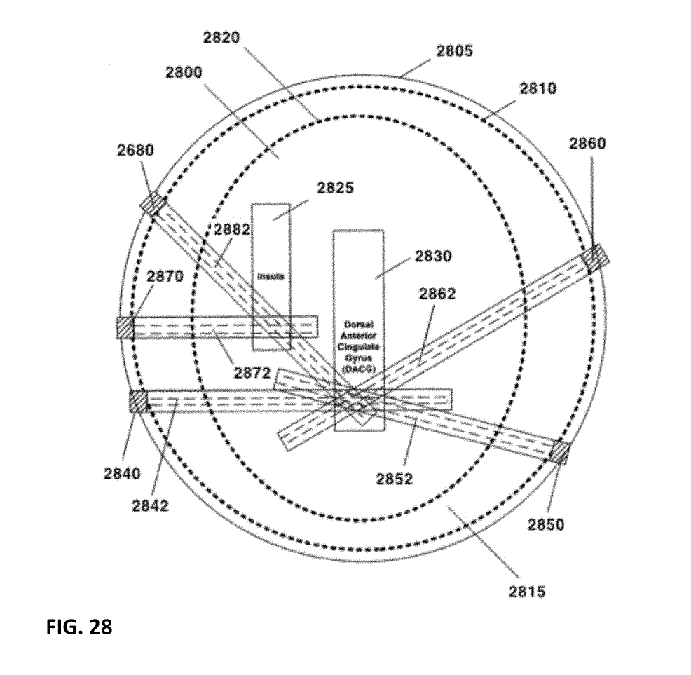

In another embodiment, a plurality of targets is each hit by intersecting ultrasound beams. FIG. 28 illustrates head 2800 containing targets Insula 2825 and Dorsal Anterior Cingulate Gyrus (DACG) 2830. Frame 2805 holds five ultrasound transducers 2840, 2850, 2860, 2870, 2880. The beam from each ultrasound transducer passes though a band of ultrasound-conduction medium 2815 although in an alternative embodiment the beams can pass through individual ultrasound-conduction media such as shown in FIG. 27. From ultrasound transducer 2840, beam 2842 passes through ultrasound-conduction medium 2815 then into the head, hitting target DACG 2830. From ultrasound transducer 2850, beam 2852 passes through ultrasound-conduction medium 2815 then into the head, hitting target DACG 2830. In like manner, from ultrasound transducer 2860, beam 2862 passes through ultrasound-conduction medium 2815 then into the head, hitting target DACG 2830. Beams 2842, 2852, and 2862 intersect in the Dorsal Anterior Cingulate Gyrus 2830, enhancing the neuromodulation at that target. Effects of beams 2842, 2852, and 2862 are additive. Ultrasound-conjunction conjunction gel (not shown) can be placed just at the interfaces between any of the ultrasound transducers and the band of ultrasonic-conduction medium 2815 and that band and head 2800 as long as the beam regions are covered. The other neural target in FIG. 28 is the Insula 2825. Targeting the Insula are ultrasound transducers 2870 and 2880. From ultrasound transducer 2870, beam 2872 passes through ultrasound-conduction medium 2815 then into the head, hitting target Insula 2825. From ultrasound transducer 2880, beam 2882 passes through ultrasound-conduction medium 2815 then into the head, hitting target Insula 2825. It also will intersect Dorsal Anterior Cingulate Gyrus 2830 but will have minimal impact because it will be the only ultrasound beam present where it passes through the DACG. Beams 2872 and 2882 intersect in the Insula 2825, enhancing the neuromodulation at that target. Beams 2872 and 2882 are additive. Beam 2882 not only neuromodulates the target Insula 2825, but also continues through to neuromodulate DACG 2830 where beam 2882 intersects beams 2842, 2852, and 2862 from ultrasound transducers 2840, 2850, and 2860. The effects of beams 2842, 2852, 2862, and 2882 are additive. The ultrasound transducers can also be used with an acoustic lens (not shown). Again, for elongated targets such as the DACG, the intersecting beams can be spread to cover a broader neural region. In addition the width of the ultrasound transducer and thus the width of the beam can be varied.

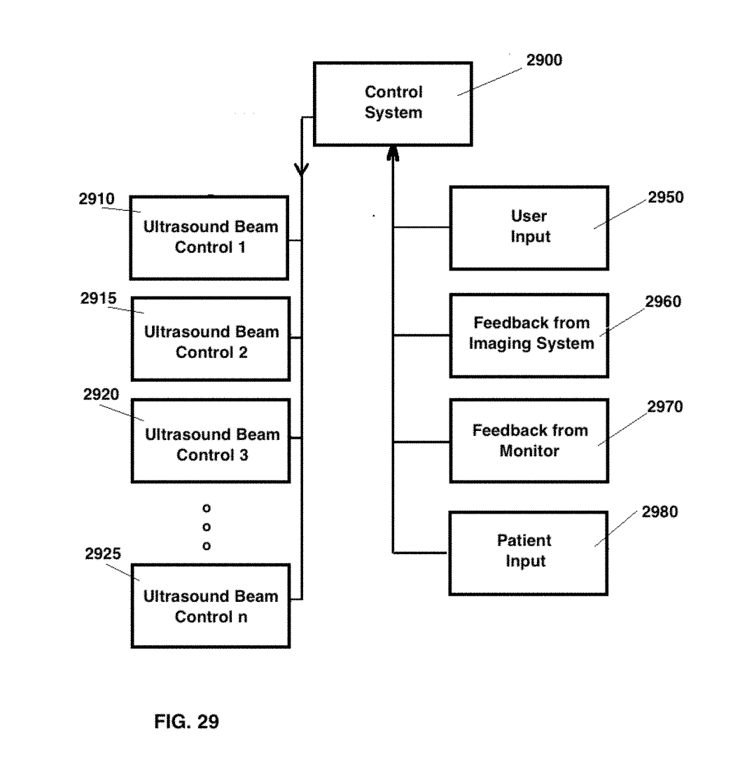

FIG. 29 shows a control block diagram of the mechanism for controlling the multiple ultrasound beams. The direction of the energy emission, intensity, frequency (carrier frequency and/or neuromodulation frequency), pulse duration, pulse pattern, mechanical perturbations, and phase/intensity relationships in targeting for the ultrasonic transducers 2910, 2915, 2920, 2925 (and, as applicable, additional ultrasound transducers as indicated by the ellipsis between ultrasound transducers 2920 and 2925) are controlled by control system 2900 with control input from user by user input 2950 and/or from feedback from imaging system 2960 (either automatically or display to the user with actual control through user input 2950), and/or feedback from a monitor (sound and/or thermal) 2970, and/or the patient 2980 and/or, in the future, other feedback. If positioning of the ultrasound transducers is included as a control element, then control system 2950 will control positioning as well.

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) or enhances acute effects (e.g., such as treatment of post-surgical pain). In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in others only one side will targeted (e.g., the right Insula in the case of addiction).

The invention allows stimulation adjustments in variables such as, but not limited to, intensity, firing pattern, and frequency, mechanical perturbations, phase/intensity relationships, and position to be adjusted so that if a target is in two neuronal circuits the output of the transducer or transducers can be adjusted to get the desired effect and avoid side effects. Position can be adjusted as well. The side effects could occur because for one indication the given target should be up regulated and for the other down regulated. An example is where a target or a nearby target would be down regulated for one indication such as pain, but up-regulated for another indication such as depression. This scenario applies to either the Dorsal Anterior Cingulate Gyrus (DACG) or Caudate Nucleus. Even when a common target is neuromodulated, adjustment of stimulation parameters may moderate or eliminate a problem.

The invention also covers contradictory effects in cases where a target is common to both two neural circuits but needs neuromodulation applied differently for each (e.g., up-regulated in one case and down-regulated in the other case). This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyrus (DACG). To counterbalance the down regulation of the DACG during treatment for pain that negatively impacts the treatment for depression, one would up regulate the Nucleus Accumbens or Hippocampus that are other targets in the depression neural circuit. A plurality of such applicable targets could be stimulated as well.

Another applicable scenario is the Nucleus Accumbens that is down regulated to treat addiction, but up regulated to treat depression. To counteract the down regulation of the Nucleus Accumbens to treat depression but will negatively impact the treatment of depression that would like the Nucleus Accumbens to be up regulated, one would up regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

### Part VI: Ultrasound Macro-Pulse and Micro-Pulse Shapes for Neuromodulation

It is one purpose of some of the inventions described herein to provide methods and systems and methods for non-invasive ultrasound stimulation of neural structures, whether the central nervous systems (such as the brain), nerve roots, or peripheral nerves using macro- and micro-pulse shaping. Positron Emission Tomography (PET) or fMRI imaging can be used to detect which areas of the brain are impacted. In addition to any acute positive effect, there will be a long-term “training effect” with Long-Term Depression (LTP) and Long-Term Potentiation (LTD) depending on the central intracranial targets to which the neuromodulated cortex is connected. In addition, the effect on a readily observable function such as stimulation of the palm and assessing the impact on finger movements can be done and the effect of changing of the macro-pulse and/or micro-pulse characteristics observed. Ultrasound stimulators are well known and widely available.

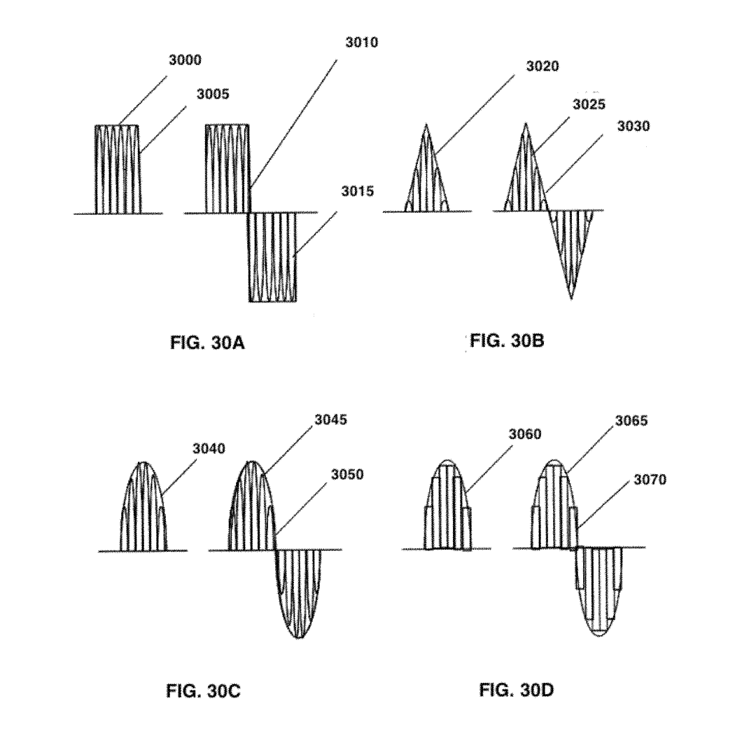

FIGS. 30A to 30D demonstrate macro-pulse shaping defined as the overall shape of the pulse burst. The individual pulses making up the macro-pulse shapes are the micro-pulse shapes. FIG. 30A shows monophasic square-wave macro-pulse 3000 and biphasic square-wave macro-pulse 3010 made up of sine-wave micro-pulses 3005. FIG. 30B illustrates monophasic triangular macro-pulse 3020 and biphasic triangular macro-pulse 3030 made up of sine-wave micro-pulses 3025. FIG. 30C illustrates monophasic sinusoidal macro-pulse 3040 and biphasic sinusoidal macro-pulse 3050 made up of sine-wave micro-pulses 3045. FIG. 30D illustrates monophasic sinusoidal macro-pulse 3060 and biphasic sinusoidal macro-pulse 3070, in this case made up of square-wave micro-pulses 3065.

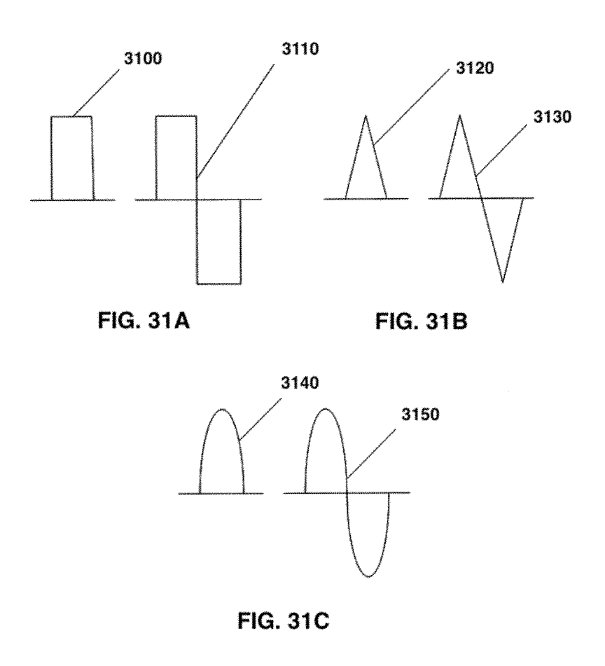

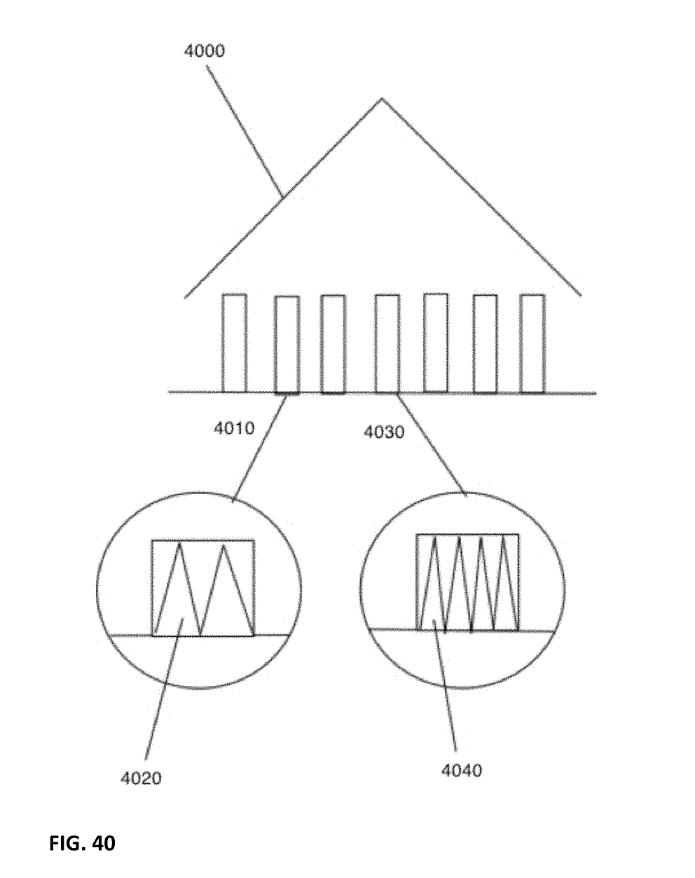

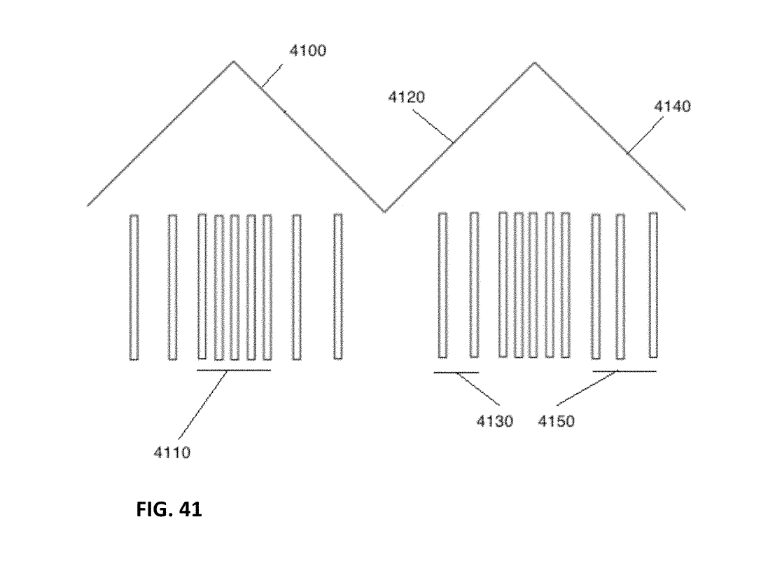

FIGS. 31A to 31C show the micro-pulse shapes that can make up the macro-pulse shapes. FIG. 31A illustrates monophasic square-wave pulse 3100 and biphasic square-wave pulse 3110. FIG. 31B illustrates monophasic triangular pulse 3120 and biphasic triangular pulse 3130. FIG. 40C illustrates monophasic sinusoidal pulse 3140 and biphasic sinusoidal pulse 3150.

Other embodiments can be used with different shapes including those created by signal generators capable of producing arbitrary shapes. The pulse shape can affect the effectiveness of the stimulation and that may vary by ultrasound target. Pulse lengths can be with initial rise times on the order of approximately, but not limited to, 100 microseconds with total pulse length of hundreds of microseconds to one millisecond or more. Another facet of the stimulation is the shape of the pulse and whether the pulse is monophasic or biphasic. As to repetition rate, rates on the order of approximately, but not limited to, 1 Hz or less typically down-regulate and several Hz. and above up-regulate.

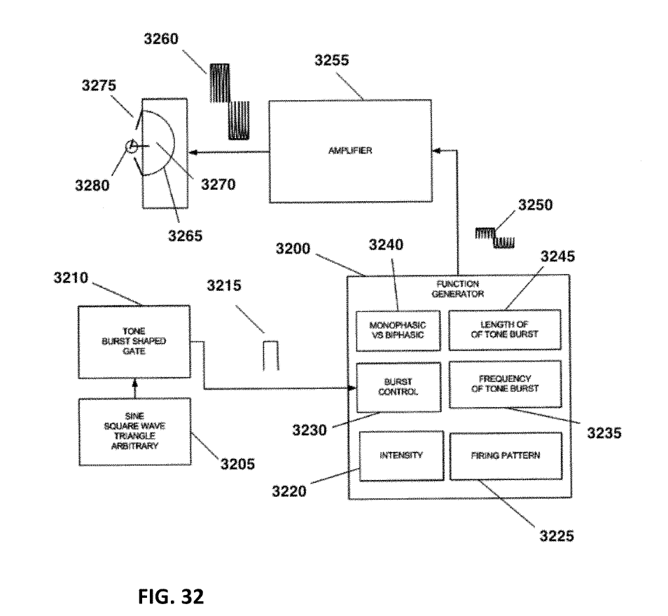

Which macro-pulse and micro-pulse shapes are most effect depends on the target. This can be assessed either by functional results (e.g., doing motor cortex stimulation and seeing which macro- and micro-pulse shape combination causes the greatest motor response) or by imaging (e.g., PET of fMRI) results. Alternatively, the effectiveness of macro-pulse or micro-pulse neuromodulation can be judged by stimulation the palm and assessing the impact of finger movements. The system for generating the macro- and micro-pulse shapes is shown in FIG. 32.

The macro-pulse shape (in this case a square wave) is generated by tone-burst-shaped gate 3210 driven by shape control (sine, square-wave, triangle, or arbitrary) 3205. The output of tone-burst-shaped gate 3210 is 3215 and provides input to burst control 3230 of function generator 3200. The other elements controlled are frequency-of-tone-burst control 3235, intensity control 3220, firing-pattern control 3225, monophasic versus biphasic control 3240, length-of-tone-burst control 3245. The ultrasound transducer is pulsed with tone burst durations of approximately (but not limited to) 25 to 500 μsec. The resulting output (in this case square-wave macro-pulse made up of sine-wave micro-pulses) 3250 provides input to amplifier (for example AB linear) 3255 that provides the increased power as output, shown as increased amplitude pulses 3260. This drives ultrasound transducer 3265 with ultrasound conduction medium 3270 generating focused ultrasound field 3275 aimed at neural target 3280. For any ultrasound transducer position, ultrasound transmission medium (e.g., Dermasol from California Medical Innovations or silicone oil in a containment pouch) and/or an ultrasonic gel layer. Depending on the focal length of the ultrasound field, the length of the ultrasound transducer assembly can be increased with a corresponding increase in the length of ultrasound-conduction-medium insert. The focus of ultrasound transducer 3265 can be purely through the physical configuration of its transducer array (e.g., the radius of the array) with an optional lens or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements. In another embodiment the pulses (macro-shaped; micro-shaping is not applicable) of Transcranial Magnetic Stimulation (TMS) are shaped.

### Intensity Modulated Pulsing

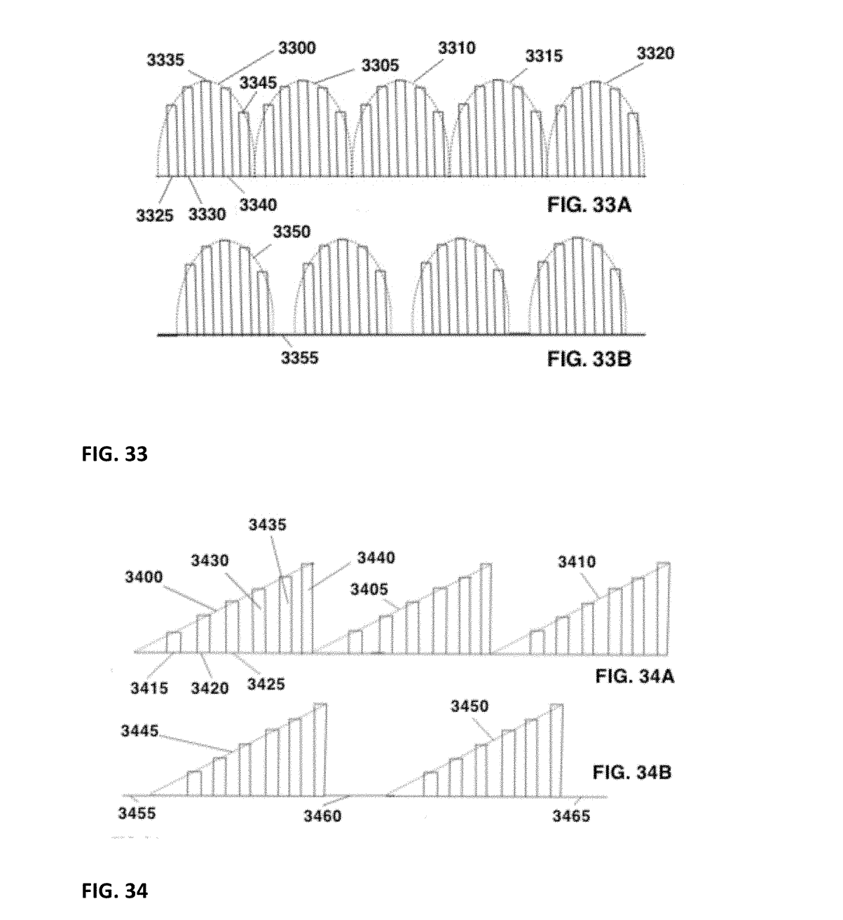

This invention includes novel elements that have not occurred previously, namely intensity modulating the pulses with the benefit of even further enhancing the state change of the neural membrane associated with the pulsing alone. This is called Intensity-Modulated Pulsing. FIG. 33A demonstrates macro pulse shaping contained in a half-sinusoidal envelopes 3300, 3305, 3310, 3315, and 3320. The intensity of the pulses varies within that envelope as indicated by the different amplitudes of square pulses 3325, 3330, 3335, 3340, and 3345. FIG. 33B illustrates an inter-envelope gap 3355 between pulse envelopes like 3350 instead of envelopes 3300 that immediately follow each other as in FIG. 33A. The shape of the envelope can be sinusoidal, triangular, saw-tooth, exponential, or arbitrary. FIG. 34A illustrates saw-tooth envelopes 3400, 3405, and 3410 containing varying amplitude pulses 3415, 3420, 3425, 3430, 3435, and 3440. FIG. 34B shows two envelopes 3445 and 3450 that are separated by inter-envelope intervals 3455, 3460, and 3465. The frequency content of the pulses within the envelope may be swept (for example in the range of approximately 0.5 Hz to approximately 150 kHz) and amplitudes of the pulses and thus the shape of envelope can be swept as well (for example from 1 to 100% of maximum amplitude at the target or varied from 1 percent to 500 percent of the nominal pulse amplitude in a sinusoidal fashion, for example, at 50 Hz). None of the ranges are limiting.

Repeated groups of the same profile may also vary in the same way (e.g., saw tooth, sinusoidal, triangular, or arbitrary fashion). This invention is applicable to all modalities of neuromodulation.

Intensity-Modulated Pulsing is applicable to a variety of the forms of neuromodulation covered in TABLE 1 except for stereotactic radiosurgery that causes a permanent structural change and tDCS that is non-pulsed. Multiple targets neuromodulated with the same or different neuromodulation modalities can have the same or different Intensity-Modulated Pulsing Profiles.

### Part VII: Patterned Control of Ultrasound for Neuromodulation

Some of the inventions described herein are ultrasound devices using non-intersecting beams or intersecting beams delivering enhanced non-invasive deep brain or superficial deep-brain neuromodulation using patterned stimulation impacting one or a plurality of points in a neural circuit providing for up-regulation or down-regulation of neural targets, as applicable, to produce acute effects (as in the treatment of post-surgical pain) or Long-Term Potentiation (LTP) or Long-Term Depression (LTD). Patterns can be applied to multiple beams that intersect to stimulate a single target. One reason for using such intersecting beams is to divide the applied power into multiple components so that the power can be utilized to adequately neuromodulate the intended target without over-stimulating the tissues between the ultrasound transducers and the target and causing undesirable side effects such as seizures.

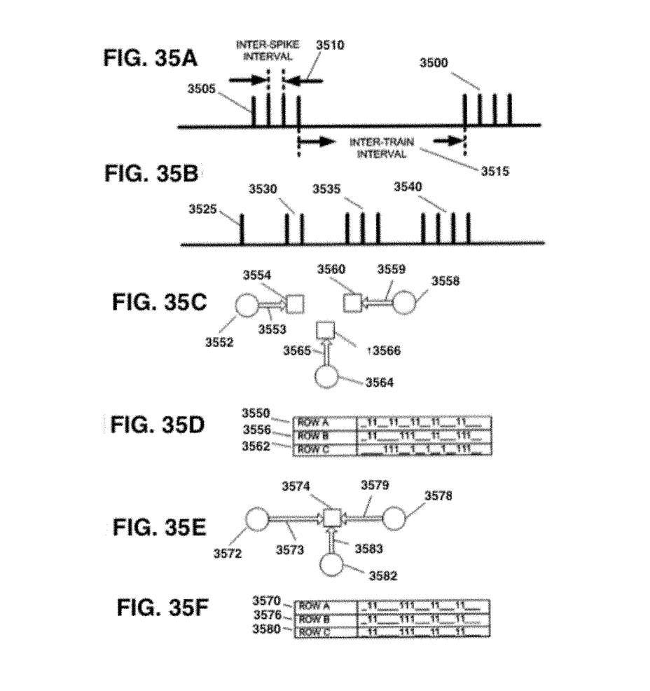

FIGS. 35A-35F illustrate examples of patterns. In FIG. 35A, Pulse trains 3500 are composed of one or a plurality of sets of pulses (e.g., singletons, pairs, triplets, etc.) made up of individual pulses 3505 with inter-spike intervals 3510 with the trains separated by inter-pulse-train intervals 3515. If the set of inter-pulse intervals 3530 is of length zero, then the train is continuous. FIG. 35B illustrates examples of an individual pulse singlet 3525 as well as pulse sets pulse pair 3530, pulse-triplet 3535, and pulse quadruplet 3540. The elements of a train may the same or they may vary. For example, a pair of pulses may alternate with a triplet of pulses and/or the inter-pulse-train intervals may vary. Patterns applied may be either fixed or random. Sample patterns include pairs, triplets, or other multiplicates, and Theta-Burst Stimulation, alternating simple patterns (e.g., alternating pairs with triplets), changing frequencies during stimulations (e.g., for a singlet ramping up the stimulation frequency from approximately 5 Hz. to approximately 20 Hz. over a period of 15 stimulations and then ramping down the stimulation from 20 Hz to 5 Hz. in the next 15 stimulations where the frequencies increase and decrease can be linear or non-linear), and others. Theta-Burst Stimulation (TBS) that consists of short bursts (e.g., three) of high-frequency pulses impulses repeated at 5 Hz (the frequency of the theta rhythm in the EEG). Deisseroth and Schneider (U.S. 2009/0112133 describe theta burst stimulation using ultrasound neuromodulation. Variable or fixed patterns can apply to individual targets or among targets. In some cases the pattern applied to a given neural target or neural circuit may constitute a natural rhythm for that target or circuit and may even include resonance. Patterns include variations in rate or intensity. The relationship between the applied frequency, timing pattern and applied intensity pattern can be independently varied, dependently varied, independently fixed, and dependently fixed. FIG. 35C shows a diagram of three ultrasound transducers 3552, 3558, and 3564 with respective ultrasound beams 3553, 3559, and 3565 impacting three targets 3554, 3560, and 3566 supporting patterned stimulation where multiple ultrasonic transducers are each aimed at different targets.

Depending on the characteristics of the targets, the stimulation patterns of each transducer in a set of transducers may be the same or different. FIG. 35D illustrates examples of stimulation patterns for the case shown in FIG. 35C. Stimulation-pattern row 3550 shows the stimulation pattern for ultrasound transducer 3552 aimed at target 3554. Stimulation-pattern row 3556 shows the stimulation pattern for ultrasound transducer 3558 aimed at target 3560. Stimulation-pattern row 3562 shows the stimulation pattern for ultrasound transducer 3564 aimed at target 3566.

FIG. 35E shows a diagram of three ultrasound transducers 3572, 3578, and 3582 with respective ultrasound beams 3573, 3579, 3583 impacting common target 3574 supporting patterned stimulation where multiple ultrasonic transducers are each aimed at the same target. FIG. 35F illustrates examples of stimulation patterns for the case shown in FIG. 35E. Stimulation-pattern row 3570 shows the stimulation pattern for ultrasound transducer 3572 aimed at target 3574. Stimulation-pattern row 3576 shows the stimulation pattern for ultrasound transducer 3578 also aimed at target 3574. Stimulation-pattern row 3580 shows the stimulation pattern for ultrasound transducer 3582 again also aimed at target 3574. Even when a common target is neuromodulated, adjustment of stimulation parameters may moderate or eliminate a problem with side effects from the neuromodulation.

In the case of synchronous patterns, the same pattern is applied to multiple targets. In the case of asynchronous patterns, different patterns are applied to different targets. In the case of independent patterns when two different patterns are applied to different targets, when one pattern is changed, the other is not changed or not in changed in the same way. If one or a plurality of targets are all up-regulated or all down-regulated or there is a mixture of such regulation, different frequencies can be used to optimize the desired effects on the various targets (e.g., one up-regulation done at 5 Hz. and another at 10 Hz.). Invention includes the concept of having different patterns for each of a pair of bilateral structures. For example, in the treatment of addiction, neuromodulating the Insula involves down regulating the Insula on the right side.

In another embodiment the ultrasound beams intersect at the targets. This can be useful where one wants to increase the intensity level at a given target, but decrease the intensity of tissue intermediate between the output interface of the ultrasound transducer and the given target. In this invention, two or more beams intersect at a given target with appropriate patterns applied to each of the beams. Use of patterns and/or intersecting ultrasound beams avoids excessive stimulation of nearby structures that need to be protected.

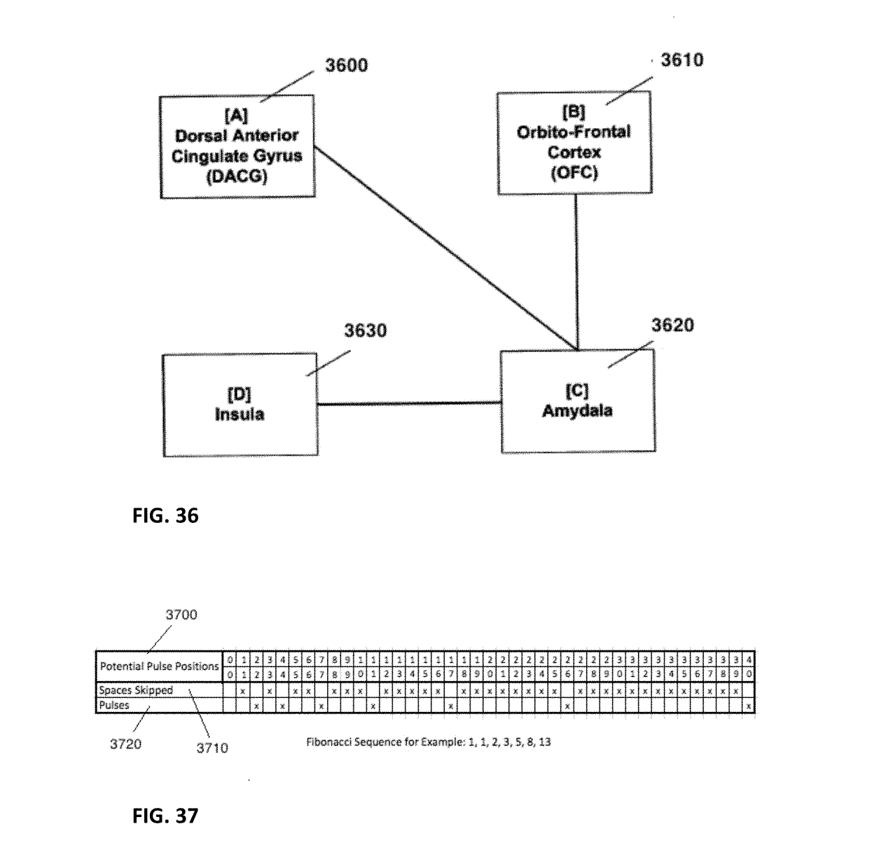

FIG. 36 illustrates the neural circuit representing the case where alternative effects can occur depending on whether the elements of the circuit are either up regulated or down regulated. Note in some cases in a given circuit not all the elements will be all up regulated or down regulated. In FIG. 36, blocks [A] 3600, [B] 3610, [C] 3620, and [D] 3630 represent neural elements that can be up regulated or down regulated. In this example, for one clinical effect, all are regulated in the direction to achieve that effect, and for the opposite clinical effect, all are regulated in the opposite direction. As a specific embodiment, for bipolar disorder, [A] 3600 represents the Dorsal Anterior Cingulate Gyms (DACG), [B] 3610 represents the Orbital-Frontal Cortex (OFC), [C] 3620 represents the Amygdala, and [D] 3630 represents the Insula. For the condition Bipolar Disorder, if the depressive phase is being treated, the OFC 3610, the Amygdala 3620, and left-located Insula 3630 are down regulated, and the DACG 3600 and right-located Insula are up regulated. On the other hand, if the manic phase is being treated, the OFC 3610, the Amygdala 3620, and left-located Insula 3630 are up regulated, and the DACG 3600 and right-located Insula 3630 are down regulated. In a sense, the circuit is sped up or advanced to treat the depressive phase and slowed down or retarded to treat the manic phase. Patterned neuromodulation as covered in this part provide the mechanism to accomplish such up- and down-regulation.

The invention allows stimulation adjustments in variables such as, but not limited to, intensity, timing, firing pattern, mechanical perturbations, phase/intensity, frequency, and position to be adjusted so that if a target is in two neuronal circuits the output of the transducer or transducers can be adjusted to get the desired effect and avoid side effects. Position can be adjusted as well. The side effects could occur because for one indication the given target should be up regulated and for the other down regulated. An example is where a target or a nearby target would be down regulated for one indication such as pain, but up-regulated for another indication such as depression.

The invention also covers contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyrus (DACG). To counterbalance the down-regulation of the DACG during treatment for pain that negatively impacts the treatment for depression, one would up-regulate the Nucleus Accumbens or Hippocampus that are other targets in the depression neural circuit. A plurality of such applicable targets could be stimulated as well. One set of applied patterns can be applied to a given neural circuit to provide treatment for one condition and an alternative set of applied patterns is applied to the given neural circuit to provide treatment for another condition.

Another applicable scenario is the Nucleus Accumbens that is down regulated to treat addiction, but up regulated to treat depression. To counteract the down-regulation of the Nucleus Accumbens to treat depression but will negatively impact the treatment of depression that would like the Nucleus Accumbens to be up regulated, one would up-regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

Additional patterns follow. They are applicable to the various modalities of neuromodulation and pulse width and frequency may vary. The various pulse patterns are shown in TABLE 5 as well as applicable figures.

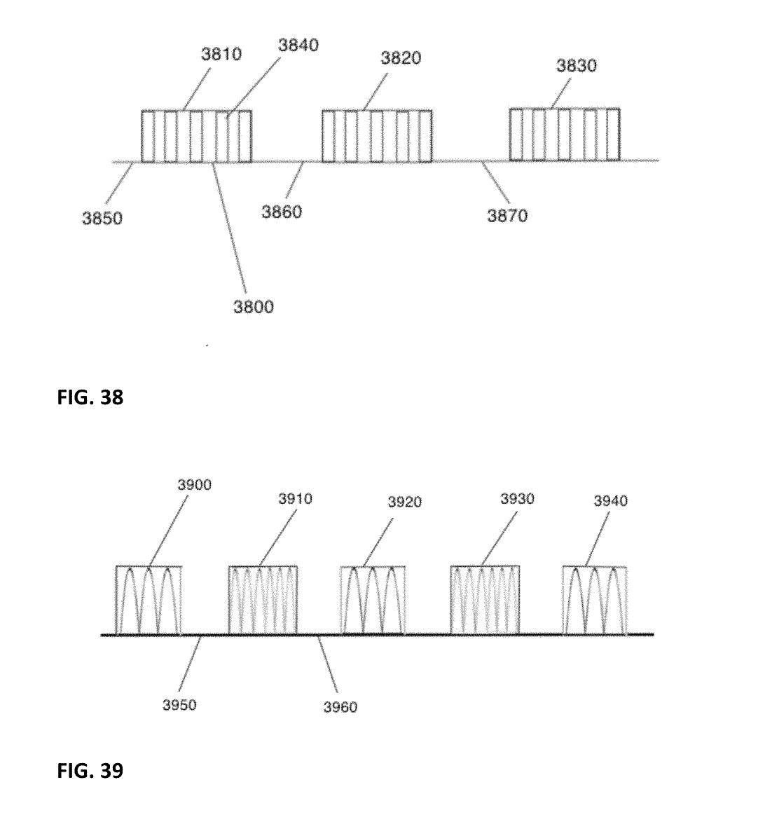

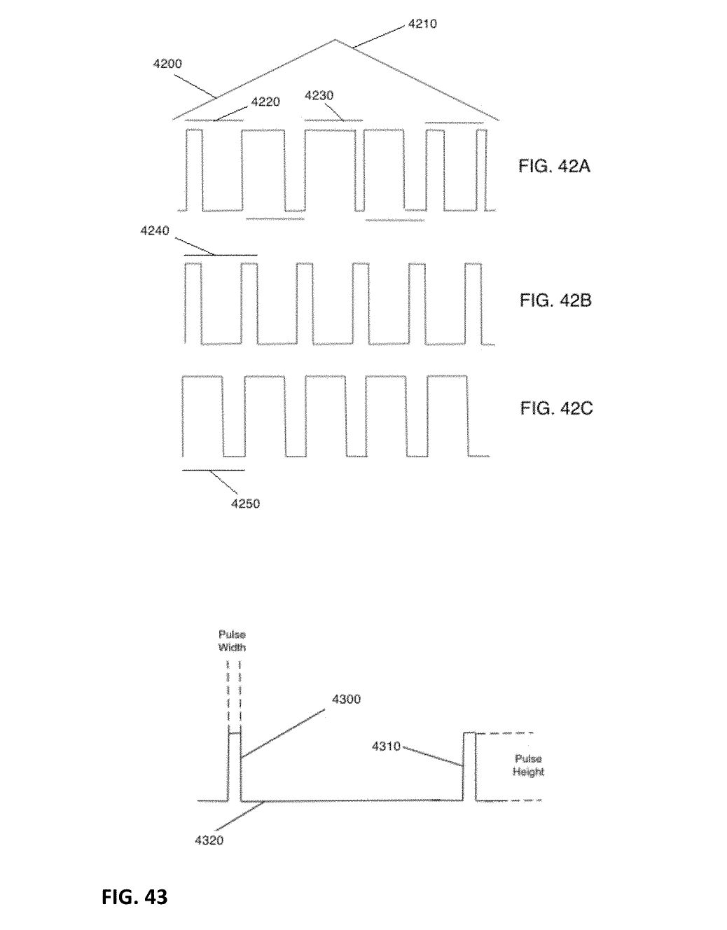

TABLE 5

| | DESCRIPTION OR |
|---|---|
| PATTERN | FIGURE REFERENCE |
| FIXED | Fixed |
| RANDOM | Pseudo Random Number Generator |
| | produces random number for which |
| | potential slot a given pulse |
| | will occur (see text). |
| FIBONACCI PULSING | See FIG. 37 |
| CONTINUOUS | Continuous non-pulsed |
| BURST-MODE PATTERN | See FIG. 38 |
| MULTIPLE FREQUENCY | See FIG. 39 |
| NEUROMODULATION | |
| SWEEP NEUROMODULATION | See FIG. 40 |
| FREQUENCY | |
| SWEEP PULSE FREQUENCY | See FIG. 41 |
| DUTY CYCLE | See FIG. 42 |

### Fixed Pulse Pattern

In this embodiment, list in the second row of TABLE 5 above, both the pulse width and inter-pulse interval are fixed.

### Random Pulse Pattern

Random pulsation is listed in the second row of TABLE 5 above. Random pulses are generated using a computer running a pseudo-random-number-generator program generating random numbers in the range of 1 to whatever the whole range of the target average pulse interval divided by the pulse width. An example is where the average is 2 Hz or on the average one pulse every 500 ms. With a pulse width of 0.2 ms, there would be 500 ms/0.2 ms equals 2500 potential slots that a pulse could occur within that 500 ms period and still have an average of 2 Hz. The randomly generated number would designate in which one of the 2500 potential slots that pulse would occur within the given 500 ms period.

### Fibonacci Pulse Pattern

The application of a Fibonacci-Sequence pulse pattern is shown in FIG. 37. Potential pulse positions are designated in the top two rows of the table, 3700. The spaces skipped are marked with x appearing in row 3710 and the pulses delivered are marked with an x appearing in row 3720. The novel pattern generated by Fibonacci sequence used in this type of neuromodulation is determined by a Fibonacci sequence applied to the number of space elements between pulse elements. The duration of each space element can vary between approximately 0.1 ms and approximately 5 sec. The duration of each pulse element can vary between approximately 0.01 ms and approximately 1 sec. In a given pattern, the duration of each space element need not be the same as the duration of each pulse element and the durations of each pulse element need not be equal. In the Fibonacci sequence shown in FIG. 37, the beginning numbers are 1, 1, so the designated eight terms in this case cause the sequence 1, 1, 2, 3, 4, 5, 8, and 13 to be generated. In the example for this figure they are applied to the number of spaces to be skipped in the order they were generated, not randomly from those eight numbers. The generated pulses, starting at time 0 will occur at positions 2, 4, 7, 11, 17, 26, and 40. This is because there will be one space between positions 0 and 2, one space between positions 2 and 4, 2 spaces between positions 4 and 7, 3 spaces between positions 7 and 11, 5 spaces between positions 11 and 17, 8 spaces between positions 17 and 26 and 13 spaces between positions 26 and 40. The actual length of time for those 40 spaces is essentially 33 spaces because the 7 pulses are likely to be so short (e.g., 0.2 ms) so if the duration of a space is 20 ms each then the length of time for the 7 pulses is 33 times 20 ms equals 660 ms. To get the average frequency in Hz, the number of pulses in one second is ((1,000 ms/sec)/660 ms) times 7=10.6 pulses/sec=10.6 Hz. This 10.6 Hz rate is in the range of up regulation. If the order to be applied as random, the average frequency would be the same.

### Continuous, Non-Pulsed Neuromodulation

Continuous (non-pulsed) neuromodulation is listed in the fourth row of TABLE 5 above. It can be employed for a modality such as optogenetics can be used in a continuous mode. In another embodiment, not shown, the amplitude/intensity of the continuous, non-pulsed neuromodulation can vary.

### Burst-Mode Pattern

As shown in FIG. 38, superimposed on baseline 3800 are bursts 3810, 3820, and 3830 made up of individual pulses 3840 and separated by inter-burst intervals 3850, 3860, and 3870. Each of the bursts would typically contain a train of pulses (say square-pulses 0.2 ms in length at 2 Hz). As an example, the bursts could be six second long and repeated every nine seconds. Any pulse pattern train can be contained within a burst.

### Multiple-Frequency Amplitude Modulation

Neuromodulation systems to date deliver pulses of a single frequency (say 900 Hz) and pulse interval (say every 0.2 ms) superimposed on a carrier frequency (say 0.65 MHz) to the target. In the current invention, pulses of two or more different frequencies (e.g., for two frequencies, 1000 Hz every 0.2 ms and 1500 Hz every 0.2 ms, but offset by 0.1 ms so they do not overlap) are delivered simultaneously on a single carrier. In FIG. 39, pulses 3900, 3930, and 3940 are made up of the same lower frequency content than pulses 3910 and 3930 that are made of the same higher frequency content. The pulses are separated by inter-pulse interval. In some embodiments there can be a mixture of frequencies and inter-pulse intervals whether directed to single or different targets of any number with recognition that with varying pulse intervals that some pulses may overlap. The range of the two or more frequencies will be between approximately 10 Hz to 400 Hz for down regulation and approximately 500 Hz to 5 MHz for up regulation. The adjective approximately is used because depending on the patient the frequency break between up regulation and down regulation (for example, in some cases the frequency for down regulation might go up to 600 Hz and the neuromodulation frequency for up regulation begin at 900 Hz, but in any case wherever the break would be determined through neuromodulation of the specific patient without reservation).

### Sweep Neuromodulation Frequency

In this embodiment the neuromodulation frequency (as contained within the envelope of the pulses) is varied or swept through a range. In FIG. 40, the profile of the change in the neuromodulation frequency 4000 controls the neuromodulation frequency content of pulses 4010 as shown in zoom-in bubble 4020 with a lower frequency of waves with the neuromodulation generator frequency corresponding to a lower spot 4040 on profile 4000 than pulse 4030 located at a higher point in profile 4000 with higher frequency of waves as seen in zoom-in bubble 4040. Such variation in time can occur over any time period from zero to 60 seconds or higher, without reservation. For example, the frequency for up regulation may be sinusoidal (or other fashion) varied periodically from 1000 Hz to 2000 Hz repeated over a period of 10 seconds. The profile can be of any shape (e.g., sinusoidal or triangular). The range through which the amplitude-modulated neuromodulation frequency will be swept will typically be between approximately 10 Hz to approximately 400 Hz for down regulation and approximately 500 Hz to approximately 5 MHz for up regulation.

The frequency content of each pulse itself can be made of square waves, sinusoidal waves, saw-tooth waves, or other waves, including those of arbitrary shape. The pulses 4010 and 4030 themselves can be fixed or variable as to inter-pulse interval or pulse width. In another embodiment, the neuromodulation pulse amplitude is varied or swept through a range. For example, the amplitude may vary in the range of 10% of full-scale power of the generator to 100% of full-scale power or varied from 1 percent to 500 percent of the nominal pulse amplitude in a sinusoidal fashion, for example, at 50 Hz.

### Sweep Pulse Frequency

FIG. 41 illustrates sweeping the neuromodulation pulse frequency with the spacing between pulses in areas 4110, 4130, and 4150 being changed according to the envelope of the controlling frequency in the pulse generator as indicated by frequency-versus-time graph as pointed to by 4100, 4120, and 4140. When the controlling frequency is high as indicated by point 4100, pulses 4110 are closer together than when the controlling frequency is lower as pointed to by 4120 and 4140, pulses 4130 and 4150 are further apart. The change in the controlling frequency can follow a variety of profiles, for example, sine wave, triangle wave, saw-tooth, or other, including arbitrary. The controlling frequency will typically be in the range of approximately 10 Hz to approximately 2 kHz.

### Duty Cycle

The neuromodulation pulse duty cycle (the proportion of the inter-pulse interval that is with filled neuromodulation pulse) may be either fixed at different values or swept through a set of values over a period of time. FIG. 42A shows sweeping of duty cycle with points 4200 and 4210 representing the control signal driving the change in duty cycle. Horizontal lines 4020 and 4030 show the length of each duty cycle with all the pulse-to-pulse intervals being the same length. The duty-cycle percentage varies with the height of the control signal. At point 4000 on the controlling frequency, the duty cycle of the related pulse 4220 has a smaller duty-cycle compared to point 4210 on the controlling frequency, the related pulse 4230 in which duty-cycle is much longer. The duty cycle is swept in the range of 1% to 100% of the inter-pulse interval with the controlling frequency sweeping occurring over approximately 1 Hz to approximately 20 kHz.

The figure set also illustrates two examples of a fixed duty cycle, FIG. 42B with one duty cycle 4240 and FIG. 42C with a different fixed duty cycle 4250 that is larger. In another embodiment, the duty cycle is not fixed but is swept in the range of 1% to 100% of the inter-pulse interval with the controlling frequency sweeping occurring over 1 Hz to 20 kHz.

### Multiple Targets

In one embodiment of neuromodulation of multiple targets, the neuromodulation of each of the multiple targets has the same pattern. In an alternative embodiment, the neuromodulation of at least one of the multiple targets has a different pattern.

### Cumulative Energy Delivered

One consideration for any of the pulse patterns, except continuous stimulation, is that the pulse width, height, and shape may vary in any given embodiment. Different pulse patterns will have different cumulative values. Energy level is relative to positioning of transducer to target, but for that position the accumulation of pulses with given width, heights and intervals will reflect total energy delivered. For example, if arbitrary energy level is one unit, take (average) pulse width over selected time period times the number of pulses in that time period and the result is the relative energy delivered. It is understood that in some cases one or both of the pulse width and interval will vary in which case either calculated average values or actual counts will be used. TABLE 6 contains pulse width and frequency for various pattern types.

TABLE 6

| PATTERN TYPE | PULSE WIDTH | FREQUENCY |
|---|---|---|
| Fixed | (Average) Pulse Width | Frequency |
| Random | (Average) Pulse Width | Average Frequency |
| Fibonacci | (Average) Pulse Width | Average Frequency |
| Continuous Non- | (Average) Pulse Width | |
| Pulsed | | |
| Burst-Mode Pattern | (Average) Pulse Width | Average Frequency |

In FIG. 43, pulses 4300 and 4310 considering interval 4320, adding up the total area of the pulses over the period of interest (for example, 50 minutes or a day) will calculate the total relative energy delivered for the given period. The selected time period would usually be the length of a session for non-invasive neuromodulation and a designated time period (such as 24 hours) for continuous invasive neuromodulation such as deep brain stimulation. The same principle applies to pulse height and pulse shape. TABLE 7 contains examples of the relative energy calculation using a time period of an hour to simply the example.

TABLE 7

| | | | Relative |
|---|---|---|---|
| | | (Average) | Energy per |
| Frequency | Number | Pulse | Hour |
| (Hz) | per Hour | Width (ms) | (ms/hr) |
| 1 | 3600 | 0.1 | 360 |
| 10 | 36000 | 0.1 | 3600 |
| 100 | 360000 | 0.1 | 36000 |
| 1 | 3600 | 1 | 3600 |
| 10 | 36000 | 1 | 36000 |
| 100 | 360000 | 1 | 360000 |
| 1 | 3600 | 2 | 7200 |
| 10 | 36000 | 2 | 72000 |
| 100 | 360000 | 2 | 720000 |

In the case of multiple targets, the cumulative value would be the sum of the values for the individual targets.

The methods and systems described here are applicable to all forms of neuromodulation, whether non-invasive or invasive.

### Part VIII: Ancillary Stimulation

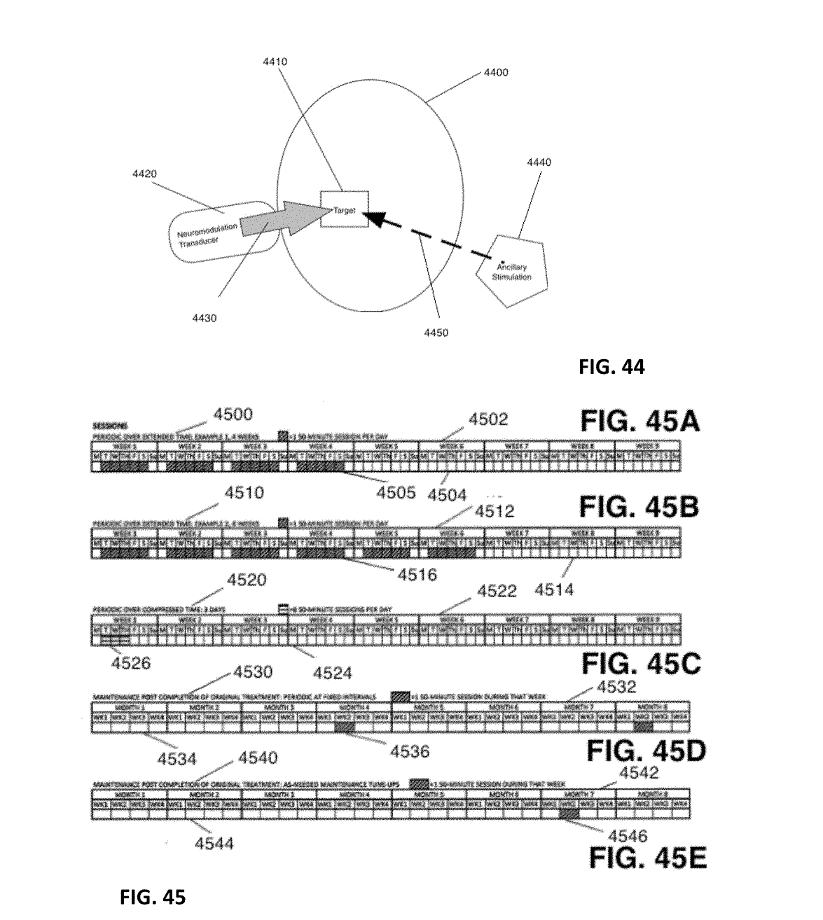

FIG. 44 illustrates an embodiment for ancillary stimulation. Target 4410 within patient head 4400 is neuromodulated by Neuromodulation transducer 4420 through its energy output 4430. Ancillary stimulation 4440 via its pathway 4450 also acts on target 4410 (or associated/connected targets) to provide the augment effect. Ancillary stimulations such as visual, auditory, tactile, vibration, pain, proprioceptive stimulation or any other form of energy input can be applied. Ancillary Audio stimulation is not restricted to a single tone or combination of tones. Music or other sounds (e.g., waves, animal sounds) can be effective for up-regulation or down-regulation. Upbeat music can aid in the treatment of depression. Soothing or downbeat music can aid in the treatment of anxiety play downbeat music. In like manner, visual stimulation can be tied to up-regulation or down-regulation. In the case of depression, for example a funny cartoon could be used while a video of a calm brook could aid in the treatment of anxiety. The part of the body may influence the effect like the affected limb in the rehabilitation of stroke.

### Part IX: Planning and Using Sessions of Ultrasound for Neuromodulation

FIGS. 45A-45E show a diagram of exemplar session types for both initial treatment and maintenance sessions. FIG. 45A illustrates example 4500, Periodic Over Extended Time with 4 weeks of treatment where time divisions are weeks 4502 divided into days 4504 with 50-minute sessions on indicated days 4506. For all of these examples, the session length could be longer or shorter than 50 minutes. FIG. 45B illustrates example 4510, Periodic Over Extended Time with 6 weeks of treatment where time divisions are weeks 4512 divided into days 4514 with 50-minute sessions on indicated days 4516. FIG. 45C illustrates example 4520, Periodic Over Compressed Time with 3 days of treatment where time divisions are weeks 4522 divided into days 4524 with 50-minute sessions on indicated days 4566. FIG. 45D illustrates example 4530, Maintenance Post Completion of Original Treatment at Fixed Intervals where time divisions are months 4532 divided into weeks 4534 with 50-minute sessions during indicated weeks 4536. FIG. 45E illustrates example 4540, Maintenance Post Completion of Original Treatment with As-Needed Maintenance Tune-Ups where time divisions are months 4542 divided into weeks 144 with 50-minute sessions during indicated week 4546. An example of one of the treatments to which sessions would be applicable is depression and bipolar disorder. Multiple targets can be neuromodulated singly or in groups to treat depression or bipolar depression. To accomplish the treatment, in some cases the neural targets will be up regulated and in some cases down regulated, depending on the given neural target. In some embodiments the maintenance or tune-up is triggered when the patient's symptoms deteriorate in the range of 5% to 1000% or more.

Sessions are routinely used in Transcranial Magnetic Stimulation (e.g., 50 minute sessions five days per week for four to six weeks). A novel approach of this part is an embodiment with application of sessions with a different number of daily sessions each week (e.g., five sessions the first week, two the second week, four the third week, three the fifth week, etc.) or to have sessions every other week, or to have the number of sessions in a given week randomly drawn from the first six terms of a Fibonacci Sequence beginning with (0, 1) namely (0, 1, 1, 2, 3, 5) or the first five terms of a Fibonacci Sequence beginning with (1, 1) namely (1, 1, 2, 3, 5).

### Part X: Patient Feedback for Control of Neuromodulation

It is the purpose of some of the inventions described herein to provide methods and systems for the adjustment of deep brain or superficial neuromodulation using ultrasound or other non-invasive modalities to impact one or multiple points in a neural circuit under patient-feedback control.

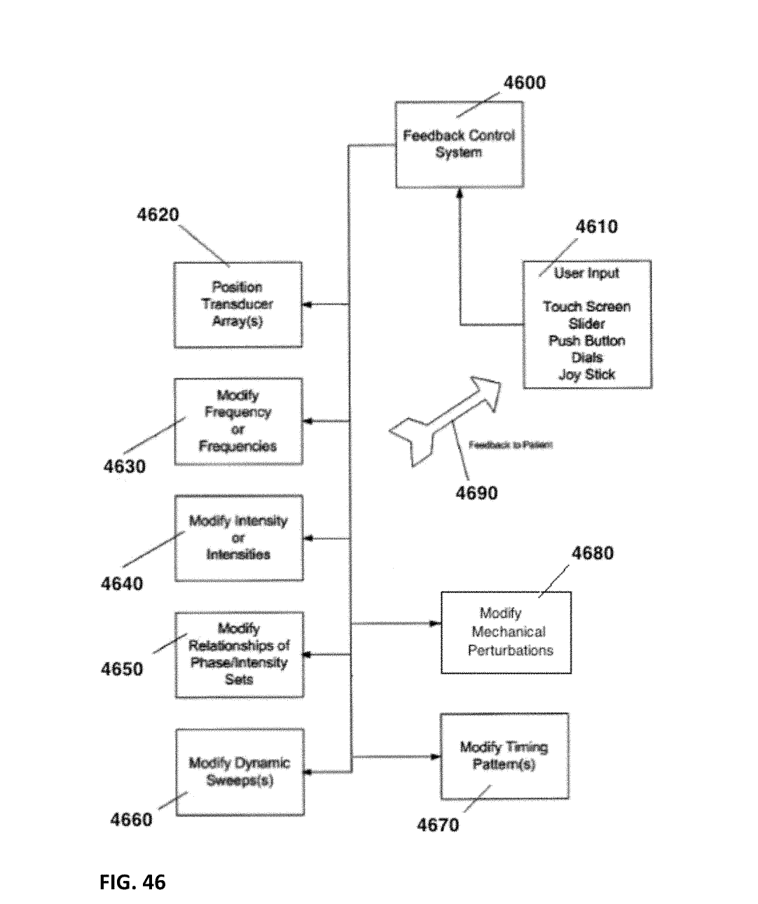

FIG. 46 shows the basic feedback circuit. Feedback Control System 4600 receives its input from User Input 4610 and provides control output for positioning ultrasound transducer arrays 4620, modifying pulse frequency or frequencies 4630, modifying intensity or intensities 4640, modifying relationships of phase/intensity sets 4650 for focusing including spot positioning via beam steering, modifying dynamic sweep patterns 4660, modifying timing patterns 4670, and/or modifying mechanical perturbations. Feedback to the patient 4690 occurs with what is the physiological effect on the patient (for example increase or decrease in pain or decrease or increase on tremor. User Input 4620 can be provided via a touch screen, slider, dials, joystick, or other suitable means. Control of the flow in FIG. 46 can occur as in FIG. 57 with it accompanying description.

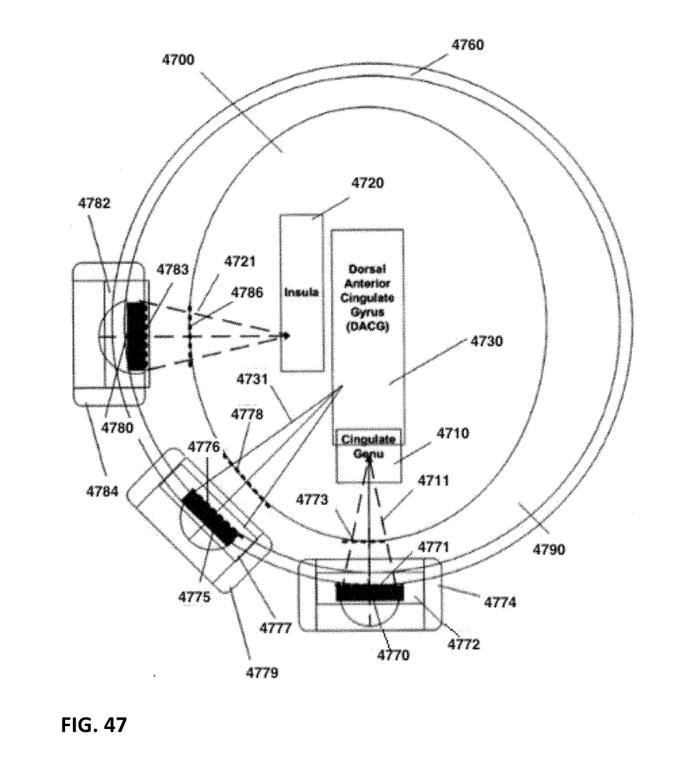

An example of a multi-target neural circuit related to the processing of pain sensation is shown in FIG. 47. Surrounding patient head 4700 is ultrasound conduction medium 4790, and ultrasound-transducer holding frame 4760. Attached to frame 4760 are transducer holders 4774, 4779, and 4784. These are oriented towards neural targets respectively holder 4774 towards the Cingulate Genu 4710, holder 4779 towards the Dorsal Anterior Cingulate Gyrus (DACG) 4730, and holder 4784 towards Insula 4720. The assembly targeting Cingulate Genu 4710, includes transducer holder 4774 containing transducer 4770 mounted on support 4772 (possibly moved in and out via a motor (not shown)) with ultrasound field 4711 transmitted though ultrasound conducting gel layer 4771, ultrasound conducting medium 4790 and conducting gel layer 4773 against the exterior of the head 4700. Examples of sound-conduction media are Dermasol from California Medical Innovations or silicone oil in a containment pouch.

The assembly targeting Dorsal Anterior Cingulate Gyrus 4730, includes transducer holder 4779 containing transducer 4775 mounted on support 4777 (possibly moved in and out via a motor (not shown)) with ultrasound field 4731 transmitted though ultrasound conducting gel layer 4776, ultrasound conducting medium 4790 and conducting gel layer 4778 against the exterior of the head 4700.

The assembly targeting Insula 4720, includes transducer holder 4784 containing transducer 4780 mounted on support 4782 (possibly moved in and out via a motor (not shown) with ultrasound field 221 transmitted though ultrasound conducting gel layer 4783, ultrasound conducting medium 4790 and conducting gel layer 4786 against the exterior of the head 4700.

With reference to FIG. 47 for the treatment of pain, the Cingulate Genu 4710, and DACG 470, and Insula 4720 would all be down-regulated. The ultrasonic firing patterns can be tailored to the response type of a target or the various targets hit within a given neural circuit.

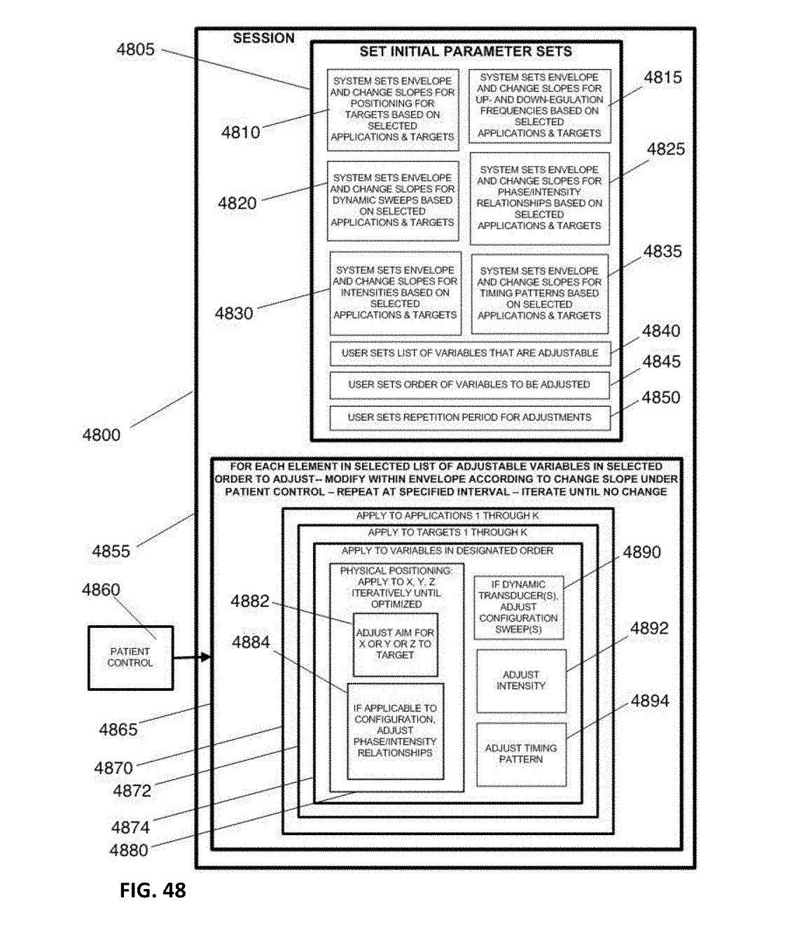

FIG. 48 shows an algorithm for processing feedback from the patient to control the ultrasound neuromodulation during a session 4800. Before the real-time session begins, the initial parameters sets are set 4805 by the system. This can be automatically, by the user healthcare professional instructing the system, or a combination of the two. These include setting the envelope and change slopes based on selected applications and targets for positioning for targets 4810, up- and down-regulation frequencies 4815, sweeps for dynamic transducers 4820, phase/intensity relationships 4825, intensities 4830, and timing patterns 4835 (other elements like mechanical perturbations can be included as well). The user setting what is to be controlled by the patient during the real-time feedback, namely list of variables that are adjustable 4840, order of those variables to be adjusted 4845, and repetition period for adjustments 4850 follows these. One with ordinary skill in the art can apply the neuromodulation variables as applicable in the order above or can change the order; individual variables can be change in the range of approximately 1 to approximately 20 percent at one time. Control of the flow in FIG. 48 can occur as in FIG. 57 with it accompanying description.

Once the initialization is complete the real-time part of the session begins based on patient-controlled input 4860 (e.g., via touch screen, slider, dials, joy stick, or other suitable mean). During real-time processing, the outer loop 4865 applies for each element in selected list of adjustable variables in selected order to adjust a modification within the envelope according to the change slope under patient control with repetition at the specified interval with iteration until there is no change felt by the patient. The process includes applying to applications 1 through k 4870, applying to targets 1 through k 4872, applying to variables in designated order 4874, physical positioning (iteratively for x, y, z) 4880 including adjusting aim towards target 4882 and, if applicable to configuration, adjust phase/intensity relationships 4884, in addition to adjustment of configuration sweeps if there is/are dynamic transducer(s) 4890, adjust intensity 4892, and adjusting timing pattern 4894.

### Guided Feedback

This invention includes the novel feature of Guided-Feedback Neuromodulation wherein a set of neuromodulation parameters/variables is applied, the patient, operator, or agent (intelligent judge of input from physiological sensors) judges the result, and based on that input an algorithm is applied to determine the neuromodulation parameters/variables to be applied in the next segment.

TABLE 8 lists the variable parameters for neuromodulation that can be used individually or make up sets that can be change on the basis of Guided Feedback and the neuromodulation modalities to which they would apply. The applicable neuromodulation modalities are both non-invasive and invasive.

TABLE 8
VARIABLE PARAMETERS VERSUS NEUROMODULATION MODALITY

| | | | | | | | Mechanical | Mechanical | |
|---|---|---|---|---|---|---|---|---|---|
| | Neuromodulation | Pulse | Pulse | | | | Perturbation | Perturbation | Light |
| | Frequency | Duration | Frequency | Pulse Patterns | Intensity | Phase | Length | Frequency | Wavelength |
| Deep Brain Stimulation | | X | X | X | X | | | | |
| Spinal Cord Stimulation | | X | X | X | X | | | | |
| TMS | | X | X | X | X | | X | X | |
| Ultrasound | X | X | X | X | X | X | X | X | |
| RF | X | X | X | X | X | | X | X | |
| VNS | | X | X | X | X | | | | |
| Optogenetics | | X | X | X | X | | | | X |

A simple example of a parameter set that would be varied during Guided Feedback processing is a combination pulse duration (varying in the range between 0.1 ms to 0.25 ms in increments of 0.05 ms), pulse frequency with choices of 15, 30, and 45 Hz for up regulation or choices 0.5, 1, and 2 Hz for down regulation, neuromodulation frequency, if applicable to the given modality, of 100, 200, or 300 Hz for down regulation and 500, 1000, and 1,500 Hz for up regulation, and pulse pattern using a the first 3 or 5 elements in Fibonacci sequence with initial elements of 0 and 1. This sample set is applicable to multiple modalities. A sample initial set for one with ordinary skill in the art is a pulse duration of 0.1 ms, pulse frequency of 15 Hz at a neuromodulation frequency of 1000 Hz for up regulation or 1 Hz at a neuromodulation frequency of 300 Hz for down regulation, and using the first 5 elements of Fibonacci sequence with initial elements of 0 and 1.

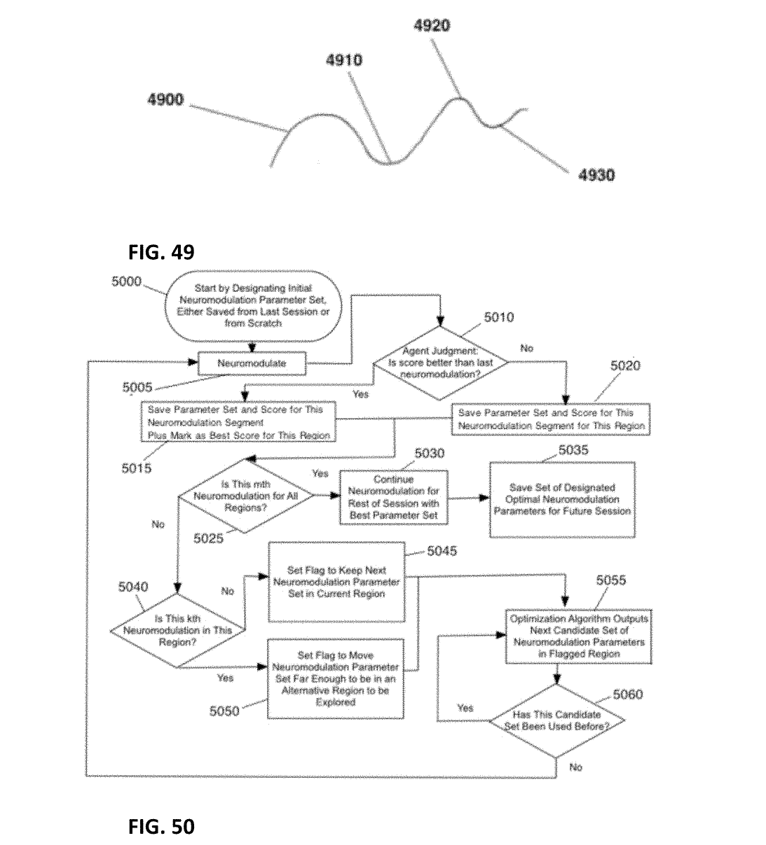

An illustration of one of the guidance algorithm appears in FIG. 49, in this case a Hill Climbing Algorithm. Line 4900 represents the physiological state of the patient (e.g., pain or tremor status) that can vary according to the applied set of neuromodulation parameters/variables. The deeper the minimum, the better the symptoms of the patient are. Therefore, minima 4910 and 4930 represent potentially better symptom states for this given patient and the objective is to locate the best minimum or at least a good minimum so the neuromodulation will better benefit the patient. In this figure minimum 4910 would be preferable to minimum 4930. Maximum 4920 indicates a region that one would want to avoid because it is a region where the patient would have worse symptoms. A region to be explored is the variation of symptoms caused by usually, but not necessarily small variations of the neuromodulation parameters. Point 4900 indicates the level of patient symptoms for the initial neuromodulation. The Hill Climbing Algorithm varies the parameter set within a region to locate the minimum giving the best result in terms of the patient having better symptoms. Say that the algorithm is exploring region around minimum 4930. The algorithm can optimize benefit in that region, perhaps finding where the patient's symptoms are best, but that region represents a local minimum rather than a global minimum 4910. A time interval in which a set of neuromodulation is applied prior to that set being applied for a long interval in which the neuromodulation are not changed is called an exploratory period. A consideration, therefore, is to periodically (typically, but not limited to) every 2 to 6 applications of parameter set) randomly jump to another region and explore that having recording results for the entire sequence of neuromodulation parameter/variable sets and associated results so neuromodulation control can return to the optimal set for long term or longer term neuromodulation after the exploratory period had been completed.

A flow chart for the process appears as FIG. 50. Initial block 5000 notes that the session may be started from scratch with a new set of neuromodulation parameters/variables or may use the parameter set from the last session (or could be another previous session). Another alternative is to just use an established parameter set that has been found to be satisfactory and not apply patient/operator/agent feedback for this session. In the case of an invasive modality such as DBS or VNS with long-term continuous stimulation, initiating another round of guided/directed feedback might only be triggered if patient symptoms deteriorate. Control of the flow in FIG. 50 can occur as in FIG. 57 with it accompanying description.

In step 5005, neuromodulation is applied and in step 5010 a decision is made as to whether the symptoms are better or worse (patient, operator, or an agent (intelligent judge of input from physiological sensors such as a tremor detector)). If the score (lower symptoms would have a higher score) is better, the step 5015 is invoked in which the parameter set is saved along with the score with a mark that this is the best score for this region. Note that the system could also be set up where better scores are lower. If the score is worse or the same then step 5020 is invoked in which the parameter set is saved along with the score. The path after steps 5015 and 5020 is the same. A segment is a time interval during which the neuromodulation parameters are not changed (typically, but not limited to 15 seconds to two minutes). In step 5025, the question is asked as to whether this is the mth Neuromodulation segment for All Regions (say one wants the exploratory period to include 25 segments). If it is, then step 5030 is invoked and the rest of the session has its neuromodulation continued using the same Best Parameter Set. A session is the time period in which neuromodulation is continuously applied (even if the parameters are changed during that time period, say 50 minutes for non-invasive neuromodulation). At the end of the given session, step 5035 is invoked and the Set of Designated Optimal Neuromodulation Parameters is saved for a Future Session. Note that the operator may choose to start with a different parameter set in a future session rather than the one that was last saved for that patient. Note that certain uses of a recorded signal played back even when neuromodulation is not being applied could have a positive benefit, for example, a soothing influence. Note that certain uses of a recorded signal played back even when neuromodulation is not being applied could have a positive benefit, for example, a soothing influence. In the case of invasive neuromodulation, the session length may be indefinite and the guided/directed feedback only triggered if the symptoms of the patient deteriorate or the operator wishes to try a different neuromodulation paradigm.

If in step 5025 the question the answer as to whether this is the mth Neuromodulation for All Regions is No, in step 5040 the question is asked as to whether this is the kth Neuromodulation in This Region (say one wants to try 20 segments in any given region before moving to try neuromodulating in another region of Parameter Sets. If the answer is No, then step 5045 is invoked with a Flag set to Keep Next Parameter Set in the Current Region. If the answer is Yes, then step 5050 is invoked with a Flag set to Move the Neuromodulation Parameters Set Far Enough Away to be in an Alternative Region to be explored. Although not limited to this, movement of at least one neuromodulation parameter by at least 50% will be sufficient to cause movement to an Alternative Region. The path after steps 5045 and 5050 is the same. In step 5055, the Optimization Algorithm is applied and outputs the Next Set of Neuromodulation Parameters in the Flagged Region. A check is made in step 5060 as to whether the output Candidate Set has Been Used Before. In the answer is Yes, then one needs a set that has not been used previously so step 5055 is invoked again and a new Candidate Parameter Set generated. If the answer to step 5060 is no then step 5005 is executed and Neuromodulation occurs using the New Parameter Set.

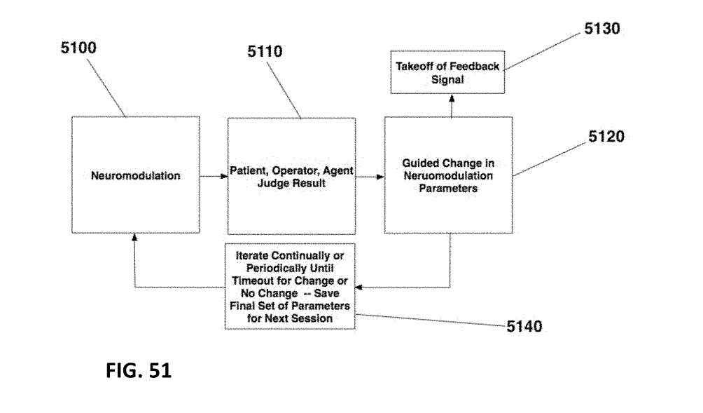

FIG. 51 puts the Guided/Directed Neuromodulation in a larger context with an additional functionality. Blocks 5100, 5110, 5120, and 5140 represent the process of FIG. 50. Block 5140 indicates that the iteration of guided change can occur continually or periodically (e.g., once every 15 neuromodulation segments) until a given number of segments has been reached (or an equivalent timeout) or change in the neuromodulation parameter set is below a designated threshold. In cases with implanted emitters such as Deep Brain Stimulation, Vagal Nerve Stimulation, Spinal Cord Stimulation, implanted versions of occipital or peripheral nerve stimulators, or optogenetics, one can apply the adjustments of Guided-Feedback Neuromodulation over a longer period of time because one is not limited in duration to sessions (e.g., 50 minutes) that occur in most applications of non-invasive neuromodulation such as ultrasound neuromodulation or Transcranial Magnetic Stimulation. The additional novel feature is to take off a feedback-derived signal 5130 that represents the change in Guided-Feedback Neuromodulation 5120 including consideration of its input from the patient symptoms/physiological response as judged by the patient, operator, or agent (or a combination thereof) 5110. This signal can be used to control an action such as the movement or other change (e.g., color) of a object on computer display screen, cause the modulation of an audio signal, or impact motion, like move a robotic arm. One approach is to provide feedback control to ameliorate tremor by counteracting the mechanical motion. Another approach is to have the feedback-derived signal drive the level of the applied ancillary feedback. Note that certain uses of a recorded signal played back even when neuromodulation is not being applied could have a positive benefit, for example, a soothing influence. This can serve as an example of using an ancillary stimulation in the context of neuromodulation. Control of the flow in FIG. 51 can occur as in FIG. 57 with it accompanying description.

### Part XI: Ultrasound Neuromodulation for Diagnosis and Other-Modality Preplanning

The embodiments as described herein provide methods and systems for non-invasive neuromodulation using ultrasound to one or more of diagnosis or to evaluate the feasibility of and preplan neuromodulation treatment using other modalities, such as drugs, electrical stimulation, transcranial ultrasound neuromodulation, surgical intervention, Sphenopalatine Ganglion stimulation, occipital nerve stimulation, peripheral nerve stimulation, transcranial Direct Current Stimulation, optogenetics, implantable devices, or implantable electrodes and combinations thereof, for example.

In many embodiments, the patient can be diagnosed by selecting one or more target sites. The one or more sites are provided with the focused ultrasound beam. An evaluation of the elicited response to the ultrasound beam may be used to distinguish between one or more patient disorders. The patient treatment can be guided by the disorder identified. The guided treatment may comprise one or more of drugs, neuromodulation, or surgery, for example.

In many embodiments confirming a treatment site encompasses determining which of one or more target neural sites can effectively treat the symptoms to be mitigated, based on identification of the one or more target sites from among a plurality of possible target sites based on a response of the patient to the focused ultrasound beam applied to one or more of the possible target sites.

In many embodiments, the confirmed target site is treated with the non-ultrasonic treatment modality after the confirmed target has been determined to be effective based on the patient's response to focused ultrasonic beam delivered to the target site. In many embodiments, the confirmed target site comprises a target site determined to be most likely to successfully treat the patient. The confirmed target site can be selected from among a plurality of possible target sites evaluated based on the response of the patient to the focused ultrasonic beam.

In many embodiments, the confirmation that treatment at a specific site is effective based on ultrasound occurs before implanting the electrode or other implantable device, for example.

The confirmation of the target site allows one to determine which neural target or targets among a plurality of potential targets will most effectively deal with the symptoms to be mitigated. Such neuromodulation systems can produce applicable acute or long-term effects. The long-term effects can occur through Long-Term Depression (LTD) or Long-Term Potentiation (LTP) via training, for example. The embodiments described herein provide control of direction of the energy emission, intensity, frequency (carrier frequency and/or neuromodulation frequency), pulse duration, pulse pattern, mechanical perturbations, and phase/intensity relationships to targeting and accomplishing up-regulation and/or down-regulation, for example.

The ultrasound neuromodulation can be administered in sessions as covered in Section I Part IX. Examples of session types include periodic sessions, such as a single session of length in the range from 15 to 60 minutes repeated daily or five days per week for one to six weeks. Other lengths of session or number of weeks of neuromodulation are applicable, such as session lengths from 1 minute up to 2.5 hours and number of weeks ranging from one to eight. Sessions occurring in a compressed time period typically means a single session of length in the range from 30 to 60 minutes repeated during with inter-session times of 15 minutes to 60 minutes over one to three days. Other inter-session times in the range between approximately 1 minute and three hours and days of compressed therapy such as one to five days are applicable. In an embodiment of the invention, sessions occur only during waking hours. Maintenance consists of periodic sessions at fixed intervals or on as-needed basis such as occurs periodically for tune-ups. Maintenance categories are maintenance post-completion of original treatment at fixed intervals and maintenance post-completion of original treatment with as-needed maintenance tune-ups as defined by a clinically relevant measurement. In an embodiment that uses fixed intervals to determine when additional ultrasound neuromodulation sessions are delivered, one or more 50-minute sessions occur during the second week the 4th and 8th months following the first treatment. In an embodiment that when additional ultrasound neuromodulation sessions are delivered based on a clinically-relevant measurement, one or more 50-minute sessions occur during week 7 because a tune up is needed at that time as indicated by the re-emergence of symptoms. Use of sessions is important for the retraining of neural pathways for change of function, maintenance of function, or restoration of function. Retraining over time, with intermittent reinforcement, can more effectively achieve desired impacts. Efficient schedules for sessions are advantageous so that patients can minimize the amount of time required for their ultrasound treatments. Such neuromodulation systems can produce applicable acute or long-term effects. The latter occur through Long-Term Depression (LTD) or Long-Term Potentiation (LTP) via training.

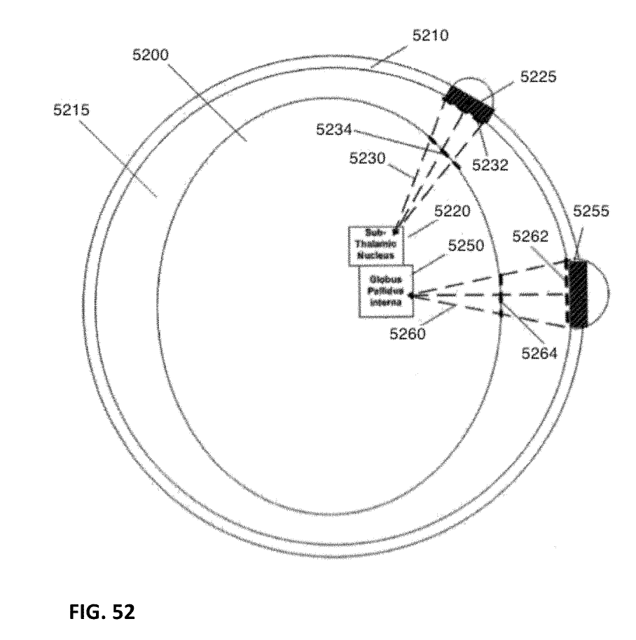

FIG. 52 shows a set of ultrasound transducers targeted to treat Parkinson's Disease. Head 5200 contains two targets, Subthalamic Nucleus 5220 and Globus Pallidus internal 5250. The targets shown are hit by ultrasound from transducers 5225 and 5255 fixed to track 5210. Ultrasound transducer 5225 with its beam 5230 is shown targeting Subthalamic Nucleus (STN) 5220 and transducer 5255 with its beam 5260 is shown targeting Globus Pallidus internal 5250. For ultrasound to be effectively transmitted to and through the skull and to brain targets, coupling must be put into place. Ultrasound transmission (for example Dermasol from California Medical Innovations) medium 5215 is interposed with one mechanical interface to the frame 5210 and ultrasound transducers 5225 and 5255 (completed by a layers of ultrasound transmission gel 5232 and 5262 respectively) and the other mechanical interface to the head 5200 (completed by a layers of ultrasound transmission gel 5234 and 5264 respectively). In another embodiment the ultrasound transmission gel is placed around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers. In still another embodiment, an alternative target can be evaluated with ultrasound neuromodulation, such the Vim (Ventral Intermediate Nucleus of the Thalamus). A diagnostic application of the invention is the differentiation between the tremor of Parkinson's Disease and essential tremor. Note that one strategy is to use DBS on both the STN and the Vim on the same side. In another embodiment, ultrasound neuromodulation of the spinal cord is used to evaluate the potential effectiveness of or parameters for Spinal Cord Stimulation (SCS) using invasive electrode stimulation for the relief of pain.

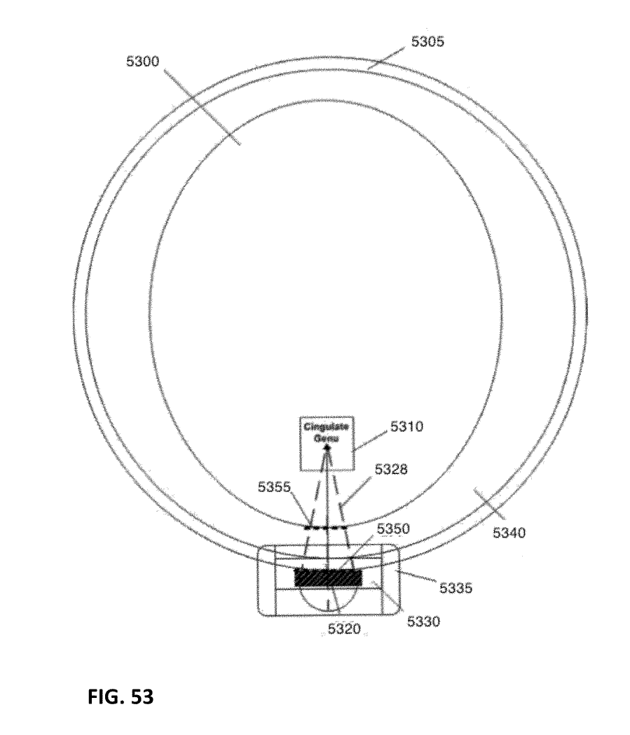

FIG. 53 illustrates the Cingulate Genu as a target for testing in a neuromodulation patient to evaluate whether neuromodulation of that target is effective for the mitigation of depression or bipolar disorder. Head 5300 is surrounded by head frame 5305 on which ultrasound neuromodulation transducer frame 5335 containing an adjustment support 5330 which moves radially in and out of transducer frame 5335. Support 5330 holds ultrasound transducer 5320 with its ultrasound beam 5328 hitting target being evaluated Cingulate Genu 5310. In order for the ultrasound beam 5328 to penetrate effectively, an ultrasound conduction path must be used. This path consists of ultrasound conduction medium 5340 (for example Dermasol from California Medical Innovations) bounded by ultrasound conduction-gel layer 5350 on the ultrasound-transducer side and layer 5355 on the head side. If the ultrasound neuromodulation is successful, then an alternative neuromodulation modality (e.g., DBS) likely can be used successfully due to smaller targeting area achieved. If the ultrasound neuromodulation of this target is not effective then it is likely that the alternative modality being considered (e.g., DBS) will not be successful with this target. Thus the probability of success with an alternative (potentially invasive) neuromodulation modality can be evaluated. If an acute session of ultrasound neuromodulation is ineffective for alleviating symptoms, then the probability is lower that the patient will benefit from a more invasive procedure such as invasive DBS, avoiding both risk for side effects in the patient and significant cost.

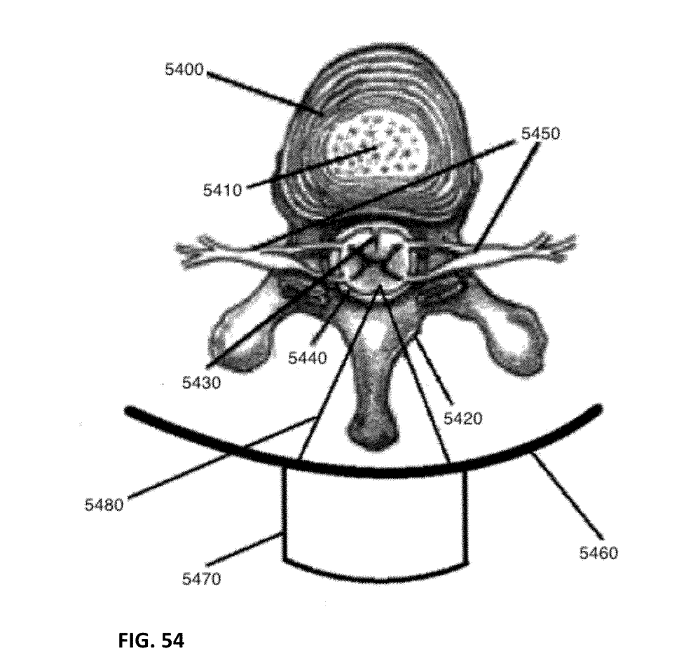

FIG. 54 shows a cross section of the spinal column and spinal cord. Applying ultrasound neuromodulation in this configuration is useful for preplanning to evaluate whether electrode-based Spinal Cord Stimulation (SCS) would be effective in a patient and how SCS should be targeted. Vertebrae disc 5400 including nucleus pulposus 5410 and other bony structures such as the lamina 5420 covers the dura 5440 that surrounds the spinal cord 5430 with its spinal nerve roots 5450. Ultrasound transducer 5470 is pressed against skin 5460 and generates ultrasound beam 5480 that neuromodulates nerves within spinal cord 5430. Bilateral neuromodulation of spinal cord 5430 can be performed. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing) is necessary, an ultrasound transmission medium (for example Dermasol from California Medical Innovations) can be used. In another embodiment, multiple ultrasound transducers whose beams intersect at that target replace an individual ultrasound transducer for that target. In still another embodiment, mechanical perturbations are applied radially or axially to move the ultrasound transducers as described in Part IV above. Ultrasound neuromodulation locations that are successful suggest sites at which application of Spinal Cord Stimulation is likely to also be successful. In an embodiment of the invention, effective parameters of the ultrasound neuromodulation can provide insight into the parameters to be used in SCS, for instance pulsing frequency, relative intensity, and whether a stimulus is monophasic or biphasic.

The operator can set the variables for preplanning or diagnostic ultrasound neuromodulation or the patient can do so in a self-actuated manner. In some self-actuated embodiments, the patient can expedite the process due to their ability to tune the ultrasound neuromodulation to obtain its best results through subjective assessments of whether a symptom or disease state is mitigated with a particular ultrasound session. The novel approaches to patient feedback are covered in Part X above. Often the user can be the best judge concerning which neuromodulation parameters are most effective, either changing one variable of ultrasound at a time or multiple ultrasound waveform variables. An example is a patient with a transected spinal cord directly turning on the neuromodulation to empty a neurogenic bladder.

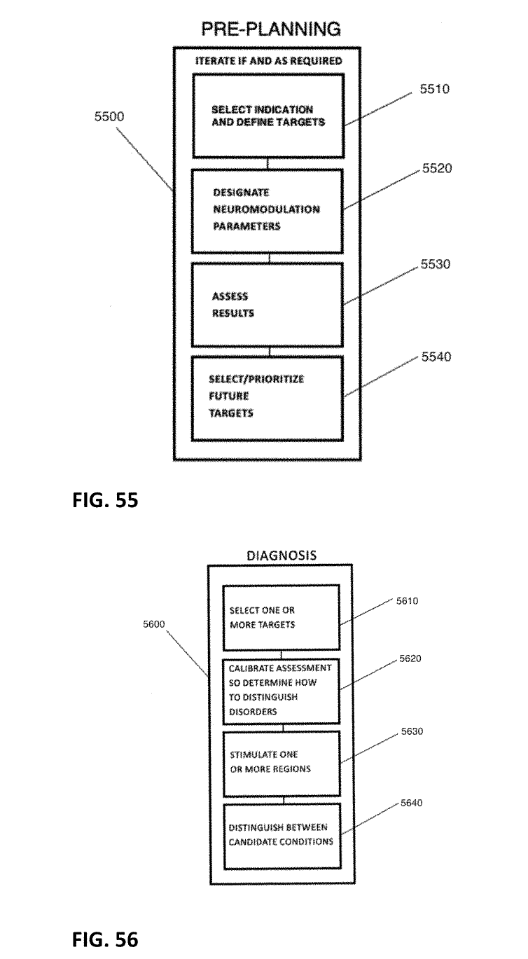

FIG. 55 shows a method 5500 of preplanning for neuromodulation therapy. The neuromodulation therapy may comprise one or more of Ultrasound Neuromodulation, Transcranial Magnetic Stimulation (TMS) or Deep Brain Stimulation (DBS)) or ablative therapy, for example. Each of the steps within method 5500 may be performed iteratively, for example. A step 5510 comprises selecting an indication for treatment and defining related targets sites. The indication may comprise one or more indications as described herein such as one or more of Parkinson's Disease, Depression/Bipolar Disorder, or Spinal Cord Pain, for example. A step 5520 comprises designating ultrasound neuromodulation parameters to apply in either one or multiple neuromodulation sessions, for example. The neuromodulation parameters may comprise one or more known parameters and can be determined by one of ordinary skill in the art based on the embodiments described herein. A step 5530 comprises assessing the results in response to the ultrasound neuromodulation in order to determine stimulation effect, if present. The presence of a stimulation effect can confirm the site as suitable for use with treatment. A step 5540 comprises one or more of selecting or prioritizing targets for future treatment based on the assessment of the results, such that the sites are confirmed prior to treatment. Control of the flow in FIG. 55 can occur as in FIG. 57 with it accompanying description.

TABLE 9 shows a table suitable for incorporation with preplanning in accordance with embodiments as described herein.

TABLE 9

| | Target Site | | |
|---|---|---|---|
| Condition-Input | Evaluated | Assessment | Subsequent Treatment |
| Depression | Cingulate Genu | Depression/Normal | DBS targeted to |
| | | | cingulate genu |
| Parkinson's | DBS, STN, GPi | Tremor | levodopa, dopamine |
| | | | agonists, MAO-B |
| | | | inhibitors, and other |
| | | | drugs such as |
| | | | amantadine and |
| | | | anticholinergics |
| Essential Tremor | (Vim) | Tremor | beta blockers, |
| | | | propranolol, |
| | | | antiepileptic agents, |
| | | | primidone, or |
| | | | gabapentin |
| Bipolar Disorder | Nucleus accumbens, | Structured Clinical | DBS, lithium, |
| | the subcallosal | Interview for DSM-IV | valproic acid, |
| | cingulate (Area 25) | (SCID), the Schedule | divalproex, |
| | | for Affective | lamotrigine, |
| | | Disorders and | quetiapine, |
| | | Schizophrenia | antidepressants, |
| | | (SADS), or other | Symbyax, |
| | | bipolar assessment | clonazepam, |
| | | tool | lorazepam, diazepam, |
| | | | chlordiazepoxide, and |
| | | | alprazolam |
| Spinal Cord Pain | Various levels of | Comparative pain | Level of the spinal |
| | the spinal column; | scale or galvanic skin | column and site for |
| | white matter and | response | electrical stimulation, |
| | ganglia | | ultrasound |
| | | | neuromodulation, or |
| | | | surgical intervention |

As to Nucleus Accumbens, supportive data can be found be one of ordinary skill in the art on the worldwide web (www.clinicaltrials.gov/ct2/show/NCT01372722). With regards to the subcallosal cingulate (Area 25), supportive data can be found be one of ordinary skill in the art on the web (www.dana.org/media/detail.aspx?id=35782). With regards to the Schedule of Affective Disorders and Schizophrenia, supportive data can be found by one of ordinary skill in the art at on the worldwide web (www.ncbi.nlm.nih.gov/pmc/articles/PMC2847794/). With regards to treatment and drugs related to bipolar disorder, supportive data can be found on the world wide web by one of ordinary skill in the art (http://www.mayoclinic.com/health/bipolar-disorder/DS00356/DSECTION=treatments-and-drugs).

The method 5500 can be used to confirm treatment of the patient based on the patient's response to target site evaluated. For the condition input and target site evaluated, a subsequent treatment can be selected that acts on the target site evaluated, for example as described herein with reference to TABLE 9.

Although the above steps show method 5500 of planning a treatment of a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

One or more of the steps of the method 5500 may be performed with the circuitry as described herein, for example one or more of the processor or logic circuitry such as a field programmable array logic for field programmable gate array. The circuitry may be programmed to provide one or more of the steps of method 5500, and the program may comprise program instructions stored on a computer readable memory or programmed steps of the logic circuitry such as the programmable array logic or the field programmable gate array, for example.

FIG. 56 shows a method 5600 of diagnosis of a patient. A step 5610 comprises selection of one or more target sites as described herein. A step 5620 comprises calibrating an assessment to determine how to distinguish candidate disorders based on elicited effects consistent with one disorder versus another disorder, for example. A step 5630 comprises neuromodulating the one or more target sites with ultrasound as described herein. A step 5640 comprises distinguishing among a plurality of candidate conditions. The process 5600 provides information for guiding treatment as described or incorporated into the treatment with control covered in FIG. 57 and its accompanying description. The treatment may comprise one or more treatments as described herein such as neuromodulation, surgery, or medication, for example. Assessments can be made by direct observation or by instruments such as the known Visual Analog Scale for pain (H. Breivik, H., Borchgrevink, P. C., Allen, S. M., Rosseland, L. A., Romundstad, L., Breivik Hals, E. K., Kvarstein, G., and A. Stubhaug, “Assessment of Pain,” Br J Anaesth. 2008; 101(1):17-24.) or motor skill assessments for Parkinson's disease (Motor Bruininks-Oseretsky Test of Motor Proficiency, Second Edition (BOT-2), Authors: Robert H. Bruininks, PhD & Brett D. Bruininks, (for ages for four through 21) and Bruininks Motor Ability Test (BMAT), Authors: Brett D. Bruininks & Robert H. Bruininks, PhD (for adults), both by Pearson Education, Inc.).

TABLE 10 shows a table suitable for incorporation with diagnosis in accordance with embodiments as described herein.

TABLE 10

| Symptom- | Target Site(s) | | Condition- |
|---|---|---|---|
| Input | Evaluated- Input | Assessment/Indicator | Output |
| Depression/ | Cingulate Genu | Depression/Normal | Depression |
| Normal | | | |
| Tremor | DBS, STN, or GPi | Tremor | Parkinson's |
| Tremor | Vim | Tremor | Essential |
| | | | Tremor |
| Bipolar | Nucleus | Structured Clinical | Bipolar |
| behavior | accumbens, the | Interview for DSM-IV | Disorder |
| | subcallosal | (SCID), the Schedule | |
| | cingulate (Area 25) | for Affective Disorders | |
| | | and Schizophrenia | |
| | | (SADS), or other | |
| | | bipolar assessment tool | |
| Pain | Spinal Cord; | Comparative pain | Spinal Cord |
| | Various levels of | scale or galvanic skin | Pain |
| | the spinal column; | response | |
| | white matter and | | |
| | ganglia | | |

Although the above steps show method 5600 of diagnosing a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

One or more of the steps of the method 5600 may be performed with the circuitry as described herein, for example one or more of the processor or logic circuitry such as programmable array logic for field programmable gate array. The circuitry may be programmed to provide one or more of the steps of method 5600, and the program may comprise program instructions stored on a computer readable memory or programmed steps of the logic circuitry such as the programmable array logic or the field programmable gate array, for example.

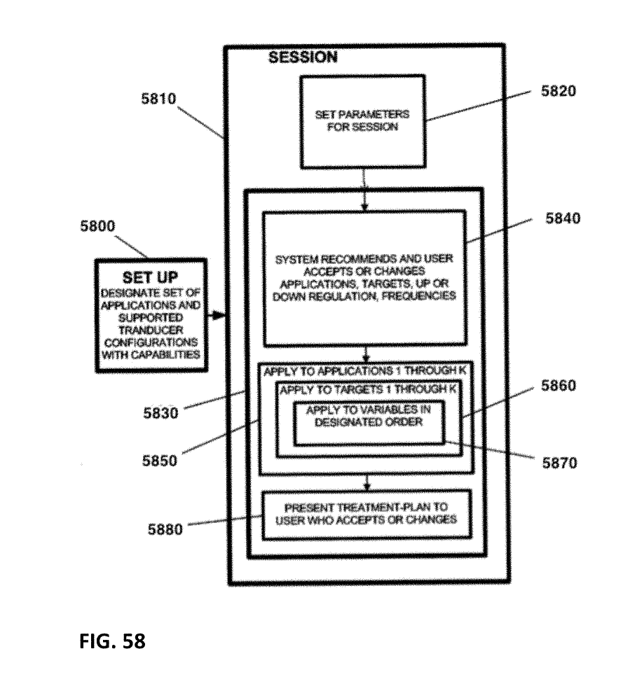

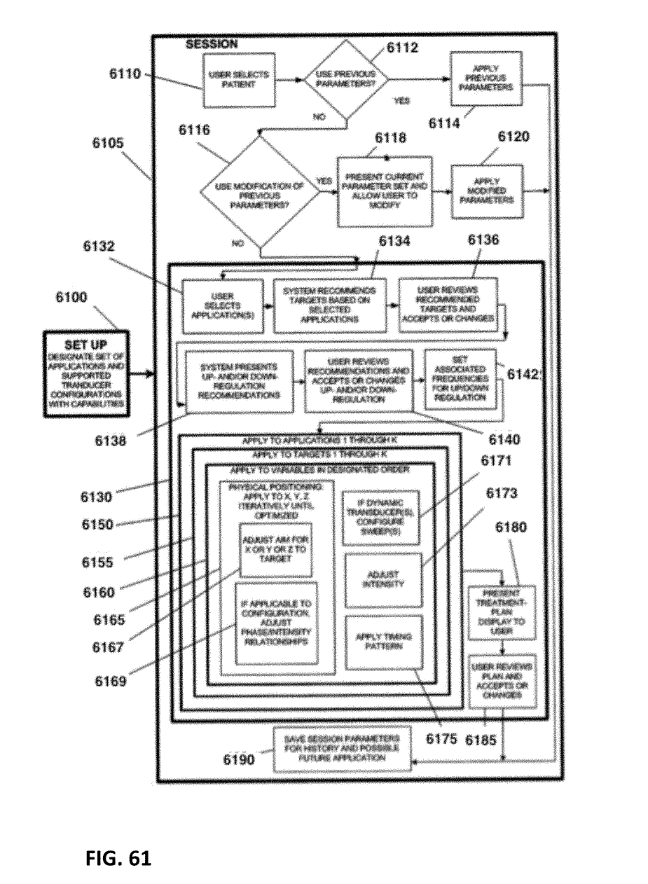

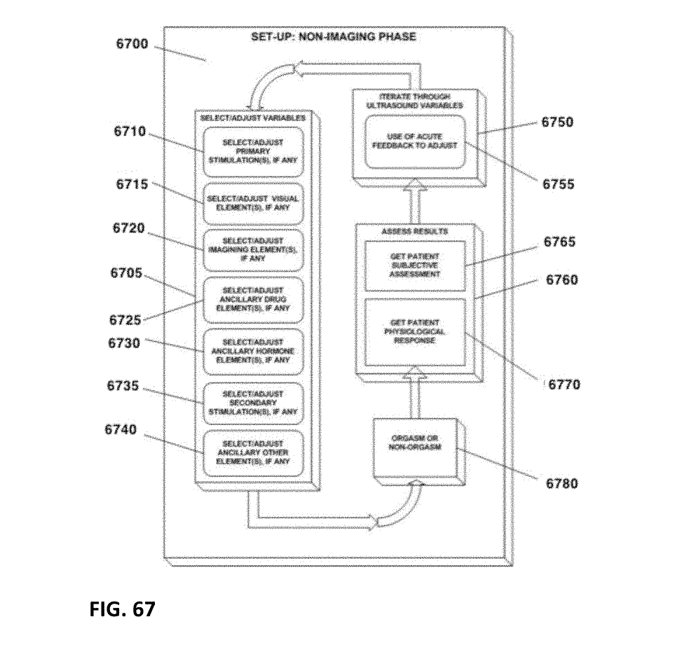

FIG. 57 shows an apparatus 5700 for one or more of multi-modality neuromodulation (FIG. 6), ultrasound neuromodulation control (FIG. 11), patient and other feedback (FIGS. 46, 48, 50, 51), preplanning for (FIG. 55) or diagnosing the patient (FIG. 56), treatment planning (FIGS. 58, 61) and Orgasm Elicitation (FIGS. 67-70) in accordance with embodiments.

The apparatus 5700 comprises an ultrasound source 5707. The ultrasound source 5707 comprises a source of ultrasound as described herein. The ultrasound source 5707 may comprise a head 5701, a transducer holding apparatus 5702, a transducer 5703, a transducer 5704, or a transducer array 5705 as described herein for example. Further, the apparatus also includes inputs 5706 for patient feedback, sensor feedback, image feedback, or other feedback.

The apparatus 5700 comprises a controller 5750 coupled to the ultrasound source 5707. The controller 5750 comprises a processer 5752 having a computer readable medium 5754. The computer readable memory 5754 may comprise instructions for controlling the ultrasound source. The controller 5750 may comprise one or more components of the control system 5708 as described herein.

The apparatus 5700 comprises a processor system 5710. The processor system 5710 is coupled with a control system. The processor 5710 comprises a computer readable memory 5712 having instructions of one or more computer programs embodied thereon. The computer readable memory 5712 comprises instructions 5755. The instructions 5755 comprise one or more instructions of the multi-modality neuromodulation system of FIG. 6 and corresponding methods as described herein. The computer readable memory 5712 comprises instructions 5760. The instructions 5760 comprise one or more instructions of the ultrasound neuromodulation control system 1110 of FIG. 11 and corresponding methods as described herein. The computer readable memory 5712 comprises instructions 5765. The instructions 5765 comprise one or more instructions of the feedback control systems of FIG. 46, FIG. 48, FIG. 50, and FIG. 51, and corresponding methods as described herein. The computer readable memory 5712 comprises instructions 5770. The instructions 5770 comprise one or more instructions to implement one or more steps of the preplanning method 5500 of FIG. 55 as described herein. The computer readable memory 5775 comprises instructions to implement one or more steps of the diagnosing a patient method 5600 of FIG. 56 as described herein. The computer readable memory 5712 comprises instructions 5780. The instructions 5780 comprise one or more instructions of the treatment planning systems of FIGS. 58 and 61, and corresponding methods as described herein. The computer readable memory 5712 comprises instructions 5785. The instructions 5785 comprise one or more instructions for Orgasm Elicitation of FIGS. 67-70 and corresponding methods as described herein.

The computer readable memory 5712 comprises instructions 5790 to coordinate the components as described herein and the methods as described herein. For example, the instructions 5790 may comprise a user responsive switch to select preplanning method 5770 or instructions to diagnose the patient 5750 based on user preference. The computer readable memory may comprise information of one or more of TABLE 9 or TABLE 10 so as to plan treatment of the patient and diagnose the patient, in accordance with embodiments as described herein.

The processor system 5710 is coupled to a user interface 5714. The user interface 5714 may comprise a display 5716 such as a touch screen display. The user interface 5714 may comprise a handheld device such as a commercially available iPhone, Android operating system device, such as, a Samsung Galaxy smart phone or other known handheld device such as an iPad, tablet computer, or the like. The user interface 5714 can be coupled with a processor system 5710 with communication methods and circuitry. The communication may comprise one or more of many known communication techniques such as WiFi, Bluetooth, cellular data connection, and the like. The processor system 5710 is configured to communicate with a measurement apparatus 5718. The measurement apparatus 5718 comprises patient measurement data storage 5719 that can be stored on a computer readable memory. The processor system 5710 is in communication with the measurement apparatus 5718 with communication that may comprise known communication as described herein. The processor system 5710 is configured to communicate with the controller 5750 to transmit the signals for use with the ultrasound source 5707 in for implementation with one or more components of control system 5708 as described herein.

The apparatus 5700 allows ultrasound stimulation adjustments in variables such as carrier frequency and/or neuromodulation frequency, pulse duration, pulse pattern, mechanical perturbations, as well as the direction of the energy emission, intensity, frequency, mechanical perturbations, phase/intensity relationships to targeting and accomplishing up-regulation and/or down-regulation, dynamic sweeps, and position. The user can input these parameters with the user interface, for example.

Reference is made to the following publications, which are provided herein to clearly and further show that the embodiments of the methods and apparatus as described herein are clearly enabled and can be practiced by a person of ordinary skill in the art without undue experimentation.

Clinical stimulation of the Cingulate Genu in humans is described by Mayberg et al. (Mayberg, Helen S., Lozano, A. M., Voon, Valerie, McNeely, Heather E., Seminowicz, D., Hamani, C., Schwalb, J. M., and S. H., Kennedy, “Deep Brain Stimulation for Treatment-Resistant Depression,” Neuron, Volume 45, Issue 5, 3 Mar. 2005, Pages 651-660), for example.

Patient response to Stimulation of the Subthalamic Nucleus and Globus Pallidus interna can produce measurable patient results suitable for one or more of diagnosis or confirmation as described herein. (Anderson et al. (Anderson, V C, Burchiel, K J, Hogarth, P, Favre, J, and J P Hammerstad, “Pallidal vs subthalamic nucleus deep brain stimulation in Parkinson disease,” Arch Neurol. 2005 April; 62(4):554-60).

### Part XII: Treatment Planning for Deep-Brain Neuromodulation

Treatment planning for non-invasive deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Ultrasound transducers or other energy sources are positioned and the anticipated effects on up-regulation and/or down-regulation of their direction of energy emission, intensity, frequency, firing/timing, mechanical perturbations, and phase/intensity relationships mapped onto treatment-planning targets. The maps of treatment-planning targets onto which the mapping occurs can be atlas (e.g., Tailarach Atlas) based or image (e.g., fMRI or PET) based. Imaged-based maps may be representative and applied directly or scaled for the patient or may be specific to the patient.

While the description of the invention focuses on ultrasound, treatment planning can be done for therapy using other modalities (e.g., Transcranial Magnetic Stimulation (TMS), Sphenopalatine Ganglion stimulation, occipital nerve stimulation, peripheral nerve stimulation, transcranial Direct Current Stimulation (tDCS), and/or Deep Brain Stimulation (DBS), Vagus Nerve Stimulation (VNS), Sphenopalatine Ganglion Stimulation and/or other local stimulation using implanted electrodes), and/or future neuromodulation means either individually or in combination.

FIG. 58 shows a block diagram of treatment planning. The set-up 5800 designates the set of applications to be considered as well as transducer configurations and capabilities. The session flow 5810 involves setting the parameters for the session 5820 that is followed by set of activities 5830 in which the system recommends and the healthcare-professional user accepts or changes 5840 the recommended applications, targets, up- or down-regulation, and frequencies to be used for neuromodulation. Setting of the basic parameters is followed by the application to clinical applications 1 through k 5850 which incorporates application to targets 1 through k 5860 within which application to variables (from among position, intensity, dynamic sweeps, mechanical perturbations, and firing/timing pattern) 5870 in the designated order. In step 5880, the resultant treatment plan is presented to the healthcare-professional who accepts or changes the plan. Control of the flow in FIG. 58 can occur as in FIG. 57 with it accompanying description. Hitting multiple targets in a neural circuit in a treatment session is an important component of fostering a durable effect through Long-Term Potentiation (LTP) and/or Long-Term Depression (LTD) and is useful for acute effects as well. In addition, this approach can decrease the number of treatment sessions required for a demonstrated effect and to sustain a long-term effect. Follow-up tune-up sessions at one or more later times may be required. The treatment-planning process can be applied to other modalities or a mixture of modalities (e.g., ultrasound used simultaneously with Deep Brain Stimulation or simultaneously or sequentially with Transcranial Magnetic Stimulation). Not all variables be planned for will be same for all modalities and in some cases they may be different than those covered.

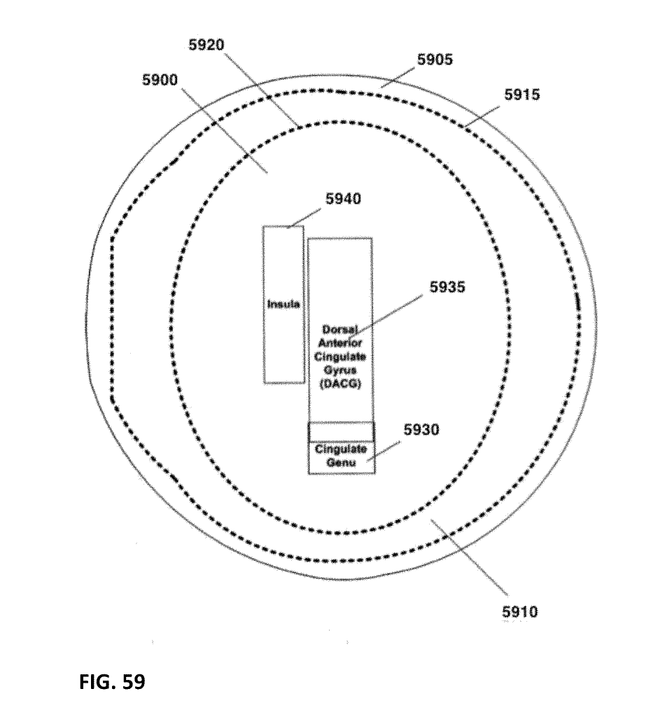

As an example of using the system, in FIG. 59, within patient head 5900, three targets related to the processing of pain, the Cingulate Genu 5930, Dorsal Anterior Cingulate Gyms (DACG) 5935, and Insula 5940. These targets, if down regulated through neuromodulation, will decrease the pain perceived by the patient. The physical context of the overall configuration is that the patient head 5900 is surrounded by frame 5905 on which the ultrasound transducers (not yet attached) will be fixed. Between frame 5905 and patient head 5900 are interposed the ultrasound-conduction medium 5910 (say silicone oil housed within a containment pouch or Dermasol from California Medical Innovations) with the interface between the frame 5905 and the ultrasound-conduction medium 5910 filled by conduction-gel layer 5915 and the interface between ultrasound-conduction medium 5910 and patient head 5900 filled by conduction-gel layer 5920. For the ultrasound to be effectively transmitted to and through the skull and to brain targets, coupling must be put into place. This is only one configuration. In the other embodiments, the ultrasound-conduction medium and the gel layers do not have to completely surround the head, but only need be placed where the ultrasound transducers are located.

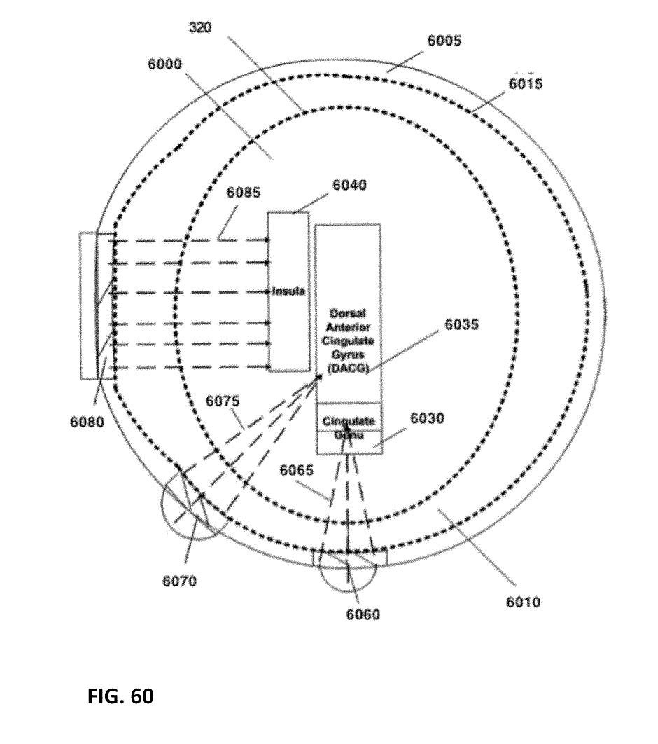

After the treatment planning of FIG. 58 is applied, the graphic as shown in FIG. 60 is displayed so the healthcare-professional can both understand the plan and place the transducers on the frame. Vertical location would be given as well (not shown) as well as sagittal and coronal views displayed (not shown). In FIG. 60, a frame 6005 with interposed elements ultrasound-transmission-gel layer 6020, ultrasound-transmission medium 6010, and ultrasound-transmission-gel layer 6015 again surrounds patient head 6000. The display shows the positioning of ultrasound transducer 6060 aimed at the Cingulate Genu target 6030 and the planned ultrasound field 6065. In like manner, the display shows the positioning of ultrasound transducer 6070 aimed at the Dorsal Anterior Cingulate Gyrus (DACG) target 6035 with the planned ultrasound field 6075. This display also shows the positioning of ultrasound transducer 6080 aimed at the Insula target 6040 with the planned ultrasound field 6085.

The treatment-planning process covered in FIG. 58 is shown in FIG. 61. Control of the flow in FIG. 61 can occur as in FIG. 57 with it accompanying description. Set up 6100 includes designation of the set of applications and supported transducer configurations. Session 6105 begins with step 6110 where the healthcare-professional user selects the patient, which is followed by decision-step 6112 as to whether or not previous parameters are to be used. If the response is yes then step 6114 is executed, the application of previous parameters, after which there is step 6190, saving the session parameters for the historical record and possible future application. If the response 6112, use of previous parameters, is no, then decision-step 6116 is executed, whether there is to be a user-supplied modification of the previous parameters. If the response is yes, step 6118 presents the current parameter set to the user and allows the user to modify them. Then in step 6120, the modified parameters are applied, after which there is step 6190, saving the session parameters for the historical record and possible future application. If the response to decision-step 6116, whether there is to be a user-supplied modification of the previous parameters is no, then the flow shown in box 6130 is followed. In the initial step 6132 the health-professional user selects the applications to be used. This is followed by step 6134, system recommending the targets based on the selected applications and step 6136 where the user reviews the recommended targets and accepts or changes them. Note that for any of the healthcare-professional user's choices that are inconsistent or otherwise cannot be safely applied, the system will notify the user and offer the opportunity for corrections to be made. Step 6136 is followed by step 6138 in which the system presents the up- and/or down-regulation recommendations and then step 6140 in which the user reviews those recommendations and accepts or changes the up- and/or down regulation designations. Down regulation means that the firing rate of the neural target has its firing rate decreased and thus is inhibited and up regulation means that the firing rate of the neural target has its firing rate increased and thus is excited. In the next step 6142, the associated frequencies for up- and down-regulation are applied followed by the iterative application of the elements in box 6150 in which in the outer loop the process is applied to applications 1 through k. In succeeding inner loop 6155, the process is applied iteratively to targets 1 through k and in its succeeding inner loop 6160; the process is applied iteratively to variables in the designated order. In step 6165, the physical positioning is applied to x, y, and z iteratively until optimized with 6167 adjustment of the aim to target, and 6169, if applicable to the configuration, adjustment of the phase/intensity relationships for beam steering and/or focus. Step 6171, configuring of sweep(s) is executed if there are dynamic transducers. In step 6173, the intensity is adjusted, and the firing/timing pattern applied in 6175. The ultrasonic firing/timing patterns can be tailored to the response type of a target or the various targets hit within a given neural circuit. In the output of box 6150, in step 6180, the treatment-plan display is presented to the user followed by step 6185 in which the user reviews the plan and accepts or changes it. Again, if the plan is inconsistent or cannot otherwise be safely executed, the system will notify the user and offer the opportunity for corrections to be made. Following acceptance of the treatment plan, there is step 6190, saving the session parameters for the historical record and possible future application.

### Part XIII: Ultrasound Neuromodulation of Spinal Cord

It is the purpose of some of the inventions described herein to provide methods and systems and methods for neuromodulation of the spinal cord to treat certain types of pain. Such pain conditions include non-cancer pain, failed-back-surgery syndroeme, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), and refractory pain due to ischemia (e.g. angina). In certain embodiments of the present invention, pain is replaced by tingling parathesias. In certain embodiments of the present invention, ultrasound neuromodulation stimulates pain inhibition pathways and can produce acute or long-term effects. The latter occur through long-term depression (LTD) or long-term potentiation (LTP) via training. Acute and chronic vasculitis can be treated as well as associated pain. In addition, sacral neuromodulation can be employed for the treatment of hyperactive bladder as well as to stimulate emptying of a neurogenic bladder. Included is control of direction of the energy emission, intensity, frequency (carrier frequency and/or neuromodulation frequency), pulse duration, pulse pattern, mechanical perturbations, and phase/intensity relationships to targeting and accomplishing up-regulation and/or down-regulation.

Target regions in the spinal cord which can be treated using the ultrasound neuromodulation protocols of the present invention comprise the same locations targeted by electrical SCS electrodes for the same conditions being treated, e.g., a lower cervical-upper thoracic target region for angina, a T5-7 target region for abdominal/visceral pain, and a T10 target region for sciatic pain. Ultrasound neuromodulation in accordance with the present invention can stimulate pain inhibition pathways that in turn can produce acute and/or long-term effects. Other clinical applications of ultrasound neuromodulation of the spinal cord include non-invasive assessment of neuromodulation at a particular target region in a patient's spinal cord prior to implanting an electrode for electrical spinal cord stimulation for pain or other conditions.

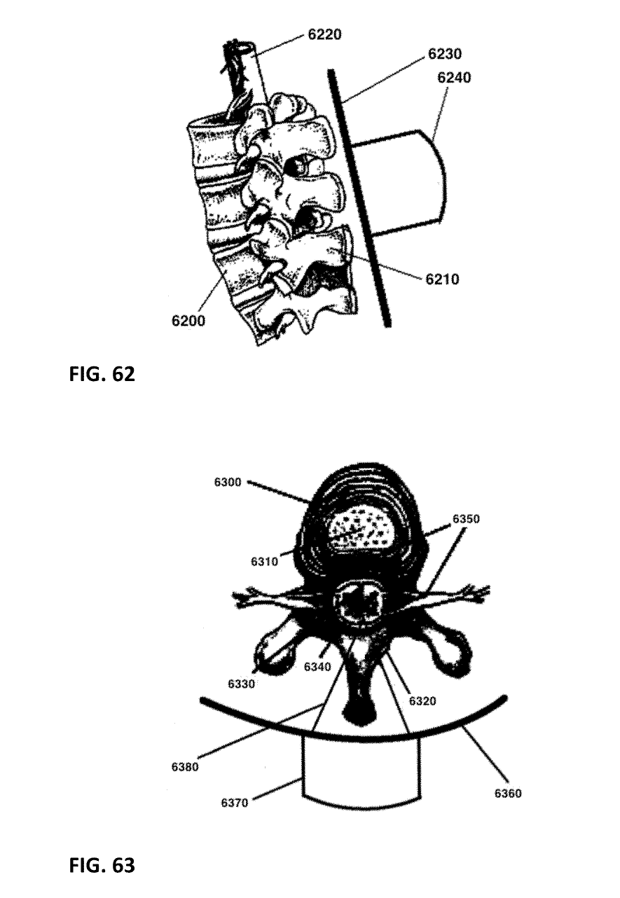

FIG. 62 shows spinal column with vertebrae 6200 and spinal process 6210 containing spinal cord 6220 covered by skin 6230. Spinal cord 6220 is neuromodulated by ultrasound transducer 6240. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing or between the transducer face and the skin), an ultrasound transmission medium (for example Dermasol from California Medical Innovations) can be used. In another embodiment, multiple ultrasound transducers whose beams intersect at that target replace an individual ultrasound transducer for that target. Transducers can be placed on both sides of the spinous processes to direct beams inwardly to integrate along the spinal cord or can be located on one side only and focused medially to target the spinal cord. In still another embodiment, mechanical perturbations are applied radially or axially to move the ultrasound transducers, as discussed in Section I, Part IV above.

FIG. 63 shows a cross section of the spinal column and spinal cord. Vertebrae disc 6300 with its nucleus pulposus 6310 with other bony structures such as the lamina 6320 surrounds the dura 6340 surrounding spinal cord 6330 with its spinal nerve roots 6350. Ultrasound transducer 6370 is pressed against skin 6360 and generates ultrasound beam 6380 that neuromodulates nerves within spinal cord 6330. Bilateral neuromodulation of spinal cord 6330 can be performed. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing), an ultrasound transmission medium (for example Dermasol from California Medical Innovations) can be used. In another embodiment, multiple ultrasound transducers whose beams intersect at that target replace an individual ultrasound transducer for that target. In still another embodiment, mechanical perturbations are applied radially or axially to move the ultrasound transducers as in Part IV above. In addition, FIG. 17A-C illustrates field shaping applicable to neuromodulation of the spinal cord. In alternative embodiments, a spot focused ultrasonic energy beam may be over any portion of the length of the spinal cord to target specific target regions or moved via steering over the target regions. In both cases, it is possible to determine over what length of a target region that the ultrasound is to be applied. For example, one could apply ultrasound to only a selected portion of the spinal cord. Transducers can be place on either side of the spinous processes or placed on one side and aimed medially. Neuromodulation shaping via mechanical perturbations is applicable to neuromodulation of the spinal cord and is covered in Part IV above.

### Part XIV: Ultrasound Neuromodulation of the Brain, Nerve Roots, and Peripheral Nerves

Some of the inventions described herein provide methods and systems and methods for ultrasound stimulation of the cortex, nerve roots, and peripheral nerves, and noting or recording muscle responses to clinically assess motor function. In addition, just like Transcranial Magnetic Stimulation, ultrasound neuromodulation can be used to treat depression by stimulating cortex and indirectly impacting deeper centers such as the cingulate gyrus through the connections from the superficial cortex to the appropriate deeper centers. Ultrasound can also be used to hit those deeper targets directly. Positron Emission Tomography (PET) or fMRI imaging can be used to detect which areas of the brain are impacted. In addition to any acute positive effect, there will be a long-term “training effect” with Long-Term Depression (LTP) and Long-Term Potentiation (LTD) depending on the central intracranial targets to which the neuromodulated cortex is connected.

Ultrasound stimulation can be applied to the motor cortex, spinal nerve roots, and peripheral nerves and generate Motor Evoked Potentials (MEPs). MEPs elicited by central stimulation will show greater variability than those elicited stimulating spinal nerve roots or peripheral nerves. Stimulation results can be recorded using evoked potential or electromyographic (EMG) instrumentation. Muscle Action Potentials (MAPs) can be evaluated without averaging while Nerve Action Potentials (NAPs) may need to be averaged because of the lower amplitude. Such measurements can be used to measure Peripheral Nerve Conduction Velocity (PNCV). Pre-activation of the target muscle by having the patient contract the target muscle can reduce the threshold of stimulation, increase response amplitude, and reduce response latency. Another test is Central Motor Conduction Time (CMCT), which measures the conduction time from the motor cortex to the target muscle. Different muscles are mapped to different nerve routes (e.g., Abductor Digiti Minimi (ADM) represents C8 and Tibialis Anterior (TA) represents L4/5). Still another test is Cortico-Motor Threshold. Cortico-motor excitability can be measured using twin-pulse techniques. Sensory nerves can be stimulated as well and Sensory Evoked Potentials (SEPs) recorded such as stimulation at the wrist (say the median nerve) and recording more peripherally (say over the index finger). Examples of applications include coma evaluation (diagnostic and predictive), epilepsy (measure effects of anti-epileptic drugs), drug effects on cortico-motor excitability for drug monitoring, facial-nerve functionality (including Bell's Palsy), evaluation of dystonia, evaluation of Tourette's Syndrome, exploration of Huntington's Disease abnormalities, monitoring and evaluating motor-neuron diseases such as amyotrophic lateral sclerosis, study of myoclonus, study of postural tremors, monitoring and evaluation of multiple sclerosis, evaluation of movement disorders with abnormalities unrelated to pyramidal-tract lesions, and evaluation of Parkinson's Disease. As evident by the conditions that can be studied with the various functions, neurophysiologic research in a number of areas is supported. Other applications include monitoring in the operating room (say before, during, and after spinal cord surgery). Cortical stimulation can provide relief for conditions such as depression, bipolar disorder, pain, schizophrenia, post-traumatic stress disorder (PTSD), and Tourette syndrome. Another application is stimulation of the phrenic nerve for the evaluation of respiratory muscle function. Clinical neurophysiologic research such as the study of plasticity.

When TMS is applied to the left dorsal lateral prefrontal cortex and depression is treated ‘indirectly” (e.g., at 10 Hz, although other rates such as 1, 5, 15, and 20 Hz have been used successfully as well) due to connections to one or more deeper structures such as the cingulate and the insula as demonstrated by imaging. The same is true for ultrasound stimulation.

A benefit of ultrasound stimulation over Transcranial Magnetic Stimulation is safety in that the sound produced is less with a lower chance of auditory damage. Ironically, TMS produces a clicking sound in the auditory range because of deformation of the electromagnet coils during pulsing, while ultrasound stimulation is significantly above the auditory range.

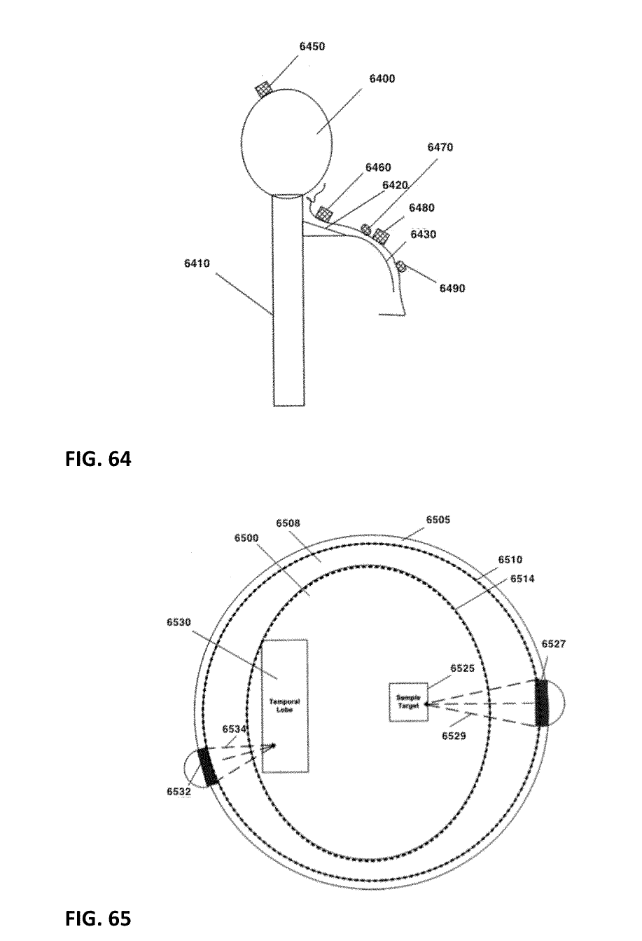

FIG. 64 illustrates placement of ultrasound stimulators and EMG sensors related to head 6400, spinal cord 6410, nerve root 6420, and peripheral nerve 6430. Ultrasound transducer 6450 is directed at superficial cortex (say motor cortex). For any ultrasound transducer position, ultrasound transmission medium (e.g., silicone oil in a containment pouch) and/or an ultrasonic gel layer. When the ultrasound transducer is pulsed [typically tone burst durations of approximately (but not limited to) 25 to 500 μsec, the conduction time to the sensor at nerve root 6470 and/or associated muscles further in the periphery 6490. Alternatively ultrasound transducer 6460 may be positioned at a nerve root 6420 and the conduction time to the electromyography sensor 6490 measured. Further, an ultrasound transducer 6480 may be positioned over peripheral nerve 6430 and the conduction time to electromyography sensor 6490 measured. Shaping of the ultrasound field in the various locations is accomplished using the techniques in Part II above on focused ultrasound, including those covered in, but not limited to, in FIG. 8 pertaining to ultrasound transducer shaping and FIG. 24 pertaining to mechanical perturbations.

Even though the target is relatively superficial, the transducer can be moved back in the holder to allow a longer focal length. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well. Other embodiments have mechanisms for focus of the ultrasound including fixed ultrasound array, flat ultrasound array with lens, non-flat ultrasound array with lens, flat ultrasound array with controlled phase and intensity relationships, and ultrasound non-flat array with controlled phase and intensity relationship. Ultrasound conduction medium will be required to fill the space. Examples of sound-conduction media are Dermasol from California Medical Innovations or silicone oil in a containment pouch. If patient sees impact, he or she can move transducer (or ask the operator to do so) in the X-Y direction (Z direction is along the length of transducer holder and could be adjusted as well).

Cortical excitability can be measured using single pulses to determine the motor threshold (defined as the lowest intensity that evokes MEPs for one-half of the stimulations). In addition, such single pulses delivered at a level above threshold can be used to study the suppression of voluntarily contracted muscle EMG activity following an induced MEP.

### Part XV: Ultrasound-Neuromodulation Techniques for Control of Permeability of the Brain Barrier

It is the purpose of some of the inventions described in this section herein to provide methods and systems using non-invasive ultrasound-neuromodulation techniques to selectively alter the permeability of the blood-brain barrier (either brain or spinal cord). If the target is a neural target as opposed to a tumor, the application of the invention may result in effective neuromodulation of that target in addition to altering the permeability of the blood-brain barrier in that region allowing more effective penetration of a drug to impact that neural target. This applies to humans or animals and in brain or spinal cord. The change can control blood-brain permeability by increasing permeability to increase the access of drugs to, for example, neurological targets or tumors or decreasing permeability to protect targets from drugs that could cause side effects. If the application of the techniques results in decreasing the permeability of the blood-brain barrier (in cases where the permeability has been increased through another mechanism), in some cases coincident neuromodulation of a target in the region will have a therapeutic benefit. Multiple conditions are aggravated by breaching of the blood-brain barrier, among which are Alzheimer's Disease, HIV Encephalitis, Multiple Sclerosis, Meningitis, and Epilepsy. Such neuromodulation systems can produce applicable acute or long-term effects. The latter occur through Long-Term Depression (LTD) or Long-Term Potentiation (LTP) via training. Included is control of direction of the energy emission, intensity, frequency (carrier and/or neuromodulation frequency), pulse duration, firing pattern, mechanical perturbations, and phase/intensity relationships for beam steering and focusing on targets and accomplishing up-regulation and/or down-regulation.

What will work for altering the permeability of the blood brain barrier in a given situation depends on a given patient and associated condition. In some situations, excitation will result in increasing the permeability of the blood-brain barrier and inhibition will result in decreasing it. In other situations, the reverse will be true.

Altering the permeability of the blood-brain barrier using ultrasound-neuromodulation techniques has significant benefits over other techniques such as Transcranial Magnetic Stimulation neuromodulation (e.g., using the Brainsway system) because ultrasound neuromodulation provides greater resolution and uses hardware that is both less expensive and portable so it can be used at home, work, school, or other non-clinical-office locations.

A notable benefit is the ability to reduce side effects by having increased permeability in applicable regions where a drug needs to be active and leave at its normal level or decrease permeability in other regions where that drug could cause side effects. This spatial selectivity depends on the ability of the neuromodulation to be selective which is true for ultrasound neuromodulation, but not true for an essentially whole-brain neuromodulation approach such as that of Brainsway or any approach using Transcranial Magnetic Stimulation. Another facet of side effects is the significant opportunity to protect structures by selectively decreasing the permeability in certain regions.

FIG. 65 shows exemplar targets for control of permeability of the blood-brain barrier for the selective penetration of drugs or other substances into the target. Head 6500 contains two targets, one a generic Sample Target 6525 and the other the Temporal Lobe 6530 as an example of a neural target for the treatment of epilepsy. For example, Sample Target 6525 may represent a malignant tumor such as glioblastoma multiforme (the subject of the work by Brainsway) to open up the path for anti-tumor drugs and Temporal Lobe 6530 would be a target for permeability change to open up the path for anti-epilepsy drugs. There can be different numbers of targets for a given condition and the appropriate targets will change as research evolves. Targets 6525 and 6530 are targeted by ultrasound from transducers 6527 and 6532 respectively, fixed to track 6505. In other embodiments the ultrasound transducer or transducers can be affixed to the patient's head using other means such as strapping to the head or holding within the framework of a swimming-cap-style structure. Ultrasound transducer 6527 with its beam 6529 is shown targeting Sample Target 6520 and transducer 6532 with its beam 6534 is shown targeting Temporal Lobe 6530. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 6508 is interposed with one mechanical interface to the frame 6505 and ultrasound transducers 6527 and 6532 (completed by a layer of ultrasound transmission gel layer 6510) and the other mechanical interface to the head 6500 (completed by a layer of ultrasound transmission gel 6514). In another embodiment, the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. If a large volume of the brain is to have its permeability altered then multiple ultrasound transducers with defocused beams can be employed.

### Part XVI: Whole Head Neuromodulation

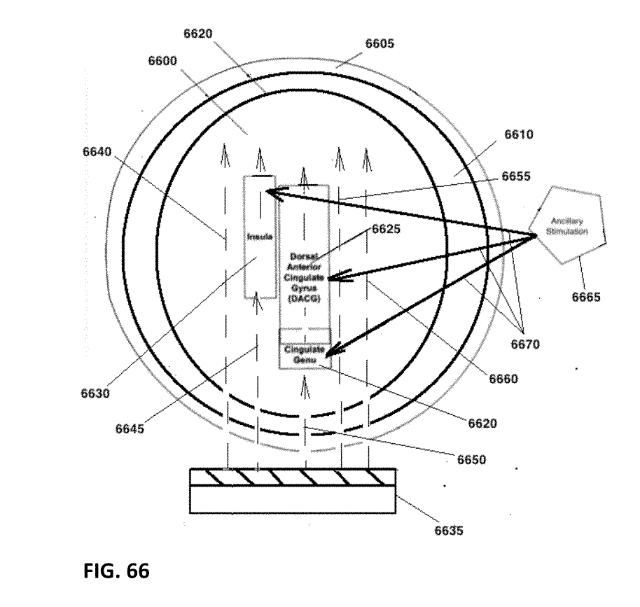

FIG. 66 shows an embodiment of whole-head modulation with defocused ultrasound transducer array 6635 supplying the whole-head neuromodulation component to head 6600 with the space between frame 6605 and head perimeter 6620 filled by gel pack 6610. Targets related to pain are Cingulate Genu 6620, Dorsal Anterior Cingulate Gyrus 6625, and Insula 6630. The whole-head-neuromodulation transducer 6635 generates sound field represented by 6640, 6645, 6650, 6655, and 6660. For the treatment of pain, Ancillary Stimulation 6665 is applied, in this case an external pain stimulus directing attention to specific targets Cingulate Genu 6620, Dorsal Anterior Cingulate Gyrus 6625, and the Insula 6630. Neural pathways 6670 connect Ancillary Stimulation 6665 to pain-related targets Cingulate Genu 6620, Dorsal Anterior Cingulate Gyrus 6625, and Insula 6630. In addition to the external pain stimulus “directing attention” of the impact of the whole-head neuromodulation to the Cingulate Genu, Dorsal Anterior Cingulate Gyrus, and the Insula, the operator can direct the patient to imagine being in decreased pain or pain free to further shape the effect to the desired result. In addition or an alternative for such focusing attention is another form of neuromodulation in addition to the whole-head ultrasound neuromodulation generated by transducer 6635.

Section I covered optimized neuromodulation, many of the parts applicable to multiple modalities of neuromodulation.

### Section II: Clinical and Physiogical-Impact Applications of Neuromodulation

The targets for the conditions described below are listed in the TABLE 11. The table is not considered exhaustive and also new targets may become identified.

TABLE 11

| | | TARGETS-PRIMARY (U = Up- | |
|---|---|---|---|
| PART | CONDITION | Regulated; D = Down-Regulated) | TARGETS-OTHER |
| I | Orgasm Elicitation | DACG (U), Left Lateral Orbito- | |
| | | Frontal Cortex (D), Insula (U), | |
| | | Amygdala (D), Cerebellum (U), | |
| | | Temporal Lobe (D), Hippocampus | |
| | | (D), Paraventricular Nucleus of | |
| | | Hypothalamus (U) | |
| II | Stroke and Stroke | See Figures | |
| | Rehabilitation | | |
| III | Pain | Rostral Anterior Cingulate Cortex | Orbitofrontal Cortex, |
| | | (ACC)(D) and the Dorsal Anterior | Insula, Amygdalae, |
| | | Cingulate Gyrus (DACG)(D). | Thalamus, |
| | | | Hypothalamus, and |
| | | | Hippocampus |
| IV | Tinnitus | Primary Auditory Cortex (D) | |
| V | Depression and | Orbito-Frontal Cortex (OFC)(U), | Pre-Frontal Cortex, |
| | Bipolar Disorder | Anterior Cingulate Cortex (ACC)(U), | Subgenu Cingulate, |
| | | and Insula (Right (U); Left (D)). | Nucleus Accumbens, |
| | | | Caudate Nucleus, |
| | | | Amygdala, and |
| | | | Hippocampus. |
| VI | Addiction | Orbito-Frontal Cortex (OFC)(D), the | Nucleus Accumbens, |
| | | Dorsal Anterior Cingulate Gyrus | and Globus Pallidus. |
| | | (DACG)(D), and the Insula (D) | |
| VII | PTSD | Amygdala (D), Hippocampus (U), | Ventro-Medial Pre- |
| | | Anterior Cingulate Cortex (U), Orbito- | Frontal Cortex |
| | | Frontal Cortex (U), and the Insula (D) | |
| VIII | Motor (Tremor) | Essential Tremor: Ventro intermedius | internal Globus |
| | Disorders | nucleus (D); Parkinson's Disease: | Pallidus (GPi |
| | | Subthalamic Nucleus (STN)(D) | |
| IX | Autism Spectrum | Anterior Cingulate Gyrus (U), | |
| | Disorders | Caudate Nucleus (U), Parietal Lobe | |
| | | (D), and Amygdala (U) | |
| X | Obesity | Orbito-Frontal Cortex (OFC)(D), | |
| | | Ventromedial Hypothalamus (VMH) | |
| | | (bilaterally)(D), the Lateral | |
| | | Hypothalamus (LH)(D), and the | |
| | | Nucleus Accumbens (NAc)(D) | |
| XI | Alzheimer's | Hippocampus (U), Fornix* (U), | |
| | Disease | Mamillary Body and Dentate Gyrus* | |
| | | (U), Posterior Cingulate Gyrus (PCG) | |
| | | (U), and Temporal Lobe (U) | |
| | | *Usually same Ultrasound transducers | |
| XII | Anxiety including | Orbito-Frontal Cortex (OFC)(U), | |
| | Panic Disorder | Posterior Cingulate Cortex (PCC)(D), | |
| | | Insula (D), and Amygdala (D) | |
| XIII | OCD | Orbito-Frontal Cortex (OFC)(D), | Right Dorsal Lateral |
| | | Temporal Lobe (D), Insula (D), | Prefrontal Cortex, |
| | | Thalamus (U), Cerebellum (U), Head | Ventral Striatum, |
| | | of Caudate Nucleus (U), and Anterior | and Cuneus |
| | | Cingulate Cortex (ACC)(D) | |
| XIV | GI Motility | Gut (U-Constipation) or (D-Diarrhea) | |
| XV | Tourette's | Hippocampus (D) and Amygdala (D) | Thalamus, Sub- |
| | Syndrome | | Thalamic Nuclei, |
| | | | and Basal Ganglia |
| XVI | Schizophrenia | Hippocampus (bilaterally) (U), | Amygdala, |
| | | Ventro-Lateral Pre-Frontal Cortex | Thalamus, Anterior |
| | | (U), Orbito-Frontal Cortex (U), | Cingulate Cortex, |
| | | Medial Pre-Frontal Cortex (D), | the Posterior |
| | | Dorsal-Lateral PFC (U), and the | Cingulate Cortex, |
| | | Temporal Lobe (Entorhinal region)(U) | the Striatum, the |
| | | | Caudate Nucleus, |
| | | | and the Fornix |
| XVII | Epilepsy | (may be bilateral) Hippocampus (D), | Amygdala, Dentate |
| | | Temporal Lobe (D), the Cerebellum | Nucleus, and |
| | | (D), and Thalamus (D) | Mamillary Body |
| XVIII | ADHD | Pre-Frontal Cortex (PFC)(U), Anterior | Superior Parietal |
| | | Cingulate Cortex (ACC)(U) | Lobe, Medial |
| | | | Temporal Lobe, |
| | | | Basal |
| | | | Ganglia/Striatum, |
| | | | Caudate Nucleus, |
| | | | Superior Colliculus, |
| | | | and the Cerebellum |
| XIX | Eating Disorders | Anorexia Nervosa: Pre-Frontal Cortex | Posterior Cingulate |
| | | (PFC)(D), Anterior Cingulate Cortex | Cortex (PCC), Right |
| | | (U); Bulimia: Caudate Nucleus (U), | Dorso-Lateral Pre- |
| | | the Dorsal Anterior Cingulate Gyrus | Frontal Cortex |
| | | (DACG)(D), Pre-Frontal Cortex | (DLPFC), Anterior |
| | | (PFC)(U), Anterior Cingulate Cortex | Cingulate Cortex |
| | | (ACC)(U), Insula (U), Temporal Lobe | (ACC), Medial Pre- |
| | | (U) | Frontal Cortex |
| | | | (MPFC) |
| XX | Cognitive | Orbito-Frontal Cortex (U), and | Left Hippocampus, |
| | Enhancement | Anterior Temporal Lobe (U) | Left Frontal Cortex, |
| | | | Left Middle |
| | | | Temporal Lobe, |
| | | | Ventral Tegmentum, |
| | | | Hypothalamus, and |
| | | | Central Thalamus |
| XXI | Traumatic Brain | TBI: Orbito-Frontal Cortex (OFC)(U), | Midbrain, Reticular |
| | Injury (TBI) | and Occipital Lobe (U); Concussion: | Activating System, |
| | including | Orbito-Frontal Cortex (OFC) (U), | Brainstem, and |
| | Concussion | Temporal Lobe (U), Thalamus (U), | Corpus Callosum |
| | | Hypothalamus (U), and Fornix (U) | |
| XXII | Compulsive | Medial Pre-Frontal Cortex (D), | Amygdala |
| | Sexual Disorders | Nucleus Accumbens (D), | |
| | | Hypothalamus (D), and Ventral | |
| | | Tegmental Area (D) | |
| XXIII | Emotional | Amygdala (U), and Hippocampus (U) | Thalamus, Sub- |
| | Catharsis | | Thalamic Nuclei, |
| | | | Basal Ganglia, and |
| | | | Pre-Frontal Cortex |
| | | | (PFC) |
| XXIV | Autonomous | Insula (U), and Superior Parietal Lobe | |
| | Sensory Meridian | (U) | |
| | Response (ASMR) | | |
| XXV | Occipital Nerve | Occipital Nerve (Unilateral or | |
| | | Bilateral)(U) | |
| XXVI | Sphenopalatine | SPG (U) | |
| | Ganglion (SPG) | | |
| XXVII | Reticular | RAS (U) or (D) | |
| | Activating System | | |
| | (RAS) | | |

Each of the parts of Section II has applicable information included in an individual table (TABLES 12 through 38) that includes the condition-to-be-treated/physiological impact, the primary and secondary patterns applicable, whether mechanical perturbations are applicable to a given target, feedback type, ancillary stimulation, if any, whether intensity modulation is applicable, whether intersecting beams (related to non-invasive neuromodulation modalities) are applicable, whether multimodal neuromodulation is applicable, and a list of other targets, if any. Key considerations are the not all of the listed primary of other targets need be neuromodulated and while the primary and secondary patterns listed represent preferred embodiments and not absolute limitations; other patterns can be employed successfully. Mechanical perturbations would naturally only apply if the neuromodulation modality to be used supports mechanical perturbations; for example, mechanical perturbations do apply to ultrasound neuromodulation but not to Deep Brain Stimulation. In the tables for each part of Section II, the heading MECH. PERTURB. stands for MECHANICAL PERTURBATIONS.

Except as indicated in specifics of the following, all of the clinical applications and neurological impacts include ultrasound neuromodulation control as shown in the block diagrams of the system for variation of ultrasound parameters in FIGS. 5, 11, 12, and associated Parts above. All of the methods and systems of Section I apply.

Ultrasound stimulation uses smaller and less expensive devices than other means of deep-brain neuromodulation such as Transcranial Magnetic Stimulation. The current invention is sufficiently portable for home, work, school, or other non-healthcare-setting use that is key to broad, practical use.

### Part I: Orgasm Elicitation

TABLE 12

| | | TARGETS- | | | |
|---|---|---|---|---|---|
| | | PRIMARY (U = Up- | | | |
| | | Regulated; | PATTERN | | MECH. |
| Part | CONDITION | D = Down-Regulated) | 1° | 2° | PERTURB. |
| I | ORGASM | DACG (U) | Mult. | Burst | Yes |
| | ELICITATION | | Freq. | Mode | |
| | | Left Lateral Orbito- | Fibonacci | Duty | No |
| | | Frontal Cortex (D) | | Cycle | |
| | | Insula (U) | Duty | Burst | No |
| | | | Cycle | Mode | |
| | | Amygdala (D) | Sweep | Fibonacci | No |
| | | | Ampl. | | |
| | | | Mod. | | |
| | | | Freq. | | |
| | | Cerebellum (U) | Burst | Random | Yes |
| | | | Mode | | |
| | | Temporal Lobe (D) | Fibonacci | Duty | Yes |
| | | | | Cycle | |
| | | Hippocampus (D) | Mult. | FiboNacci | |
| | | | Freq. | | |
| | | Paraventricular | Mult. | Sweep | No |
| | | Nucleus of | Freq. | Pulse | |
| | | Hypothalamus (U) | | Freq. | |

| | |
|---|---|
| Feedback Type | Arousal |
| Ancillary | External sexual stimulation |
| Stimulation | |
| Multimodality | Ultrasound, TMS, tDCS Optogenetics |
| Other Targets | None significant |

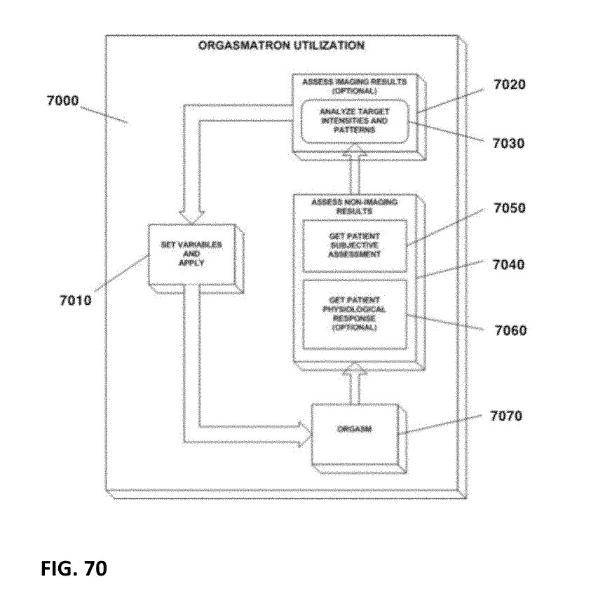

FIGS. 67, 68, and 69 show the various set-up phases, one or a plurality of which may be applied. An important positive factor is that once the response of the individual patient is determined during the one or more set-up phases will be stable but still permit tuning for greater effectiveness. In these phases and the utilization, FIG. 70, of Orgasm Elicitation, Primary Stimulation includes a selection of one or a plurality of external or internal genital stimulation using insertion, pressure, rubbing, vibration, other mechanical, electrical, thermal, ultrasound or other application of energy for tactile, pain, or other stimulation. Stimulation of any of the pelvic organs (e.g., clitoris, vagina, cervix, uterus, anus, rectum, prostate, and penis) can result in any orgasm. Visual Stimulation includes presentation of sexual partner, images of the sexual partner, self, couple together, pornography, sadomasochism, or any other excitatory material. This may be augmented by or substituted for by audio stimulation. Imagining refers to fantasizing by the subject. Ancillary Drug Elements include drugs for erectile dysfunction, mood alteration, or other applicable agents. For example drugs like bupropion that facilitate dopamine presence facilitate orgasms in both men and women. Ancillary Hormone Elements include sex-related hormones (e.g., estrogens and androgens) as well as thyroid or other applicable agents. Secondary Stimulations include non-genital stimulations such as nipples, skin areas in any application or other stimulations using energy or energies as noted under Primary Stimulations above. It is noted that healthcare personnel can view the stimulations effective relative to a specific patient/subject in a non-judgmental way. Note where longer-term changes are involved such as hormones or drugs, the process may take awhile so might want to go to imaging and/or real-time stimulation first. In any case situation may be important enough to patient for long term and/or increased benefit, may be worth the wait.

In the set-up phases the patient/subject physiologic results will include assessment of changes such as, but not limited to, blood pressure, pulse rate, respiratory rate, pupil diameter, pain threshold, and muscle contractions. The various phases for Orgasm Elicitation are shown in FIGS. 67 through 70. Control of the flow in FIGS. 67 through 70 can occur as in FIG. 57 with it accompanying description.

FIG. 67 illustrates the set-up in the non-imaging phase 6700. Set of Select/Adjust Variables steps 6705 has steps Select/Adjust Primary Stimulation(s), If Any 6710, Select/Adjust Visual Element(s), If Any 6715, Select/Adjust Imagining Element(s), If Any 6720, Select/Adjust Ancillary Drug Element(s), If Any 6725, Select/Adjust Ancillary Hormone Element(s), If Any 6730, Select/Adjust Secondary Stimulation(s), If Any 6735, and Select/Adjust Ancillary Other Element(s), If Any 6740. Set Ultrasound neuromodulation is then applied (first time through with its default settings) followed by Orgasm or Non-Orgasm 6780 which in turn is followed by set of steps in Assess Results 6760 which consists of step Get Patient Physiological Response 6770 and Get Patient Subjective Assessment 6765. The final phase is Iterate Through Ultrasound Variables 6750 with Use of Acute Feedback To Adjust 6755 that iterates through the ultrasound variables (e.g., positions, intensity, frequency, phase/intensity relationships, mechanical perturbations, pulse duration, firing pattern) which in turn is followed by Selection/Adjustment Variables 6705. Note that as noted previously that Visual Element(s), If Any 6715 may be replaced by or augmented with auditory-stimulation elements. In any of the figures in this part on elicitation of orgasms, Guided Feedback as covered in Section I, Part X can be effectively applied. One of ordinary skill in the art is capable of following the steps outline in the figures.

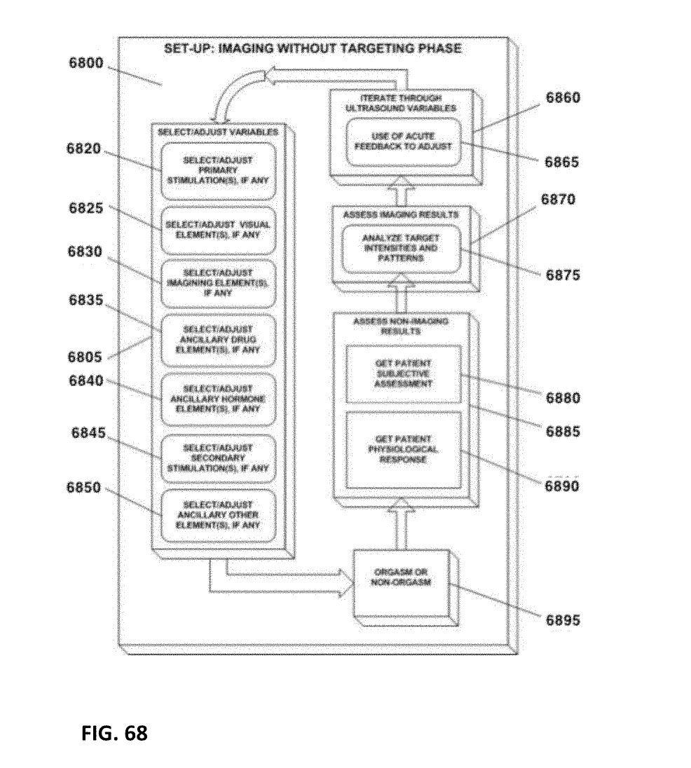

Note that while imaging is covered in the following sections, the invention can be used without imaging. FIG. 68 shows the Set-Up Imaging Without Targeting Phase 6800, which can be done without or with the Set-Up Non-Imaging Phase 6800 preceding it. Set-Up Imaging Phase 6800 Set of Select/Adjust Variables steps 6805 has steps Select/Adjust Primary Stimulation(s), If Any 6820, Select/Adjust Visual Element(s), If Any 6825, Select/Adjust Imagining Element(s), If Any 6830, Select/Adjust Ancillary Drug Element(s), If Any 6835, Select/Adjust Ancillary Hormone Element(s), If Any 6840, Select/Adjust Secondary Stimulation(s), If Any 6845, and Select/Adjust Ancillary Other Element(s), If Any 6850. Ultrasound neuromodulation is then applied (first time through with its default settings) followed by Orgasm or Non-Orgasm 6895 which in turn is followed by set of steps in Assess Non-Imaging Results 6885 which consists of step Get Patient Physiological Response 6890 and Get Patient Subjective Assessment 6880. Assess Non-Imaging Results 6885 is followed by Assess Imaging Results 6870 which is Analyze Target Intensities and Patterns 6875. The final phase is Iterate Through Ultrasound Variables 6860 with Use of Acute Feedback To Adjust 6865 that iterates through the ultrasound variables (e.g., positions, intensity, frequency, mechanical perturbations, phase/intensity relationships, pulse duration, firing pattern) which in turn is followed by Selection/Adjustment Variables 6805. Note that as noted previously that Visual Element(s), If Any 6825 may be replaced by or augmented with auditory-stimulation elements. Note that imaging overall is optional, but may be particularly important in certain cases (e.g., for anorgasmic women or in anorgasmic or hypo-orgasmic post-prostate-surgical men). One can check for best target candidates even without orgasm based on the images resulting from the various forms of stimulations.

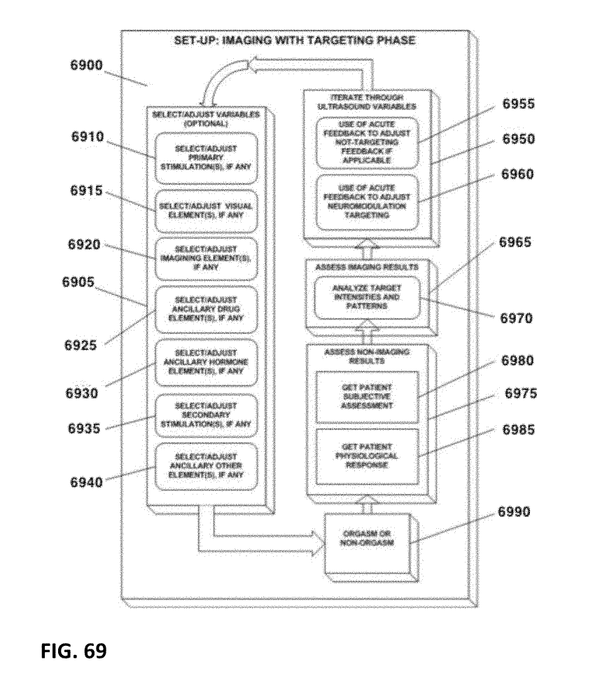

FIG. 69 shows the Set-Up Imaging With Targeting Phase 6900 can be done alone or can follow either the Set-Up Imaging Without Targeting Phase 6800 or Set-Up Non-Imaging Phase 6700, or both. Set-Up Imaging With Targeting Phase 6900 Set of Select/Adjust Variables (Optional) steps 6905 has steps Select/Adjust Primary Stimulation(s), If Any 6910, Select/Adjust Visual Element(s), If Any 6915, Select/Adjust Imagining Element(s), If Any 6920, Select/Adjust Ancillary Drug Element(s), If Any 6925, Select/Adjust Ancillary Hormone Element(s), If Any 6930, Select/Adjust Secondary Stimulation(s), If Any 6935, and Select/Adjust Ancillary Other Element(s), If Any 6940. Ultrasound neuromodulation is then applied (first time through with its default settings) followed by Orgasm or Non-Orgasm 6990 which in turn is followed by set of steps in Assess Non-Imaging Results 6975 which consists of step Get Patient Physiological Response 6985 and Get Patient Subjective Assessment 6980. Assess Non-Imaging Results 6985 is followed by Assess Imaging Results 6965 which is Analyze Target Intensities and Patterns 6970 is performed. The final phase is Iterate Through Ultrasound Variables 6950 with application of both Use of Acute Feedback to Adjust Neuromodulation 6960 and Use of Acute Feedback to Adjust with Non-Targeting Feedback, If Applicable 6955. These iterate through the ultrasound variables (e.g., positions, intensity, frequency, phase/intensity relationships, pulse duration, mechanical perturbations, firing pattern). Iterate through Ultrasound Variables 6950 is then in turn followed by Selection/Adjustment Variables 6905, which is optional. Note that as noted previously that Visual Element(s), If Any 6915 may be replaced by or augmented with auditory-stimulation elements. Note again that imaging overall is optional, but may be particularly important in certain cases (e.g., for anorgasmic women or in anorgasmic or hypo-orgasmic post-prostate-surgical men). Again, one can check for best target candidates even without orgasm.

The left columns of set-up figures (FIG. 67, Non-Imaging, FIG. 68, Imaging without Targeting, and FIG. 69, Imaging with Targeting) list the order in which non-ultrasound variables are to be modified.

FIG. 70 illustrates Orgasm Elicitation Utilization 7000. Based on the previous set-up phases covered in FIGS. 67-69, Set Variables and Apply 7010 is followed by Orgasm 7070 which is followed by in turn followed by Assessment of Non-Imaging Results 7040 which includes optional Get Patient Physiological Response 7060 and Get Patient Subjective Assessment 7050. Get Patient Subjective Assessment 7050 could be optional, but is for practical purposes inherent. Assess Non-Imaging Results 7040 is followed Assess Imaging Results (Optional) 7020 which includes Analyzing Target Intensities and Patterns 7030. This can includes Set Variables and Apply 7010 in future sessions, which may be based on the application of one or a plurality of the set-up phases covered above in FIGS. 67 through 69. Note that previous studies by Komisaruk et al. have included assessment of imaging of couples in an open-frame scanner.

There are a number of user options available but the operator with ordinary skill can operate very effectively by applying the recommended order in making selections, once, with reference to TABLE 12, modality or modalities have been determined, the targeting of the transducers has been completed including whether mechanical perturbations are to be applied, feedback as covered in Section I Part X (including Guided Feedback) and ancillary stimulation selected. The recommended order (incorporated within Guide Feedback if used) is neuromodulation pattern, frequency, pulse duration, intensity, and phase/intensity relationships (if applicable to given neuromodulation modality) to elicit an orgasm.

There are a number of recommended candidate targets. Which will be selected will be selected base on the neuromodulation equipment available to the operator for use with the given patient. There is some inherent patient specificity because orgasms are inherently complex and there is no cookbook recipe for eliciting an orgasm. An important positive factor is that once the response of the individual patient is determined during one or more set-up phases will be stable but still permit tuning for greater effectiveness. It is appropriate to note that while the number of variables that can be adjusted is large, one of ordinary skill can choose to deal with only a subset. More variables can be added if required if and as necessary and as one of ordinary skill gets more comfortable in applying the method.

While primary stimulation of the genitals is the primary applicable form of ancillary stimulation and could elicit an orgasm, the reason for using ancillary stimulation is to aid in tuning the ultrasound neuromodulation so orgasms can be successfully elicited without being triggered by non-ultrasound stimuli although not all individuals are capable of achieving orgasm by the application of non-ultrasound stimuli alone or doing so either easily or with a level of effort acceptable to that individual.

### Part II: Stroke and Rehabilitation

TABLE 13

| | | TARGETS- | | | |
|---|---|---|---|---|---|
| | | PRIMARY | | | |
| | | (U = Up- | | | |
| | | Regulated; | | | |
| | | D = Down- | PATTERN | | MECH. |
| Part | CONDITION | Regulated) | 1° | 2° | PERTURB. |
| II | STROKE & | See Figures | Duty | Burst | Yes |
| | REHABILITATION | | Cycle | Mode | |

| | |
|---|---|
| Feedback Type | Movement of affected limb(s) |
| Ancillary | Movement of affected limb(s) |
| Stimulation | |
| Intensity | Yes |
| Modulation | |
| Multimodality | Ultrasound, TMS, tDCS, Optogenetics |
| Other Targets | Primary Sensory Cortex |

FIG. 71 shows a brain 7100 with cerebral folds with shaded area 7110 denoting the location of the Primary Motor Cortex (designated as M1). The intervening bony skull is not shown. All or part of the motor cortex can be damaged by a stroke and other areas may be damaged by ischemic or hemorrhagic stroke as well. Typically the edges of an area impacted by a stroke are viable and neuromodulation of these edges can mitigate further loss of tissue acutely. In the longer term, neuromodulation of this viable tissue can foster post-stroke rehabilitation. Besides Primary Motor Cortex, strokes cause lesions in the Primary Sensory Cortex, Wernicke's Area, posterior limb of internal capsule, basis pontis, corona radiate, and other neural centers.

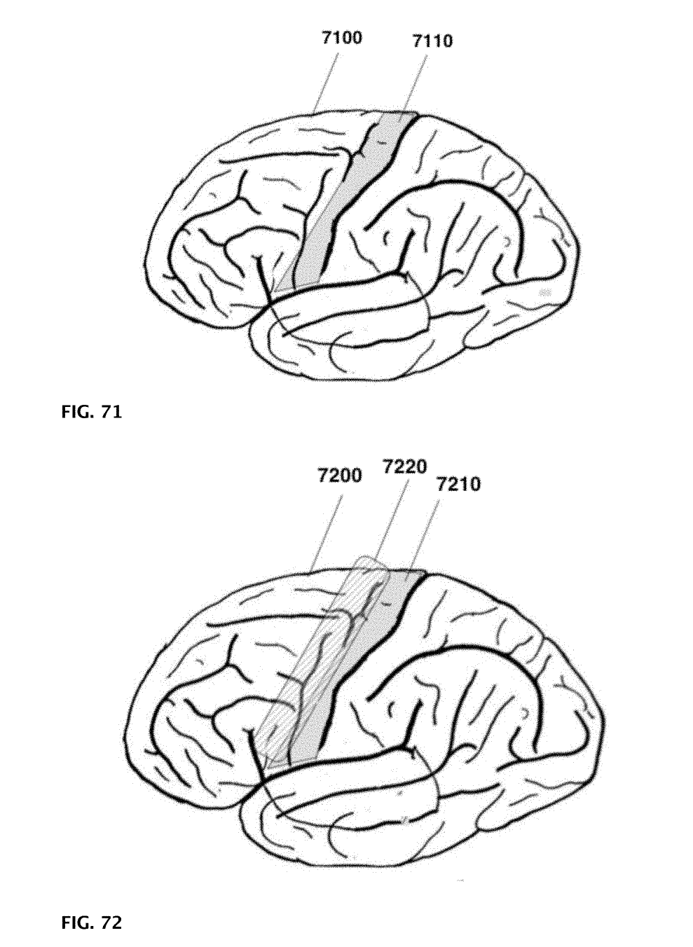

FIG. 17, above, shows an ultrasound transducer array configured to produce an elongated pencil-shaped focused field. Such an array would he applied to stimulate an elongated target such as the motor cortex. Note that one embodiment is a swept-beam transducer with the capability of sweeping the sound field over any portion of the length of the ultrasound transducer. Thus it is possible to determine over what length of a target that the ultrasound is applied. For example, one could apply ultrasound to only the superior portion of the target. In FIG. 17A, an end view of the array is shown with curved-cross section ultrasonic array 1700 forming a sound field 1720 focused on target 1710. FIG. 17B shows the same array in a perspective view, again with ultrasound array 1700, target 1710, and focused field 1720. FIG. 72 shows for brain 7200, the positioning of an ultrasound transducer 7220 over Primary Motor Cortex 7210. The intervening bony skull is not shown. The space between the surface of the ultrasound transducer and the surface of the head is filled with ultrasound conduction medium (e.g., Dermasol from California Medical Innovations)(not shown) with a layer of ultrasound conduction gel between the surface of the ultrasound conduction medium and the surface of the head. One or more such ultrasound transducers may be aimed at other areas of the brain damaged by stroke. Stimulation can be unilateral or bilateral. It has been found using rTMS that there can be advantages to exciting the motor cortex ipsilateral to the brain lesion and inhibiting the motor cortex contralateral to the brain region.

The location of the stroke is immaterial from the perspective of neuromodulation. It can be applied to strokes located in cortical, subcortical, brainstem, and other regions. The region impacted by stroke can be a single one such as a large infarct or multiple small ones. It also does not matter whether the stroke is ischemic and hemorrhagic. Not only does neuromodulation foster metabolic changes, the repetitive neuromodulation can retrain neural pathways to allow restore function.

Stimulation can be done unilaterally or bilaterally to see diagnostically which muscle or muscle groups are affected. Therapeutically, the ultrasound neuromodulation can be used to stimulate muscles to exercise them.

Another consideration is combination with neuromodulation of regions other than Motor Cortex. For example, neuromodulation of the Reticular Activating System to keep the general level of brain and base central activity up to prevent Central Nervous System failure.

The invention can be applied for a variety of stroke-related clinical purposes such as reversibly putting a patient into a coma (for example for the purpose of protecting the brain of the patient after a stroke or head injury). Effects can be either acute or durable effect through Long-Term Potentiation (LTP) and/or Long-Term Depression (LTD). Since the effect is reversible putting the patient in even a vegetative state is safe if handled correctly. The application of LTP or LTD provides a mechanism for adjusting the bias of patient activity up or down.

### Part III: Pain

TABLE 14

| | | TARGETS-PRIMARY | | | |
|---|---|---|---|---|---|
| | CON- | (U = Up-Regulated; | PATTERN | | MECH. |
| Part | DITION | D = Down-Regulated) | 1° | 2° | PERTURB. |
| III | PAIN | Rostral Anterior | Mult. | Burst | No |
| | | Cingulate Cortex | Freq. | Mode | |
| | | (ACC)(D) | | | |
| | | Dorsal Anterior | Mult. | Burst | yes |
| | | Cingulate Gyrus | Freq. | Mode | |
| | | (DACG)(D) | | | |

| | |
|---|---|
| Feedback | Pain Characterization (e.g., Visual Analog Scale) |
| Type | |
| Ancillary | Soothing sound |
| Stimulation | |
| Multi- | Ultrasound, TMS, tDCS, VNS, Optogenetics, |
| modality | Occipital, Sphenopalatine Ganglion |
| Other | Orbitofrontal Cortex, Insula, Amygdalae, Thalamus, |
| Targets | Hypothalamus, and Hippocampus |

Pain targets are known to be involved in pain processing and can be down regulated at a frequency on the order of approximately, but not limited to, 1 Hz.

The invention can be applied for a variety of clinical purposes such as treatment of acute or chronic post-operative pain, acute or chronic pain related to dental procedures, chronic pain related to conditions like fibromyalgia, low-back pain, headache, neurogenic pain, cancer pain, arthritis pain, and psychogenic pain. Effects can be either acute or durable effect through Long-Term Potentiation (LTP) and/or Long-Term Depression (LTD).

### Part IV: Tinnitus

TABLE 15

| | | TARGETS- | | | |
|---|---|---|---|---|---|
| | | PRIMARY | | | |
| | | (U = Up- | | | |
| | | Regulated; | | | |
| | | D = Down- | PATTERN | | MECH. |
| Part | CONDITION | Regulated) | 1° | 2° | PERTURB. |
| IV | TINNITUS | Primary Auditory | Sweep | Duty | No |
| | | Cortex (D) | Pulse | Cycle | |
| | | | Freq. | | |

| | |
|---|---|
| Feedback Type | Tinnitus level |
| Ancillary | Masking sound |
| Stimulation | |
| Multimodality | Ultrasound, TMS, Optogenetics |

The primary auditory cortex is essentially in the same region as Brodmann areas 41 and 42. It is located in the posterior half of the superior temporal gyms and also dives into the lateral sulcus as the transverse temporal gyri.

### Part V: Depression and Bipolar Disorder

TABLE 16

| | | TARGETS-PRIMARY | | | |
|---|---|---|---|---|---|
| | | (U = Up-Regulated; | PATTERN | | MECH. |
| Part | CONDITION | D = Down-Regulated) | 1° | 2° | PERTURB. |
| V | DEPRESSION & | Orbito-Frontal Cortex | Fibonacci | Duty | No |
| | BIPOLAR | (OFC)(U) | | Cycle | |
| | DISORDER | Anterior Cingulate | Mult. | Burst | Yes |
| | | Cortex (ACC)(U) | Freq. | Mode | |
| | | Insula (Right (U); Left | Duty | Burst | No |
| | | (D)) | Cycle | Mode | |

| | |
|---|---|
| Feedback Type | Depression scale |
| Ancillary | Upbeat Music (e.g., Tchaikovsky 1812 Overture) |
| Stimulation | |
| Multimodality | Ultrasound, TMS, tDCS, VNS, Optogenetics |
| Other Targets | Pre-Frontal Cortex, Subgenu Cingulate, Nucleus ns, |
| | Caudate Nucleus, Amygdala, and Hippocampus. |

Multiple targets can be neuromodulated singly or in groups to treat depression or bipolar disorder. The specific targets and/or whether the given target is up regulated or down regulated, can depend on the individual patient and relationships of up regulation and down regulation among targets, and the patterns of stimulation applied to the targets. In some cases neuromodulation will be bilateral and in others unilateral.

### Part VI: Addiction

TABLE 17

| | | TARGETS-PRIMARY | | | |
|---|---|---|---|---|---|
| | | (U = Up-Regulated; | PATTERN | | MECH. |
| Part | CONDITION | D = Down-Regulated) | 1° | 2° | PERTURB. |
| VI | ADDICTION | Orbito-Frontal Cortex | Fibonacci | Duty | No |
| | | (OFC)(D) | | Cycle | |
| | | Dorsal Anterior Cingulate | Mult. | Burst | Yes |
| | | Gyrus (DACG)(D) | Freq. | Mode | |
| | | Insula (D) | Duty | Burst | No |
| | | | Cycle | Mode | |

| | |
|---|---|
| Feedback Type | Level of craving for applicable substance in light of image or |
| | odor of addictive substance |
| Ancillary | Image or odor of addictive substance; Visual or auditory |
| Stimulation | praise for restraint |
| Intersecting | Dorsal Anterior Cingulate Gyrus and Insula from upward- |
| Beams | directed lateral transducers |
| Multimodality | Ultrasound, TMS, tDCS, Optogenetics |
| Other Targets | Nucleus Accumbens, and Globus Pallidus. |

### Part VII: Post Traumatic Stress Disorder (PTSD)

TABLE 18

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| VII | PTSD | Amygdala (D) | Sweep | Fibonacci | No |
| | | | Ampl. | | |
| | | | Mod. | | |
| | | | Freq. | | |
| | | Hippocampus (U) | Mult. | Fibonacci | No |
| | | | Freq. | | |
| | | Anterior Cingulate | Mult. | Burst | No |
| | | Cortex (U) | Freq. | Mode | |
| | | Orbito-Frontal Cortex | Fibonacci | Duty | No |
| | | (U) | | Cycle | |
| | | Insula (D) | Duty | Burst | No |
| | | | Cycle | Mode | |

| | |
|---|---|
| Feedback Type | Response to viewing applicable inciting image |
| Ancillary | Soothing sound |
| Stimulation | |
| Intersecting | Hippocampus, Amygdala, and Insula from upward-directed |
| Beams | lateral transducers |
| Multimodality | Ultrasound, TMS, tDCS, Optogenetics |
| Other Targets | Ventro-Medial Pre-Frontal Cortex |

In the application of the therapeutic ultrasound, the hyperactive Amygdala would be down regulated, the Anterior Cingulate Cortex (ACC) up regulated, the Orbito-Frontal Cortex (OFC) up regulated, the Hippocampus up regulated, and the Insula down regulated. If the Ventro-Medical Pre-Frontal Cortex were targeted it would be up regulated.

### Part VIII: Motor Disorders

TABLE 19

| | | TARGETS- | | | |
|---|---|---|---|---|---|
| | | PRIMARY | | | |
| | | (U = Up- | | | |
| | | Regulated; | | | |
| | | D = Down- | PATTERN | | MECH. |
| Part | CONDITION | Regulated) | 1° | 2° | PERTURB. |
| VIII | MOTOR | Essential Tremor: | Burst | Random | No |
| | (TREMOR) | Ventro | Mode | | |
| | DISORDERS | intermedius | | | |
| | | nucleus (D); | | | |
| | | Parkinson's | Burst | Random | No |
| | | Disease: | Mode | | |
| | | Subthalamic | | | |
| | | Nucleus | | | |
| | | (STN)(D) | | | |

| | |
|---|---|
| Feedback Type | Measured amplitude of tremor |
| Ancillary | Restraint of tremor |
| Stimulation | |
| Multimodality | Ultrasound, TMS, tDCS, DBS, Optogenetics |
| Other Targets | internal Globus Pallidus (GPi) |

In the case of motor (tremor) disorders, based on experience with Deep-Brain Stimulation (DBS) with implanted electrodes, treatment for Parkinson's Disease or Essential Tremor would typically be 130 pulse per second and Dystonia in the range of 135-185 pulses per second (all superimposed on the carrier frequency of say 0.5 MHz or similar and may be divided into pulses 0.1 to 20 msec. repeated at intervals of 2 Hz or shorter) although this will be both patient and condition specific. For example in difficult cases it may be that rates up to 250 Hz or down to 50 Hz may be more effective. Below 50 pulses per second, the tremor can get worse.

For essential tremor (ET), the structure is the ventro intermediate nucleus of the thalamus (Vint), and for dystonia, the GPi or STN is stimulated. Unilateral DBS is used for essential tremor (e.g., for suppression of upper-extremity tremor) and bilateral DBS is used for PD and dystonia.

As to contraindications, Dementia is a contraindication for DBS treatment, but need not be so for ultrasound neuromodulation. Other DBS contradictions include exposure to MRI using a full-body RF coil or a head transmit coil that extends over the chest area, diathermy, and other devices such as cardiac pacemakers, cardioverter/defibrillators, external defibrillators, ultrasonic equipment, electrocautery, or radiation therapy. Again, these need not be contraindications for ultrasound neuromodulation.

Feedback as covered in Section I, Part X can be applied, including taking the feedback-derived signal in FIG. 51 and using it as input to a mechanical actuator to counteract the tremor.

### Part IX: Autism Spectrum Disorders

TABLE 20

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| IX | AUTISM | Anterior Cingulate | Mult. | Burst | No |
| | SPECTRUM | Gyrus (U) | Freq. | Mode | |
| | DISORDERS | Caudate Nucleus (U) | Mult. | Sweep | No |
| | | | Freq. | Pulse | |
| | | | | Freq. | |
| | | Parietal Lobe (D) | Fibonacci | Duty | Yes |
| | | | | Cycle | |
| | | Amygdala (U) | Sweep | Fibonacci | No |
| | | | Ampl | | |

| | |
|---|---|
| Feedback Type | Test response to spontaneous situation |
| Ancillary | Being tightly held, pressure stimulation, vibration |
| Stimulation | |
| Intersecting | Caudate Nucleus and Amygdala from downward-directed |
| Beams | lateral transducers |
| Multimodality | Ultrasound, TMS, tDCS, Optogenetics |

### Part X: Obesity

TABLE 21

| | | TARGETS-PRIMARY | | | |
|---|---|---|---|---|---|
| | | (U = Up-Regulated; D = Down- | PATTERN | | MECH. |
| Part | CONDITION | Regulated) | 1° | 2° | PERTURB. |
| X | OBESITY | Orbito-Frontal Cortex | Fibonacci | Duty | No |
| | | (OFC)(D) | | Cycle | |
| | | Ventromedial Hypothalamus | Mult. | Sweep | No |
| | | (VMH) (bilaterally)(D) | Freq. | Pulse | |
| | | | | Freq. | |
| | | Lateral Hypothalamus | Mult. | Sweep | No |
| | | (LH)(D) | Freq. | Pulse | |
| | | | | Freq. | |
| | | Nucleus Accumbens | Fibonacci | Burst | No |
| | | (NAc)(D) | | Mode | |

| | |
|---|---|
| Feedback | Level of craving for applicable food in light of image or odor of |
| Type | that food |
| Ancillary | Praise for restraint |
| Stimulation | |
| Multi- | Ultrasound, TMS, tDCS, Optogenetics, DBS |
| Multimodality | |

Part XI: Alzheimer's Disease

### Part XII: Anxiety Including Panic Disorder

TABLE 23

| | | TARGETS-PRIMARY | | | |
|---|---|---|---|---|---|
| | | (U = Up-Regulated; | PATTERN | | MECH. |
| Part | CONDITION | D = Down-Regulated) | 1° | 2° | PERTURB. |
| XII | ANXIETY | Orbito-Frontal Cortex | Fibonacci | Duty | No |
| | INCLUDING | (OFC)(U) | | Cycle | |
| | PANIC | Posterior Cingulate | Mult. | Burst | No |
| | DISORDER | Cortex (PCC)(D) | Freq. | Mode | |
| | | Insula (D) | Duty | Burst | No |
| | | | Cycle | Mode | |
| | | Amygdala (D) | Sweep | Fibonacci | No |
| | | | Ampl. | | |
| | | | Mod. | | |
| | | | Freq. | | |

| | |
|---|---|
| Feedback Type | Response to frenetic images and/or audio |
| Ancillary | Soothing music |
| Stimulation | |
| Intersecting | Insula and Amygdala from downward-directed lateral |
| Beams | transducers |
| Multimodality | Ultrasound, TMS, tDCS, Optogenetics |

### Part XIII: Obsessive Compulsive Disorder

TABLE 24

| | | TARGETS-PRIMARY | | | |
|---|---|---|---|---|---|
| | | (U = Up-Regulated; | PATTERN | | MECH. |
| Part | CONDITION | D = Down-Regulated) | 1° | 2° | PERTURB. |
| XIII | OCD | Orbito-Frontal Cortex | Fibonacci | Duty | No |
| | | (OFC)(D) | | Cycle | |
| | | Temporal Lobe (D) | Fibonacci | Duty | Yes |
| | | | | Cycle | |
| | | Insula (D) | Duty | Burst | No |
| | | | Cycle | Mode | |
| | | Thalamus (U) | Burst | Random | No |
| | | | Mode | | |
| | | Cerebellum (U) | Burst | Random | Yes |
| | | | Mode | | |
| | | Head of Caudate | Mult. | Sweep | No |
| | | Nucleus (U) | Freq. | Pulse | |
| | | | | Freq. | |
| | | Anterior Cingulate | Mult. | Burst | No |
| | | Cortex (ACC)(D) | Freq. | Mode | |

| | |
|---|---|
| Feedback Type | Response to video of obsessive behavior |
| Ancillary | Soothing music |
| Stimulation | |
| Intersecting | Cerebellum and Thalamus from posterior transducer(s) and |
| Beams | Temporal Lobe, Insula, and Head of Caudate Nucleus from |
| | downward- and laterally-directed transducer(s) from midline- |
| | superior location |
| Multimodality | Ultrasound, TMS, tDCS, Optogenetics |
| Other Targets | Right Dorsal Lateral Prefrontal Cortex, Ventral Striatum, and |
| | Cuneus |

### Part XIV: Gastrointestinal Motility

TABLE 25

| | | TARGETS- | | | |
|---|---|---|---|---|---|
| | | PRIMARY (U = | | | |
| | | Up-Regulated; | | | |
| | | D = Down- | PATTERN | | MECH. |
| Part | CONDITION | Regulated) | 1° | 2° | PERTURB. |
| XIV | GI MOTILITY | Gut (U- | Burst | Duty | Yes |
| | | Constipation) | Mode | Cycle | |
| | | Gut (D-Diarrhea) | Burst | Duty | Yes |
| | | | Mode | Cycle | |

| | |
|---|---|
| Feedback Type | Response to inciting food for diarrhea or intestinal |
| | feeling for constipation |
| Ancillary | Pressure on abdomen |
| Stimulation | |
| Intensity | Yes; from all transducers, irrespective of modality |
| Modulation | |
| Intersecting | Yes; from all transducers |
| Beams | |
| Multimodality | Ultrasound, TMS, Optogenetics |

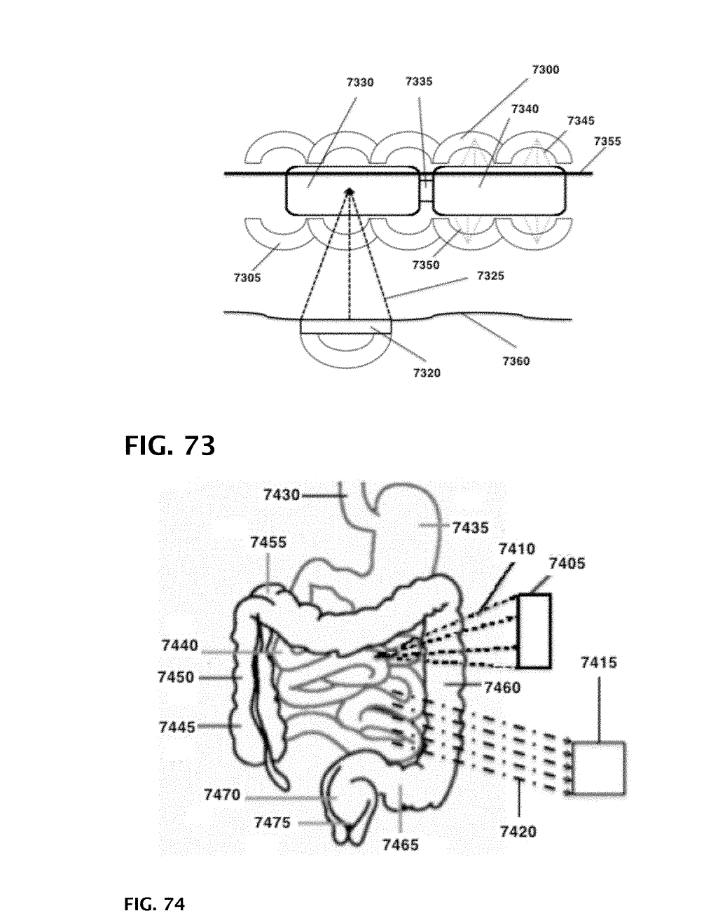

FIG. 73 shows gastrointestinal lumen 7300 within body 7360. Ultrasound neuromodulation transducer capsule 7340 with ultrasonic beam 7345 hitting one side of the lumen and ultrasonic beam 7350 hitting the other. In fact, the beams generated will be 360 degrees around the transducer and longitudinal along the length of the transducer. Power to the ultrasound transducer is provided from power-supply capsule 7330 through connection 7335. Power-supply capsule 7330 could contain a battery allowing low-power stimulation by ultrasound transducer 7340, but in most embodiments will contain an antenna and power transducer. The electromagnetic energy source 7320 is connected to a higher-level power source with power control, not shown. The output of electromagnetic energy source 7320 (e.g., Witricity) is beam 7325 whose power is absorbed by power-supply capsule 7330. The activity of the lumen can be monitored in some cases by probe 7355, either for determination of neuromodulation variables, or for real-time feedback. Examples of physiological feedback measurement are internal electrodes, electronic pressure transducers, or manometic instrumentation. Endoscopically placed probe 7355 is connected to the monitoring instrumentation (not shown) by a cable, also not shown. In another embodiment (not shown), the sensors (e.g., myoelectric sensors or pressure sensors) are built into the ultrasonic transducer 7340 and/or power-supply capsule 7330. Data may be collected continuously or between pulses or between pulse trains. In another embodiment, electrodes on the surface of the body of the patient detect the myoelectric activity of the colon. While the FIG. 73 refers to the colon, the invention applies to the small intestine or the rectum as well.

FIG. 74 shows gastrointestinal organs that could be neuromodulated including the esophagus 7430, the stomach 7435, the small intestine 7440, the cecum 7445, the ascending colon, 7450, the transverse colon 7455, the descending colon 260, the sigmoid colon 7465, the rectum 7470, and the anus 7475. An additional target is the vagal nerve. Ultrasound transducer 7405 with its ultrasound beam 7410 (shown neuromodulating the small intestine) provides neuromodulation. Other embodiments include multiple ultrasound transducers focusing on one or more targets. The signals indicating level of gastrointestinal motility (e.g., by electrogastroenterogram) is detected by sensor 7415. The control diagram for taking this feedback and controlling the level of neuromodulation is shown in FIG. 51. FIG. 74 illustrates the internal view of the body, but each ultrasound transducer will be applied to the skin of the body (not shown). For ultrasound to be effectively applied to the external body surface and transmitted to and through the body, coupling must be put into place. Ultrasound transmission (for example Dermasol from California Medical Innovations) medium placed, if applicable, within the ultrasound transducer cavity so that a contiguous surface is presented to the surface of the skin. This is true whether the ultrasound transducer is applied to the anterior surface of the abdomen, and/or one or both surfaces of the abdomen, and/or the back, and or the surface surrounding the rectum. This contiguous surface is not sufficient, however. To “complete the circuit,” in FIG. 74, an ultrasound-conduction gel layer (not shown) is placed between ultrasound transducer/lens 7405 and the surface of the body (not shown). In other embodiments, multiple ultrasound transducers whose beams intersect at that target replace an individual ultrasound transducer for that target. In other embodiments, both internal and external ultrasound transducers provide neuromodulation.

With respect to the control of motility of the small intestine as could be done as in either FIG. 73 or FIG. 74, there can be acceleration of the carriage of the output of the contents of the stomach through the small intestine where absorption of nutrients occurs so less such absorption occurs and the therapeutic malabsorption fosters weight loss. An additional approach is the use of tagged food or drugs combined with imaging to judge the results.

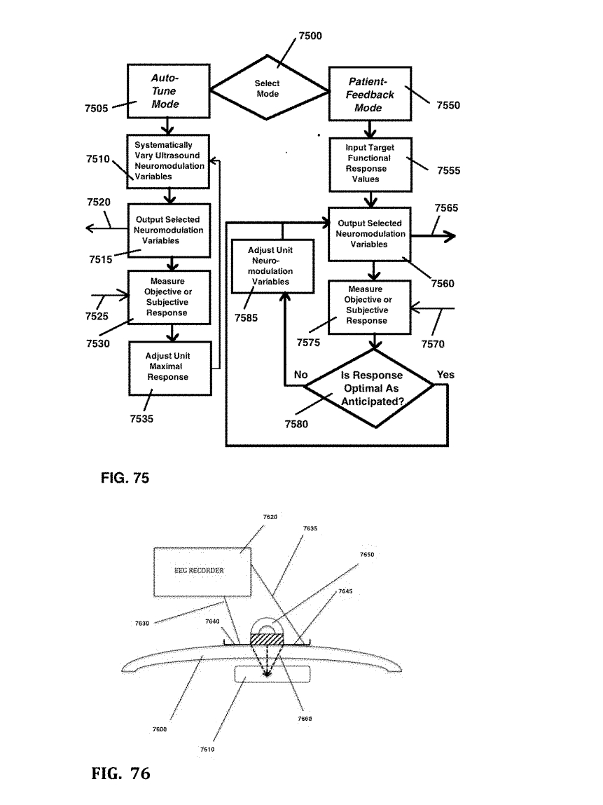

FIG. 75 shows a block diagram for a control of the neuromodulation based on feedback as to level of gastrointestinal motility. A key element for effective neuromodulation is to tune it to the specific patient at the specific time of treatment. As shown in FIG. 75, in Select Mode 7500, two modes are available, Auto-Tune Mode 7505 and Patient-Feedback Mode 7550. Auto-Tune Mode 7505 is used when the ultrasound neuromodulation is being initially set up for the particular patient. Patient-Feedback Mode 7550 is used during the subsequent treatment sessions. Auto-Tune Mode Mode 7505 or Patient-Feedback Mode 7550 may include Guided Feedback as covered in Part 10.

In Auto-Tune Mode 7505, the neuromodulation variables (carrier frequency, neuromodulation frequency, transducer direction, intensity, pulse pattern including pulse rate, pulse duration, intensity, mechanical perturbations, and phase/frequency relationships for ultrasound-beam steering and/or mechanically redirecting the position and/or direction of ultrasound beams) are varied, not necessarily all in a given session. Hill climbing or other algorithms like the greedy algorithm or simulated annealing are used for optimization. Neuromodulation at the current set of variable values is output via block 7515 through output channel 7520. The physiological evidence of bowel activity (e.g., via electrogastrography (electrogastrogram, EGG) or electrocologram (intra-colonic recording (see FIG. 73) or external recording from external cutaneous electrodes) or subject patient-report results come back through channel 7525 and measured in block 7530. Based on whether maximal response has been achieved as judged in block 7535, control is exercised to either maintain the current values if the response has been judged as satisfactory or to vary the neuromodulation variables in 7510 if not. In some implementations, only objective feedback is used and patient feedback is not utilized.

In the Patient-Feedback Mode 7550 of FIG. 75, the treatment planner inputs target functional response values in 7555 resulting in the output of the selected neuromodulation variables in 7560 through output channel 7565. The objective response and the subjective input (e.g., level of feeling or motility or hearing gurgling) come back through channel 7570 in Measure Objective or Subjective Response 7575 where subsequently the response is evaluated in block 7580 (“Is Response Optimal As Anticipated?”). If the response is optimal, then the neuromodulation variables are left as they were; if the response is not optimal, the variables are adjusted in 7585 and output via block 360 through output channel 7565. One embodiment of the mode is to provide the patient the capability of turning the level of motility up or down, including when sitting on a toilet. In some implementations, only subjective feedback is used and objective feedback is not utilized. Motility feedback can be obtained from surface electrodes detecting myoelectric activity, internal electrodes inserted into the GI tract, imaging (likely ultrasound imaging), internal pressure sensors, or other suitable means. The latter can include using one or more microphones to detect evidence of motility such as gurgling or detection of releasing of fluid through the wall of the gut. Electromyographic activity can be obtained using an electrogastroenterogram for the small intestine or the stomach (an electrogastrogram is used for the stomach alone), and an electrocologram for the large intestine. Maximum muscle contraction rates in waves per minute are approximately three for the stomach, 12 for the duodenum, 8 for the ileum, 11 for the jejunum, and 17 for the rectum. The use of electromyography, electrogastrography, and imaging of one form or another can not only be used for feedback-control purposes and tuning, but also to see how well the neuromodulation is working by looking inside the body and seeing its impact on the GI-system components.

The use of electromyography, electrogastrography, and imaging of one form or another can not only be used for feedback-control purposes and tuning, but also to see how well the neuromodulation is working by looking inside the body and seeing its impact on the GI-system components.

### Part XV: Tourette's Syndrome

TABLE 26

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| XV | TOURETTE'S | Hippocampus (D) | Mult. | Fibonacci | Yes |
| | SYNDROME | | Freq. | | |
| | | Amygdala (D) | Sweep | Fibonacci | No |
| | | | Ampl. | | |
| | | | Mod. | | |
| | | | Freq. | | |

| | |
|---|---|
| Feedback Type | Measurement of verbal outburst to inciting situation |
| Ancillary | Soothing music |
| Stimulation | |
| Intersecting | Hippocampus and Amygdala from upward-directed lateral |
| Beams | transducers |
| Multimodality | Ultrasound, TMS, tDCS, DBS, Optogenetics |
| Other Targets | Thalamus, Sub-Thalamic Nuclei, and Basal Ganglia |

Multiple targets can be neuromodulated singly or in groups to treat Tourette's Syndrome, whether motor tics or vocalizations.

### Part XVI: Schizophrenia

TABLE 27

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| XVI | SCHIZOPHRENIA | Hippocampus | Mult. | Fibonacci | Yes |
| | | (bilaterally) (U), | Freq. | | |
| | | Ventro-Lateral Pre- | Fibonacci | Duty | No |
| | | Frontal Cortex (U) | | Cycle | |
| | | Orbito-Frontal | Fibonacci | Duty | No |
| | | Cortex (U) | | Cycle | |
| | | Medial Pre-Frontal | Fibonacci | Duty | No |
| | | Cortex (D) | | Cycle | |
| | | Dorsal-Lateral PFC | Fibonacci | Duty | No |
| | | (U) | | Cycle | |
| | | Temporal Lobe | Fibonacci | Duty | Yes |
| | | (Entorhinal | | Cycle | |
| | | region)(U) | | | |

| | |
|---|---|
| Feedback Type | Level of paranoia response to inciting visual and/or |
| | audio |
| Ancillary Stimulation | Soothing sound |
| Intersecting Beams | Temporal Lobe and Hippocampus from downward- |
| | directed lateral transducer(s) |
| Multimodality | Ultrasound, TMS, tDCS, Optogenetics |
| Other Targets | Amygdala, Thalamus, Anterior Cingulate Cortex, the |
| | Posterior Cingulate Cortex, the Striatum, the Caudate |
| | Nucleus, and the Fornix |

### Part XVII: Epilepsy

TABLE 28

| | | TARGETS-PRIMARY | | | |
|---|---|---|---|---|---|
| | | (U = Up-Regulated; | PATTERN | | MECH. |
| Part | CONDITION | D = Down-Regulated) | 1° | 2° | PERTURB. |
| XVII | EPILEPSY | Hippocampus (D) | Mult. | Fibonacci | Yes |
| | | (may be bilateral) | Freq. | | |
| | | Temporal Lobe (D) | Fibonacci | Duty | Yes |
| | | | | Cycle | |
| | | Cerebellum (D) | Burst | Random | Yes |
| | | | Mode | | |
| | | Thalamus (D) | Burst | Random | No |
| | | | Mode | | |

| | |
|---|---|
| Feedback Type | Level of reaction to eliciting image |
| Ancillary | Soothing sound |
| Stimulation | |
| Intensity | Yes; any transducer(s) for any modalities |
| Modulation | |
| Intersecting | Cerebellum and Thalamus from posterior- and upward- |
| Beams | directed transducer(s) and Temporal Lobe and Hippocampus |
| | from downward-directed lateral transducer(s) |
| Multimodality | Ultrasound, TMS, tDCS, VNS, DBS, Optogenetics |
| Other Targets | Amygdala, Dentate Nucleus, and Mamillary Body |

FIG. 76 shows neuromodulation target 7610 within patient head 7600. Transducer 7650 with its beam 7660 neuromodulates target 7610. A layer of ultrasound conduction gel (not shown) is placed between the face of transducer 7650 and head surrounded by skull segment 7600. The target can be one of the targets shown in FIG. 6 or others. Multiple transducers can be aimed at multiple targets. Alternatively multiple transducers with beams intersecting at a single target can be used. EEG signals are taken from electrodes 7640 and 7645 through conductors 7630 and 7635 respectively to EEG recorder 7620. When an incipient seizure is detected in EEG recorder 7620, a circuit (not shown) is activated where a trigger is provided to the control unit (not shown) providing neuromodulation output to ultrasound transducer 7650 to stop the seizure. EEG signals can also be detected in the ear as taught in a system that included such a detection device combined with a stimulator by Fischell and Upton (US Patent Application Publication US 2003/0195588, “External Ear Canal Interface for the Treatment of Neurological Disorders”).

Neuromodulation can be applied continually for frequent seizures of status epilepticus, but another mode of great utility is to use an EEG signal to detect when a seizure is about to occur and have the neuromodulation turned on at the time. As described by Neuropace (Pless, U.S. Pat. No. 6,466,822, “Multimodal Neural Stimulator and Process of Using It”), applying such a mechanism can prevent a full-blown seizure. This approach uses implanted electrodes, placed during an invasive procedure. Ultrasound neuromodulation has the distinct benefits of being non-invasive, less expensive, and portable so it can be used at home, in school, or while working.

### Part XVIII: Attention Deficit Hyperactivity Disorder (ADHD)

TABLE 29

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| XVIII | ADHD | Pre-Frontal Cortex | Fibonacci | Duty | No |
| | | (PFC)(U) | | Cycle | |
| | | Anterior Cingulate | Mult. | Burst | No |
| | | Cortex (ACC)(U) | Freq. | Mode | |

| | |
|---|---|
| Feedback Type | Level of hyperactivity response to inciting visual and/or |
| | audio |
| Ancillary | Soothing sound or viewing structured calendar of activities |
| Stimulation | |
| Intersecting | Pre-Frontal Cortex and Anterior Cingulate Cortex from |
| Beams | anterior transducer(s) |
| Multimodality | Ultrasound, TMS, tDCS, Optogenetics |
| Other Targets | Superior Parietal Lobe, Medial Temporal Lobe, Basal |
| | Ganglia/Striatum, Caudate Nucleus, Superior Colliculus, |
| | and the Cerebellum |

A selection from the same set of targets can be neuromodulated to treat Disruptive Mood Dysregulation Disorder (DMDD).

Part XIX: Eating Disorders

### Part XX: Cognitive Enhancement

TABLE 31

| | | TARGETS- | | | |
|---|---|---|---|---|---|
| | | PRIMARY (U = Up- | | | |
| | | Regulated; | | | |
| | | D = Down- | PATTERN | | MECH. |
| Part | CONDITION | Regulated) | 1° | 2° | PERTURB. |
| XX | COGNITIVE | Orbito-Frontal | Fibonacci | Duty | No |
| | ENHANCEMENT | Cortex (U) | | Cycle | |
| | | Anterior Temporal | Fibonacci | Duty | Yes |
| | | Lobe (U) | | Cycle | |

| | |
|---|---|
| Feedback Type | Performance on probem-solving test or video gaming |
| Ancillary | Presentation of cognitive test like memory or problem- |
| Stimulation | solving examination |
| Multimodality | Ultrasound, TMS, tDCS, Optogenetics |
| Other Targets | Left Hippocampus, Left Frontal Cortex, Left Middle |
| | Temporal Lobe, Ventral Tegmentum, Hypothalamus, and |
| | Central Thalamus |

Part XXI: Traumatic Brain Injury Including Concussion

### Part XXII Compulsive Sexual Disorders

TABLE 33

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| XXII | COMPULSIVE | Medial Pre-Frontal | Fibonacci | Duty | No |
| | SEXUAL | Cortex (D) | | Cycle | |
| | DISORDERS | Nucleus Accumbens | Fibonacci | Burst | No |
| | | (D) | | Mode | |
| | | Hypothalamus (D) | Mult. Freq. | Sweep | No |
| | | | | Pulse | |
| | | | | Freq. | |
| | | Ventral Tegmental | Random | Fibonacci | No |
| | | Area (D) | | | |

| | |
|---|---|
| Feedback Type | Level of reaction to explicit visual and/or audio sexual material |
| Ancillary | Soothing sounds |
| Stimulation | |
| Intersecting | Nucleus Accumbens and Hippocampus from downward- and |
| Beams | posterior-directed transducer |
| Multimodality | Ultrasound, TMS, tDCS, DBS, Optogenetics |
| Other Targets | Amygdala |

### Part XXIII: Emotional Catharsis

TABLE 34

| | | TARGETS- | | | |
|---|---|---|---|---|---|
| | | PRIMARY (U = Up- | | | |
| | | Regulated; | PATTERN | | MECH. |
| Part | CONDITION | D = Down-Regulated) | 1° | 2° | PERTURB. |
| XXIII | EMOTIONAL | Amygdala (U) | Sweep | Fibonacci | No |
| | CATHARSIS | | Ampl. | | |
| | | | Mod. | | |
| | | | Freq. | | |
| | | Hippocampus (U) | Mult. | Fibonacci | Yes |
| | | | Freq. | | |

| | |
|---|---|
| Feedback Type | Level of reaction to release trigger |
| Ancillary | Triggering stimulus |
| Stimulation | |
| Multimodality | Ultrasound, TMS, tDCS, Optogenetics |
| Other Targets | Thalamus, Sub-Thalamic Nuclei, Basal Ganglia, and Pre- |
| | Frontal Cortex (PFC) |

### Part XXIV: Autonomous Sensory Meridian Response (ASMR)

TABLE 35

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| XXIV | AUTONOMOUS | Insula (U) | Duty | Burst | No |
| | SENSORY | | Cycle | Mode | |
| | MERIDIAN | Superior Parietal Lobe | Fibonacci | Duty | Yes |
| | RESPONSE | (U) | | Cycle | |
| | (ASMR) | | | | |

| | |
|---|---|
| Feedback Type | Level of reaction to ASMR-eliciting known phenomenon |
| | for the given individual |
| Ancillary | Video of ASMR-eliciting activity like napkin folding |
| Stimulation | |
| Multimodality | Ultrasound, TMS, tDCS, Optogenetics |

### Part XXV: Occipital Nerve

TABLE 36

| | | TARGETS- | | | |
|---|---|---|---|---|---|
| | | PRIMARY | | | |
| | | (U = Up- | | | |
| | | Regulated; | | | |
| | | D = Down- | PATTERN | | MECH. |
| Part | CONDITION | Regulated) | 1° | 2° | PERTURB. |
| XXV | OCCIPITAL | Occipital Nerve | Duty | Fibonacci | Yes |
| | NERVE | (Unilateral or | Cycle | | |
| | | Bilateral)(U) | | | |

| | |
|---|---|
| Feedback Type | Pain-level measurement (e.g., Visual Analog |
| | Scale) |
| Ancillary | Soothing sounds |
| Stimulation | |
| Multimodality | Ultrasound, TMS, local Electrical Stimulation |

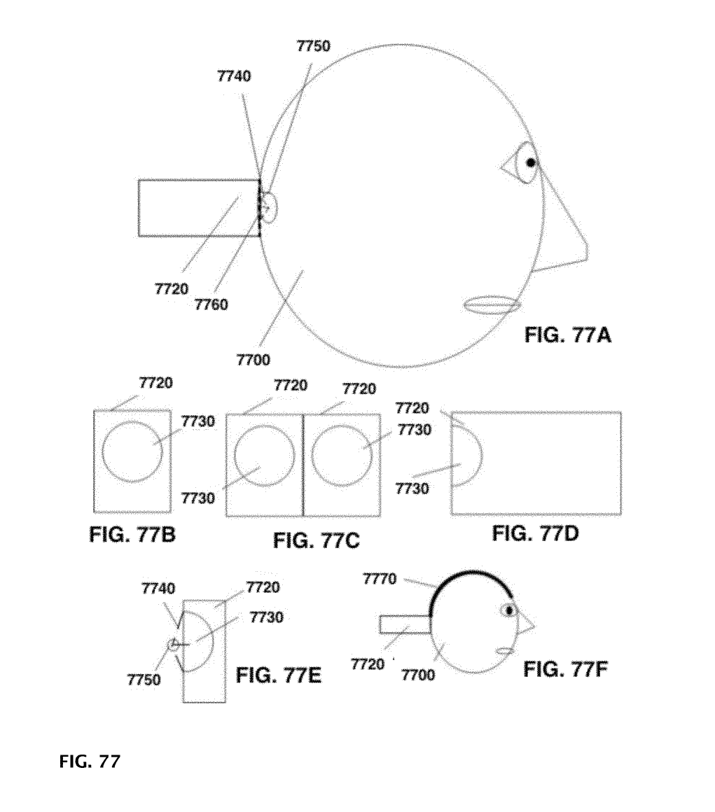

FIG. 77 shows a sagittal view of the configuration for neuromodulation of the occipital nerve. In FIG. 77A, patient head 7700 contains occipital nerve bundle 7750. Ultrasound transducer 7720 focuses sound field 7740 on occipital nerve bundle 7750. For the ultrasound to be effectively transmitted through intervening tissue to the neural targets, coupling must be put into place. Ultrasound transmission medium (e.g., Dermasol from California Medical Innovations or silicone oil in a containment pouch) is used as insert within the ultrasonic transducer (7730 in FIGS. 77B-77E). Ultrasound gel layer 7760 that provides the interface for ultrasound conduction between ultrasound transducer 7720 and head 7700 completes the conduction pathway.

If patient sees impact, he or she can move transducer in the X-Y direction (Z direction is along the length of transducer holder and could be adjusted as well). The elongated shape is convenient for the patient to hold and also for use with a positioning headband as shown in FIG. 77F showing patient head 7700 with ultrasound transducer 7720 and anterior-posterior headband 7770. A hat style or open frame with side-to-side stabilization (neither shown) can be employed as alternative embodiments. Ultrasound transducer 7720 is moved in and out of a holder (not shown) to provide the appropriate distance between ultrasonic transducer 7720 and occipital nerve bundle target 7750. In other embodiments, alternative fixed configurations, either of different ultrasonic transducer focal lengths or of different fixed positions in holders, are available for selection for specific patients.

Ultrasound transducer 7720 with ultrasound-conduction-medium insert 7730 are shown in front view in FIG. 77B for a single transducer 7720 for unilateral and in FIG. 77C for pair of transducers 7720 for bilateral stimulation. A side view of the same elements in shown in FIG. 77D. FIG. 77E again shows a side view of ultrasound transducer 7720 and ultrasound-conduction medium insert 7730 with ultrasound field 7740 focused on the occipital nerve bundle target 7750. The focus of ultrasound transducer 7720 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

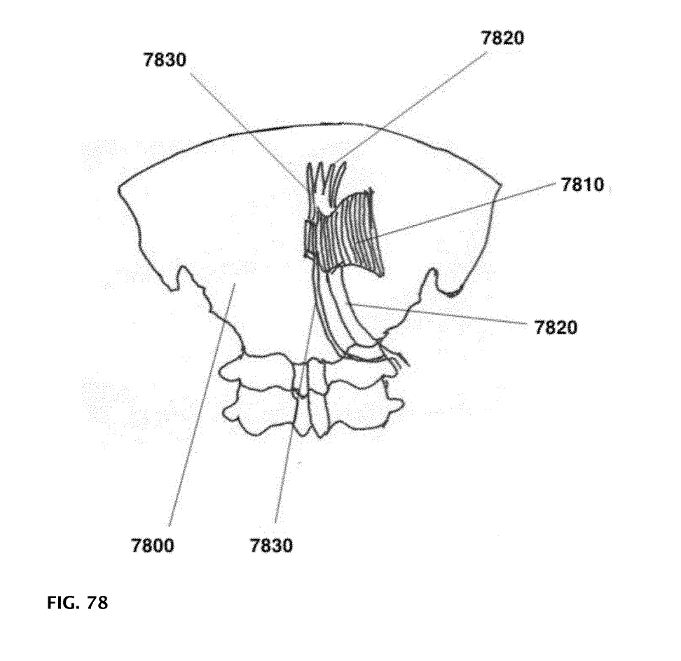

FIG. 78 shows anatomy of the occiput illustrating the location of occipital nerves. Occipital bone section 7800 has trapezius muscle complex 7810 through which the Greater Occipital Nerve 7820 and the Third Occipital Nerve 7830 pass. The occipital nerves occur bilaterally. Neuromodulation of which side will be most effective is headache specific and patient specific. In an alternative embodiment, bilateral neuromodulation will be supplied and this will be the usual situation. In another embodiment, the current invention will be applied to one side of the patient and an alternative treatment to the other side. Alternative invasive treatments have been electrical stimulation, local anesthetic blocks, surgical transection, surgical resection, radiofrequency, alcohol/phenol infiltration, radiosurgery, and cryotherapy. Medications and other non-invasive treatments such as avoidance of triggers, diet modification, physical therapy, chiropractic manipulation, and acupuncture have been used as well.

### Part XXVI: Sphenopalatine Ganglion (SPG)

TABLE 37

| | | TARGETS- | | | |
|---|---|---|---|---|---|
| | | PRIMARY | | | |
| | | (U = Up- | | | |
| | | Regulated; | | | |
| | | D = Down- | PATTERN | | MECH. |
| Part | CONDITION | Regulated) | 1° | 2° | PERTURB. |
| XXVI | SPHENO- | SPG (U) | Duty | Burst | No |
| | PALATINE | | Cycle | Mode | |

| | |
|---|---|
| GANGLION | Pain-level measurement (e.g., Visual Analog |
| | Scale) and/or measurement of aura |
| Ancillary | Soothing sounds or images |
| Stimulation | |
| Multimodality | Ultrasound, TMS, tDCS, Optogenetics |

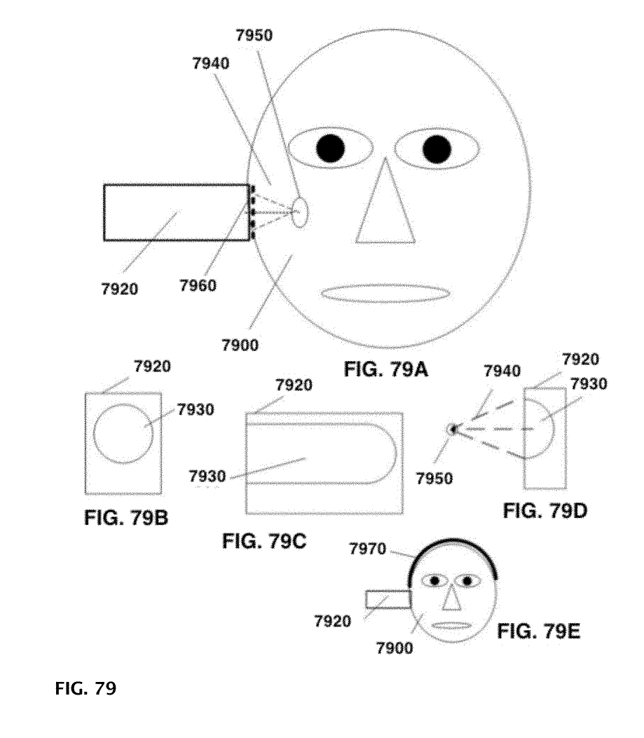

FIG. 79 shows a frontal view of the configuration for neuromodulation of the Sphenopalatine Ganglion (SPG) and related structures such as the sphenopalatine nerve and the vidian nerve. For the purpose of this discussion that the additional structures could be included. Patient head 7900 contains Sphenopalatine Ganglion 7950. Ultrasound transducer 7920 focuses sound field 7940 on Sphenopalatine Ganglion 7950. For the ultrasound to be effectively transmitted through intervening tissue to the neural targets, coupling must be put into place. Ultrasound transmission medium (e.g., Dermasol from California Medical Innovations or silicone oil in a containment pouch) is used as insert within the ultrasonic transducer (7930 in FIGS. 79B-79D). Ultrasound gel layer 7960 that provides the interface for ultrasound conduction between ultrasound transducer 7920 and head 7900 completes the conduction pathway. In the illustrated embodiment, ultrasound transducer 7920 is elongated to allow a longer focal length to be employed. The elongated shape is convenient for the patient to hold and also for use with a positioning headband as shown in FIG. 79E showing patient head 7900 with ultrasound transducer 7920 and headband 7970. Ultrasound transducer 7920 is moved in and out of a holder (not shown) to provide the appropriate distance between ultrasonic transducer 7920 and Sphenopalatine Ganglion target 7950. In other embodiments, alternative fixed configurations, either of ultrasonic transducer focal lengths or of different fixed positions in holder, are available for selection for specific patients. As to X-Y position on the head, the treatment for a specific patient can be planned using physical landmarks on the patient (for example, positioning the ultrasound transducer at lower edge of the zygomatic arch at the point anteriorly-posteriorly where the frontal process of the zygomatic bone meets the temporal process of the zygomatic bone). Alternatively, a standard x-ray examination based on bone can be done; taking an MRI or other scan is not necessary. In addition, the patient can adjust positioning based on effect. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well.

Ultrasound transducer 7920 with ultrasound-conduction-medium insert 7930 is shown in front view in FIG. 79B and in a side view in FIG. 79C. FIG. 79D again shows a side view of ultrasound transducer 7920 and ultrasound-conduction-medium insert 7930 with ultrasound field 7940 focused on the Sphenopalatine Ganglion target 7950. The focus of ultrasound transducer 7920 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

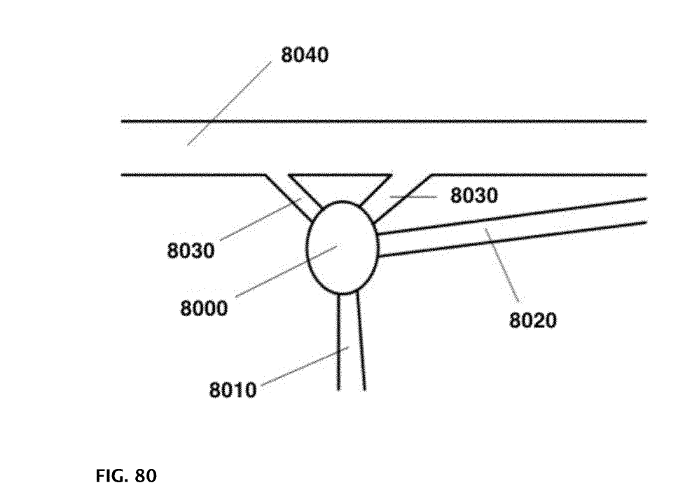

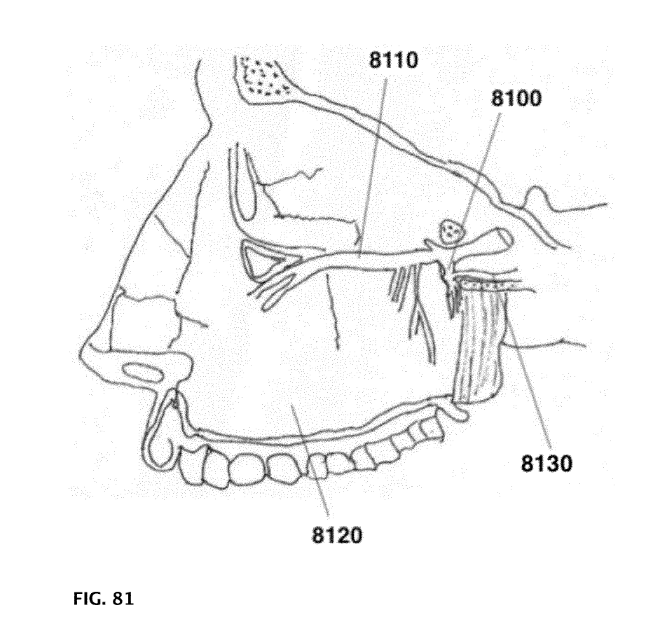

FIG. 80 shows the configuration of nerves surrounding Sphenopalatine Ganglion 8000. Sphenopalatine Ganglion 8000 is contained within the Sphenopalatine (or Pterygopalatine) fossa (not shown) and hangs down from maxillary nerve 8040 connected to it by Sphenopalatine Nerves 8030 with connections to vidian nerve 8020 and palatine nerves 8010. The vidian nerve 8020 connects to the Sphenopalatine Ganglion 8000. Vidian nerve 8020 contains parasympathetic fibers (which synapse to Sphenopalatine Ganglion 8000). The vidian nerve also contains sympathetic fibers and sensory fibers, transmitting sensation from part of the nasal septum. The sphenopalatine nerves 8030 are sensory nerves physically connect the Sphenopalatine Ganglion 8000 to the maxillary nerve 8040, but pass through and do not synapse with Sphenopalatine Ganglion 8000. These structures are located bilaterally. Neuromodulation of which side will be most effective is headache specific and patient specific. In an alternative embodiment, bilateral neuromodulation will be supplied. In another embodiment, the current invention will be applied to one side of the patient and an alternative treatment to the other side. Alternative invasive treatments have been electrical stimulation, local anesthetic blocks, surgical transection, surgical resection, radiofrequency, alcohol/phenol infiltration, radiosurgery, and cryotherapy. Medications and other non-invasive treatments such as avoidance of triggers, diet modification, physical therapy, chiropractic manipulation, and acupuncture have been used as well. FIG. 81 shows selected physical relationships with anterior skull 8120 showing Sphenopalatine Ganglion 8100, maxillary nerve 8110, and vidian nerve 8130.

While the parasympathetic nervous system is subject to Long-Term Potentiation (LTP) such that in addition to the acute effect that there is the potential for a long-term training effect, there can be Long-Term Potentiation (LTP) and Long-Term Depression (LTD) at the intracranial targets to which the Sphenopalatine Ganglion and associated neural structures are attached.

### Part XXVII: Reticular Activating System

TABLE 38

| | | TARGETS- | | | |
|---|---|---|---|---|---|
| | | PRIMARY (U = Up- | | | |
| | | Regulated; D = Down- | PATTERN | | MECH. |
| Part | CONDITION | Regulated) | 1° | 2° | PERTURB. |
| XXVII | RETICULAR | RAS (U) or (D) | Burst | Fibonacci | Yes |
| | ACTIVATING | | Mode | | |
| | SYSTEM (RAS) | | | | |

| | |
|---|---|
| Feedback Type | Physiological reaction to pain stimulation or measurement |
| | of ocular microtremor (OMT) |
| Ancillary | Soothing sounds for down regulation; rousing music for up |
| Stimulation | regulation |
| Intensity | Yes; from all modalities if multiple |
| Modulation | |
| Intersecting | Yes; from multiple transducers |
| Beams | |
| Multimodality | Ultrasound, TMS, Optogenetics |

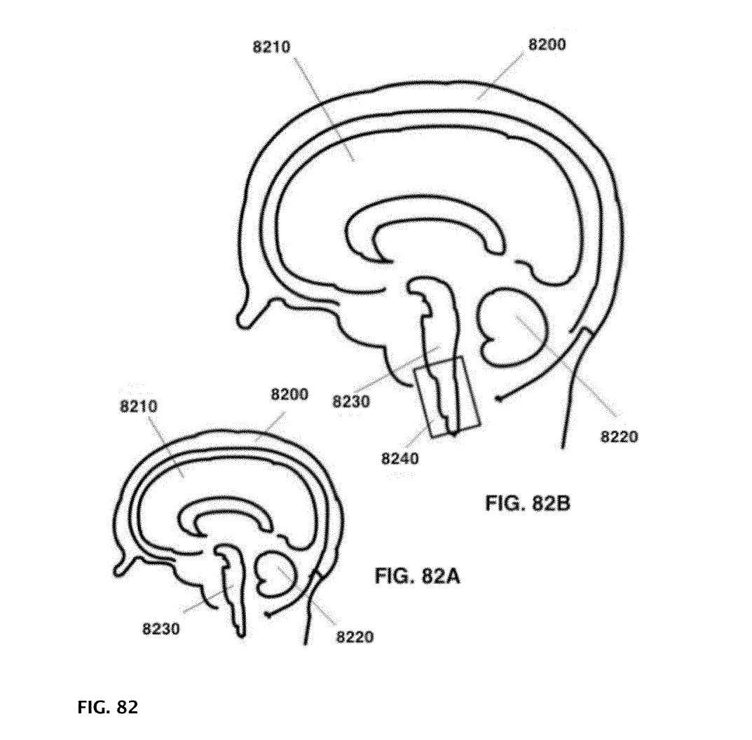

FIG. 82A shows sagittal view of brain highlighting the Reticular Activating System (RAS) 8230 including skull 8200 with cerebrum 8210 along with cerebellum 8220. FIG. 82B again shows the Reticular Activating System 8230 including skull 8200 with cerebrum 8210 along with cerebellum 8220, but this time with ultrasound transducer 8240 approximately aligned along the axis of the Reticular Activating System and placed against the neck. Both Part III, Shaped and Steered Ultrasound, and Part IV, Mechanical Perturbations, are applicable to the RAS. The ultrasound transducer 8240 does not cover the entire length of the Reticular Activating System (RAS) first because the upper part of the is not physically accessible (although the top of the outline 8230 is the midbrain which is outside the RAS) and second because the ultrasound field can be steered to a point above the top of the ultrasound transducer 8240. In another embodiment, the ultrasound transducer is perturbed laterally, up and down, and/or in and out causing enhanced change in the target neural tissue.

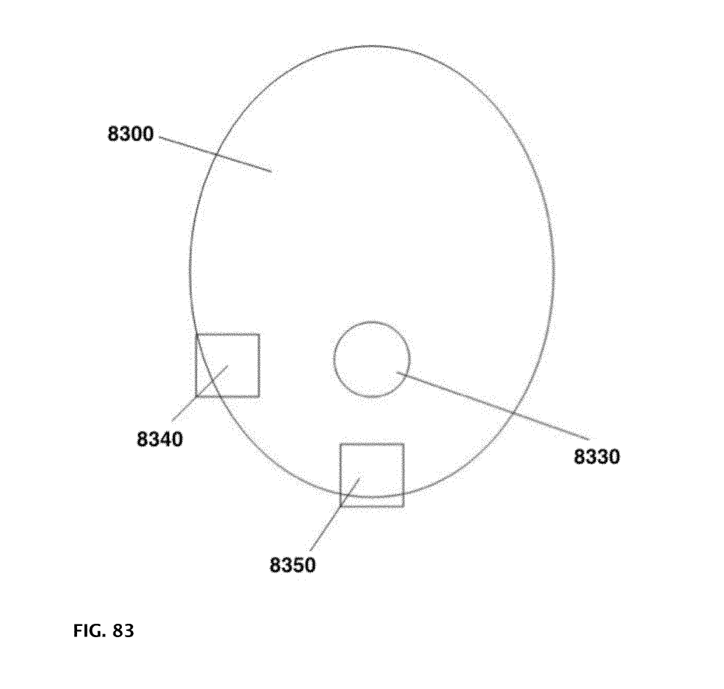

FIG. 83 shows the top view of patient head 8300 showing two embodiments of ultrasound transducer placements with respect to Reticular Activating System 8330, the first in which the ultrasound transducer is placed laterally 8340 to RAS 8330 and against the patient's neck and the second in which the ultrasound transducer 8350 is placed against the patient's neck posterior to RAS 8330. Note that the placement of lateral ultrasound transducer 8340 can be to the right of RAS 8330 or to its left. For the ultrasound to be effectively transmitted through the tissues to the RAS target, coupling must be put into place. Ultrasound transmission medium (e.g., Dermasol from California Medical Innovations or silicone oil in a containment pouch) (not shown) is interposed with one mechanical interface to the ultrasound transducer, either 8340 or 8350 completed by a layer of ultrasound transmission gel (not shown). The depth of the point where the ultrasound is focused depends on the shape of the transducer and setting of the phase and amplitude relationships of the elements of the ultrasound transducer array discussed in relation to the Control Circuit in FIG. 11. In other embodiments, ultrasound transducers may be place on both sides of the patient's neck. In a further embodiment, multiple ultrasound transducers may be used either in the vertical direction, horizontal direction, or both. A representative ultrasound neuromodulation configuration appears in FIGS. 20 to 22 with explanations in associated text.

In still another embodiment movement of the transducer and/or controlling stimulation parameters and seeing the physiological response of the patient is used to correctly locate the Reticular Activating System. This includes the use of Guided-Feedback Neuromodulation covered in Section I, Part X.

For example, neuromodulation of the Reticular Activating System to keep the general level of brain and base central activity up to prevent Central Nervous System failure.

The invention can be applied for a variety of clinical purposes such as reversibly putting a patient to sleep or waking them up (for example, for the purpose of anesthesia) or reversibly putting a patient into a coma (for example for the purpose of protecting or rehabilitating the brain of the patient after a stroke or head injury). Effects can be either acute or durable effect through Long-Term Potentiation (LTP) and/or Long-Term Depression (LTD). Since the effect is reversible putting the patient in even a vegetative state is safe if handled correctly. The application of LTP or LTD provides a mechanism for adjusting the bias of patient activity up or down. Appropriate radial (in-out) positions can be determined through patient-specific imaging (e.g., PET or fMRI) or set based on measurements to the mid-line. The positions can set manually or via a motor (not shown). The invention allows stimulation adjustments in variables such as, but not limited to, intensity, firing pattern, pulse duration, frequency, mechanical perturbations, phase/intensity relationships, dynamic sweeps, and position.

While preferred embodiments of the present invention have been shown and described herein, it will be obvious to those skilled in the art that such embodiments are provided by way of example only and changes may be made to the present invention without strictly following the exemplary embodiments and applications illustrated and described herein. Numerous variations, changes, and substitutions will now occur to those skilled in the art without departing from the invention. Such modifications and changes do not depart from the true spirit and scope of the present invention. It should be understood that various alternatives to the embodiments of the invention described herein may be employed in practicing the invention. It is intended that the following claims define the scope of the invention and that methods and structures within the scope of these claims and their equivalents be covered thereby.

In general, when a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for neuromodulation by one or more neuromodulation modalities of one or a plurality of neural targets comprising:
a. providing one or a plurality of neuromodulation transducers;
b. aiming the energy of said ultrasound transducers at one or a plurality of applicable neural targets; and
c. neuromodulating the ultrasound transducers with a control unit using guided-feedback neuromodulation wherein a set of neuromodulation parameters/variables is applied in a given time segment, the patient, operator, or agent judges the result, and based on that feedback result an optimization algorithm is applied to determine the neuromodulation parameters/variables to be applied in the next time segment.

2. The method of claim 1 where the optimization algorithm guiding the neuromodulation is not limited to but generally selected from the group consisting of hill climbing algorithm, the greedy algorithm, and simulated annealing.

3. The method of claim 1 where the result of feedback from neuromodulation due to the algorithmic change in the current time segment providing by the mechanism selected from the group consisted from patient, operator, and physiological sensor is compared to the result of the neuromodulation in the previous time segment where the comparison is selected from the group consisting of better, worse, unchanged, and relative level on a numerical scale and input to the optimization algorithm.

4. The method of claim 1 where the judgments are made on the basis selected from the group consisting of continuous and periodic.

5. The method of claim 4 where the lengths periodic time-segments after which strategy is changed are not limited to but typically will be in the range of 0.5 to 6 minutes.

6. The method of claim 1 where the successful parameters and strategy used in one session are saved and used at the start of the next neuromodulation session.

7. The method of claim 1 where signal derived from guided feedback is recorded and played back at a subsequent time.

8. The method of claim 1 in which a derived signal from the guided feedback is generated representing one or more factors selected from the group consisting the change in neuromodulation parameters, the relative change in symptoms based on input from the group from the consisting of as judged by the patient, as judged by the operator, and one or a plurality of sensors.

9. The method of claim 8 in which the derived signal is applied for the purpose selected from the group consisting of driving ancillary neuromodulation, driving a physical action such as counteracting tremor, operating a bionic limb or other actuator, and driving a display on a computer screen.

10. The method of claim 1 where one or a plurality of neuromodulation-modality specific parameters to be varied are not limited to but are selected as applicable from the group consisting of stimulation frequency, pulse duration, pulse frequency, pulse pattern, intensity, phase, phase-intensity relationships, intensity envelopes, frequency envelopes, sweeps of dynamic transducers, mechanical-perturbation length, mechanical perturbation frequency, and wavelength of light.

11. The method of claim 10 where the order in which changes are to be applied by one with ordinary skill in the art, while not limited to this order, higher priority change to be made as applicable to a given neuromodulation modality is pulse repetition, pulse duty cycle, wavelength, and neuromodulation frequency, changing the shape of the neuromodulation by using mechanical perturbations, and changing the aiming of the energy transducers.

12. The method of claim 3 where the result is indicated by the operator/patient using a mechanism not limited to or selected from mouse, joystick, bars, spinner, voice-command input, and touchscreen.

13. The method of claim 1 where the one or a plurality of neural targets are each neuromodulated by a modality selected from the group consisting of deep brain stimulation, spinal cord stimulation, vagal nerve stimulation, sphenopalatine ganglion stimulation, occipital nerve stimulation, transcranial magnetic stimulation, ultrasound neuromodulation, radiofrequency stimulation, optogenetics, and ancillary stimulation.

14. The method of claim 1 where the clinical condition to be treated or physiological effect is selected from the group consisting of orgasm elicitation, stroke and rehabilitation, pain, tinnitus, depression and bipolar disorder, addiction, Post Traumatic Stress Disorder, motor disorders, Autism Spectrum, obesity, Alzheimer's Disease, anxiety including panic disorder, Obsessive Compulsive Disorder, gastrointestinal motility, Tourette's Syndrome, schizophrenia, epilepsy, Attention Deficit Hyperactivity Disorder, eating disorders, cognitive enhancement, traumatic brain injury including concussion, compulsive sexual disorders, emotional catharsis, Autonomous Sensory Meridian Response (ASMR), occipital nerve neuromodulation, Sphenopalatine Ganglion neuromodulation, and Reticular Activating System (RAS).

15. The method of claim 14 where the condition-assessment pairs are not limited to but selected from the group consisting of orgasm elicitation-arousal, stroke and stroke rehabilitation-movement of affected area, pain-pain Visual Analog Scale, tinnitus-tinnitus level, depression and bipolar disorder-affect, addiction-craving, post-traumatic stress disorder-reaction to inciting image, motor disorder-tremor magnitude, autism spectrum disorders-reaction to spontaneous situation, obesity-food craving, Alzheimer's Disease-performance on memory test, anxiety including panic disorder-response to frenetic images and/or audio, obsessive compulsive disorder-response to video, GI motility-response to food inciting for diarrhea, Tourette's Syndrome-response to inciting situation, schizophrenia-response to inciting visual or audio, eating disorders-reaction to food, cognitive enhancement-performance on problem-solving test or video gaming, traumatic brain injury including concussion-ability to perform repetitive physical activity, compulsive sexual disorders-reaction to explicit material, emotional catharsis-reaction to release trigger, autonomous sensory meridian response (ASMR)-reaction to ASMR-eliciting known phenomenon for the given individual, occipital nerve-pain Visual Analog Scale, sphenopalatine ganglion-pain Visual Analog Scale, and reticular activating system-ocular microtremor.

16. The method of claim 15 where an ancillary stimulation such as applying pressure to the point of causing pain is used to facilitate assessment of feedback.

17. The method of claim 8 where instead of patient symptoms or the effect being sought being judged, a surrogate for the symptom or effect is judged.

18. The method of claim 17 where the symptom is chronic pain and the surrogate is acute pain.
